(19) United States
(12) Patent Application Publication
(10) Pub. No.: US 2023/0103444 A1
(43) Pub. Date: Apr. 6, 2023

(54) SMALL MOLECULE INHIBITORS OF ONCOGENIC CHD1L WITH PRECLINICAL ACTIVITY AGAINST COLORECTAL CANCER

(71) Applicant: The Regents of the University of Colorado, a Body Corporate, Denver, CO (US)

(72) Inventors: Daniel V. LaBARBERA, Aurora, CO (US); Joshua M. ABBOTT, Silver Spring, MD (US); Qiong ZHOU, Aurora, CO (US); Adedoyin D. ABRAHAM, Thornton, CO (US); Hector ESQUER, Aurora, CO (US); Brett Joseph PRIGARO, Broomfield, CO (US); Laura Anne PIKE, Aurora, CO (US); Paul AWOLADE, Aurora, CO (US)

(21) Appl. No.: 17/953,221

(22) Filed: Sep. 26, 2022

Related U.S. Application Data
(63) Continuation of application No. PCT/US2021/023981, filed on Mar. 24, 2021.
(60) Provisional application No. 62/994,259, filed on Mar. 24, 2020.
(60) Provisional application No. 63/139,394, filed on Jan. 20, 2021.
(60) Provisional application No. 63/250,803, filed on Sep. 30, 2021.

(51) Int. Cl. C07D 265/30; C07D 417/12; C07D 263/46; C07D 409/14; C07D 471/04; C07D 403/14; C07D 403/04; C07D 409/12; C07D 413/04; C07D 519/00; A61P 35/00

(52) U.S. Cl. CPC C07D 265/30; C07D 417/12; C07D 263/46; C07D 409/14; C07D 471/04; C07D 403/14; C07D 403/04; C07D 409/12; C07D 413/04; C07D 519/00; A61P 35/00

(57) ABSTRACT

Treatment of CHD1L-driven cancers, including TCF transcription-driven cancers and EMT-driven cancers using CHD1L inhibitors. Small molecule inhibitors of CHDL1 which inhibit CHD1L ATPase and inhibit CHD1L-dependent TCF-transcription have been identified. CHD1L inhibitors prevent the TCF-complex from binding to Wnt response elements and promoter sites. More specifically, CHD1L inhibitors induce the reversion of EMT. CHD1L inhibitors are useful in the treatment of various cancers and particularly CRC and m-CRC. The CHD1L-driven cancer is among others, CRC, breast cancer, glioma, liver cancer, lung cancer or gastrointestinal (GI) cancers. CHD1L inhibitors of formulas I and XX and salts thereof as defined herein are provided as well as pharmaceutical compositions containing CHD1L inhibitors. Synergistic combinations of CHD1L inhibitors with other antineoplastic agents are also described.

### CROSS-REFERENCE TO RELATED APPLICATIONS

This application claims the benefit of U.S. provisional application 63/250,803, filed Sep. 30, 2021 and is a continuation-in-part of PCT/US21/23981, filed Mar. 24, 2021, which in turn claims the benefit of U.S. provisional applications 62/994,259, filed Mar. 24, 2020, and 63/139,394, filed Jan. 20, 2021. Each of the listed applications is incorporated by reference herein in its entirety.

### STATEMENT REGARDING U.S. GOVERNMENT SUPPORT

This invention was made with Government support under grant number W81XWH1810142 awarded by the Department of Defense (DoD). The U.S. Government has certain rights in this invention.

### BACKGROUND

The integrity of the genome is maintained by conformational changes to chromatin structure that 20 regulate accessibility to DNA for gene expression and replication. Chromatin structure is maintained by post-translational modifications of histones and rearrangement of nucleosomes. [Lorch et al., 2010; Kumar et al., 2016; Swygert et al., 2014] ATP-dependent chromatin remodelers are enzymes that alter chromatin by changing histone composition, and by evicting or translocating nucleosomes along DNA. Their activity plays a critical role in cellular function by regulating gene expression and the accessibility of DNA for replication, transcription, and DNA repair. [Erdel et al., 2011; Brownlee et al., 2015] Dysregulation of chromatin remodeling is associated with human disease, particularly cancer. [Zhang et al., 2016; Valencia & Kadoch, 2019]

In the last decade, the chromatin remodeler known as CHD1L (chromodomain helicase/ATPase DNA binding protein 1-like), also known as ALC1 (amplified in liver cancer 1), has emerged as an oncogene implicated in the pathology of prominent human cancers. (Ma et al., 2008; Cheng et al., 2013] CHD1L is also involved in multi-drug resistance, ranging from upregulation of drug resistance efflux pumps (e.g., ABCB1) [Li et al., 2019] to PARP1 mediated DNA repair [Pines et al., 2012; Tsuda et al., 2017], and anti-apoptotic activity. [Li et al., 2013; Chen et al., 2009] Moreover, amplification or overexpression of CHD1L are correlated with poor prognoses for patients, including low overall survival (OS) and metastatic disease. [He et al., 2015; Hyeon et al., 2013; Su et al., 2014; Mu et al., 2015; Su et al., 2015; Li et al., 2013; He et al., 2012; Chen et al., 2010] CHD1L overexpression has also been implicated in tumor progression and as a predictor of poor patient survival. [Ji et al., 2013] The multifunctional oncogenic mechanisms of CHD1L make it an attractive therapeutic target in cancer. While the cancer driving mechanisms of CHD1L have been studied in liver [Li et al., 2019], breast [Wu et al., 2014], and lung [Li et al., 2019] cancer, little is known about the pathological mechanisms associated with CHD1L in colorectal cancer (CRC).

CRC is the third most prevalent cancer diagnosed each year and CRC patients have the second highest mortality rate worldwide. [Jemal et al., 2011] Early detection of CRC combined with surgery and 5-fluorouracil (5FU) based combination chemotherapy has minimally improved the overall survival rate. [Jemal et al., 2011; Fakih, 2015] The current chemo and targeted therapies are largely ineffective against metastatic CRC (mCRC), evidenced by a low 11% 5-year overall survival rate. [Jemal et al., 2011; Fakih, 2015] There is an unmet need in the art to identify and characterize targets involved in the pathology of CRC tumor progression and metastasis.

A majority of CRC patients possess mutations in the Wnt signaling pathway, leading to aberrant T-Cell Factor/Lymphoid Enhancer Factor-transcription or TCF-complex. [Kinzler & Voelstein, 1996; Cancer Genome Atlas, 2012] Such mutations can lead to constitutive β-catenin translocation and transactivation of TCF-transcription. [Clevers, 2006; Korinek et al., 1997] The TCF-complex is orchestrated by TCF4 (a.k.a. TCFL2), which is activated through interactions with an array of coactivators such as β-catenin, PARP1, and CREB Binding protein (CBP). [Shitashige et al., 2008] Recently, TCF4 was shown to be a specific driver of both early metastasis from adenomas (i.e. polyps) and from late stage mCRC. [Hyeo et al., 2013; Su et al., 2014]

It has been reported that TCF transcription functions as a master regulator of epithelial-mesenchymal transition (EMT) [Senchez-Tille et al., 2011; Zhou et al., 2016; Abraham et al., 2019]. This process can transform relatively benign epithelial tumor cells into mesenchymal cells with increased cancer stem cell (CSC) stemness and other malignant properties that drive mCRC. [Chaffer et al., 2016] It has recently been reported that alterations in certain CRC driver genes are common in both primary and metastatic tumor pairs. [Hu et al., 2019] More specifically, aberrant TCF4 is reported to be a specific driver of mCRC. [Hu et al., 2019] and CRC can metastasize in early adenomas (i.e., polyps [see also Magri & Bardelli, 2019] which is likely caused by TCF-driven EMT. [Chaffer et al. 2016; Chaffer & Weinberg, 2011] These reports indicate that TCF-transcription is a driving force at all stages of CRC progression and metastasis.

EMT is a major driving force in numerous human diseases, especially solid tumor progression, drug and radiation therapy resistance, evasion of the immune response and immunotherapy, and promotion of metastasis. [Chaffer et al. 2016; Chaffer & Weinberg, 2011; Scheel & Weinberg, 2012]

Due to the significance of the Wnt signaling pathway and TCF-transcription in cancer and other diseases [Clevers, 2006], small molecule drugs that inhibit the Wnt signaling pathway and TCF-transcription have been examined. [Lee et al., 2011; Polakis, 2012] Therapeutic strategies considered include receptor targets (e.g., Frizzled); preventing Wnt ligand secretion (e.g., porcupine); inhibiting β-catenin destruction complex (e.g., tankyrases); and protein-protein inhibition (PPI) with β-catenin and co-activators (e.g., CBP). While clinical trials may be underway, no drug has as yet been clinically approved that targets the Wnt/TCF pathway. [Lu et al., 2016] In contrast, the present invention describes a new therapeutic strategy, particularly for identifying small molecule drugs, for treatment of Wnt/TCF driven CRC in which CHD1L is identified as a DNA binding factor required for TCF-transcription regulating the malignant phenotype in CRC.

For example, U.S. Pat. No. 9,616,047 reports small molecule inhibitors of β-catenin or disruptors of a β-catenin/TCF-4 complex which are said to attenuate colon carcinogenesis. Inhibitors of β-catenin reported therein include esculetin, as well as, compounds designated HI-B1-HI-B20, HI-B22-HI-B-24, HI-B26, HI-B32 and HI-B34, the structures of each of which is provided in the patent. The patent further describes, in a number of generic chemical formulae therein, compounds said to be useful as β-catenin inhibitors and for the treatment of colon carcinogenesis. This patent is incorporated by reference herein in its entirety for the structures of specific compounds, generic formulae and variable definitions of compounds said therein to be useful in the invention therein. The compounds identified herein are structurally distinct from those described herein.

CN109761909 published May 17, 2019 reports (as described in the Espacenet Eng. Abstract thereof) certain N-(4-(pyrimidine-4-amino)phenyl)sulfonamide compounds or salts of a certain formula inhibit Hsp90-Cdc37 (heat shock protein Hsp90 and its auxillary chaperone Cdc37) interactive client protein expression, and are reported useful for treating or preventing various diseases mediated by an Hsp90 signal channel. The formula given in the published application is:

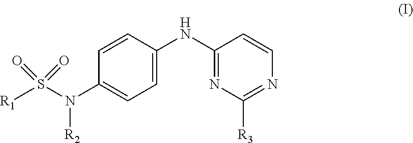

where variables are defined according to the Espacenet English machine translation as follows: R₁ is mes-trimethylphenyl, 4-methylphenyl, 4-trifluoromethylphenyl, naphthyl, 2,3,4,5, -tetramethylphenyl, 4-methoxyphenyl, 4-tert-butylphenyl, 2,4-dimethoxyphenyl, 2,5-dimethoxyphenyl or 4-phenoxyphenyl; R₂ is hydrogen, methyl acetate, acetate, aminoacetyl, 4-formic acid benzyl, 4-isopropylbenzyl, 4-chlorobenzyl or 4-methoxybenzyl; and R₃ is chlorine, —ORa or —NRbRc, where, Ra is a chain C1-3 alkyl, C5-6 cycloalkyl, C1-2 alkoxy, mono- or di-C1-2 alkylamino, or C5-6 nitrogen-containing or oxygen-containing heterocyclic group; and Rb and Rc are C1-5 alkyl groups, respectively. More specifically, R₃ is chlorine, 2-hydroxytetrahydropyrrolyl, ethanolamino, 2,3-dihydroxy-1-methylpropylamino, 2,3-dihydroxypropylamino, piperazinyl, N-methylpiperazinyl, azepyl, piperidinyl, 2-methylpropylamino, propoxy, methylamino, ethylamino, cyclopropylamino, 1-ethylpropylamino, tetrahydropyran-4-ylmethoxy or 2-methoxyethoxy. The reference also refers to a compound of formula I-5:

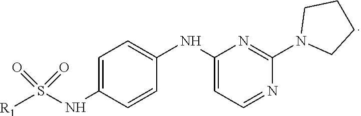

This published application is incorporated by reference herein in its entirety for the structures of specific compounds, generic formulae and variable definitions of compounds said therein to be useful in the invention therein. Structures disclosed in this published application can be excluded from any chemical formula of the present application.

The present invention examines the clinicopathological characteristics of CHD1L in CRC, and the results herein indicate that CHD1L is a druggable target involved in TCF-transcription. A mechanism for CHD1L-mediated TCF-transcription is also proposed herein. Small molecule inhibitors of CHD1L are identified herein which are able to prevent TCF transcription, reverse EMT, and other malignant properties in a variety of cell models including tumor organoids and patient derived tumor organoids (PDTOs). Certain CHD1L inhibitors identified herein display drug-like pharmacological properties, including in vivo pharmacokinetic (PK) and pharmacodynamic (PD) profiles, important for translational development towards the treatment of CRC and other cancers.

### SUMMARY

This invention relates to the treatment of CHD1L-driven cancers, more specifically TCF transcription-driven cancers and yet more specifically EMT-driven cancers. CHD1L is found to be an essential component of the TCF transcription complex. Small molecule inhibitors of CHDL1 which inhibit CHD1L ATPase and inhibit CHD1L-dependent TCF-transcription have been identified. CHD1L inhibitors are believed to prevent the TCF-complex from binding to Wnt response elements and promoter sites. More specifically, CHD1L inhibitors induce the reversion of EMT. CHD1L inhibitors are useful in the treatment of various cancers and particularly CRC and m-CRC. Particularly with respect to CRC, CHD1L inhibitors are shown in embodiments to inhibit cancer stem cell (CSC) stemness and invasive potential. In embodiments, CHD1L inhibitors induce cytotoxicity in CRC PDTOs. In specific embodiments, the CHD1L-driven cancer is CRC, breast cancer, including BRCA-mutated breast cancer and metastatic breast cancer, ovarian cancer, including BRCA-mutated ovarian cancer, pancreatic cancer, including BRCA-mutated pancreatic cancer, glioma, liver cancer, lung cancer, prostate cancer, or gastrointestinal (GI) cancers. In specific embodiments, the TCF transcription-driven cancer is CRC, including mCRC. In specific embodiments, the EMT-driven cancer is CRC, including mCRC. In specific embodiments, the cancer that is treated is breast cancer, including BRCA-mutated breast cancer and metastatic breast cancer. In specific embodiments, the cancer that is treated is ovarian cancer. In specific embodiments, the cancer that is treated is pancreatic cancer.

The invention provides a method for treatment of CHD1L-driven cancers, more specifically TCF transcription-driven cancers and yet more specifically EMT-driven cancers, including GI cancer, particularly CRC and mCRC, which comprises administration to a patient in need thereof of an amount of a CHD1L inhibitor which is effective for CHD1L inhibition, effective inhibition of aberrant TCF transcription or effective for induction of EMT reversion. In embodiments, the CHD1L inhibitor is a compound of any one of formulas I-XXIII or XXX-XVII. In embodiments, the CHD1L inhibitor is a compound of any one of formulas I, II, or XX-XXIII. In embodiments, the CHD1L inhibitor is a compound of either of formulas XLV or XLVI. In an embodiment, the CHD1L inhibitor is any one of compounds 1-177. In an embodiment, the CHD1L inhibitor is any one of compounds 8-177 or any one of compounds 9-117. In an embodiment, the CHD1L inhibitor is any one of compounds 28, 31, 52, 54, 57, 75, 118, 126, 131, 150, or 169. In more specific embodiments, the compound is selected from compounds 52, 118, 126, 131, 150, or 169. In embodiments, the compound is selected from compounds 28, 31, 54, 57, or 75. In embodiments, the compound is one or more of compounds 28, 31, 52, 54, 57, 75, 118, 126, 131, 150, or 169. In embodiments, the compound is one of compounds 28, 31, 52, 54, 57, 75, 118, 126, 131, 150, or 169. More specifically, the invention provides a method of inhibiting aberrant TCF-transcription, particularly in CRC, by administration of an effective amount of a CHD1L inhibitor. Yet more specifically, the invention provides a method of inducing reversion of EMT, particularly in CRC or mCRC, by administration of an effective amount of a CHD1L inhibitor. The invention provides a method of inhibiting Cancer Stem Cell (CSC) stemness and/or invasive potential, particularly in CRC, by administration of an effective amount of a CHD1L inhibitor. The invention provides a method for treatment of cancerous tumors of CHD1L-driven cancers, or TCF transcription-driven cancers or EMT-driven cancers, particularly in CRC, by administration of an effective amount of a CHD1L inhibitor. The invention provides a method for treatment of cancerous solid tumors of CHD1L-driven cancers, or TCF transcription-driven cancers or EMT-driven cancers, particularly in CRC, by administration of an effective amount of a CHD1L inhibitor. The invention provides a method for treatment of breast cancer, including BRCA-mutated breast cancer, by administration of an effective amount of a CHD1L inhibitor.

The invention provides a method for treatment of ovarian cancer by administration of an effective amount of a CHD1L inhibitor. The invention provides a method for treatment of pancreatic cancer by administration of an effective amount of a CHD1L inhibitor.

In embodiments, CHD1L inhibitors are selective for inhibition of CHD1L. In embodiments, CHD1L inhibitors herein are not PARP inhibitors. In embodiments, CHD1L inhibitors herein are not inhibitors of topoisomerases. In particular, CHD1L inhibitors herein are not inhibitors of DNA topoisomerase. In particular, CHD1L inhibitors herein are not inhibitors of topoisomerase type IIα. In embodiments, CHD1L inhibitors herein are not inhibitors of β-catenin, particularly inhibitors such as described in U.S. Pat. No. 9,616,047. In embodiments, CHD1L inhibitors herein are not inhibitors of Hsp90-Cdc37 interactive client protein expression, particularly inhibitors as described in CN109761909.

In embodiments, invention also provides a method to prevent tumor growth, invasion and/or metastasis in CHD1L-driven, TCF-transcription, or EMT-driven cancers by administering to a patient in need thereof of an amount of a CHD1L inhibitor of this invention which is effective for CHD1L inhibition, inhibition of aberrant TCF transcription, or effective for reversion of EMT. In specific embodiments, tumors are solid tumors. In specific embodiments, tumors are those associated with GI cancer. In embodiments, tumors are those associated with CRC. In embodiments, tumors are those associated with mCRC. In embodiments, tumors are those associated with breast cancer. In embodiments, tumors are those associated with BRAC-mutated breast cancer. In embodiments, tumors are those associated with ovarian cancer. In embodiments, tumors are those associated with pancreatic cancer. In embodiments, tumors are those associated with lung cancer. In embodiments, tumors are those associated with liver cancer.

In specific embodiments, the invention provides a method for treatment of CRC, including mCRC, which comprises administration to a patient in need thereof of an amount of a CHD1L inhibitor which is effective for inhibition of CHD1L. In specific embodiments, the invention provides a method for treatment of CRC, including mCRC, which comprises administration to a patient in need thereof of an amount of a CHD1L inhibitor which is effective for inhibition of aberrant TCF transcription. In specific embodiments, the invention provides a method for treatment of CRC, including mCRC, which comprises administration to a patient in need thereof of an amount of a CHD1L inhibitor which is effective for induction of reversion of EMT.

In specific embodiments, the invention provides a method for inducing apoptosis in cancer cells which comprises contacting a cancer cell with an effective amount of a CHD1L inhibitor. In an embodiment, the CHD1L inhibitor is provided in an amount effective for inhibition of aberrant TCF transcription. In an embodiment, the CHD1L inhibitor is provided in an amount effective for induction of reversion of EMT. In an embodiment, the cancer cells are CRC cancer cells. In an embodiment, the cancer cells are mCRC cancer cells. In an embodiment, the cancer cells are breast cancer cells. In an embodiment, the cancer cells are breast cancer cells carry a BRCA mutation. In an embodiment, the cancer cells are ovarian cancer cells. In an embodiment, the cancer cells are pancreatic cancer cells. In an embodiment, the cancer cells are lung cancer cells. In an embodiment, the cancer cells are liver cancer cells. In an embodiment, the method is applied in vivo. In an embodiment, the method is applied in vivo in a patient. In an embodiment, the method is applied in vitro.

In embodiments of the methods herein comprising administration of the CHD1L inhibitor, the CHD1L inhibitor is administered by any known administration method and dosing schedule to achieve desired benefits. In an embodiment, administration is oral administration. In an embodiment, administration is by intravenous injection.

In embodiments, oral administration employs oral dosage forms comprising pharmaceutically acceptable polyethylene glycol (PEG). In such embodiments, the pharmaceutically acceptable PEG may be combined with a pharmaceutically acceptable organic solvent, particularly a pharmaceutically acceptable polar, aprotic solvent. In embodiments, the organic solvent is pharmaceutically acceptable DMSO. In embodiments, oral administration employs oral dosage forms comprising low molecular weight polyethylene glycol having molecular weight less than 600 g/mole. In more specific embodiments, oral administration employs PEG 400. In more specific embodiments, oral administration employs PEG 200.

In embodiments, the invention in addition1i provides a method of treatment of drug-resistant cancer which comprises administering to a patient in need thereof of an amount of a CHD1L inhibitor, which is effective for CHD1L inhibition, inhibition of aberrant TCF transcription or induction of reversion of EMT, in combination with a known treatment to which the cancer has become resistant. In specific embodiments, the treatment to which the cancer has become resistant is conventional chemotherapy and other targeted therapies. In specific embodiments, the invention provides a method of increasing the efficacy of a DNA-damaging drug in cancer which comprises combined treatment of the cancer with the DNA damaging drug and a CHD1L inhibitor where the CHD1L is administered in an amount effective for decreasing resistance to the DNA-damaging drug. In an embodiment, the DNA-damaging drug is a topoisomerase inhibitor. In particular, the DNA-damaging drug is a DNA topoisomerase inhibitor. In particular, the DNA-damaging drug is a topoisomerase type IIα inhibitor. In particular, the DNA-damaging drug is etoposide or teniposide. In particular, the DNA-damaging drug is SN38 or a prodrug thereof. In an embodiment, the DNA-damaging drug is a thymidylate synthase inhibitor. In an embodiment, the thymidylate synthase inhibitor is a folate analogue. In an embodiment, the thymidylate synthase inhibitor is a nucleotide analogue. In specific embodiments, the thymidylate synthase inhibitor is raltitrexed, pemetrexed, nolatrexed or ZD9331. In a particular embodiment, the DNA-damaging drug is 5-fluorouracil or capecitabine.

In an embodiment, the drug-resistant cancer is a CHD1L-driven cancer. In an embodiment, the drug-resistant cancer is a TCF transcription-driven cancer. In an embodiment, the drug-resistant cancer is an EMT-driven cancer. In an embodiment, the treatment is for drug-resistant CRC. In an embodiment, the treatment is for drug-resistant mCRC. In embodiments, the treatment is for drug-resistant breast cancer, drug-resistant ovarian cancer, drug-resistant pancreatic cancer, drug-resistant lung cancer or drug-resistant liver cancer. In embodiments, the DNA-damaging drug and the CHD1I inhibitor are administered by any known method on a dosing schedule appropriate for realizing the combined therapeutic benefit. In embodiments, the CHD1L inhibitor is administered orally and the DNA-damaging drug is administered by any known administration method and dosing schedule. In embodiments, the CHD1L inhibitor is administered prior to administration of the DNA-damaging drug. In embodiments, the CHD1L inhibitor is administered prior to and optionally after administration of the DNA-damaging drug. In embodiments, the CHD1L inhibitor is administered orally prior to and optionally after administration of the DNA-damaging drug by intravenous injection.

The invention provides methods for treatment of CHD1L-driven cancer, TCF-transcription-driven cancer, or EMT-driven cancer which comprises administration to a patient in need thereof of an amount of a CHD1L inhibitor which is effective for CHD1L inhibition or inhibition of aberrant TCF transcription or induction of reversion of EMT in combination with an alternative method of treatment for the cancer. In an embodiment, the cancer is GI cancer or more specifically CRC cancer and yet more specifically is mCRC. In an embodiment, the alternative method for treatment is administration of one or more of 5-fluorouracil, 5-fluorouracil in combination with folinic acid (also known as leucovorin), a topoisomerase inhibitor, or a cytotoxic or antineoplastic agent. In embodiments, the CHD1L inhibitor is administered in combination with 5-fluorouracil or in combination with 5-fluorouracil and folinic acid. In embodiments, the CHD1L inhibitor is administered in combination with a topoisomerase inhibitor and in particular with irinotecan (a prodrug of SN38 also known as camptothecin) or any other known prodrug of SN38. In embodiments, the combined treatment using a CHD1L inhibitor and a topoisomerase inhibitor exhibits at least additive activity against the cancer. In embodiments, the combined treatment of a CHD1L inhibitor with a topoisomerase inhibitor exhibits synergistic activity (greater than additive activity) against the cancer.

In embodiments, the CHD1L inhibitor is administered in combination with a cytotoxic or antineoplastic agent, in particular a platinum-based antineoplastic agent and more particularly cisplatin, carboplatin or oxaliplatin. In embodiments, the combined treatment using a CHD1L inhibitor and a platinum-based antineoplastic agent exhibits at least additive activity against the cancer. In embodiments, the combined treatment of a CHD1L inhibitor with a platinum-based antineoplastic agent exhibits synergistic activity (greater than additive activity) against the cancer. In embodiments, the platinum-based antineoplastic agent and the CHD1I inhibitor are administered by any known method on a dosing schedule appropriate for realizing the combined therapeutic benefit. In embodiments, the CHD1L inhibitor is administered orally and the platinum-based neoplastic agent is administered by any known administration method and dosing schedule. In embodiments, the CHD1L inhibitor is administered prior to administration of the platinum-based neoplastic agent. In embodiments, the CHD1L inhibitor is administered prior to and optionally after administration of the platinum-based antineoplastic agent. In embodiments, the CHD1L inhibitor is administered orally prior to and optionally after administration of the platinum-based neoplastic agent by intravenous injection.

In embodiments, the CHD1L inhibitor is administered in combination with a chemotherapy regimen (administration of an alternative cancer cytotoxic agent or antineoplastic agent or adminintration of an antineoplastic procedure) for treatment of cancer, including without limitation GI cancer, particularly CRC, and mCRC. In embodiments, the CHD1L inhibitor is administered in combination with the chemotherapy regimen designated FOLFOX. In embodiments, the CHD1L inhibitor is administered in combination with the chemotherapy regimen designated FOLFIRI. In embodiments, the chemotherapy regime and the CHD1I inhibitor are administered by any known method on a dosing schedule appropriate for realizing the combined therapeutic benefit. In embodiments, the CHD1L inhibitor is administered orally and the chemotherapy regime is administered by any known administration method and dosing schedule. In embodiments, the CHD1L inhibitor is administered prior to administration of the chemotherapy regime. In embodiments, the CHD1L inhibitor is administered prior to and optionally after administration of the chemotherapy regime. In embodiments, the CHD1L inhibitor is administered orally prior to and optionally after administration of the PARP inhibitor by intravenous injection.

The invention provides a method for treatment of cancers that are sensitive to Poly(ADP)-ribose) polymerase I (PARPI) in which a CHD1L inhibitor is used in combination with a PARP inhibitor. In embodiments, an amount of a CHD1L inhibitor effective for CHD1L inhibition, inhibition of aberrant TCF transcription or induction of reversion of EMT is used in combination with an amount of a PARP inhibitor effective for treating cancer to at least enhance the effectiveness of the cancer treatment. In embodiments, the combined treatment using a CHD1L inhibitor and a PARP inhibitor exhibits at least additive activity against the cancer. In embodiments, the combined treatment of a CHD1L inhibitor with a PARP inhibitor exhibits synergistic activity (greater than additive activity) against the cancer. In embodiments, the cancer is a cancer sensitive to treatment by a PARP inhibitor. In embodiments, the cancer is a cancer that is or has become resistant to treatment by a PARP inhibitor. In embodiments, the cancer is a cancer sensitive to treatment by a PARP inhibitor or which has become resistant to treatment by a PARP inhibitor and which is a CHD1L-driven, a TCF-driven or an EMT-driven cancer. In embodiments, the cancer is a homologous recombination deficient cancer (see, for example, Zhou et al. BioRxiv 2020). In embodiments, the cancer treated is a cancer sensitive to a PARP inhibitor and more particularly is breast or ovarian cancer. In specific embodiments, the cancer is a BRCA-deficient cancer (BRCA-mutated cancer), for example, BRCA-deficient breast cancer (BRCA-mutated breast cancer), BRCA-deficient ovarian cancer (BRCA-mutated ovarian cancer or BRCA-defcient pancreatic cancer (BRCA-mutated pancreatic cancer). In specific embodiments, the cancer is pancreatic cancer. In specific embodiments, the cancer is lung or liver cancer. In embodiments, the cancer is prostate cancer. In embodiments, the cancer treated is GI cancer, stomach cancer, CRC or mCRC. In embodiments, combined treatment of the CHD1L inhibitor with the PARP inhibitor reverses resistance of the cancer to treatment by the PARP inhibitor. In embodiments, the PARP inhibitor is olaparib, veliparib or talozoparib. In embodiments, the PARP inhibitor is rucaparib or niraparib. The invention also provides a method for treating a cancer which comprises administration of an amount of a PARP inhibitor effective for treatment of the cancer combined with administration of an amount of a CHD1L inhibitor effective for inhibiting CHD1L. In embodiments, the PARP inhibitor and the CHD1L inhibitor are administered by any known method on a dosing schedule appropriate for realizing the combined therapeutic benefit. In embodiments, the CHD1L inhibitor is administered orally and the PARP inhibitor is administered by intravenous injection. In embodiments, the CHD1L inhibitor and the PARP inhibitor are both administered by intravenous injection. In embodiments, the CHD1L inhibitor is administered prior to administration of the PARP inhibitor. In embodiments, the CHD1L inhibitor is administered prior to and optionally after administration of the PARP inhibitor. In embodiments, the CHD1L inhibitor is administered after administration of the PARP inhibitor. In embodiments, the CHD1L inhibitor is administered orally prior to and optionally after administration of the PARP inhibitor by intravenous injection.

In embodiments, CHD1L inhibitors are combined with one or more agent that induces DNA damage to treat neoplastic disease, including various cancers. In specific embodiments, CHD1L inhibitors exhibit more than additivity anticancer activity when combined with one or more agent that induces DNA damage. In specific embodiments, CHD1L inhibitors exhibit synergistic anticancer activity when combined with one or more agent that induces DNA damage. This combined axtivity of CHD1L inhibitors can be assessed in combination with methylmethane sulfonate (an alkylating agent), which is an exemplary agent that induces DNA damage.

The invention also provides a method for identifying a CHD1L inhibitor, which inhibits CHD1L-dependent TCF transcription which comprises determining if a selected compound inhibits a CHD1L ATPase, as described in examples herein. In specific embodiments, inhibition of cat-CHD1L ATPase is determined. In embodiments, compounds exhibiting % inhibition of 30% or greater are selected as inhibiting a CHD1L ATPase. In embodiments, compounds exhibiting % inhibition of 80% or greater are selected as inhibiting a CHD1L ATPase. In specific embodiments, CHD1L inhibitors exhibit IC₅₀ less than 10 μM in dose response assays against CHD1L ATPase, particularly cat-CHD1L ATPase. In specific embodiments, CHD1L inhibitors exhibit IC₅₀ less than 5 μM in dose response assays against CHD1L ATPase, particularly cat-CHD1L ATPase. In specific embodiments, CHD1L inhibitors exhibit IC₅₀ less than 5 μM in dose response assays against CHD1L ATPase, particularly cat-CHD1L ATPase. In specific embodiments, CHD1L inhibitors exhibit IC₅₀ less than 5 μM. In specific embodiments, CHD1L inhibitors exhibit IC₅₀ less than 3 μM. In specific embodiments, CHD1L inhibitors exhibit IC₅₀ less than 1 μM.

In specific embodiments, CHD1L inhibitors are assessed for inhibition of TCF-transcription in a 2D cancer cell model, particularly using one or more CRC cell lines, such as described in examples herein. In specific embodiments, inhibition of TCF-transcription is determined using a TOPflash reporter construct and more specifically a TOPflash luciferase reporter construct as described herein. In specific embodiments, inhibition of TCF-transcription by CHD1L inhibitors in the cell model is dose-dependent. In specific embodiments, inhibition of TCF-transcription by CHD1L inhibitors in the cell model is dose-dependent in the range of 1 to 50 μM. In specific embodiments, a CHD1L inhibitor exhibits % TCF-transcription normalized to cell viability of 75% or less at 20 μM. In specific embodiments, a CHD1L inhibitor exhibits % TCF-transcription normalized to cell viability of 50% or less at 40 μM. In specific embodiments, CHD1L inhibitors exhibit dose dependent inhibition of TCF-transcription with IC₅₀ less than 10 μM assayed with TOPflash reporter in a cancer cell line. In specific embodiments, CHD1L inhibitors exhibit dose dependent inhibition of TCF-transcription with IC₅₀ less than 5 μM assayed with TOPflash reporter in a cancer cell line. In specific embodiments, CHD1L inhibitors exhibit dose dependent inhibition of TCF-transcription with IC₅₀ less than 3 μM assayed with TOPflash reporter in a cancer cell line. In embodiments, the cancer cell line is a CRC cancer cell, a breast cancer cell, a glioma cell, a liver cancer cell, a lung cancer cell or a GI cancer cell. In an embodiment, the cancer cell line is a CRC cancer cell line. In a specific embodiment, the CRC cancer cell line is SW620.

In specific embodiments, CHD1L inhibitors are assessed for their ability to reverse or inhibit EMT. In specific embodiments, CHD1L inhibitors are assessed for their ability to reverse EMT in tumor organoids. In embodiments, reversion or inhibition of EMT is assessed in tumor organoids expressing vimentin where dose-dependent decrease in vimentin expression indicates reversion or inhibition of EMT. In embodiments, reversion of EMT is assessed in tumor organoids expressing E-cadherin where dose-dependent increase in E-cadherin expression indicates reversion or inhibition of EMT. In embodiments, reversion of EMT is assessed in tumor organoids expressing E-cadherin, vimentin or both, where dose-dependent decrease in vimentin and dose-dependent increase in E-cadherin expression indicates reversion or inhibition of EMT. In specific embodiments, dose-dependent reversion or inhibition of EMT is measured over a compound concentration of 0.1 to 100 μM. In specific embodiments, dose-dependent reversion of EMT is measured over a compound concentration of 0.3 to 50 μM.

In specific embodiments, CHD1L inhibitors are assessed for their ability to inhibit clonogenic colony formation which is a well-established assay to measure cancer stem cell stemness. In embodiments, cells are pretreated with a selected concentration of CHD1L inhibitors prior to plating. In embodiments, cells are cultured at low density such that only CSC will form colonies over 10 days in culture. In embodiments, cells are pretreated for 12-36 h. In embodiments, cells are pretreated for 24 h. In embodiments, cells are pretreated with CHD1L inhibitors at concentration in the range of 0.5-50 μM with appropriate controls. In embodiments, CHD1L inhibitors exhibit 40% or more inhibition of clonogenic colony counts, compared to no compound control, for CHD1L concentration of 40 μM. In embodiments, CHD1L inhibitors exhibit 40% or more inhibition of clonogenic colony counts, compared to no compound control, for CHD1L concentration of 20 μM. In embodiments, CHD1L inhibitors exhibit 40% or more inhibition of clonogenic colony counts, compared to no compound control, for CHD1L concentration of 2 μM. In embodiments, inhibition of clonogenic colony formation lasts over 10 days in culture.

In specific embodiments, CHD1L inhibitors are further assessed for loss of invasive potential employing any known method and particularly employing a method as described in the examples herein.

In specific embodiments, CHD1L inhibitors are further assessed for antitumor activity as measured by induction of cytotoxicity in tumor organoids. In embodiments, cells are treated for a selected time (e.g., 24-96 h, preferably 72 h) with selected concentration of CHD1L inhibitor (1-100 μM). In embodiments, cytotoxicity is measured using any of a variety of cytotoxicity reagents known in the art, such as small molecules which, enter damaged cells and exhibit a measurable change on entry (e.g., fluorescence, such as, CellTox™ Green reagent (Promega, Madison, Wis.) or IncuCyteCytotox reagents (Sartorius, France). In embodiments, cytotoxicity is measured by measurement of LDH (lactate dehydrogenase) released from dead cells.

In embodiments, the CHD1L inhibitors useful in methods of treatment, pharmaceutical compositions and pharmaceutical combinations herein are those of formulas I-XXIII, XXX-XLII and XLV-XLVI or pharmaceutically acceptable salts or solvates thereof. In embodiments, the invention provides novel compounds of any formula herein and in particular of formulas I-XXIII, XXXV-XLII or salts or solvates thereof. In embodiments, the CHD1L inhibitors are those of formula I, II, XX-XXIII. In embodiments, the CHD1L inhibitors are those of formula XX. In embodiments, the CHD1L inhibitors are those of formulas I-IX, XI-XIX, XX, XXI, XXII, XXIII or XXXV-XLII. In embodiments, the CHD1L inhibitors are those of formula XLV or XLVI.

In specific embodiments, the methods, pharmaceutical compositions and pharmaceutical combinations of the invention employ CHD1L inhibitors that are selected from one or more of compounds 1-177 or pharmaceutically acceptable salts or solvates thereof. Two or more CHD1L inhibitors can be employed in combination in the methods herein. In specific embodiments, the CHD1L inhibitors employed in the invention are selected from one or more of compounds 6-39 or pharmaceutically acceptable salts thereof. In specific embodiments, the CHD1L inhibitors employed in the methods of the invention are selected from one or more of compounds 40-51 or pharmaceutically acceptable salts or solvates thereof. In specific embodiments, the CHD1L inhibitors employed in the methods of the invention are selected from one or more of compounds 52-68 or pharmaceutically acceptable salts or solvates thereof. In specific embodiments, the CHD1L inhibitors employed in the methods of the invention are selected from one or more of compounds 70-73 or pharmaceutically acceptable salts or solvates thereof. In specific embodiments, the CHD1L inhibitors employed in the methods of the invention are selected from one or more of compounds 74-101 or pharmaceutically acceptable salts or solvates thereof. In specific embodiments, the CHD1L inhibitors employed in the methods of the invention are selected from one or more of compounds 102-103 or pharmaceutically acceptable salts or solvates thereof. In specific embodiments, the CHD1L inhibitors employed in the methods of the invention are selected from one or more of compounds 104-116 or pharmaceutically acceptable salts or solvates thereof. In specific embodiments, the CHD1L inhibitors employed in the methods of the invention are selected from one or more of compounds 117-142 or pharmaceutically acceptable salts or solvates thereof. In specific embodiments, the CHD1L inhibitors employed in the methods of the invention are selected from one or more of compounds 143-177 or pharmaceutically acceptable salts or solvates thereof. In specific embodiments, the CHD1L inhibitors employed in the methods of the invention are selected from one or more of compounds 150-154 or pharmaceutically acceptable salts or solvates thereof. In specific embodiments, the CHD1L inhibitors employed in the methods of the invention are selected from one or more of compounds 155-159 or pharmaceutically acceptable salts or solvates thereof. In specific embodiments, the CHD1L inhibitors employed in the methods of the invention are selected from one or more of compounds 28-39, 74-75, 52, 54, 62-66 or 74-75 or pharmaceutically acceptable salts or solvates thereof. In embodiments, the compound is selected from compounds 28, 31, 52, 54, 57, 75, 118, 126, 131, 150, or 169 or pharmaceutically acceptable salts or solvates thereof. In more specific embodiments, the compound is selected from compounds 52, 118, 126, 131, 150, or 169 or pharmaceutically acceptable salts or solvates thereof. In embodiments, the compound is selected from compounds 28, 31, 54, 57, or 75 or pharmaceutically acceptable salts or solvates thereof. In embodiments, the compound is one or more of compounds 28, 31, 52, 54, 57, 75, 118, 126, 131, 150, or 169 or pharmaceutically acceptable salts or solvates thereof. n embodiments, the compound is one of compounds 28, 31, 52, 54, 57, 75, 118, 126, 131, 150, or 169 or pharmaceutically acceptable salts or solvates thereof. In specific embodiments, the CHD1L inhibitors employed in the methods of the invention are selected from compound 52 or pharmaceutically acceptable salts or solvates thereof. In specific embodiments, the forgoing specifically recited CHD1L inhibitors can be combined with one or more alternative cancer cytoxic or antineoplastic agents for treatment or pharmaceutical combination. More specifically the alternative cancer cytoxic or antineoplastic agents include, without limitation, one or more PARP inhibitor, one or more topoisomerase inhibitor, one or more thymidylate synthase inhibitor or one or more platinum-based antineoplastic agent.

In specific embodiments, the CHD1L inhibitors employed in methods of this invention are compounds 6, 8, 52, 54, 56, 61, 62, 65 or 66 or pharmaceutically acceptable salts or solvates thereof. In specific embodiments, the CHD1L inhibitors employed in methods of this invention are compounds 6, 8 or pharmaceutically acceptable salts or solvates thereof. In specific embodiments, the CHD1L inhibitors employed in methods of this invention are compounds 52, 54 or pharmaceutically acceptable salts or solvates thereof. In specific embodiments, the CHD1L inhibitors employed in methods of this invention are compounds 22, 23, 26 or 27 or pharmaceutically acceptable salts thereof.

In specific embodiments, the methods of the invention employ CHD1L inhibitors of formula II and include all embodiments described herein for formula II. The invention also provides novel compounds of formula II, salts thereof and pharmaceutical compositions contains such compounds and salts.

In specific embodiments, the methods of the invention employ CHD1L inhibitors of formula XX and include all embodiments described herein for formula XX. The invention also provides novel compounds of formula XX, salts thereof and pharmaceutical compositions containing such compounds and salts.

In specific embodiments, the methods of the invention employ CHD1L inhibitors of formula XXI and include all embodiments described herein for formula XXI. The invention also provides novel compounds of formula XXI, salts thereof and pharmaceutical compositions containing such compounds and salts.

In specific embodiments, the methods of the invention employ CHD1L inhibitors of formula XXII and include all embodiments described herein for formula XXII. The invention also provides novel compounds of formula XXII, salts thereof and pharmaceutical compositions containing such compounds and salts.

In specific embodiments, the methods of the invention employ CHD1L inhibitors of formula XXIII and include all embodiments described herein for formula XXIII. The invention also provides novel compounds of formula XXII, salts thereof and pharmaceutical compositions containing such compounds and salts.

In specific embodiments, the methods of the invention employ CHD1L inhibitors of formula XLV and include all embodiments described herein for formula XXIII. The invention also provides novel compounds of formula XLV salts thereof and pharmaceutical compositions containing such compounds and salts.

In specific embodiments, the methods of the invention employ CHD1L inhibitors of formula XLVI and include all embodiments described herein for formula XLVI. The invention also provides novel compounds of formula XXII, salts thereof and pharmaceutical compositions containing such compounds and salts.

In embodiments, the invention is also directed to CHD1L inhibitors of this invention and pharmaceutically-acceptable compositions comprising any such inhibitors. In embodiments, pharmaceutically-acceptable compositions comprise one or more CHD1L inhibitors and a pharmaceutically-acceptable excipient.

In embodiments, the invention is directed to any compound or pharmaceutically acceptable salt or solvate thereof as described in chemical formulas herein which is novel. In particular, the invention is directed to CHD1L inhibitors and pharmaceutically acceptable salts thereof as described in formulas herein with the exception that the CHD1L inhibitor is other than compounds 1-8 or salts or solvates thereof. In particular, the invention is directed to CHD1L inhibitors and pharmaceutically acceptable salts thereof as described in formulas herein with the exception that the CHD1L inhibitor is other than compounds 1-9 or salts thereof. In embodiments, the invention is directed to any one of compounds 9-39, 40-68, 69-73, 74-101, 102-103, 104-116, 117-142, or 143-177 or pharmaceutically acceptable salts or solvates thereof or pharmaceutically acceptable compositions that contains such compounds or salts or solvates. In embodiments, the invention is directed to any one of compounds 10-39, 40-73, 74-116, 117-142 or 43-177 or pharmaceutically acceptable salts or solvates thereof or pharmaceutically acceptable compositions that contains such compounds or salts or solvates.

In embodiments, the invention is directed to any one of compounds 52-73 or pharmaceutically acceptable salts or solvates thereof or pharmaceutically acceptable compositions that contains such compounds or salts or solvates. In embodiments, the invention is directed to any one of compounds 28-39, 74, 75, 52, 54, 62-66, or 74-75 or pharmaceutically acceptable salts or solvates thereof or pharmaceutically acceptable compositions that contains such compounds or salts or solvates. In embodiments, the invention is directed to any one of compounds 10-39 or pharmaceutically acceptable salts or solvates thereof or pharmaceutically acceptable compositions that contains such compounds or salts or solvates. In embodiments, the invention is directed to any one of compounds 40-73 or pharmaceutically acceptable salts or solvates thereof or pharmaceutically acceptable compositions that contains such compounds or salts or solvates. In embodiments, the invention is directed to any one of compounds 74-116 or pharmaceutically acceptable salts or solvates thereof or pharmaceutically acceptable compositions that contains such compounds or salts or solvates. In embodiments, the invention is directed to any one of compounds 117-142 or pharmaceutically acceptable salts or solvates thereof or pharmaceutically acceptable compositions that contains such compounds or salts or solvates. In embodiments, the invention is directed to any one of compounds 143-177 or pharmaceutically acceptable salts or solvates thereof or pharmaceutically acceptable compositions that contains such compounds or salts or solvates. In embodiments, the invention is directed to one or more of compounds 10-177 of Scheme 1 or pharmaceutically acceptable salts or solvates thereof or pharmaceutically-acceptable compositions that contains such compounds or salts or solvates. In embodiments, the invention is directed to one or more of compounds 150-154 or pharmaceutically acceptable salts or solvates thereof or pharmaceutically acceptable compositions that contains such compounds or salts or solvates. In embodiments, the invention is directed to one or more of compounds 155-159 or pharmaceutically acceptable salts or solvates thereof or pharmaceutically acceptable compositions that contains such compounds or salts or solvates. In embodiments, the compound is selected from compounds 28, 31, 52, 54, 57, 75, 118, 126, 131, 150, or 169 or pharmaceutically acceptable salts or solvates thereof or pharmaceutically acceptable compositions that contains such compounds or salts or solvates. In more specific embodiments, the compound is selected from compounds 52, 118, 126, 131, 150, or 169 or pharmaceutically acceptable salts or solvates thereof or pharmaceutically acceptable compositions that contains such compounds or salts or solvates. In embodiments, the compound is selected from compounds 28, 31, 54, 57, or 75 or pharmaceutically acceptable salts or solvates thereof or pharmaceutically acceptable compositions that contains such compounds or salts or solvates. In embodiments, the compound is one or more of compounds 28, 31, 52, 54, 57, 75, 118, 126, 131, 150, or 169 or pharmaceutically acceptable salts or solvates thereof or pharmaceutically acceptable compositions that contains such compounds or salts or solvates. In embodiments, the compound is one of compounds 28, 31, 52, 54, 57, 75, 118, 126, 131, 150, or 169 or pharmaceutically acceptable salts or solvates thereof or pharmaceutically acceptable compositions that contains such compounds or salts or solvates.

In specific embodiments, the CHD1L inhibitors employed in the methods of the invention are selected from compound 52 or pharmaceutically acceptable salts or solvates thereof. In embodiments, a CHD1L inhibitor of the invention has Clog P of 5 or less. In embodiments, a CHD1L inhibitor of the invention has Clog P of 3-4.

In specific embodiments the invention is directed to the following compounds and to methods herein employing these compounds for the treatment of cancer, particularly CRC and mCRC: any one of compounds 52-73; compound 52 or 53; compounds 54, 55 or 67; compounds 57, 58 or 59; or pharmaceutically acceptable salts or solvates thereof; any one of compound 8, compound 52, compound 53, compound 54, compound 55, compound 56, compound 57, compound 58, compound 59, compound 61, compound 62, compound 65, compound 66, or compound 67 or pharmaceutically acceptable salts or solvates thereof. In specific embodiments the invention is directed to the following compounds and to methods herein employing these compounds for the treatment of cancer, particularly CRC and mCRC: any one of compounds 28, 31, 52, 54, 57, 75, 118, 126, 131, 150, or 169; any one of compounds 52, 118, 126, 131, 150, or 169; any one of compounds 28, 31, 54, 57, or 75; any one of compounds 28, 31, 52, 54, 57, 75, 118, 126, 131, 150, or 169; one or more of compounds 28, 31, 52, 54, 57, 75, 118, 126, 131, 150, or 169.

In embodiments, the invention provides a pharmaceutical combination of one or more CHD1L inhibitor and one or more alternative cancer cytotoxic or antineoplastic agent. In embodiments, the components of the pharmaceutical combination can be together or separate. In embodiments, the pharmaceutical combination is a pharmaceutical compositions containing one or more CHDL1 inhibitor and one or more PARP inhibitor or one or more topoisomerase inhibitor or one or more thymidylate synthase inhibitor. In embodiments, the pharmaceutical combination is a pharmaceutical compositions containing one or more CHDL1 inhibitor and one or more platinum-based antineoplastic agent. In embodiments, the pharmaceutical combination is two or more separate pharmaceutical compositions each containing different components of the pharmaceutical combination. In embodiments, the pharmaceutical combination is two separate pharmaceutical compositions, one containing one or more CHD1L inhibitors and one containing one or more PARP inhibitors or one or more topoisomerase inhibitor or one or more thymidylate synthase inhibitor. In embodiments, the pharmaceutical combination is two separate pharmaceutical compositions, one containing one or more CHD1L inhibitors and one containing one or more platinum-based antineoplastic agent. In embodiments, the pharmaceutical combination is a single pharmaceutical composition, containing one or more CHD1L inhibitors and one containing one or more PARP inhibitor. In embodiments, the pharmaceutical combination is a single pharmaceutical composition, containing one or more CHD1L inhibitors and one containing one or more topoisomerase inhibitor. In embodiments, the pharmaceutical combination is a single pharmaceutical composition, containing one or more CHD1L inhibitors and one containing one or more thymidylate synthase inhibitor. More specifically, the invention relates to pharmaceutical combinations as described herein which comprise one or more CHD1L inhibitor of any one of formulas I-XXIII, XXX-XLII anf XLV-XLVI or pharmaceutically acceptable salts or solvates thereof. More specifically, the invention relates to pharmaceutical combinations as described herein which comprise one or more CHD1L inhibitor of any one of formulas I, II, XX-XXIII or pharmaceutically acceptable salts or solvates thereof. More specifically, the invention relates to pharmaceutical combinations as described herein which comprise one or more CHD1L inhibitor of any one of formulas XLV-XLVI or pharmaceutically acceptable salts or solvates thereof. More specifically, the invention relates to pharmaceutical combinations as described herein which comprise one or more of CHDL1 inhibitors of any of compounds 1-177 or any one of compounds 9-177 or pharmaceutically acceptable salts or solvates thereof. In specific embodiments, the CHD1L inhibitors of the pharmaceutical combination are compounds 52-73; compound 52 or 53; compounds 54, 55 or 67; or compounds 57, 58 or 59; or pharmaceutically acceptable salts or solvates thereof; any one of compound 8, compound 52, compound 53, compound 54, compound 55, compound 56, compound 57, compound 58, compound 59, compound 61, compound 62, compound 65, compound 66, or compound 67 or pharmaceutically acceptable salts or solvates thereof. In embodiments, the CHD1L inhibitors of the pharmaceutical combination are any one of compounds 28, 31, 52, 54, 57, 75, 118, 126, 131, 150, or 169; any one of compounds 52, 118, 126, 131, 150, or 169; any one of compounds 28, 31, 54, 57, or 75; any one of compounds 28, 31, 52, 54, 57, 75, 118, 126, 131, 150, or 169; one or more of compounds 28, 31, 52, 54, 57, 75, 118, 126, 131, 150, or 169.

In embodiments, the invention also relates to the use of a CHD1L inhibitor in the manufacture of a medicament for the treatment of cancer, particularly for the treatment of CHD1L-driven cancer, TCF-driven cancer, or EMT-driven cancer, particularly GI cancer, and more particularly CRC or mCRC. In embodiments, the cancer to be treated is breast cancer, particularly BRCA-mutated breast cancer, ovarian cancer, particularly BRCA-mutated ovarian cancer, pancreatic cancer, particularly BRCA-mutated pancreatic cancer, lung cancer, prostate cancer or liver cancer. More specifically, the invention relates to the use of a CHD1L inhibitor of any one of formulas I-XX, XXI, XXII, XXIII, XXX-XLII and XLV-XLVI or pharmaceutically acceptable salts or solvates thereof in the manufacture of a medicament for the treatment of cancer, CHD1L-driven cancer, TCF-driven cancer, or EMT-driven cancer, particularly GI cancer, and more particularly CRC or mCRC. In embodiments, the CHD1L inhibitors are those of formulas I-IX, XI-XIX, XX, XX1, XXII, XXIII, XXXV-XLII and XLV-XLVI. In embodiments, the CHD1L inhibitors are those of formula I, formula II, formula XX, formula XXI, formula XXII or formula XXIII. In embodiments, the CHD1L inhibitors are those of formula XLV or XLVI. In embodiments, the CHD1L inhibitor is one or more of the compounds 1-117 of Scheme 1. In embodiments, the CHD1L inhibitor is one or more of the compounds 118-177 of Scheme 1. In embodiments, the CHD1L inhibitors are compounds 52-73; compound 52 or 53; compounds 54, 55 or 67; or compounds 57, 58 or 59; or pharmaceutically acceptable salts or solvates thereof; any one of compound 8, compound 52, compound 53, compound 54, compound 55, compound 56, compound 57, compound 58, compound 59, compound 61, compound 62, compound 65, compound 66, or compound 67 or pharmaceutically acceptable salts or solvates thereof. In embodiments, the CHD1L inhibitors of the pharmaceutical combination are any one of compounds 28, 31, 52, 54, 57, 75, 118, 126, 131, 150, or 169; any one of compounds 52, 118, 126, 131, 150, or 169; any one of compounds 28, 31, 54, 57, or 75; any one of compounds 28, 31, 52, 54, 57, 75, 118, 126, 131, 150, or 169; one or more of compounds 28, 31, 52, 54, 57, 75, 118, 126, 131, 150, or 169 or pharmaceutically acceptable salts or solvates thereof.

In embodiments, the invention also relates to the use of a CHD1L inhibitor in combination with an alternative cancer cytooxic or antineoplastic agent in the manufacture of a medicament for the combination treatment of cancer, particularly for the treatment of CHD1L-driven cancer, TCF-driven cancer, or EMT-driven cancer, particularly GI cancer, and more particularly CRC or mCRC. In embodiments, the cancer to be treated is breast cancer, particularly BRCA-mutated breast cancer or metastatic breast cancer, ovarian cancer, particularly BRCA-mutatedovarian cancer, pancreatic cancer, particularly BRCA-mutated pancreatic cancer, lung cancer, prostate cancer, or liver cancer. More specifically, the invention relates to the use of a CHD1L inhibitor of any one of formulas I-XXIII, XXX-XLII and XLV-XLVI or pharmaceutically acceptable salts or solvates thereof in the manufacture of a medicament for the combination treatment of cancer, CHD1L-driven cancer, TCF-driven cancer, or EMT-driven cancer, particularly GI cancer, and more particularly CRC or mCRC. In embodiments, the CHD1L inhibitors are those of formula I, formula II, formula XX, formula XXI, formula XXII or formula XXIII. In embodiments, the CHD1L inhibitors are those of formula XLV-XLVI. In embodiments, the CHD1L inhibitors are those of formulas I-IX, XI-XIX, XX, XXI, XXII, XXIII, XXII, XXIII, XXXV-XLII or XLV-XLVI. In embodiments, the CHD1L inhibitor is one or more of the compounds 1-117 or one or more of compounds 8-177, compounds 9-177 or compounds 118-177 of Scheme 1. In embodiments, the CHD1L inhibitors are compounds 52-73; compound 52 or 53; compounds 54, 55 or 67; or compounds 57, 58 or 59; or pharmaceutically acceptable salts or solvates thereof; any one of compound 8, compound 52, compound 53, compound 54, compound 55, compound 56, compound 57, compound 58, compound 59, compound 61, compound 62, compound 65, compound 66, or compound 67 or pharmaceutically acceptable salts or solvates thereof. In embodiments, the CHD1L inhibitors of the pharmaceutical combination are any one of compounds 28, 31, 52, 54, 57, 75, 118, 126, 131, 150, or 169; any one of compounds 52, 118, 126, 131, 150, or 169; any one of compounds 28, 31, 54, 57, or 75; any one of compounds 28, 31, 52, 54, 57, 75, 118, 126, 131, 150, or 169; one or more of compounds 28, 31, 52, 54, 57, 75, 118, 126, 131, 150, or 169 or pharmaceutically acceptable salts or solvates thereof. In embodiments, the one or more CHD1L inhibitors are combined in the medicament with one or more PARP inhibitors, one or more topoisomerase inhibitors, one or more thymidylate synthase inhibitors or one or more platinum-based antineoplastic agents.

In embodiments, the invention further relates to a CHD1L inhibitor in combination with one or more alternative cancer cytotoxic or antineoplastic agent for use in the treatment of cancer, CHD1L-driven cancer, TCF-driven cancer, or EMT-driven cancer, particularly GI cancer, and more particularly CRC or mCRC. In embodiments, the cancer to be treated is breast cancer, ovarian cancer, and pancreatic cancer, particularly BRCA-mutated breast cancer, BRCA-mutated ovarian cancer, BRCA-mutated pancreatic cancer, prostate cancer, stomach cancer, lung cancer, or liver cancer. More specifically, the invention relates to the use of a CHD1L inhibitor of any one of formulas I-XXIII, XXX-XLII and XLV-XLVI or pharmaceutically acceptable salts or solvates thereof in the manufacture of a medicament for the combination treatment of cancer, CHD1L-driven cancer, TCF-driven cancer, or EMT-driven cancer, particularly GI cancer, and more particularly CRC or mCRC. In embodiments, the CHD1L inhibitors are those of formula I, formula II, formula XX, formula XXI, formula XXII or formula XXIII. In embodiments, the CHD1L inhibitors are those of formula XLV-XLVI. In embodiments, the CHD1L inhibitors are those of formulas I-IX, XI-XIX, XX, XXI, XXII, XXIII, XXII, XXIII, XXXV-XLII or XLV-XLVI. In embodiments, the CHD1L inhibitor is one or more of the compounds 1-117 or one or more of compounds 8-177, compounds 9-177 or compounds 118-177 of Scheme 1. In embodiments, the CHD1L inhibitors are compounds 52-73; compound 52 or 53; compounds 54, 55 or 67; or compounds 57, 58 or 59; or pharmaceutically acceptable salts or solvates thereof; any one of compound 8, compound 52, compound 53, compound 54, compound 55, compound 56, compound 57, compound 58, compound 59, compound 61, compound 62, compound 65, compound 66, or compound 67 or pharmaceutically acceptable salts or solvates thereof. In embodiments, the CHD1L inhibitors of the pharmaceutical combination are any one of compounds 28, 31, 52, 54, 57, 75, 118, 126, 131, 150, or 169; any one of compounds 52, 118, 126, 131, 150, or 169; any one of compounds 28, 31, 54, 57, or 75; any one of compounds 28, 31, 52, 54, 57, 75, 118, 126, 131, 150, or 169; one or more of compounds 28, 31, 52, 54, 57, 75, 118, 126, 131, 150, or 169 or pharmaceutically acceptable salts or solvates thereof. In embodiments, the alternative cancer cytotoxic or antineoplastic agent is one or more PARP inhibitors, one or more topoisomerase inhibitors, one or more thymidylate synthase inhibitors or one or more platinum-based antineoplastic agents.

Other embodiments and aspects of the invention will be readily apparent to one of ordinary skill in the art on review of the drawings, detailed description and examples herein.

### BRIEF DESCRIPTION OF THE DRAWINGS

FIGS. 1A-B: Validation of CHD1L inhibitors identified from HTS. (FIG. 1A) cat-CHD1L ATPase C50 dose responses with hits 1-7. Mean IC₅₀ values are calculated from three independent experiments and representative graphs are shown. (FIG. 1B) SW620, HCT-16, and DLD1CHD1L-OE cells with TOPflash reporter were used to measure inhibition of TCF transcription using 3 doses over 24h.

FIGS. 2A-2D: CHD1L inhibitors reverse EMT and the malignant phenotype in CRC. Dose responses for CHD1L inhibitors that modulate EMT measured by high-content imaging of (FIG. 2A) downregulation of VimPro-GFP reporter and (FIG. 2B) upregulation of EcadPro-RFP reporter. Mean EC₅₀ values ±SEM are calculated from three independent experiments (FIG. 2C) CSC stemness measured by clonogenic colony formation after pretreatment with CHD1L inhibitors in DLD1CHD1L-OE and HCT-116 cells. (FIG. 2D) Inhibition of invasive potential of HCT-116 cells after treatment of CHD1L inhibitors. Welch's t-test statistical analysis was used to determine significance, where *=P≤0.05, **=P≤0.01, ***=P≤0.001, ****=P≤0.0001.

FIGS. 3A-C: Compound 6 induces apoptosis in CRC cell lines and PDTOs. (FIG. 3A) Time course evaluation of the induction E-cadherin expression using Ecad-ProRFP reporter assay and cytotoxicity using Cell-Tox™ Green cytotoxicity assay (Promega, Madison, Wis.). (FIG. 3B) Annexin V-FITC staining analysis of apoptosis after treatment of SN-38 and 6 for 12 hours. (FIG. 3C) Cytotoxicity of 6 in PDTO CRC102 using CellTiter-Blue® cell viability assay (Promega, Madison, Wis.). Mean EC₅₀ values ±s.d. are calculated from six independent experiments and representative graph is shown with inset of a representative PDTO. Welch's t-test statistical analysis was used to determine significance, where *=P≤0.05, **=P≤0.01, ***=P≤0.001, ****=P≤0.0001.

FIG. 4: Accumulation of Compound 6 in SW620 xenograft tumors. Compound 6 was administered by i.p. injection to athymic nude mice QD for 5 days to measure accumulation in SW620 xenograft tumors.

FIG. 5: Proposed mechanism of action of CHD1L mediated TCF-transcription. CHD1L is activated through binding TCF-complex members PARP1 and TCF4 [Abbott et al., 2020] (1) Once activated, CHD1L is directed to hindered WREs localized on chromatin. (2) Chromatin remodeling and DNA translocation occurs exposing WRE sites. (3) TCF-complex binds to exposed WREs facilitated by CHD1L, promoting EMT genes and other genes associated with mCRC. CHD1L ATPase inhibitors effectively prevent step 1, leading to the reversion of EMT and other malignant properties of CRC.

FIGS. 6A-E Evaluation of Compound 8. (FIG. 6A) Compound 8 displays potent low μM dose-dependent inhibition of TCF-transcription based on TOPFlash reported in SW260 cells cultures in 2D and over a 24 h time course. Compound 8 effectively reverses EMT in dual reporter SW620 tumor organoids over 72 h evidenced by downregulation of vimentin (FIG. 6B) and (FIG. 6C) upregulation of E-cadherin promoter activity in a dose-dependent manner. Compound 8 significantly inhibits (FIG. 6D) clonogenic colony formation over 10 days after pre-treatment for 24 h and (FIG. 6E) HCT116 invasive potential over 48 h. The students t-test indicates* P≤0.05

FIGS. 7A-B: Viability of Colorectal Cancer Tumor Organoids after Treatment with Exemplary CHDIL Inhibitors. The figures illustrate representative graphs of % viability as a function of log concentration of the indicated compound. (FIG. 7A) Treatment with Compound 6.9; (FIG. 7B) Treatment with Compound 6.11; Alternative compound numbers as used in Scheme 1 are given in parenthesis. IC₅₀, in some cases average IC₅₀, are provided in each figure. Viability data for a number of exemplary compounds are provided in Table 3.

FIGS. 8A-B: Assessment of CHD1L-mediated DNA repair and “on target” effects of CHD1L inhibitor 6 alone and in combination with irinotecan (prodrug of SN38). CHD1L is known to be essential for PARP-1-mediated DNA repair, causing resistance to DNA damaging chemotherapy [Ahel et al., 2009; Tsuda et al., 2017]. DLD1 CRC cells that have low level expression of CHD1L (DLD1 Empty Vector, EV) compared to DLD1 cells that were engineered to overexpress CHD1L (DLD1 Overexpressing, OE) were used. FIG. 8A is a Western blot comparing expression of CHD1L in DLD1(EV) to DLD1(OE) in view of control expression of α-tubulin in these cells. FIG. 8B presents a graph of γ-H2AX intensity (relative to DMSO) for compound alone, SN38 alone, and a combination of the two in DLD1 empty vector cells and DLD1 overexpressing cells. Compound 6.0 alone does not induce significant DNA damage, nor does it synergize with SN38 in DLD1 cells with low expression of CHD1L. This graph demonstrates CHD1L inhibitor “on target” effects that synergize with SN38 inducing DNA damage in DLD1 cells overexpressing CHD1L.

FIGS. 9A-9C: Synergy studies with exemplary CHD1L inhibitors and irinotecan (Prodrug of SN38). (FIG. 9A) Synergy studies with compounds 6 and 6.3 in SW620 Colorectal Cancer (CRC) Tumor Organoids. (FIG. 9B) Synergy studies with compound 6.9 in SW620 Colorectal Cancer (CRC) Tumor Organoids. (FIG. 9C) Synergy studies with compound 6.11 in SW620 Colorectal Cancer (CRC) Tumor Organoids. SN38 combinations of 6, and 6.3 are 50-fold, and 150-fold more potent, respectively, than SN38 alone in killing colon SW620 tumor organoids. SN38 combination of 6.9 and 6.11 are both over 100-fold more potent than SN38 alone. Each of compounds 6, 6.3, 6.9 and 6.11 exhibit synergism with irinotecan (and SN38) for killing SW620 tumor organoids.

FIG. 10: In vivo synergy studies of compound 6 in combination with irinotecan in mice. FIG. 10 includes a graph of tumor volume (fold) SW620 tumor xenografts as a function of days (up to 28 days) of treatment with compound 6 alone (2), irinotecan alone (3) or a combination thereof (4), compared to control (1). A data Table is also provided showing data statistical significance. The combination of irinotecan and compound 6 significantly inhibit colon SW620 tumor xenografts to almost no tumor volume within 28 days of treatment compared to the single agent treatment groups.

FIG. 11: In vivo synergy of CHD1L inhibitor compound 6 and irinotecan continues post treatment. FIG. 11 includes a graph of tumor volume (fold) SW620 tumor xenografts as a function of days (up to 41 days) of treatment with irinotecan alone (1) or a combination of compound 6 and irinotecan (2). A data Table is also provided showing data statistical significance. The combination of irinotecan and compound 6 significantly inhibits colon SW620 tumors to almost no tumor volume beyond the last treatment (day 28) compared to irinotecan alone. Within 2-weeks of the last treatment of irinotecan alone tumor volume rose to above the volume of the last treatment, signifying tumor recurrence. In contrast the combination maintained a lower tumor volume.

FIG. 12: Compound 6 alone and in combination with irinotecan significantly increases the survival of CRC tumor-bearing mice compared to vehicle and irinotecan alone. FIG. 12 includes a graph of survival (%) as a function of time up to 52 days after last treatment on day 28 with compound 6 alone (2), irinotecan alone (3) or a combination thereof (4), compared to control (1). A data Table is also provided showing data statistical significance. Survival rate was significantly higher with the combination treatment compared to single dosage compounds or control.

FIG. 13: In vivo synergy studies of compound 6.11 in combination with irinotecan in mice. FIG. 13 includes a graph of tumor volume (fold) SW620 tumor xenografts as a function of days (up to 20 days) of treatment with compound 6.11 alone (2), irinotecan alone (3) or a combination thereof (4), compared to control (1). A data Table is also provided showing data statistical significance. The combination of irinotecan and compound 6.11 significantly inhibit colon SW620 tumor xenografts to almost no tumor volume within 20 days of treatment compared to the irinotecan alone.

FIG. 14: In vivo synergy of CHD1L inhibitor compound 6.11 and irinotecan continues post treatment. FIG. 14 includes a graph of tumor volume (fold) SW620 tumor xenografts as a function of days (up to 41 days) of treatment with irinotecan alone (1) or a combination of compound 6 and irinotecan (2). Treatment was stopped at day 33 (Tx released). The combination of irinotecan and compound 6.11 significantly inhibits colorectal SW620 tumors beyond the last treatment (day 33) compared to irinotecan alone.

FIG. 15: In vivo synergy of CHD1L Inhibitor 6.11 and irinotecan significantly increases survival benefit. Compound 6.11 in combination with irinotecan significantly Increases the survival of CRC tumor-bearing mice compared to vehicle and irinotecan alone. FIG. 15 includes a graph of survival (%) as a function of time up to 50 days after last treatment on day 33 with compound 6 alone (2), irinotecan alone (3) or a combination thereof (4), compared to control (1). A data Table is also provided showing data statistical significance. Survival rate was significantly higher with the combination treatment compared to irinotecan alone or control.

FIGS. 16A and 16B. Enzymatic inhibition of CHD1L and SW620 tumor organoid cytotoxicity. (FIG. 16A) Quantification of the catalytic domain of CHD1L recombinant protein. (FIG. 16B) Dose-response of CHD1L inhibitor compounds measuring SW620 tumor organoid viability. Data is normalized to DMSO control and is shown as mean±SEM of triplicate experiments.

FIGS. 17A-17C. CHD1L Inhibitors downregulate CHD1L mediated TCF-transcription in M-Phenotype cells. (FIG. 17A) TCF-transcriptional activity in isolated SW620 and HCT116 EMT phenotypes. P-values were calculated by one-way ANOVA where *P<0.05. Dose-response graphs of SW620 (FIG. 17B) and HCT116 (FIG. 17C) M-Phenotype monolayer cell culture treated with listed CHD1L inhibitors for 24h, measuring TCF-transcription via the TOPflash luminescent reporter assay. Data is normalized to cell viability and is shown as mean±SEM of duplicate experiments.

FIGS. 18A-18D. CHD1L inhibitors are potent cytotoxic agents in CRC cell line and patient tumor organoids. (FIGS. 18A and 18B) Dose-response graphs of lead CHD1Li, measuring cell viability after 72 h of treatment of isolated M-phenotype SW620 and HCT116 tumor organoids. (FIGS. 18C and 18D) Dose-response graphs of lead CHD1Li, measuring cell viability after 72 h of treatment of CRC042 and CRC102 patient-derived tumor organoids (PDTO). The data was normalized to DMSO (vehicle) and is shown as the mean±SEM of triplicate experiments with technical replicates (n=3) for each experiment.

FIGS. 19A-19E. CHD1Li induce MET in M-phenotype SW620 and HCT116 tumor organoids. (FIGS. 19A and 19C) Dose-response graphs of the downregulation of VimPro-GFP promoter activity measured by EGFP fluorescence of SW620 and HCT116 tumor organoids treated with lead CHD1L inhibitors. (FIGS. 19B and 19D) Fold change upregulation of EcadPro-RFP promoter activity measured through RFP fluorescent signal in SW620 and HCT116 tumor organoids after treatment with lead CHD1L inhibitors. (FIG. 19E) Representative maximum projection confocal images of HCT116 tumor organoids after treatment with compound 6.5 for both VimPro-GFP and EcadPro-RFP promoter activity. Data is shown in mean±SEM of duplicate experiments.

FIGS. 20A-20B. Cancer cell stemness is greatly reduced by CHD1L inhibitors. (FIG. 20A) Number of clonogenic colonies formed after continuous lead CHD1Li treatment in SW620 cells. (FIG. 20B) Number of clonogenic colonies formed after continuous treatment with lead CHD1L inhibitors in HCT116 cells. The data is represented as the mean±SEM of duplicate experiments using triplicate technical replicates.

FIGS. 21A and 21B. Oral Bioavailable Efficacy of Compound 6.11 Against SW620 Quasi-Mesaenchymal (GFP+) Tumor Xenographs. FIG. 21A is a graph of tumor volume as a function of days after initiation of treatment for control vehicle only (•, closed circles), 6.11 75 mg/kg (squares) and 6.11 125 mg/kg. Data point significance assessed using 2-Way ANOVA (multiple comparison), where *P<0.05, **P<0.01, ***P<0.001, ****P<0.0001, #P<0.05, #P<0.0001. FIG. 21B is a graph of average mouse body weight as a function of days after initiation of treatment.

### DETAILED DESCRIPTION

The invention relates generally to the characterization of a relatively new oncogene, CHD1L, as a tumorigenic factor associated with poor prognosis and survival in CRC. A new biological function for CHD1L as a DNA binding factor for the TCF transcription complex required for promoting TCF-driven EMT and other malignant properties has been demonstrated. Abbott et al., 2020 and the supplementary information for this article, which is available from the journal web site (mct.aacrjournals.org), provide description of a portion of the experiments and data presented herein and are each incorporated by reference herein in its entirety. Prigaro et al., 2022 and the supporting information for this article, which is available from the journal web site (pubs.acs.org/doi/10.1021/acs.jmedchem.1c01778) provide additional description of a portion of the experiments and data presented herein and are each incorporated by reference herein in its entirety.

CHD1L is amplified (Chr1q21) and overexpressed in many types of cancer (e.g., breast, bladder, colorectal, esophageal, fibrosarcoma, liver, ovarian, and gastrix cancer). [Ma et al., 2008; Cheng et al., 2013] CHD1L overexpression has been characterized as a marker for poor prognosis and metastasis in numerous cancers. [Ma et al., 2008; Cheng et al., 2008; Hyeon et al., 2013; Su et al., 2014] While the collective literature demonstrating CHD1L as an oncogene and driver of malignant cancer is compelling, the rigor of the prior research and the hypothesis that CHD1L is an oncogene with potential as a molecular target in CRC is tested herein. In silico analyses of transcriptome data from a large cohort of 585 CRC patients obtained over 15 years was reported. [Marisa et al., 2013] CHD1L expression was correlated with poor survival, with low-CHD1L patients living significantly longer than high-CHD1L patients. Using the same cohort, Marisa et al., 2013 identified six distinct subtypes for improved clinical stratification of CRC and CHD1L is universally expressed in all six subtypes, indicating its potential as a therapeutic target for CRC. CHD1L also correlated with tumor node metastasis, with increased expression moving from NO (no regional spread) to N3 (distant regional spread). Transcriptome data from a UCCC patient cohort (n=25) was analyzed and it was found that CHD1L expression significantly correlated with stage IV and mCRC. Literature reports and the work herein demonstrate that CHD1L is an oncogene promoting malignant CRC and its high expression correlates with poor prognosis and survival of CRC patients.

A new biological function for CHD1L as a DNA binding factor for the TCF-transcription complex required for promoting TCF-driven EMT and other malignant properties is demonstrated herein. Using HTS drug discovery the first known inhibitors of CHD1L have been identified and characterized which display good pharmacological efficacy in cell-based models of CRC, including PDTOs. CHD1L inhibitors effectively prevent CHD1L-mediated TCF-transcription, leading to the reversion of EMT and other malignant properties, including CSC stemness and invasive potential. Notably, CHD1L inhibitor 6 displays the ability to induce cell death that is consistent with the reversion of EMT and induction of cleaved E-cadherin mediated extrinsic apoptosis through death receptors. Furthermore, compound 6 synergizes with SN38 (i.e., irinotecan) displaying potent DNA damage induction compared to SN38 alone, which is consistent with the inhibition of PARP1/CHD1L mediated DNA repair. CHD1L inhibitors having drug-like physicochemical properties and favorable in vivo PK/PD disposition with no acute liver toxicity have been identified.

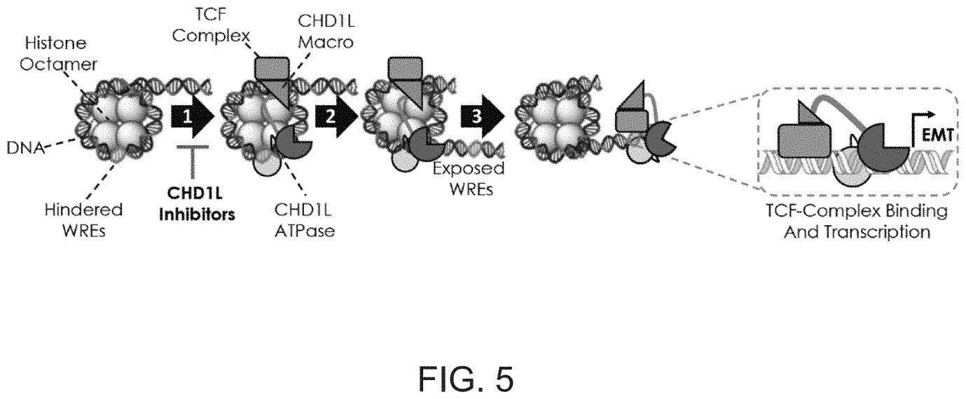

Based on the data presented herein, a mechanism of action for CHD1L-mediated TCF-driven EMT involved in CRC tumor progression and metastasis is presented (FIG. 5). In this mechanism, TCF-complex specifically recruits CHD1L to dynamically regulate metastatic gene expression. Central to this mechanism, CHD1L binds to nucleosome hindered WREs when directed by the TCF-complex via protein interactions with PARP1 and TCF4. Importantly, PARP1 is characterized as the major cellular activator of CHD1L through macro domain binding that releases auto inhibition. [Lehmann et al., 2017; Gottschalk et al., 2009] Moreover, PARP1 is a required component of the TCF-complex forming interactions with β-catenin and TCF4. [Idogawa et al., 2005] Therefore, the mechanism indicates that CHD1L is recruited by the TCF-complex and activated by PARP1 and TCF4. Once activated, CHD1L exposes WREs by nucleosome translocation, facilitating TCF-complex binding to WREs and transcription of malignant genes promoting EMT. CHD1L inhibitors have a unique mechanism of action by inhibiting CHD1L ATPase activity, which prevents exposure of WREs to the TCF-complex, inhibiting transcription of TCF-target genes associated with EMT and particularly with mCRC.

Small molecule inhibitors of CHD1L, as described herein, have been identified in screens based on inhibition of CHD1L ATPase activity. Certain inhibitors identified exhibit drug-like physicochemical properties and favorable in vivo PK/PD disposition with no acute liver toxicity. Such inhibitors are effective as a treatment for CRC and mCRC (metastatic CRC) among other CHD1L-driven cancers.

Well-known methods for assessment of drugability can be used to further assess active compounds of the invention for application to given therapeutic application. The term “drugability” relates to pharmaceutical properties of a prospective drug for administration, distribution, metabolism and excretion. Drugability is assessed in various ways in the art. For example, the “Lipinski Rule of 5” for determining drug-like characteristics in a molecule related to in vivo absorption and permeability can be applied [Lipinski et al., 2001; Ghose, et al., 1999]

The invention provides methods for combination therapy in which administration of CHD1L inhibitor is combined with administration of one or more anticancer agent which is not a CHD1L inhibitor. In embodiments, the other anticancer agents is a topoisomerase inhibitor, a platinum-based antineoplastic agent, a PARP inhibitor or combinations of two or more of such inhibitors and agents. In embodiments, the combination therapy combines administration of a CHD1L inhibitor with a topoisomerase inhibitor. In embodiments, the combination therapy combines administration of a CHD1L inhibitor with a platinum-based antineoplastic agent. In embodiments, the combination therapy combines administration of a CHD1L inhibitor with a PARP inhibitor. In embodiments, the combination therapy combines administration of a CHD1L inhibitor with a topoisomerase inhibitor and administration of a PARP inhibitor. In embodiments, the combination therapy combines administration of a CHD1L inhibitor with chemotherapy for the specific cancer being treated. In embodiments herein, the combination of a CHD1L inhibitor and the other antineoplastic agent exhibits synergistic activity in combination.

In embodiments herein, therapy employing CHD1L can be combined with radiation therapy suitable for a given cancer.

Various PARP inhibitors are known in the art. [See, for example Rouleau et al., 2010; Yi et al., 2019; Zhou et al., 2020; Wahlberg et al., 2012; D'Andrea, 2018] Each of these references is incorporated by reference herein it is entirety for descriptions of PARP inhibitors, the mechanism of PARP inhibitor action, cancers treated using PARP inhibitors, and resistance to PARP inhibitors. In a specific embodiment herein, PARP-resistance cancer is treated with a combination of a CHD1L inhibitor and the PARP inhibitor.

Various topoisomerase inhibitors are known in the art and have been employed clinically. (See, for example, Hevener, 2018; Bailly, 2012; Nitiss J, 2009) “Targeting DNA topoisomerase II in cancer chemotherapy,” Nature Rev. Cancer, 9:338-350). Each of these references is incorporated by reference herein in its entirety for descriptions of types of topoisomerase inhibitors, specific topoisomerase inhibitors, mechanisms of topoisomerase inhibition, cancers treated using topoisomerase inhibitors and combination therapies using topoisomerase inhibitors. In embodiments, topoisomerase inhibitors useful in methods and compositions herein are topoisomerase I inhibitors. In embodiments, topoisomerase inhibitors useful in methods and compositions herein include camptothecin and prodrugs thereof, irinotecan, topotecan, belotecan, indotecan, or indimitecan. In embodiments, topoisomerase inhibitors useful in methods and compositions herein include etoposide or teniposide. In embodiments, topoisomerase inhibitors useful in methods, pharmaceutical combinations and combined cancer therapy herein include namitecan, silatecan, vosaroxin, aldoxorubicin, doxorubicin, becatecarin, or edotecarin.

In embodiments, topoisomerase inhibitors useful in methods and compositions herein are topoisomerase I inhibitors. Exemplary topoisomerase II-alpha inhibitors are, for example, reported in Published PCT application WO2020/0205991, published Oct. 8, 2020, and its priority document U.S. provisional application 62/827,818, filed Apr. 1, 2019. Each of these references is incorporated by reference herein in its entirety for descriptions of types of topoisomerase inhibitors, specific topoisomerase inhibitors, mechanisms of topoisomerase inhibition, cancers treated using topoisomerase inhibitors and combination therapies using topoisomerase inhibitors.

Various platinum-based antineoplastic agents (also called platins) are known in the art and have been employed clinically or are in clinical trials. [See, for example, Wheate et al., 2010] This reference is incorporated by reference herein in its entirety for descriptions of types of platinum-based antineoplastic agents, specific platinum-based antineoplastic agents, mechanisms of action of such agents, cancers treated using such agents and combination therapies using platinum-based antineoplastic agents. In embodiments, platinum-based antineoplastic agents useful in methods and compostions herein include cisplatin, carbon platin, oxaliplatin, nedaplatin, lobaplatin, or heptaplatin. In embodiments, platinum-based antineoplastic agents include satraplatin, or picoplatin. Platinum-based antineoplastic agents may be liposomally encapsulated (e.g., Lypoplatin™) or bound in nanopolymers (e.g., ProLindac™) Various thymidylate synthase inhibitors are known in the art and have been employed clinically particularly in the treatment of CRC [Papamichael, 2009; Lehman, 2002]. Thymidylate synthase inhibitors useful in the methods and compostions herein include without limitation folate analogues and nucleotide analogues. In specific embodiments, the thymidylate synthase inhibitor is raltitrexed, pemetrexed, nolatrexed or ZD9331. In more specific embodiments, the thymidylate synthase inhibitor is 5-fluorouracil or capecitabine.

The invention provides CHD1L inhibitors of the following formulas: Compounds useful in the methods, pharmaceutical compositions or pharmaceutical combinations of this invention include those of formula I:

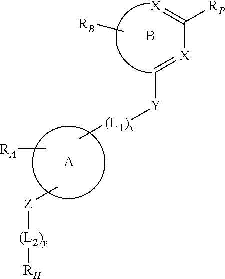

or salts, or solvates thereof,
where:
the B ring is an optionally-substituted at least divalent heteroaryl ring or ring system having one, two or three 5- or 6-member rings, any two or three of which can be fused rings, where the rings are carbocyclic, heterocyclic, aryl or heteroaryl rings and at least one of the rings is heteroaryl; in the B ring, each X is independently selected from N or CH and at least one X is N; R_{P} is an optionally-substituted primary or secondary amine group [—N(R₂)(R₃)] or is a -(M)ₓ-P group, where P is —N(R₂)(R₃) or an aryl or heteroaryl group, where x is 0 or 1 to indicate the absence or present of M and M is an optionally substituted linker —(CH₂)ₙ— or —N(R)(CH₂)ₙ—, where each n is independently an integer from 1-6 (inclusive);
Y is a divalent atom or group selected from the group consisting of —O—, —S—, —N(R₁)—, —CON(R₁)—, —N(R₁)CO—, —N(R₁)CON(R₁)—, —SO₂N(R₁)—, or —N(R₁)SO₂—;
L₁ is an optional 1-4 carbon linker that is optionally substituted and is saturated or contains a double bond (which can be cis or trans), where x is 0 or 1 to indicate the absence or presence of L₁;
the A ring is an optionally-substituted at least divalent carbocyclic or heterocyclic ring or ring system having one, two or three rings, two or three of which can be fused, each ring having 3-10 carbon atoms and optionally 1-6 heteroatoms and wherein each ring is optionally saturated, unsaturated or aromatic;
Z is a divalent group containing at least one nitrogen substituted with a R′ group,
where in embodiments, Z is a divalent group selected from —N(R′)—, —CON(R′)—, —N(R′)CO—, —CSN(R′)—, —N(R′)CS—, —N(R′)CON(R′)—, —SO₂N(R′)—, —N(R′)SO₂—, —CH(CF₃)N(R′)—, —N(R′)CH(CF₃)—, —N(R′)CH₂CON(R′)CH₂—, —N(R′)COCH₂N(R′)CH₂—,

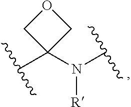

or the divalent Z group comprises a 5- or 6-member heterocyclic ring having at least one nitrogen ring member, for example,

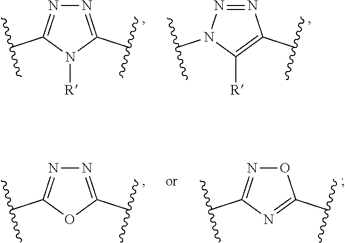

L2 is an optional 1-4 carbon linker that is optionally substituted and is saturated or contains a double bond (which can be cis or trans), where z is 0 or 1 to indicate the absence or presence of L₂;

R is selected from the group consisting of hydrogen, an aliphatic group, a carbocyclyl group, an aryl group, a heterocyclyl group and a heteroaryl group, each of which groups is optionally substituted;

each R′ is independently selected from the group consisting of hydrogen, an aliphatic group, a carbocyclyl group, an aryl group, a heterocyclyl group and a heteroaryl group, each of which groups is optionally substituted;

R₁ is selected from the group consisting of hydrogen, an aliphatic group, a carbocyclyl group, an aryl group, a heterocyclyl group and a heteroaryl group, each of which groups is optionally substituted;

R₂ and R₃ are independently selected from the group consisting of hydrogen, an aliphatic group, a carbocyclyl group, an aryl group, a heterocyclyl group and a heteroaryl group, each of which groups is optionally substituted or

R₂ and R₃ together with the N to which they are attached form an optionally substituted 5- to 10-member heterocyclic ring which is a saturated, partially unsaturated or aromatic ring;

R_{A} and R_{B} represent hydrogens or 1-10 non-hydrogen substituents on the indicated A and B ring or ring systems, respectively, wherein R_{A} and R_{B} substituents are independently selected from hydrogen, halogen, hydroxyl, cyano, nitro, amino, mono- or disubstituted amino (—NR_{C}R_{D}), alkyl, alkenyl, cycloalkyl, cycloalkenyl, aryl, heterocyclyl, alkoxy, acyl, haloalkyl, —COOR_{C}, —OCOR_{C}, —CONR_{C}R_{D}, —OCONR_{C}R_{D}, —NR_{C}COR_{D}, —SR_{C}, —SOR_{C}, —SO₂R_{C}, and —SO₂NR_{C}R_{D}, where alkyl, alkenyl, cycloalkyl, cycloalkenyl, aryl, heterocyclyl, alkoxy, and acyl, are optionally substituted;

each R_{C} and R_{D} is selected from hydrogen, alkyl, alkenyl, cycloalkyl, cycloalkenyl, heterocyclyl, aryl, or heteroaryl, each of which groups is optionally substituted with one or more halogen, alkyl, alkenyl, haloalkyl, alkoxy, aryl, heteroaryl, heterocyclyl, aryl-substituted alkyl, or heterocyclyl-substituted alkyl; and

R_{H} is an optionally substituted aryl or heteroaryl group;

wherein optional substitution includes, substitution with one or more halogen, nitro, cyano, amino, mono- or di-C1-C3 alkyl substituted amino, C1-C3 alkyl, C2-C4 alkenyl, C3-C6 cycloalkyl, C3-C6-cycloalkenyl, C1-C3 haloalkyl, C1-C6 acyl. C1-C6 acyloxy, C1-C6 alkoxylcarbonyl. C6-C12 aryl, C5-C12 heteroaryl, C3-C12 heterocyclyl. C1-C3 alkoxy, C1-C6 acyl, —COOR_{E}, —OCOR_{E}, —CONR_{E}R_{F}, —OCONR_{E}R_{D}, —NR_{E}COR_{F}, —SR_{E}, —SOR_{E}, —SO₂R_{E}, and —SO₂NR_{E}R_{F}, where alkyl, alkenyl, cycloalkyl, cycloalkenyl, aryl, heterocyclyl, alkoxy, and acyl, are optionally substituted and

each R_{E} and R_{F} is selected from hydrogen, C1-C3 alkyl, C2-C4 alkenyl, C3-C6 cycloalkyl, C3-C6-cycloalkenyl, C1-C3 haloalkyl, C6-C12 aryl, C5-C12 heteroaryl, C3-C12 heterocyclyl. C1-C3 alkoxy, C1-C6 acyl, each of which groups is optionally substituted with one or more halogen, nitro, cyano, amino, mono- or di-C1-C3 alkyl substituted amino, C1-C3 alkyl, C2-C4 alkenyl, C3-C6 cycloalkyl, C3-C6-cycloalkenyl, C1-C3 haloalkyl, C6-C12 aryl, C5-C12 heteroaryl, C3-C12 heterocyclyl. C1-C3 alkoxy, C1-C6 acyloxy, C1-C6 alkoxycarbonyl and C1-C6 acyl.

In embodiments of formula I:

R is selected from hydrogen, alkyl, alkenyl, cycloalkyl, cycloalkenyl, heterocyclyl, aryl, or heteroaryl, each of which groups is optionally substituted;

each R′ is independently selected from hydrogen, alkyl, alkenyl, cycloalkyl, cycloalkenyl, heterocyclyl, aryl, or heteroaryl, each of which groups is optionally substituted;

R₁—R₃ are independently selected from hydrogen, alkyl, alkenyl, cycloalkyl, cycloalkenyl, heterocyclyl, aryl, or heteroaryl, each of which groups is optionally substituted;

One or more of R₁-R₃ is cycloalkyl substituted alkyl, for example, a cyclopropylmethyl, a cyclopentylmethyl, or a cyclohexylmethyl;

R is hydrogen or a C1-C3 alkyl;

each R′ is independently hydrogen or C1-C3 alkyl;

R₁ is hydrogen or C1-C3 alkyl;

R₂ and R₃ are independently selected from hydrogen, or a C1-C3 alkyl; or

R₂ and R₃ together with the N to which they are attached form a 5-7member heterocycliuc ring which is saturated.

In embodiments of formula I, the A ring is divalent and is a single 6-member aromatic ring which can contain 1 or 2 heteroatoms, particularly 1 or 2 nitrogen. In an embodiment, the divalent Aring is 1,4-phenylene or 2,5-pyridylene.

In an embodiment of formula I, the divalent B ring is substituted with at least one electronegative substituent. In an embodiment, the electronegative substituent is a halogen. In an embodiment, the electronegative substituent is a haloalkyl group having 1-3 carbon atoms. In an embodiment, the electronegative substituent is fluorine. In an embodiment, the electronegative substituent is tifluoromethyl (CF₃—). In further embodiments of the forgoing embodiments, x is 1. In related embodiments of formula I, the B ring is substituted with at least one halogen, and x is 1 and L₁ is —CH₂—. In related embodiments of formula I, the B ring is substituted with at least one fluorine, x is 1 and L₁ is —CH₂—.

In an embodiment of formula I, the divalent B ring is substituted with at least one C1-C3 alkyl group. In an embodiment of formula I, the B ring is substituted with at least one methyl group.

In specific embodiments of formula I:

Ring A is an optionally substituted phenylene;

Ring A is an optionally substituted 1,4-disubstituted phenylene

Ring A is an optionally substituted naphthylene;

Ring A is an optionally substituted 2,6-disubstituted naphthylene;

Ring A is an optionally substituted pyridylene;

Ring A is an optionally substituted 2,5-pyridylene;

Ring B is an optionally substituted pyridylene,

Ring B is an optionally substituted pyrimidylene;

Ring B is an optionally substituted pyrazinylene;

Ring B is an optionally substituted triazinylene;

Ring B is an optionally substituted quinazolinylene;

Ring B is an optionally substituted pteridinylene;

Ring B is an optionally substituted quinolinylene;

Ring B is an optionally substituted isoquinolinyenel;

Ring B is an optionally substituted naphthyridinylene;

Ring B is an optionally substituted pyridopyrimidylene;

Ring B is an optionally substituted pyrimidopyridylene;

Ring B is an optionally substituted pryanopyridylene;

Ring B is an optionally substituted pyranopyrimidylene;

Ring B is an optionally substituted purinylene;

Ring B is an optionally substituted 6,8-disubstituted purinylene;

Ring A is an optionally substituted phenylene and Ring B is an optionally substituted pyrimidinylene; or

Ring A is an optionally substituted phenylene and Ring B is an optionally substituted pteridinylene.

In embodiments, R_{A} represents H at all available ring positions.

In embodiments, R_{A} represents one C1-C3 alkyl substituebt at an available ring position.

In embodiments, R_{A} represents one methyl substituebt at an available ring position.

In embodiments, R_{A} represents one halogen substituted at an available ring position.

In embodiments, R_{A} represents one fluorine substituted at an available ring position.

In embodiments, R_{A} represents one C1-C3 haloalkyl substituted at an available ring position;

In embodiments, R_{A} represents one trifluoromethyl group at an available ring position.

In embodiments, R_{B} represents H at all available ring positions.

Preferred A and B ring substitution includes one or more C1-C3 alkyl, C3-C7 cycloalkyl, C4-C10 cycloalkyl substituted alkyl, C2-C4 alkenyl, C1-C3 alkoxy, C1-C3 acyl, a C1-C4 alkoxycarbonyl, a C1-C4 acyloxy, carboxyl, halogen, hydroxyl, C1-C3 haloalkyl, mono- or disubstituted phenyl or mono- or disubstituted benzyl. More specific A and B ring substitution includes methyl, ethyl, isopropyl, cyclopropyl, cyclopropylmethyl, methoxy, ethoxy, phenyl, benzyl, halophenyl, halobenzyl, C1, Br, F, CF₃—, HO—, CF₃O—, CH₃CO—, HOOC—, CH₃OCO— and CHCO—.

In an embodiment, the divalent A ring is other than a phenyl ring or a benzyl ring. In an embodiment, the A ring is other than a phenyl ring. In an embodiment, the A ring is other than an unsubstituted phenyl ring or an unsubstituted benzyl ring. In an embodiment, the A ring is other than an unsubstituted phenyl ring.

In embodiments, the divalent B ring has one of the structures illustrated in Scheme 4, RB1-RB17. In embodiments, the divalent B ring has structure RB2-RB5, wherein R_{B} represents optional substitution as described for formula I. The ring is bonded to R_{P} or Y at positions indicated. More specifically, R_{B} represents optional substitution at ring carbons with one or more of C1-C3 alkyl, halogen or C1-C3 haloalkyl and more specifically C1-C3 fluoroalkyl and more specifically with one or more methyl, trifluoromethyl or fluorine. In embodiments, the divalent B ring has structure RB6, which is bonded to R_{P} or Y are the positions indicated and wherein R_{B} represents optional substitution as described for formula I. More specifically, R_{B} represents optional substitution at ring carbons with one or more of C1-C3 alkyl, halogen or C1-C3 haloalkyl and more specifically C1-C3 fluoroalkyl and more specifically with one or more methyl, trifluoromethyl or fluorine. In embodiments, the B ring is as illustrated in RB7-RB17 which is bonded to RP and bonded to Y at the position indicated and wherein R_{B} represents optional substitution as described for formula I. More specifically, R_{B} represents optional substitution at ring carbons with one or more of C1-C3 alkyl, halogen or C1-C3 haloalkyl and more specifically C1-C3 fluoroalkyl and more specifically with one or more methyl, trifluoromethyl or fluorine. In specific embodiments, the B ring is as shown in RB14-17.

In embodiments, the divalent B ring has structure as shown in Scheme 4, formula RB1, where X¹ and X² are selected from CH and N and at least one of X¹ and X² is N and X³—X⁶ are selected from CH, CH₂, O, S, N and NH where the illustrated B ring is saturated, unsaturated or aromatic, dependent upon choice of 1-X⁶ and R_{B} represents optional substitution as defined for formula I. In embodiments, R_{B} represents hydrogens and the B ring is unsubstituted. In embodiments, R_{B} represents one or more halogen, C1-C3 alkyl, C1-C3 acyl, C1-C3 alkoxy. In embodiments, R_{B} represents one or more F, Cl or Br, methyl, ethyl, acetyl or methoxy or combinations thereof. In embodiments, of formula I the B ring is selected from any of RB2-RB5, as shown in Scheme 4.

In embodiments of formula I:

x is 1 and L₁ is —(CH₂)ₙ—, where n is 1, 2 or 3;

x is 1 and L₁ is —(CH₂)ₙ—, where n is 1 or 2;

x is 0 and L1 is absent;

y is 1 and L₂ is —(CH₂)ₙ—, where n is 1, 2 or 3;

y is 1 and L₂ is —(CH₂)ₙ—, where n is 1 or 2;

y is 1, and L₂ is —CH═CH—;

y is 1, and L₂ is trans —CH═CH—;

both of x and y are 0;

x is 1 and y is 0 and L₁ is —(CH₂)ₙ—, where n is 1 or 2;

y is 1 and x is 0 and L₂ is —(CH₂)ₙ—, where n is 1 or 2; or

both of x and y are 1 and both of L₂ and L₁ are —(CH₂)ₙ—, where n is 1 or 2.

In embodiments of formula I:

Y is —O—, —S—, —N(R₁)—, —CON(R₁)—, —N(R₁)CO— or —N(R₁)CON(R₁)—;

Y is —O—, —S—, —NH—, —CONH—, or —NHCO— or —N(R₁)CON(R₁)—;

Y is —N(R₁)—, —CON(R₁)—, —N(R₁)CO— or —N(R₁)CON(R₁)—;

Y is —N(R₁)—, —CON(R₁)—, or —N(R₁)CO—

Y is —N(R₁)CON(R₁)—;

Y is —N(H)—, —CON(H)—, —N(H)CO— or —N(H)CON(H)—;

Y is —N(H)—, —CON(H)—, or —N(H)CO—

Y is —N(H)CON(H)—;

R₁ is hydrogen, a C1-C3 alkyl or a C1-C3 haloalkyl, particularly C1-C3 fluoroalkyl;

R₁ is hydrogen, a methyl group or CF₃—;

R₁ is hydrogen;

Y is —N(R₁)—, —CON(R₁)—, or —N(R₁)CO— and R₁ is hydrogen, methyl or CF₃—;

Y is —N(R₁)— and R₁ is hydrogen, C1-C3 alkyl or C1-C3 haloalkyl, particularly C1-C3 fluoroalkyl; or

Y is —N(R₁)— and R₁ is hydrogen, methyl or CF₃—.

In embodiments of formula I, both x and y are 0 and Y is —N(R₁)—.

In embodiments of formula I, both x and y are 0 and Y is —NH—.

In embodiments of formula I, both x and y are 0 and Y is —CONH—.

In embodiments of formula 1, both x and y are 0 and Y is —NHCO—.

In embodiments of formula 1, both x and y are 0 and Y is —NHCONH—.

In embodiments of formula I:

Z is —N(R′)—, —CON(R′)—, or —N(R′)CO—;

Z is —CH(CF₃)N(R′)—;

Z is —SO₂N(R′)—;

Z is —N(R′)CON(R′)—;

Z is —N(R′)CH₂CON(R′)CH₂—;

Z is

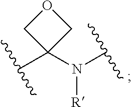

Z is

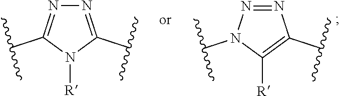

Z is

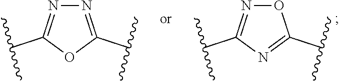 [SMILES: *c1nnc(*)o1 ; *c1noc(*)n1]

R′ is hydrogen, a C1-C6 alkyl or a C1-C3 haloalkyl, particularly a C1-C3 fluoroalkyl;

R′ is hydrogen or a C1-C3 alkyl;

R′ is hydrogen, methyl or CF₃—;

R′ is hydrogen or methyl;

R′ is hydrogen;

Z is —N(R′)—, —CON(R′)—, or —N(R′)CO— and R′ is hydrogen or methyl;

Z is —N(R′)—, —CON(R′)—, —N(R′)CO— or —N(R′)CON(R′) and R′ is hydrogen;

Z is —CON(R′)— or —N(R′)CO— and R′ is hydrogen or methyl;

Z is —N(R′)CON(R′)— and both R′ are hydrogen;

Z is

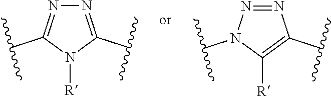

and R′ is hydrogen; or

is

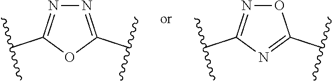 [SMILES: *c1nnc(*)o1 ; *c1noc(*)n1]

and R′ is hydrogen.

In embodiments of formula I,

x is 0;

x is 1 and L₂ is —(CH₂)ₙ—, where n is 1-3;

y is 0, x is 1 and L₂ is —(CH₂)ₙ—, where n is 1-3;

x is 0 and Z is —N(R′)—, —CON(R′)—, —N(R′)CO— or —N(R′)CON(R′)—;

x is 0, and Z is —N(R′)—, —CON(R′)—, —N(R′)CO— or —N(R′)CON(R′)— and R′ is hydrogen or methyl;

x is 0, and Z is —N(H)—, —CON(H)—, —N(H)CO— or —N(H)CON(H)—;

x is 0 and Z is —CON(R′)—;

x is 0, and Z is —CON(R′)— or —N(R′)CO— and R′ is hydrogen or methyl;

x is 0, and Z is —CONH— or —NHCO—;

x is 0 and Z is —CONH—, —NHCO— or —NHCONH—.

x is 1, L₂ is —(CH₂)ₙ—, where n is 1-3, and Z is —N(R₁)—, —CON(R′)—, —N(R′)CO— or —N(R′)CON(R′)—;

x is 1, L₂ is —(CH₂)ₙ—, where n is 1-3, and Z is —NH—, —CONH—, —NHCO— or —NHCONH—;

x is 1, L₂ is —CH₂— and Z is —NH—, —CONH—, —NHCO— or —NHCONH—;

x is 1, L₂ is —CH₂—CH₂—, and Z is —NH—, —CONH—, —NHCO— or —NHCONH—;

x is 1, L₂ is —CH₂—CH₂—CH₂—, and Z is —NH—, —CONH—, —NHCO— or —NHCONH—;

x is 1, L₂ is —(CH₂)ₙ—, where n is 1-3, and Z is —CON(R′)—;

x is 1, L₂ is —CH₂— and Z is —CON(R′)—;

x is 1, L₂ is —CH₂—CH₂— and Z is —CON(R′)—;

x is 1, L₂ is —CH₂— and Z is —CONH—;

x is 1, L₂ is —CH₂—CH₂— and Z is —CONH—;

x is 1, L₂ is —CH₂—CH₂—CH₂— and Z is —CONH—;

x is 0 or 1, L₂, if present, is —CH₂— or —CH₂—CH₂— and Z is —CON(R′)—;

x is 0 or 1, L₂, if present, is —CH₂— or —CH₂—CH₂—, and Z is —CONH—;

x is 0 or 1, L₂, if present, is —CH₂— or —CH₂—CH₂— and Z is —N(R′)CO—;

x is 0 or 1, L₂, if present, is —CH₂— or —CH₂—CH₂—, and Z is —NHCO—;

x is 0 or 1, L₂ if present, is —CH₂— or —CH₂—CH₂—, and Z is —NHCONH—;

y is 0, x is 0 or 1, L₂, if present, is —CH₂— or —CH₂—CH₂—, and Z is —CONH—;

y is 0, Y is —N(R₁)—, x is 0 or 1, L₂, if present, is —CH₂— or —CH₂—CH₂—, and Z is —CONH—;

y is 0, Y is —NH—, x is 0 or 1, L₂, if present, is —CH₂— or —CH₂—CH₂—, Z is —CONH—;

y is 0, Y is —NH—, x is 0 or 1, L₂, if present, is —CH₂— or —CH₂—CH₂—, Z is —CONH—, —NHCO—, or —NHCONH—;

In embodiments of formula I,

R_{P} contains at least one nitrogen; or

when R_{P} is -(M)x-P, and x=0, then P is —N(R₂)(R₃) or P is a heterocyclic or heteroaryl group having at least one ring N; or when R_{P} is -(M)x-P, x=1, and M=—(CH₂)ₙ—, then P is —N(R₂)(R₃) or P is a heterocyclic or heteroaryl group having at least one ring N.

In embodiments of formula I, R_{P} is:

—N(R₂)(R₃);

-(M)-N(R₂)(R₃), where M is an optionally substituted linker —(CH₂)ₙ— or —N(R)(CH₂)ₙ—, where each n is independently an integer from 1-6 (inclusive) and R is hydrogen or an optionally substituted alkyl group having 1-3 carbon atoms;

-(M)-N(R₂)(R₃), M is an optionally substituted linker —(CH₂)ₙ—, where each n is independently an integer from 1-6 (inclusive) and R is hydrogen or an optionally substituted alkyl group having 1-3 carbon atoms;

-(M)-N(R₂)(R₃), M is an optionally substituted linker —(CH₂)ₙ—, where each n is independently an integer from 1-6 (inclusive) and R is hydrogen or an optionally substituted alkyl group having 1-3 carbon atoms, where optional substitution is substitution with one or more halogen or one or more C1-C3 alkyl groups;

(M)-N(R₂)(R₃), M is optionally substituted —N(R)(CH₂)ₙ—, where each n is independently an integer from 1-6 (inclusive and R is hydrogen) and R is hydrogen or an optionally substituted alkyl group having 1-3 carbon atoms;

-(M)-N(R₂)(R₃), M is optionally substituted —N(R)(CH₂)ₙ—, where each n is independently an integer from 1-6 (inclusive) and R is H or an optionally substituted alkyl group having 1-3 carbon atoms, where optional substitution is substitution with one or more halogen or one or more C1-C3 alkyl groups;

-(M)-N(R₂)(R₃), where M is an optionally substituted linker —(CH₂)ⁿ— and n is 1, 2 or 3;

-(M)-N(R₂)(R₃), where M is an optionally substituted linker —N(R)(CH₂)ₙ— and n is 1, 2 or 3;

-(M)-N(R₂)(R₃), where M is —(CH₂)ₙ— and n is 1, 2 or 3;

-(M)-N(R₂)(R₃), where M is —N(R)(CH₂)ₙ— and n is 1, 2 or 3;

-(M)ₓ-P group, where P is a aryl or heteroaryl group, where x is 0 or 1 to indicate the absence or presence of M and M is an optionally substituted linker —(CH₂)ₙ— or —N(R)(CH₂)ₙ—, where each n is independently an integer from 1-6 (inclusive) and R is H or an optionally substituted alkyl group having 1-3 carbon atoms;

-(M)-P group, where P is a aryl or heteroaryl group, and M is an optionally substituted linker —(CH₂)ₙ— or —N(R)(CH₂)ₙ—, where each n is independently an integer from 1-6 (inclusive) and R is H or an optionally substituted alkyl group having 1-3 carbon atoms;

-(M)-P group, where P is a aryl or heteroaryl group, and M is an optionally substituted linker —N(R)(CH₂)ₙ—, where each n is independently an integer from 1-6 (inclusive) and R is H or an optionally substituted alkyl group having 1-3 carbon atoms;

-(M)-P group, where P is a aryl or heteroaryl group, and M is an optionally substituted linker —(CH₂)ₙ—, where each n is independently an integer from 1-3 (inclusive) and R is H or an optionally substituted alkyl group having 1-3 carbon atoms;

P is an optionally substituted phenyl or naphthyl;

P is an optionally substituted phenyl or naphthyl and optional substitution is with one or more halogen, C1-C3 alkyl, C1-C3 alkoxy or C1-C3 haloalkyl;

P is an optionally substituted heteroaryl group having a 5- or 6-member ring or two fused 5- or 6-member rings;

P is an optionally substituted heteroaryl group having a 5- or 6-member ring or two fused 5- or 6-member rings and having 1 to 3 nitrogen ring members;

R₂ in R_{P} is hydrogen (i.e., —N(R₂)(R₃) is a primary amine group);

both R₂ and R₃ in R_{P} are groups other than hydrogen (i.e., —N(R₂)R₃) is a secondary amine group);

R₂ is hydrogen and R₃ is an optionally substituted 3-8-member cycloalkyl group;

R₂ is hydrogen and R3 is a C1-C3 alkyl group substituted with a 3-8-member cycloalkyl group;

R₂ is hydrogen and R₃ is an optionally substituted aryl group having 6-12 carbon atoms;

R₂ is hydrogen and R₃ is an optionally substituted heteroaryl group having 6-12 carbon atoms and 1-3 heteroatoms (N, O, or S);

R₂ is hydrogen and R₃ is an optionally substituted heteroaryl group having 6-12 carbon atoms and 1-3 ring nitrogens;

R₂ and R₃ together with the N to which they are attached form an optionally substituted 5- to 10-member heterocyclic ring which is a saturated, partially unsaturated or aromatic ring;

R₂ and R₃ together with the N to which they are attached form a 5- to 10-member heterocyclic sultam ring;

R_{P} is —(CH₂)ₙ—N(R₂)(R₃), where n is 1 or 2 and R₂ and R₃ together with the N to which they are attached form an optionally substituted 5- to 10-member heterocyclic ring which is a saturated, partially unsaturated or aromatic ring;

R_{P} is —N(R)(CH₂)ₙ—N(R₂)(R₃), where n is 1 or 2, R is hydrogen or methyl and R₂ and R₃ together with the N to which they are attached form an optionally substituted 5- to 10-member heterocyclic ring which is a saturated, partially unsaturated or aromatic ring;

R_{P} is -M-N(R₂)(R₃) and R₂ and R₃ together with the N to which they are attached form an optionally substituted 5- to 10-member heterocyclic ring which is a saturated, partially unsaturated or aromatic ring;

R₂ and R₃ together with the N to which they are attached form an optionally substituted 5- to 10-member heterocyclic ring which contains no double bonds;

R_{P} is —N(R₂)(R₃) and R₂ and R₃ together with the N to which they are attached form an optionally substituted 5- to 10-member heterocyclic ring which contains no double bonds;

R₂ and R₃ together with the N to which they are attached form an optionally substituted 5- to 10-member heterocyclic ring which contains one, two or three double bonds;

R_{P} is —N(R₂)(R₃) and R₂ and R₃ together with the N to which they are attached form an optionally substituted 5- to 10-member heterocyclic ring which contains one, two or three double bonds;

R₂ and R₃ together with the N to which they are attached form an optionally substituted 5- to 10-member heteroaryl ring; or

R_{P} is —N(R₂)(R₃) and R₂ and R₃ together with the N to which they are attached form an optionally substituted 5- to 10-member heteroaryl ring.

In specific embodiments of formula I, R_{P} or —N(R₂)(R₃) is:

any one of R_{N}1-R_{N}39 of Scheme 2;

R_{N}1; R_{N}3; R_{N}2 or R_{N}4; R_{N}5 or R_{N}6; R_{N}7 or R_{N}8; R_{N}9; R_{N}10; R_{N}11; R_{N}12; R_{N}13; R_{N}14; R_{N}15; R_{N}16; R_{N}17 or R_{N}18; R_{N}19 or R_{N}20; R_{N}21; R_{N}22; R_{N}23 or R_{N}24; R_{N}25; R_{N}26-R_{N}29; R_{N}27-R_{N}32; R_{N}30; R_{N}31; R_{N}33-R_{N}36; R_{N}37; R_{N}38; R_{N}39; or

R_{N}1, R_{N}2, R_{N}3, R_{N}4, R_{N}11, R_{N}13, or R_{N}14; or

R_{N}1-R_{N}31 which is unsubstituted.

In embodiments of formula I, R_{H} is:

optionally substituted phenyl; other than optionally substituted pheny; unsubstituted phenyl; other than unsubstituted pheny; optionally substituted naphthyl; unsubstituted naphthyl; optionally substituted naphthy-2-yl; optionally substituted naphthy-1-yl; naphthy-2-yl; naphthy-1-yl; optionally substituted thiophenyl; halogen substituted thiophenyl; bromine substituted thiophenyl; optionally substituted thiophen-2-yl; halogen substituted thiophen-2-yl; bromine substituted thiophen-2-yl; 4-halothiophen-2-yl; 4-bromothiophen-2-yl; optionally substituted furyl; optionally substituted fur-2-yl; optionally substituted indolyl; unsubstituted indolyl; indol-3-yl; indol-2-yl; indol-1-yl; optionally substituted pyridinopyrrolyl; optionally substituted pyridinopyrrol-2-yl; optionally substituted pyridinopyrrolyl; optionally substituted pyridinopyrrol-3-yl; optionally substituted quinolinyl; optionally substituted quinolin-4-yl; optionally substituted isoquiolinyl; optionally substituted isoquinolin-4-yl, optionally substituted benzoimidazolyl; optionally substituted benzoimidazol-1-yl; optionally substituted 1H-pyrrolo[2,3-b]pyridin-3-yl: optionally substituted pyridine-2-yl; optionally substituted pyridine-3-yl; optionally substituted pyridine-4-yl; 1 H-imidazol-1-yl, 1 H-imidazol-2-yl; or 1 H-imidazole-5-yl.

In specific embodiments, optional substitution of R_{H} is substitution with one or more halogen, C1-C3 alkyl, C1-C3 alkoxyl, C1-C3 haloalkyl, C1-C3 fluoroalkyl, C4-C7 cycloalkylalkyl, OH, amino, C1-C6 acyl, —COOR_{E}, —OCOR_{E}, —CONR_{E}R_{F}, —OCONR_{E}R_{D}, —NR_{E}COR_{F}, —SR_{E}, —SOR_{E}, —SO₂R_{E}, and —SO₂NR_{E}R_{F}, where R_{E} and Rₑ are as defined above and in particular are hydrogen, C1-C3 alkyl, phenyl or benzyl. More specifically, optional substitution of R_{H} is substitution with one or more halogen (particularly Br or Cl), C1-C3 alkyl, C1-C3 alkoxyl, C1-C3 fluoroalkyl (particularly CF₃—).

In embodiments, R_{H} has formula:

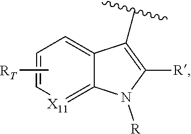

where:

X₁₁ is CH, CR_{T} or N; R_{T} is optional R_{H} ring substitution as described above and R and R′ are independently hydrogen, C1-C6 alkyl group, C4-C10 cycloalkylalkyl group, aryl group, heterocyclyl group, or heteroaryl group each of which groups are optionally substituted. In specific embodiments, R_{T} is hydrogen or substitution with one or more of halogen, OH, C1-C3 alkyl, C1-C3 alkoxy, or C1-C3 alkyl substituted with a C3-C6 cycloalkyl; R′ is hydrogen, C1-C3 alkyl, C1-C3 alkoxy, or C1-C3 alkyl substituted with a C3-C6 cycloalkyl; and R is hydrogen, C1-C3 alkyl, C1-C3 alkoxy, or C1-C3 alkyl substituted with a C3-C6 cycloalkyl.

In embodiments, R_{H} has formula:

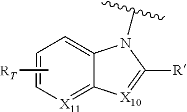

where:

X₁₁ is CH, CR_{T} or N; X₁₀ is CH, CR_{T} or N; R_{T} is R_{H} ring optional substitution as described above and R and R′ are independently hydrogen, C1-C6 alkyl group, C4-C10 cycloalkylalkyl group, aryl group, heterocyclyl group, or heteroaryl group each of which groups are optionally substituted. In specific embodiments, R_{T} is hydrogen or substitution with one or more of halogen, OH, C1-C3 alkyl, C1-C3 alkoxy, or C1-C3 alkyl substituted with a C3-C6 cycloalkyl; R′ is hydrogen, C1-C3 alkyl, C1-C3 alkoxy, or C1-C3 alkyl substituted with a C3-C6 cycloalkyl; and R is hydrogen, C1-C3 alkyl, C1-C3 alkoxy, or C1-C3 alkyl substituted with a C3-C6 cycloalkyl.

In embodiments, R_{H} in formula I or R12 in formula XX is selected from any one of formulas R12-1 to R12-84. In embodiments, R_{H} is selected from the following formulas in Scheme 3:

R12-79 or R12-80; or

R12-81-R12-84; or

R12-70, R12-71, or R12-75-R12-78; or

R12-3, R12-4, R12-5, R12-7, R12-8, R12-10, R12-23, R12-25, R12-27, R12-29, or R12-31; or R12-12, R12-13, R2-145, R12-15, R12-16, R12-17, R12-18, R12-19, R12-20, R12-21, R12-21 or R12-22, where p is 0; or

R12-33, R12-34, R12-35, R12-36, R12-37, R12-38, R12-39 R12-40, R12-41, R12-42, where p is 0; or

R12-70 or R12-71, where p is 0; or

R12-75, R12-76, R12-77 or R12-78, where p is 0.

In embodiments, R_{H} is selected from 5-membered heterocyclic groups of general formula:

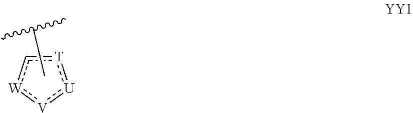

where:

T, U, V, and Ware selected from O, S, C(R″)(R″), C(R″)-/, C(R″), C-/, N(R″), or N-/;

where the group contains one or two double bonds dependent upon choice of T, U, V, and W;

where the R_{H} group is bonded to the -(L₂)y-Z-moiety in the compound of formula I through C-/, C(R″)-/, or N-/; and

where R″ indicates optional substitution on N or C.

More specifically, R_{H} is selected from 5-membered heterocyclic groups of formula:

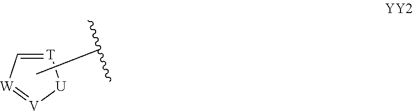

where:

T is C(R″), C-/, or N; or

U is O, S, C(R″)(R″), C(R″)-/, N(R″), or N-/;

V is CR″, C-/, or N and

W is CR″, C-/, N, where the R_{H} group is bonded to the -(L₂)y-Z-moiety in the compound of formula I through C-/, C(R″)-/, or N-/,

where the R_{H} group is bonded to the -(L₂)y-Z-moiety in the compound of formula I through C-/, C(R″)-/, or N-/; and

where R″ indicates optional substitution on N or C. The symbol “-/” indicates a monovalent bond through which the heterocyclic group is bonded in the compounds herein e.g., C-/ indicates a monovalent bond from a ring carbon through which the heterocyclic group is bonded into compounds herein.

In embodiments, R_{H} is a fused ring heterocyclic group of formula:

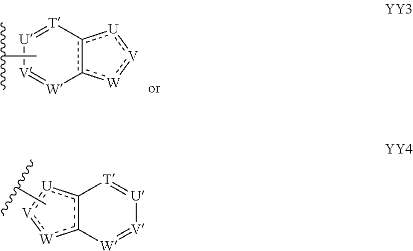

where:

U, V and W are selected from O, S, N, C(R″)(R″), C(R″)-/, C(R″), C-/, N(R″), or N-/;

T, U′, V and W are selected from C(R″), C-/, N(R″), or N-/;

where the R_{H} group is bonded to the -(L₂)y-Z-moiety in the compound of formula I through C-/, C(R″)-/, or N-/ in the indicated ring;

where the group contains bonds dependent upon choice of, U, V, and W; and

where R″ indicates optional substitution on N or C.

More specifically, R_{H} is a fused heterocyclic group of formula:

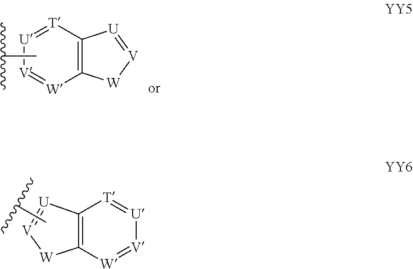

where:

U, and V are selected from N, C(R″), or C-/,/;

W is selected from O, S, C(R″)(R″), C(R″)-/, N(R″), or N-/;

T′, U′, V and W are selected from C(R″), C-/, N(R″), or N-/;

where the R_{H} group is bonded to the -(L₂)y-Z-moiety in the compound of formula I through C-/, C(R″)-/, or N-/ in the indicated ring; and

where R″ indicates optional substitution on N or C.

Each R″, independently, is selected from hydrogen, halogen, nitro, cyano, amino, mono- or di-C1-C3 alkyl substituted amino, C1-C3 alkyl, C2-C4 alkenyl, C3-C6 cycloalkyl, C3-C6-cycloalkenyl, C1-C3 haloalkyl, C6-C12 aryl, C5-C12 heteroaryl, C3-C12 heterocyclyl. C1-C3 alkoxy, C1-C6 acyl, —COOR_{E}, —OCOR_{E}, —CONR_{E}R_{F}, —OCONR_{E}R_{D}, —NR_{E}COR_{F}, —SR_{E}, —SOR_{E}, —SO₂R_{E}, and —SO₂NR_{E}R_{F}, where alkyl, alkenyl, cycloalkyl, cycloalkenyl, aryl, heterocyclyl, alkoxy, and acyl, are optionally substituted;

where each R_{E} and R_{F} is selected from hydrogen, C1-C3 alkyl, C2-C4 alkenyl, C3-C6 cycloalkyl, C3-C6-cycloalkenyl, C1-C3 haloalkyl, C6-C12 aryl, C5-C12 heteroaryl, C3-C12 heterocyclyl. C1-C3 alkoxy, C1-C6 acyl, each of which groups is optionally substituted with one or more halogen, nitro, cyano, amino, mono- or di-C1-C3 alkyl substituted amino, C1-C3 alkyl, C2-C4 alkenyl, C3-C6 cycloalkyl, C3-C6-cycloalkenyl, C1-C3 haloalkyl, C6-C12 aryl, C5-C12 heteroaryl, C3-C12 heterocyclyl. C1-C3 alkoxy, and C1-C6 acyl.

In more specific embodiments, R_{H} is selected from any one of:

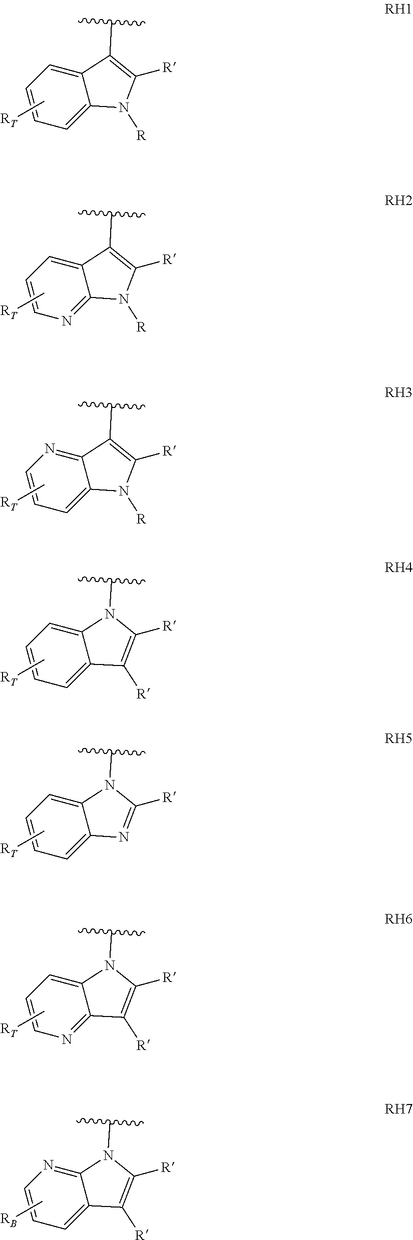

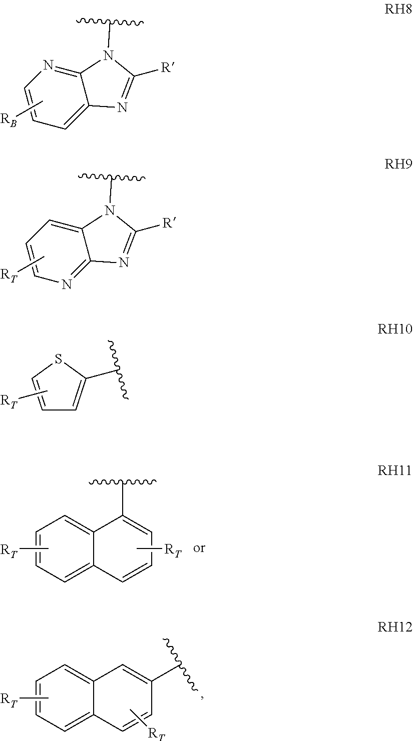

where:

R_{T} is R_{H} ring optional substitution as described above and R and R′ are independently hydrogen, C1-C6 alkyl group, C4-C10 cycloalkylalkyl group, aryl group, heterocyclyl group, or heteroaryl group each of which groups are optionally substituted. In specific embodiments, R_{T} is hydrogen or substitution with one or more of halogen, OH, C1-C3 alkyl, C1-C3 alkoxy, or C1-C3 alkyl substituted with a C3-C6 cycloalkyl; R′ is hydrogen, C1-C3 alkyl, C1-C3 alkoxy, or C1-C3 alkyl substituted with a C3-C6 cycloalkyl; and R is hydrogen, C1-C3 alkyl, C1-C3 alkoxy, or C1-C3 alkyl substituted with a C3-C6 cycloalkyl. In specific embodiments, R and R′ are independently hydrogen, C1-C3 alkyl or C4-C7 cycloalkylalkyl. In specific embodiments, R_{T} represents hydrogens or substitution with one halogen, particularly Br.

In embodiments, R_{H} is a 6-member optionally substituted heterocyclic or heteroaryl group having 1-3 nitrogen in the ring, 1 or 2 oxygens, sulfurs or both in the ring, or 1 or 2 nitrogens and one oxygen or sulfur in the ring, where optional substitution is defined as in formula I. The heterocyclic group can be unsaturated, partially unsaturated or a heteroaryl group.

In embodiments, R_{H} is an optionally substituted fused heterocyclic or heteroaryl group having two fused 6-member rings having 1-5 nitrogens in the fused rings, 1-3 oxygens, sulfurs or both in the fused rings or 1-4 nitrogens and 1 or 2 oxygens, sulfurs or both in the fused rings, where optional substitution is defined as in formula I. In more specific embodiments, the fused rings have 1, 2, 3 or 4 nitrogens in the fused rings. In more specific embodiments, the fused rings have 1 or 2 oxygens or sulfurs in the fused rings. In more specific embodiments the fused rings have 1 or 2 nitrogens and one oxygen or sulfur in the fused rings. The fused ring heterocyclic group can be unsaturated, partially unsaturated or a heteroaryl group.

In specific embodiments, the R_{H} group is selected from phenyl, oxazinyl, pyridinyl, pyrimidinyl, thionyl, pyranyl, thiazinyl, 4H-pyranyl, naphthyl, quinolinyl, isoquinolinyl, quinoxalinyl, quinazolinyl, pteridinyl, purinyl and chromanyl, where the R_{H} group is attached to the -(L₂)y-Z-moiety in the compound of formula I at any available ring position. In specific embodiments, the R_{H} group is attached to the -(L₂)y-Z-moiety in the compound of formula I at a carbon in the ring.

In embodiments of formula I, R_{P} is selected from the group of moieties R_{N}1, R_{N}2, R_{N}3, R_{N}9, R_{N}10, R_{N}11, R_{N}13, R_{N}14, R_{N}36, R_{N}37, R_{N}38 or R_{N}39 and R_{H} is selected from the group of moieties R12-3, R12-5, R12-44. R13-45, R12-48, R12-58, R12-70, R12-72, R12-73, R12-75, R12-79, R12-80, R12-82, R12-83, or R12-84. In embodiments of formula I, R_{P} is selected from the group of moieties R_{N}1, R_{N}2, R_{N}3, R_{N}9, R_{N}10, R_{N}11, R_{N}13, R_{N}14, R_{N}36, R_{N}37, R_{N}38 or R_{N}39, R_{H} is selected from the group of moieties R12-3, R12-5, R12-44. R13-45, R12-48, R12-58, R12-70, R12-72, R12-73, R12-75, R12-79, R12-80, R12-82, R12-83, or R12-84, and the A ring is unsubstituted 1,4-phenylene or 2,5-pyridylene. In embodiments of formula I, Re is selected from the group of moieties R_{N}1, R_{N}2, R_{N}3, R_{N}9, R_{N}10, R_{N}11, R_{N}13, R_{N}14, R_{N}36, R_{N}37, R_{N}38 or R_{N}39, R_{H} is selected from the group of moieties R12-3, R12-5, R12-44. R13-45, R12-48, R12-58, R12-70, R12-72, R12-73, R12-75, R12-79, R12-80, R12-82, R12-83, or R12-84, the A ring is unsubstituted 1,4-phenylene or 2,5-pyridylene, Y is —NH—, —CONH, —NH—CO— or —NH—CO—NH— and x is 0 or 1 and L₁, if present, is —(CH₂)—. In embodiments of formula I, R_{P} is selected from the group of moieties R_{N}1, R_{N}2, R_{N}3, R_{N}9, R_{N}10, R_{N}11, R_{N}13, R_{N}14, R_{N}36, R_{N}37, R_{N}38 or R_{N}39, R_{H} is selected from the group of moieties R12-3, R12-5, R12-44. R13-45, R12-48, R12-58, R12-70, R12-72, R12-73, R12-75, R12-79, R12-80, R12-82, R12-83, or R12-84, the A ring is unsubstituted 1,4-phenylene or 2,5-pyridylene, Y is —NH—, —CONH, —NH—CO— or —NH—CO—NH—, x is 0 or 1, L₁, if present, is —(CH₂)—, Z is —CONH, —NH—CO— or —NH—CO—NH—, y is 0 or 1 and L₂, if present is —(CH₂)—. In more specific embodiments of the forgoing embodiments, the A ring is unsubstituted 1,4-phenylene. In more specific embodiments of the forgoing embodiments, Y is —NH—. In more specific embodiments of the forgoing embodiments, Z is —CONH—. In more specific embodiments of the forgoing embodiments, y is 1. In more specific embodiments of the forgoing embodiments, x is 1.

In specific embodiments of formula I, —Z-(L₂)y-R_{H} is a group other than —NH—SO₂—R_{W}, where R_{W} is R₁ is mes-trimethylphenyl, 4-methylphenyl, 4-trifluoromethylphenyl, naphthyl, 2,3,4,5, -tetramethylphenyl, 4-methoxyphenyl, 4-tert-butylphenyl, 2,4-dimethoxyphenyl, 2,5-dimethoxyphenyl or 4-phenoxypheny. In specific embodiments of formula I, —Z-(L₂)y- is a moiety other than —NR_{X}—SO₂—, where R_{X} is H, hydrogen, methyl acetate, acetate, aminoacetyl, 4-formic acid benzyl, 4-isopropylbenzyl, 4-chlorobenzyl or 4-methoxybenzyl. In embodiments of formula I, —Z— is other than —NR_{X}—SO₂—, where R_{X} is H, hydrogen, methyl acetate, acetate, aminoacetyl, 4-formic acid benzyl, 4-isopropylbenzyl, 4-chlorobenzyl or 4-methoxybenzyl.

In embodiments of formula I, R_{H} is other than a phenyl group or an optionally substituted phenyl group. I_{N} embodiments of formula I, R_{H} is a heterocyclic group that is substituted with a single halogen, particularly a Br.

In embodiments of formula I, R_{P} or —N(R₂)(R₃) are optionally substituted amine groups illustrated in Scheme 2, R_{N}1-R_{N}39. Exemplary optional substitution of groups is illustrated in Scheme 2. The illustrated R substituent groups can be positioned on any available ring position. In the moieties of Scheme 2, preferred alkyl are C1-C3 alkyl, acyl includes formyl, preferred acyl are C1-C6 acyl and more preferably acetyl, acyloxy are preferably C1-C4 acyloxy, alkoxycarbonyl are preferably C2-C5 alkoxycarbonyl, hydroxyalkyl are C1-C6 hydroxyalkyl and preferably are —CH₂—CH₂—OH, for amine groups, preferred alkyl are C1-C3 alkyl, preferred alkyl for —SO₂alkyl are C1-C3 alkyl and more preferred is methyl.

In specific embodiments of formula I, —N(R₂)(R₃) is R_{N}1. In specific embodiments, —N(R₂)(R₃) is R_{N}3. In specific embodiments, —N(R₂)(R₃) is R_{N}2 or R_{N}4. In specific embodiments, —N(R₂)(R₃) is R_{N}5 or R_{N}6. In specific embodiments, —N(R₂)(R₃) is R_{N}7 or R_{N}8. In specific embodiments, —N(R₂)(R₃) is R_{N}9. In specific embodiments, —N(R₂)(R₃) is R_{N}10. In specific embodiments, —N(R₂)(R₃) is R_{N}11. In specific embodiments, —N(R₂)(R₃) is R_{N}12. In specific embodiments, —N(R₂)(R₃) is R_{N}13. In specific embodiments, —N(R₂)(R₃) is R_{N}14. In specific embodiments, —N(R₂)(R₃) is R_{N}15. In specific embodiments, —N(R₂)(R₃) is R_{N}16. In specific embodiments, —N(R₂)(R₃) is R_{N}17 or R_{N}18. In specific embodiments, —N(R₂)(R₃) is R_{N}19 or R_{N}20. In specific embodiments, —N(R₂)(R₃) is R_{N}21. In specific embodiments, —N(R₂)(R₃) is R_{N}22. In specific embodiments, —N(R₂)(R₃) is R_{N}23 or R_{N}24. In specific embodiments, —N(R₂)(R₃) is R_{N}25. In an embodiment, —N(R₂)(R₃) is R_{N}1, R_{N}2, R_{N}3, R_{N}4, R_{N}11, R_{N}13, or R_{N}14. In an embodiment, —N(R₂)(R₃) is R_{N}26-R_{N}29. In an embodiment, —N(R₂)(R₃) is R_{N}30. In an embodiment, —N(R₂)(R₃) is R_{N}31.

In embodiments of formula I, R_{H} is a moiety illustrated in Scheme 3 R12-1 to R12-78. In embodiments of formula I, R_{H} is a moiety illustrated in Scheme 3 R12-1 to R12-69. In embodiments of formula I, R_{H} is a moiety illustrated in Scheme 3 R12-1 to R12-71. In embodiments of formula I, R_{H} is a moiety illustrated in Scheme 3 R12-72 to R12-78. In an embodiment, R_{H} is R12-35-R12-42. In embodiments, R_{H} is any of R12-43-R12-69. In embodiments, R_{H} is any of R12-43-R12-45. In embodiments, R_{H} is any of R12-46-R12-48. In embodiments, R_{H} is any of R12-49-R12-51. In embodiments, R_{H} is any of R12-52-R12-54. In embodiments, R_{H} is any of R12-55-R12-58. In embodiments, R_{H} is any of R12-59-R12-62 In embodiments, R_{H} is any of R12-63-R12-66. In embodiments, R_{H} is any of R12-67-R12-69. In embodiments, R_{H} is R12-72 or R12-73. In embodiments, R_{H} is R12-74. In embodiments, R_{H} is t12-75 or R12-76. In embodiments, R_{H} is R12-77. In embodiments, R_{H} is R12-78. In moieties of Scheme 3, preferred alkyl groups are C-C6 alkyl groups or more preferred C1-C3 alkyl groups, preferred halogen are F, Cl and Br, acyl includes formyl and preferred acyl are —CO—C1-C6 alky and more preferred is acetyl, phenyl is optionally substituted with one or more halogen, alkyl or acyl. More preferred alkyl are methyl, ethyl. Methyl cyclopropyl and cyclopropyl. More preferred halogen are Cl and Br.

In specific embodiments, compounds useful in the methods herein include those of formula II:

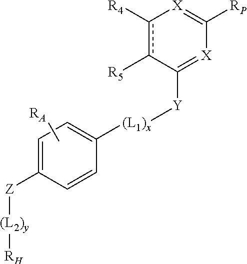

or salts, or solvates thereof, where both X, R_{P}, Y, x, L₁, R_{A}, Z, y, L₂ and R_{H} are as defined in formula I, R₄ and R₅ are independently selected from hydrogen, halogen, alkyl group, alkenyl group, cycloalkyl group, cycloalkenyl group, or heterocyclyl group, each of which groups is optionally substituted or

R₄ and R₅ together form an optionally substituted 5- or 6-member heterocyclic ring which can contain one or two double bonds or be aromatic; and

the dotted line is a single or double bond dependent upon choice of R₄ and R₅.

In embodiments, x is 1, and y is 1. In embodiments, both X are nitrogens. In embodiments, R_{P} is —N(R₂)(R₃). In embodiments, L₁ and L₂ are —(CH₂)ₙ—, where n are independently is 1, 2 or 3. In embodiments, R_{H} is a heterocyclic or heteroaryl group. In embodiments, Y is —N(R₁)—, —CON(R₁)—, or —N(R₁)CO—. In embodiments, Z is —CON(R′)— or —N(R′)CO—. In embodiments, R′ is hydrogen, a C1-C3 alkyl or a C1-C3 haloalky. In embodiments, R′ is hydrogen, methyl or trifluoromethyl. In embodiments, R_{A} is hydrogen, halogen C1-C3 alkyl, C1-C3 alkoxyl, C1-C3 acyl, or C1-C3 haloalkyl. In embodiments, R′ is hydrogen, methyl, methoxy or trifluoromethyl. In embodiments, R₄ and R₅ are selected from hydrogen, halogen, C1-C3 alkyl, C1-C3 alkoxyl, or C1-C3 haloalkyl. In embodiments, R₄ and R₅ together form a 5- or 6-member carbocyclic or heterocyclic ring which is saturated, partially unsaturated or is heteroaromatic. In embodiments, R_{H} is any one of RH1-RH12.

In embodiments of formula II, Y is NH. In embodiments of formula II, Y is NH, and x is 0. In embodiments of formula II, Y is NH, x is 0 and R₅ is other than an electronegative group. In embodiments of formula II, Y is NH, x is 0 and R₅ is hydrogen. In embodiments of formula II, Y is NH, x is 0, R₅ is hydrogen and R₄ is a C1-C3 alkyl. In embodiments of formula II, Y is NH, x is 0, R₅ is hydrogen and R₄ is methyl.

In embodiments of formula II, Y is NH, x is 1 and L₁ is —(CH₂)ₙ—, where n is 1 or 2. In embodiments of formula II, Y is NH, x is 1 and L₁ is —(CH₂)ₙ—, where n is 1 or 2, and R₅ is an electronegative group. In embodiments of formula II, Y is NH, x is 1 and L₁ is —(CH₂)ₙ—, where n is 1 or 2, and R₅ is a halogen. In embodiments of formula II, Y is NH, x is 1 and Li is —(CH₂)—, and R₅ is a halogen. In embodiments of formula II, Y is NH, x is 1 and L₁ is —(CH₂)ₙ—, where n is 1 or 2, and R₅ is a fluorine. In embodiments of formula II, Y is NH, x is 1 and L₁ is —(CH₂)—, and R₅ is a fluorine. In embodiments of formula II, Y is NH, x is 1 and L₁ is —(CH₂)—, R₅ is a halogen and R₄ is C1-C3 alkyl. In embodiments of formula II, Y is NH, x is 1 and L₁ is —(CH₂)—, R₅ is a halogen and R₄ is methyl. In embodiments of formula II, Y is NH, x is 1 and L₁ is —(CH₂)—, R₅ is a fluorine and R₄ is C1-C3 alkyl. In embodiments of formula II, Y is NH, x is 1 and L₁ is —(CH₂)—, R₅ is a fluorine and R₄ is methyl.

In specific embodiments, compounds useful in the methods herein include those of formula III:

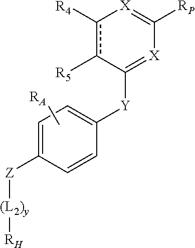

or salts, or solvates thereof,

where variables are as defined in formula I and II and the dotted line represent a single or double bond.

In embodiments, y is 1. In embodiments, y is 0. In embodiments, both X are nitrogens. In embodiments, R_{P} is —N(R₂)(R₃). In embodiments, L₂ is —(CH₂)ₙ—, where n is 1, 2 or 3. In embodiments, R_{H} is a heterocyclyl or heteroaryl group. In embodiments, Y is —N(R₁)—, —CON(R₁)—, or —N(R₁)CO—. In embodiments, Z is —CON(R′)— or —N(R′)CO—. In embodiments, R′ is hydrogen, a C1-C3 alkyl or a C1-C3 haloalky. In embodiments, R′ is hydrogen, methyl or trifluoromethyl. In embodiments, R_{A} is hydrogen, halogen C1-C3 alkyl, C1-C3 alkoxyl, C1-C3 acyl, or C1-C3 haloalkyl. In embodiments, R′ is hydrogen, methyl, methoxy or trifluoromethyl. In embodiments, R₄ and R₅ are selected from hydrogen, halogen, C1-C3 alkyl, C1-C3 alkoxyl, or C1-C3 haloalkyl. In embodiments, R₄ and R₅ together form a 5- or 6-member carbocyclic or heterocyclic ring which is saturated, partially unsaturated or is heteroaromatic. In embodiments, R_{H} is any one of RH1-RH12.

In specific embodiments, compounds useful in the methods herein include those of formula IV:

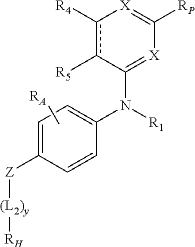

or salts or solvates thereof;

where variables are as defined in formula I and II and the dotted line represents a single or double bond.

In embodiments, y is 1. In embodiments, y is 0. In embodiments, both X are nitrogens. In embodiments, R_{P} is —N(R₂)(R₃). In embodiments, L₂ is —(CH₂)ₙ—, where n is 1, 2 or 3. In embodiments, R_{H} is a heterocyclyl or heteroaryl group. In embodiments R₁ is hydrogen In embodiments, R1 is hydrogen, methyl or trifluoromethyl. In embodiments, Z is —CON(R′)— or —N(R′)CO—. In embodiments, R′ is hydrogen, a C1-C3 alkyl or a C1-C3 haloalky. In embodiments, R′ is hydrogen, methyl or trifluoromethyl. In embodiments, R_{A} is hydrogen, halogen C1-C3 alkyl, C1-C3 alkoxyl, C1-C3 acyl, or C1-C3 haloalkyl. In embodiments, R′ is hydrogen, methyl, methoxy or trifluoromethyl. In embodiments, R₄ and R₅ are selected from hydrogen, halogen, C1-C3 alkyl, C1-C3 alkoxyl, or C1-C3 haloalkyl. In embodiments, R₄ and R₅ together form a 5- or 6-member carbocyclic or heterocyclic ring which is saturated, partially unsaturated or is heteroaromatic. In embodiments, R_{H} is any one of RH1-RH12.

In specific embodiments, compounds useful in the methods herein include those of formula V:

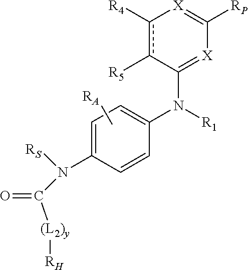

or salts or solvates thereof;

where variables are as defined in formula I and II and the dotted line represents a single or double bond.

In embodiments, y is 1. In embodiments, y is 0. In embodiments, both X are nitrogens. In embodiments, R_{P} is —N(R₂)(R₃). In embodiments, L₂ is —(CH₂)ₙ—, where n is 1, 2 or 3. In embodiments, R_{H} is a heterocyclyl or heteroaryl group. In embodiments R₁ is hydrogen In embodiments, R₁ is hydrogen, methyl or trifluoromethyl. In embodiments, Rs is hydrogen, C1-C3 alkyl, optionally substituted C1-C3 alkyl, or aryl. In embodiments, R_{A} is hydrogen, halogen C1-C3 alkyl, C1-C3 alkoxyl, C1-C3 acyl, or C1-C3 haloalkyl. In embodiments, R′ is hydrogen, methyl, methoxy or trifluoromethyl. In embodiments, R₄ and R₅ are selected from hydrogen, halogen, C1-C3 alkyl, C1-C3 alkoxyl, or C1-C3 haloalkyl. In embodiments, R₄ and R₅ together form a 5- or 6-member carbocyclic or heterocyclic ring which is saturated, partially unsaturated or is heteroaromatic. In embodiments, R_{H} is any one of RH1-RH12.

In specific embodiments, compounds useful in the methods herein include those of formula VI:

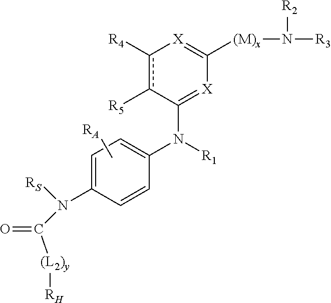

or salts or solvates thereof;

where variables are as defined in formula I and II and the dotted line represents a single or double bond.

In embodiments, y is 1. In embodiments, y is 0. In embodiments, both X are nitrogens. In embodiments, x is 1 and —N—(CH₂)ₙ—, where n is 1, 2 or 3. In embodiments, y is 0. In embodiments, L₂ is —(CH₂)ₙ—, where n is 1, 2 or 3. In embodiments, R_{H} is a heterocyclyl or heteroaryl group. In embodiments R₁ is hydrogen In embodiments, R₁ is hydrogen, methyl or trifluoromethyl. In embodiments, Rs is hydrogen, C1-C3 alkyl, optionally substituted C1-C3 alkyl, or aryl. In embodiments, R_{A} is hydrogen, halogen C1-C3 alkyl, C1-C3 alkoxyl, C1-C3 acyl, or C1-C3 haloalkyl. In embodiments, R′ is hydrogen, methyl, methoxy or trifluoromethyl. In embodiments, R₄ and R₅ are selected from hydrogen, halogen, C1-C3 alkyl, C1-C3 alkoxyl, or C1-C3 haloalkyl. In embodiments, R₄ and R₅ together form a 5- or 6-member carbocyclic or heterocyclic ring which is saturated, partially unsaturated or is heteroaromatic. In embodiments, R_{H} is any one of RH1-RH12.

In specific embodiments, compounds useful in the methods herein include those of formula VII:

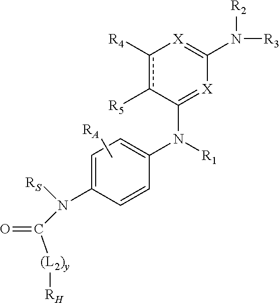

or salts or solvates thereof;

where variables are as defined in formula I and II and the dotted line represents a single or a double bond.

In embodiments, y is 1. In embodiments, y is 0. In embodiments, both X are nitrogens. In embodiments, x is 1 and —N—(CH₂)ₙ—, where n is 1, 2 or 3. In embodiments, y is 0. In embodiments, L₂ is —(CH₂)ₙ—, where n is 1, 2 or 3. In embodiments, R_{H} is a heterocyclyl or heteroaryl group. In embodiments R₁ is hydrogen In embodiments, R₁ is hydrogen, methyl or trifluoromethyl. In embodiments, Rs is hydrogen, C1-C3 alkyl, optionally substituted C1-C3 alkyl, or aryl. In embodiments, R_{A} is hydrogen, halogen C1-C3 alkyl, C1-C3 alkoxyl, C1-C3 acyl, or C1-C3 haloalkyl. In embodiments, R′ is hydrogen, methyl, methoxy or trifluoromethyl. In embodiments, R₄ and R₅ are selected from hydrogen, halogen, C1-C3 alkyl, C1-C3 alkoxyl, or C1-C3 haloalkyl. In embodiments, R₄ and R₅ together form a 5- or 6-member carbocyclic or heterocyclic ring which is saturated, partially unsaturated or is heteroaromatic. In embodiments, R_{H} is any one of RH1-RH12.

In specific embodiments, compounds useful in the methods herein include those of formula VIII:

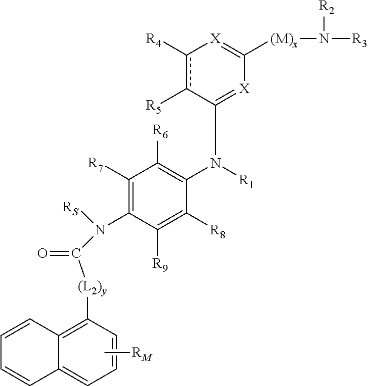

or salts or solvates thereof;

where variables are as defined in formula I and II, the dotted line represents a single or a double bond, R₆-R₉ are independently selected from hydrogen and R_{A} groups defined in formula I. R_{M} represents optional substitution on the fused ring and R_{M} takes the values of R_{A} in formula I.

In embodiments, y is 1. In embodiments, y is 0. In embodiments, both X are nitrogens. In embodiments, x is 1 and —N—(CH₂)ₙ—, where n is 1, 2 or 3. In embodiments, x is 0. In embodiments, L₂ is —(CH₂)ₙ—, where n is 1, 2 or 3. In embodiments R₁ is hydrogen In embodiments, R₁ is hydrogen, methyl or trifluoromethyl. In embodiments, R₇-R₉ are independently selected from hydrogen, C1-C3 alkyl, optionally substituted C1-C3 alkyl, or aryl. In embodiments, R₇-R₉ are independently selected from hydrogen, halogen C1-C3 alkyl, C1-C3 alkoxyl, C1-C3 acyl, or C1-C3 haloalkyl. In embodiments, R₇-R₉ are all hydrogens. In embodiments, R₄ and R₅ are selected from hydrogen, halogen, C1-C3 alkyl, C1-C3 alkoxyl, or C1-C3 haloalkyl. In embodiments, R₄ and R₅ together form a 5- or 6-member carbocyclic or heterocyclic ring which is saturated, partially unsaturated or is heteroaromatic. In embodiments, R_{M} is one or more hydrogen, halogen, C1-C3 alkyl group, C4-C7 cycloalkylalkyl group or C1-C3 haloalkyl group. In embodiments, R_{M} is one or more hydrogen, halogen, particularly Br, methyl or trifluoromethyl. In embodiments, R_{M} is hydrogen.

In specific embodiments, compounds useful in the methods herein include those of formula IX:

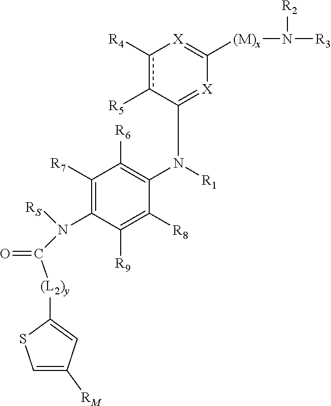

or salts or solvates thereof; where variables are as defined in formula I, the dotted line represents a single or a double bond. R₆-R₉ are independently selected from hydrogen and R_{A} groups defined in formula I and R_{M} represents optional substitution as defined in formula I.

In embodiments, y is 1. In embodiments, y is 0. In embodiments, both X are nitrogens. In embodiments, x is 1 and M is —N—(CH₂)ₙ—, where n is 1, 2 or 3. In embodiments, x is 1 and M is —(CH₂)ₙ—, where n is 1, 2 or 3. In embodiments, x is 0. In embodiments, L₂ is —(CH₂)ₙ—, where n is 1, 2 or 3. In embodiments R₁ is hydrogen In embodiments, R₁ is hydrogen, methyl or trifluoromethyl. In embodiments, R₇-R₉ are independently selected from hydrogen, C1-C3 alkyl, optionally substituted C1-C3 alkyl, or aryl. In embodiments, R₇-R₉ are independently selected from hydrogen, halogen C1-C3 alkyl, C1-C3 alkoxyl, C1-C3 acyl, or C1-C3 haloalkyl. In embodiments, R₇-R₉ are all hydrogens. In embodiments, R₄ and R₅ are selected from hydrogen, halogen, C1-C3 alkyl, C1-C3 alkoxyl, or C1-C3 haloalkyl. In embodiments, R₄ and R₅ together form a 5- or 6-member carbocyclic or heterocyclic ring which is saturated, partially unsaturated or is heteroaromatic. In embodiments, R_{M} is hydrogen, halogen, C1-C3 alkyl group or C1-C3 haloalkyl group. In embodiments, R_{M} is hydrogen, halogen, particularly Br, methyl or trifluoromethyl.

In other embodiments, the invention provides a compound of formula XI:

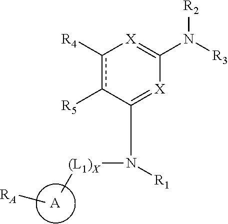

or salts, or solvates thereof,

where:

each X is independently selected from N or CH and at least one X is N;

the A ring is a carbocyclic or heterocyclic ring having 3-10 carbon atoms and optionally 1-6 heteroatoms and which optionally is saturated, unsaturated or aromatic;

L₁ is an optional 1-3 carbon linker that is optionally substituted, where x is 0 or 1 to indicate the absence of presence of L₁;

R₁ is selected from the group consisting of hydrogen, alkyl group. alkenyl group, cycloalkyl group, cycloalkenyl group, heterocyclyl group, or aryl group, each of which groups is optionally substituted;

R₂ and R₃ are independently selected from hydrogen, alkyl, alkenyl, cycloalkyl, cycloalkenyl, aryl or heterocyclyl, each of which groups is optionally substituted or

R₂ and R₃ together form an optionally substituted 5- to 8-member heterocyclic ring which is a saturated, partially unsaturated or aromatic ring;

R₄ and R₅ are independently selected from hydrogen, halogen, alkyl, alkenyl, cycloalkyl, cycloalkenyl, aryl or heterocyclyl, each of which groups is optionally substituted or

R₄ and R₅ together form an optionally substituted 5- or 6-member ring which optionally contains one or two double bonds or is aromatic and optionally contains 1-3 heteroatoms;

where the dotted line is a single or double bond dependent upon selection of R₄ and R₅;

and

R_{A} represents hydrogens or 1-10 substituents on the indicated ring, wherein R_{A} substituents are independently selected from hydrogen, halogen, hydroxyl, cyano, nitro, amino, mono- or disubstituted amino, alkyl, alkenyl, cycloalkyl, cycloalkenyl, aryl, heterocyclyl, —OR₁₅, —COR₁₅, —COOR₁S, —OCOR₁₅, —CO—NR₁₆R₁₇, —OCON R₁₆R₁₇, —NR₁₆—CO—R₁₅, —SR₁₅, —SOR₁₅, —SO₂R₁₅, —SO₂—NR₁₆R₁₇, R10, —NH—CO-(L₂)y-R₁₂, or —NH—CO-(L₂)y-R₁₂, where L₂ is an optional 1-6 carbon atom linker group which linker is optionally substituted and wherein one or two carbons of the liker are optionally replaced with 0 or S, where y is 0 or 1 to show the absence or presence of L₂;

R₁₀ is selected from alkyl, alkenyl, cycloalkyl, cycloalkenyl, heterocyclyl, or aryl, each of which groups is optionally substituted with one or more halogen, alkyl, alkenyl, haloalkyl, alkoxy, aryl, heteroaryl, heterocyclyl, aryl-substituted alkyl, or heterocyclyl-substituted alkyl;

R₁₂ is selected from cycloalkyl, cycloalkenyl, heterocyclyl, heteroaryl, or aryl, each of which groups is optionally substituted, or R₁₂ is a C1-C3 alky substituted with cycloalkyl, cycloalkenyl, heterocyclyl, heteroaryl, or aryl each of which is optionally substituted and where optional substitution is one or more halogen, alkyl, alkenyl, haloalkyl, alkoxy, aryl, heteroaryl, or heterocyclyl;
each R₁₅ is independently selected from hydrogen, alkyl, alkenyl, cycloalkyl, cycloalkenyl, aryl or heterocyclyl, arylalkyl (an alkyl group substituted with an aryl) and heterocyclylalkyl, cycloalkylalkyl, cycloalkenylalkyl, each of which groups is optionally substituted; and

each R₁₆ and R₁₇ is independently selected from hydrogen, alkyl, alkenyl, cycloalkyl, cycloalkenyl, aryl or heterocyclyl, arylalkyl (an alkyl group substituted with an aryl) and heterocyclylalkyl, cycloalkylalkyl, cycloalkenylalkyl, each of which groups is optionally substituted;

wherein optional substitution includes substitution with one or more halogen, nitro, cyano, amino, mono- or di-C1-C3 alkyl substituted amino, C1-C3 alkyl, C2-C4 alkenyl, C3-C6 cycloalkyl, C3-C6-cycloalkenyl, C6-C12 aryl, and C6-C12 heterocyclyl.

In embodiments of formula X, R₁ is H. In embodiments of formula X, R₁ is H, and x is 0. In embodiments of formula X, R₁ is H, x is 0 and R₅ is other than an electronegative group. In embodiments of formula X, R₁ is H, x is 0 and R₅ is hydrogen. In embodiments of formula XI, R₁ is H, x is 0, R₅ is hydrogen and R₄ is a C1-C3 alkyl. In embodiments of formula X, R₁ is H, x is 0, R₅ is hydrogen and R₄ is methyl.

In embodiments of formula XI, R₁ is H, x is 1 and L₁ is —(CH₂)ₙ—, where n is 1 or 2. In embodiments of formula XI, R₁ is H, x is 1 and L₁ is —(CH₂)ₙ—, where n is 1 or 2, and R₅ is an electronegative group. In embodiments of formula XI, R₁ is H, x is 1 and _{L1} is —(CH₂)ₙ—, where n is 1 or 2, and R₅ is a halogen. In embodiments of formula XI, R₁ is H, x is 1 and L₁ is —(CH₂)—, and R₅ is a halogen. In embodiments of formula XI, R₁ is H, x is 1 and L₁ is —(CH₂)ₙ—, where n is 1 or 2, and R₅ is a fluorine. In embodiments of formula XI, R₁ is H, x is 1 and L₁ is —(CH₂)—, and R₅ is a fluorine. In embodiments of formula XI, R₁ is H, x is 1 and Li is —(CH₂)—, R₅ is a halogen and R₄ is C1-C3 alkyl. In embodiments of formula XI, R₁ is H, x is 1 and L₁ is —(CH₂)—, R₅ is a halogen and R₄ is methyl. In embodiments of formula XI, R₁ is H, x is 1 and _{L1} is —(CH₂)—, R₅ is a fluorine and R₄ is C1-C3 alkyl. In embodiments of formula XI, R₁ is H, x is 1 and L₁ is —(CH₂)—, R₅ is a fluorine and R₄ is methyl.

In an embodiment, the compound has formula XII:

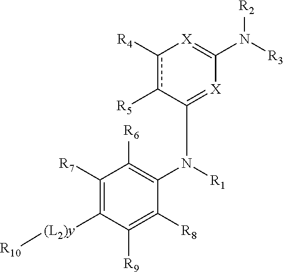

or a salt or solvate thereof where variables are as defined for formula XI.

In an embodiment, the compound has formula XIII:

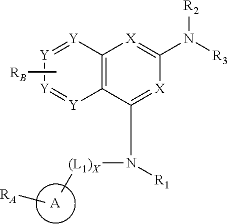

or a salt, or a solvate thereof, wherein variables are as defined in formula XI and where;

each Y is independently selected from N or CH;

R_{B} represents hydrogens or 1-10 substituents on the indicated ring, wherein R_{A} substituents are independently selected from hydrogen, halogen, hydroxyl, cyano, nitro, amino, mono- or disubstituted amino, alkyl, alkenyl, cycloalkyl, cycloalkenyl, aryl, heterocyclyl, —OR₁₅, —COR₁₅, —COOR₁₅, —OCOR₁₅, —CO—NR₁₆R₁₇, —OCON R₁₆R₁₇, —NR₁₆—CO—R₁₅, —SR₁₅, —SOR₁₅, —SO₂R₁₅, —SO₂—NR₁₆R₁₇, or -(L₂)y-R₁₀, where L₂ is an optional 1-6 carbon atom linker group which linker is optionally substituted, and where y is 0 or 1 to show the absence or presence of L₂.

In embodiments of formula XIII, R₁ is H. In embodiments of formula XIII, R₁ is H, and x is 0. In embodiments of formula XIII, R₁ is H, x is 0 and the B ring is substituted with other than an electronegative group. In embodiments of formula XIII, R₁ is H, x is 0 and the B ring is substituted with one or more hydrogens or C1-C3 alkyl groups. In embodiments of formula XIII, R₁ is H, x is 0, the B ring is substituted with one or more hydrogens or methyl groups.

In embodiments of formula XIII, R₁ is H, x is 1 and Li is —(CH₂)ₙ—, where n is 1 or 2. In embodiments of formula XIII, R₁ is H, x is 1 and Li is —(CH₂)ₙ—, where n is 1 or 2, and the B ring is substituted with an electronegative group. In embodiments of formula XIII, R₁ is H, x is 1 and L₁ is —(CH₂)ₙ—, where n is 1 or 2, and the B ring is substituted with a halogen. In embodiments of formula XIII, R₁ is H, x is 1 and L₁ is —(CH₂)—, and the B ring is substituted with a halogen. In embodiments of formula XIII, R₁ is H, x is 1 and L₁ is —(CH₂)ₙ—, where n is 1 or 2, and the B ring is substituted with a fluorine. In embodiments of formula XIII, R₁ is H, x is 1 and L₁ is —(CH₂)—, and the B ring is substituted with a fluorine. In embodiments of formula XIII, R₁ is H, x is 1 and L₁ is —(CH₂)—, the B ring is substituted a halogen and a C1-C3 alkyl. In embodiments of formula XIII, R₁ is H, x is 1 and L₁ is —(CH₂)—, and the B ring is substituted a halogen. In embodiments of formula XIII, R₁ is H, x is 1 and _{L1} is —(CH₂)—, the B ring is substituted with a fluorine and R₄ is C1-C3 alkyl. In embodiments of formula XIII, R₁ is H, x is 1 and L₁ is —(CH₂)—, the B ring is substituted a halogen and a methyl.

In embodiments, the compound has formula XIV or XV:

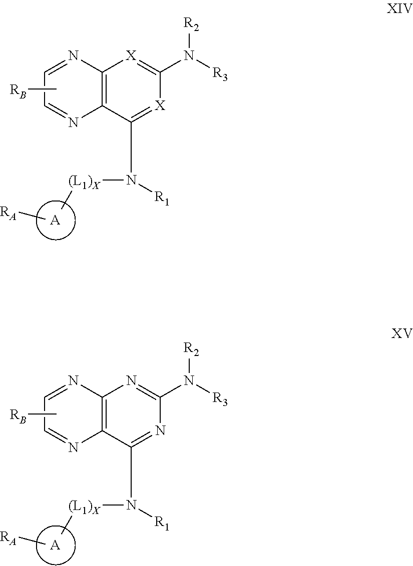

or a salt or solvate thereof,

where variables are as defined in formula XI, XII or XIII.

In embodiments of these formulas, x is 0 and R₁ is hydrogen. In embodiments of these formulas, x is 1, L₁ is —(CH₂)— and R₁ is hydrogen.

In embodiments, the compound has formula XVI or XVII:

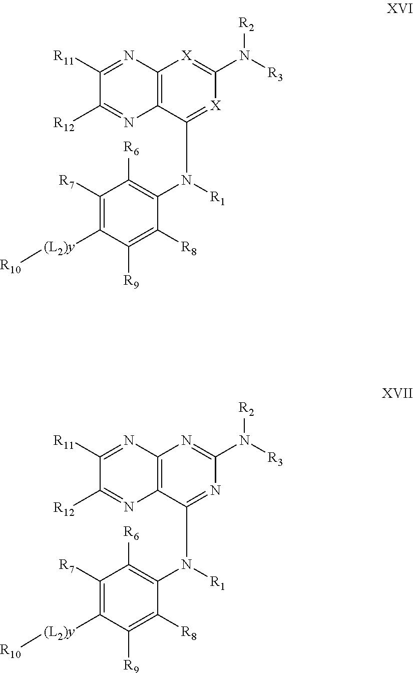

or a salt or solvate thereof,

where variables are as defined in formula XI or XV, and

R₁₁ and R₁₂ are independently selected from hydrogen, halogen, alkyl group, alkenyl group, cycloalkyl group, cycloalkenyl group, or heterocyclyl group, each of which groups is optionally substituted.

In embodiments, the compound has formula XVIII:

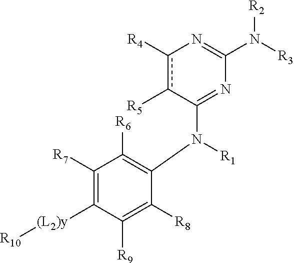

or salts (or solvates) thereof,

wherein:

R₁ is selected from the group consisting of hydrogen, alkyl group. alkenyl group, cycloalkyl group, cycloalkenyl group, heterocyclyl group, or aryl group, each of which groups is optionally substituted (need to define substitution);

R₂ and R₃ together form an optionally substituted 5- or 6-member heterocyclic ring which can contain one or two double bonds or be aromatic;

R₄ and R₅ are independently selected from hydrogen, halogen, alkyl group, alkenyl group, cycloalkyl group, cycloalkenyl group, or heterocyclyl group, each of which groups is optionally substituted or

R₄ and R₅ together form an optionally substituted 5- or 6-member heterocyclic ring which can contain one or two double bonds or be aromatic;

the dotted line is a single or double bond dependent upon choice of R₄ and R₅;

R₆-R₉ are independently selected from hydrogen, halogen, alkyl group, alkenyl group, cycloalkyl group, cycloalkenyl group, or heterocyclyl group, each of which groups is optionally substituted;

L is an optional 1-6 atom linker group, where x is 1 or 0 to show the presence or absence of the L group; and

R₁₀ is selected from alkyl group. alkenyl group, cycloalkyl group, cycloalkenyl group, heterocyclyl group, or aryl group, each of which groups is optionally substituted.

For example, L is a 2-6 atom linker group; (e.g., —CH₂—O—, —CH₂—CH₂—O—, —O—CH₂—, —O—CH₂—CH₂—, —CO—NH—, —NH—CO—, —CH₂—CO—NH—, —CH₂—CH₂—CO—NH—)

In an embodiment, the compound is of formula XIX:

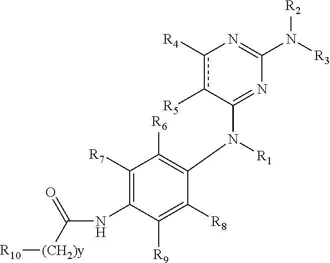

or salts (or solvates) thereof,

where:

R₁-R₉ are as defined above; the dotted line represents a single or double bond dependent on choice of R₄ and R₅;

y is 0 or an integer ranging from 1-3 inclusive; and

R₁₀ is selected from alkyl group. alkenyl group, cycloalkyl group, cycloalkenyl group, heterocyclyl group, or aryl group, each of which groups is optionally substituted.

In embodiments, the CHD1L inhibitor is a compound of formula XX, XXI, XXII or XXIII:

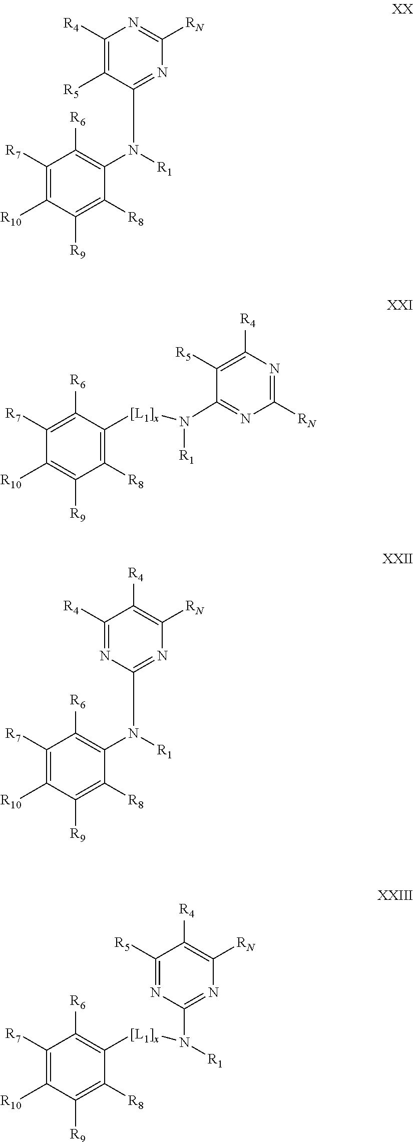

and salts or solvates thereof, where R₁-R₉ represent hydrogen or optional substituents, R₁₀ is a moiety believed to be associated with potency; and R_{N} is a moiety believed to be associated with physicochemical properties such as solubility. Li is as defined for formula I above and x is 0 or 1 to show the absence of presence of the Li group. In embodiments, R₅ is a substituent other than hydrogen which is believed to be associated with metabolic stability. In specific embodiments, R₅ is a halogen, particularly F or Cl, a C1-C3 alkyl group, particularly a methyl group. In specific embodiments, when x is 1, R₅ is an electronegative substituent, particularly a halogen, and more preferably F or Cl. In specific embodiments, R₅ is a halogen, particularly F or Cl, and R₄ is a C1-C3 alkyl group, particularly a methyl group. In specific embodiments, when x is 1, R₅ is an electronegative substituent, particularly a halogen, and more preferably F or Cl. In embodiments, R₄ is a substituent other than hydrogen and in particular is a C1-C3 alkyl group, and more particularly is a methyl group. In a specific embodiment, R₅ is F and R₄ is methyl. In specific embodiments Li is —(CH₂)ₙ—, where n is 1 or 2 and more specifically where n is 1. In embodiments, R₆-R₉ are selected from hydrogen, C1-C3-alkyl, halogen, hydroxyl, C1-C3 alkoxy, formyl, or C₁-C₃ acyl. In embodiments, one or two of R₆-R₉ are moieties other than hydrogen. In an embodiment, one of R₆-R₉ is a halogen, particularly fluorine. In specific embodiments, all of R₆-R₉ are hydrogen. In embodiments, R_{N} is an amino moiety —N(R₂)(R₃). In specific embodiments, R_{N} is an optionally substituted heterocyclic group having a 5- to 7-member ring optionally containing a second heteroatoms (N, S or O). In embodiments, R_{N} is optionally substituted pyrrolidin-1-yl, piperidin-1-yl, azepan-1-yl, piperazin-1-yl, or morpholino. In R_{N} is substituted with one substituent selected from C1-C3 alkyl, formyl, C1-C3 acyl (particularly acetyl), hydroxyl, halogen (particularly F or Cl), hydroxyl, C1-C3 alkyl (particularly —CH₂—CH₂—OH). In embodiments, R_{N} is unsubstituted pyrrolidin-1-yl, piperidin-1-yl, azepan-1-yl, piperazin-1-yl, or morpholino.

In embodiments, R₁₀ is —NRy-CO-(L₂)y-R₁₂ or —CO—NRy--(L₂)y-R₁₂, where y is 0 or 1 to indicate the absence of presence of L₂ which is an optional 1-6 carbon atom linker group which linker is optionally substituted and wherein one or two, carbons of the linker are optionally replaced with O, NH, NRy or S, where Ry is hydrogen or a 1-3 carbon alkyl, and R₁₂ is an aryl group, cycloalkyl group, heterocyclic group, or heteroaryl group, each of which is optionally substituted. I_{N} embodiments, y is 1. L₂ is —(CH₂)p-, where p is 0-3. In embodiments, R₁₂ is thiophen-2-yl, thiophen-3-yl, furany-2-yl, furan-3-yl, pyrrol-2-yl, pyrrol-3-yl, oxazol-4-yl, oxazol-5-yl, oxazol-2-yl, indol-2-yl, indol-3-yl, benzofuran-2-yl, benzofuran-3-yl, benzo[b]thiophen-2-yl, benzo[b]thiophen-3-yl, isobenzofuran-1-yl, isoindol-1-yl, or benzo[c]thiophen-1-yl. In embodiments, R₁ is hydrogen or methyl. In embodiments, R₁₂ is thiophen-2-yl, furany-2-yl, pyrrol-2-yl, oxazol-4-yl, indol-2-yl, benzofuran-2-yl, or benzo[b]thiophen-2-yl. In embodiments, R₁₂ is thiophen-2-yl or indol-2-yl. In embodiments, R₁ is hydrogen or methyl.

In more general embodiments of formula XX-XXIII:

R₁ is selected from the group consisting of hydrogen, alkyl group, alkenyl group, cycloalkyl group, cycloalkenyl group, heterocyclyl group, or aryl group, each of which groups is optionally substituted;

R_{N} is —NR₂R3, R₂ and R₃ are independently selected from hydrogen, alkyl, alkenyl, cycloalkyl, cycloalkenyl, aryl or heterocyclyl, each of which groups is optionally substituted or R₂ and R₃ together form an optionally substituted 5- to 8- member heterocyclic ring which is a saturated, partially unsaturated or aromatic ring;

R₄-R₉ are independently selected from hydrogen, halogen, hydroxyl, cyano, nitro, amino, mono- or dialkyl substituted amino, optionally substituted alkyl, optionally substituted alkenyl, optionally substituted cycloalkyl, optionally substituted cycloalkenyl, optionally substituted aryl, optionally substituted heterocyclyl, —OR₁₅, —COR₁₅, —COOR₁₅, —OCOR₁₅, —CO—NR₁₅R₁₆, —OCONR₁₅R₁₆, —NR₁₅—CO—R₁₆, —SR₁₅, —SOR₁₅, —SO₂R₁₅, and —SO₂—NR₁₅R₁₆;

R₁₀ is —NRy-CO-(L₂)y-R₁₂, —CO—NRy-(L₂)y-R₁₂, where L₂ is an optional 1-6 carbon atom linker group which linker is optionally substituted and wherein one or two, carbons of the liker are optionally replaced with O or S, where y is 0 or 1 to show the absence or presence of L₂;

R₁₂ is selected from cycloalkyl, cycloalkenyl, heterocyclyl, heteroaryl, or aryl, each of which groups is optionally substituted, or R₁₂ is a C1-C3 alky substituted with cycloalkyl, cycloalkenyl, heterocyclyl, heteroaryl, or aryl each of which is optionally substituted and where optional substitution is one or more halogen, alkyl, alkenyl, haloalkyl, alkoxy, aryl, heteroaryl, or heterocyclyl;

each R₁₅ and R₁₆ is independently selected from hydrogen, alkyl, alkenyl, cycloalkyl, cycloalkenyl, aryl or heterocyclyl, arylalkyl and heterocyclylalkyl, cycloalkylalkyl, cycloalkenylalkyl, each of which groups is optionally substituted; and

wherein optional substitution includes, substitution with one or more halogen, nitro, cyano, amino, mono- or di-C1-C3 alkyl substituted amino, C1-C3 alkyl, C2-C4 alkenyl, C3-C6 cycloalkyl, C3-C6-cycloalkenyl, C6-C12 aryl, C6-C12 heterocyclyl, —OR₁₇, —COR₁₇, —COOR₁₇, —OCOR₁₇, —CO—NR₁₇R₁₈, —OCONR₁₇R₁₈, —NR₁₇—CO—R₁₈, —SR₁₇, —SOR₁₇, —SO₂R₁₇, and —SO₂—NR₁₇R₁₃, where R₁₇ and R₁₈ are independently hydrogen or a C1-C6 alkyl.

In embodiments of formula XX and XXI, R_{N} is an optionally substituted cyclic amine group selected from any of R_{N}1-R_{N}39 (Scheme 2). Exemplary optional substitution of groups is illustrated in Scheme 2. The illustrated R substituent groups can be positioned on any available ring position. In the moieties of Scheme 2, preferred alkyl are C1-C3 alkyl, acyl includes formyl, preferred acyl are C1-C6 acyl and more preferably acetyl, hydroxyalkyl are C1-C6 hydroxyalkyl and preferably are —CH₂—CH₂—OH, for amine groups, preferred alkyl are C1-C3 alkyl, preferred alkyl for —SO₂alkyl are C1-C3 alkyl and more preferred is methyl.

In specific embodiments of formula XX, or XXI, R_{N} is R_{N}1. In specific embodiments, R_{N} is R_{N}3. In specific embodiments, R_{N} is R_{N}2 or R_{N}4. In specific embodiments, R_{N} is R_{N}5 or R_{N}6. In specific embodiments, R_{N} is R_{N}7 or R_{N}8. In specific embodiments, R_{N} is R_{N}9. In specific embodiments, R_{N} is R_{N}10. In specific embodiments, R_{N} is R_{N}11. In specific embodiments, R_{N} is R_{N}12. In specific embodiments, R_{N} is R_{N}13. In specific embodiments, R_{N} is R_{N}14. In specific embodiments, R_{N} is R_{N}15. In specific embodiments, R_{N} is R_{N}16. In specific embodiments, R_{N} is R_{N}17 or R_{N}18. In specific embodiments, R_{N} is R_{N}19 or R_{N}20. In specific embodiments, R_{N} is R_{N}21. In specific embodiments, R_{N} is R_{N}22. In specific embodiments, R_{N} is R_{N}23 or R_{N}24. In specific embodiments, R_{N} is R_{N}25. In an embodiment, R_{N} is R_{N}1, R_{N}2, R_{N}3, R_{N}4, R_{N}11, R_{N}13, or R_{N}14. In an embodiment, R_{N} is R_{N}26-R_{N}29. In an embodiment, R_{N} is R_{N}30. In an embodiment, R_{N} is R_{N}31.

In embodiments of formula XX-XXIII, R₁₂ is an optionally-substituted thienyl, thienylmethyl, furyl, furylmethyl, indolyl or methylindolyl. In embodiments, R₁₂ is a moiety illustrated in Scheme 3 R12-1 to R12-69, R12-1-R12-71 or R12-72-R12-78. In moieties of Scheme 3, preferred alkyl groups are C-C6 alkyl groups or more preferred C1-C3 alkyl groups, preferred halogen are F, Cl and Br, acyl includes formyl and preferred acyl are —CO—C1-C6 alky and more preferred is acetyl, phenyl is optionally substituted with one or more halogen, alkyl or acyl. In embodiments, R₁₂ is a methyl, ethyl group or propyl substituted with a moiety as illustrated in Scheme 3 R12-1 to R12-22. In an embodiment, R₁₂ is R12-1. In an embodiment, R₁₂ is R12-2. In an embodiment, R₁₂ is R12-3. In an embodiment, R₁₂ is R12-4. In an embodiment, R₁₂ is R12-5. In an embodiment, R₁₂ is R12-6. In an embodiment, R₁₂ is R12-7. In an embodiment, R₁₂ is R12-8. In an embodiment, R₁₂ is R12-9. In an embodiment, R₁₂ is R12-10. In an embodiment, R₁₂ is R12-11. In an embodiment, R₁₂ is R12-12. In an embodiment, R₁₂ is R12-13. In an embodiment, R₁₂ is R12-14. In an embodiment, R₁₂ is R12-15. In an embodiment, R₁₂ is R12-16. In an embodiment, R₁₂ is R12-17. In an embodiment, R₁₂ is R12-18 In an embodiment, R₁₂ is R12-19. In an embodiment, R₁₂ is R12-20. In an embodiment, R₁₂ is R12-21. In an embodiment, R₁₂ is R12-22. In an embodiment, R₁₂ is one of R12-23-R12-26. In an embodiment, R₁₂ is one of R12-27-R12-30. In an embodiment, R₁₂ is one of R12-31-R12-34. In an embodiment, R₁₂ is one of R12-35-R12-42. In embodiments, R12 is any one of R12-43-R12-69. In embodiments, R₁₂ is a methyl, ethyl group or propyl group substituted with a moiety R12-43-R12-69, as illustrated in Scheme 3. In embodiments, R12 is any one of R12-43-R12-45. In embodiments, R₁₂ is a methyl, ethyl group or propyl group substituted with a moiety R12-43-R12-45 as illustrated in Scheme 3. In embodiments, R12 is any one of R12-46-R12-48. In embodiments, R₁₂ is a methyl, ethyl group or propyl group substituted with a moiety R12-46-R12-48 as illustrated in Scheme 3. In embodiments, R12 is any one of 12-49-R12-51. In embodiments, R₁₂ is a methyl, ethyl group or propyl group substituted with a moiety R12-49-R12-51 as illustrated in Scheme 3. In embodiments, R12 is any one of R12-52-R12-54. In embodiments, R₁₂ is a methyl, ethyl group or propyl group substituted with a moiety R12-52-R12-54 as illustrated in Scheme 3. In embodiments, R12 is any one of R12-55-R12-58. In embodiments, R₁₂ is a methyl, ethyl group or propyl group substituted with a moiety R12-55-R12-58 as illustrated in Scheme 3. In embodiments, R12 is any one of R12-59-R12-62 In embodiments, R₁₂ is a methyl, ethyl group or propyl group substituted with a moiety R12-59-R12-62 as illustrated in Scheme 3. In embodiments, R₁₂ is any one of R12-63-R12-66. In embodiments, R₁₂ is a methyl, ethyl group or propyl group substituted with a moiety R12-63-R12-66 as illustrated in Scheme 3. In embodiments, R₁₂ is any one of R12-67-R12-69. In embodiments, R₁₂ is a methyl, ethyl or propyl group substituted with a moiety R12-67-R12-69 as illustrated in Scheme 3. In embodiments, R₁₂ is a moiety R12-70 or R12-71 as illustrated in Scheme 3. In embodiments, R₁₂ is a moiety R12-72 or R12-73 as illustrated in Scheme 3. In embodiments, R₁₂ is a moiety R12-74 as illustrated in Scheme 3. In embodiments, R₁₂ is a moiety R12-75 or R12-76 as illustrated in Scheme 3. In embodiments, R₁₂ is a moiety R12-75 or R12-76, where p is 1 or 2 as illustrated in Scheme 3. In embodiments, R₁₂ is a moiety R12-77 as illustrated in Scheme 3. In embodiments, R₁₂ is a moiety R12-77, where p is 1 or 2 as illustrated in Scheme 3. In embodiments, R₁₂ is a moiety R12-78 as illustrated in Scheme 3. In embodiments, R₁₂ is a moiety R12-78, where p is 1 or 2 as illustrated in Scheme 3.

In embodiments herein of formula XX-XXIII, R_{N} is an optionally substituted cyclic amine group selected from any of R_{N}1-R_{N}25 or R_{N}26-R_{N}39 (Scheme 2) and R₁₂ is a thienyl, thienylmethyl, furyl, furylmethyl, indolyl or methylindoyl. In embodiments of formula XX-XXIII, R_{N} is R_{N}1, R_{N}2, R_{N}3, R_{N}4, R_{N}11, R_{N}13, R_{N}14 or R_{N}25 and R₁₂ is a thienyl, thienylmethyl, furyl, furylmethyl, indolyl or methylindoyl. In embodiments, R_{N} is R_{N}37 and R₁₂ is a thienyl, thienylmethyl, furyl, furylmethyl, indolyl or methylindoyl. In embodiments, R_{N} is R_{N}38 or R_{N}39 and R₁₂ is a thienyl, thienylmethyl, furyl, furylmethyl, indolyl or methylindoyl. In embodiments, R_{N} is R_{N}26 and R₁₂ is a thienyl, thienylmethyl, furyl, furylmethyl, indolyl or methylindoyl. In embodiments, R_{N} is R_{N}27-R_{N}32 and R₁₂ is a thienyl, thienylmethyl, furyl, furylmethyl, indolyl or methylindoyl. In embodiments, R_{N} is R_{N}33-R_{N}35 and R₁₂ is a thienyl, thienylmethyl, furyl, furylmethyl, indolyl or methylindoyl. In embodiments, R_{N} is R_{N}36 and R₁₂ is a thienyl, thienylmethyl, furyl, furylmethyl, indolyl or methylindoyl.

In embodiments herein of formula XX-XXIII, R_{N} is an optionally substituted cyclic amine group of formula R_{N}37 (Scheme 2) and R₁₂ is a thienyl, thienylmethyl, furyl, furylmethyl, indolyl or methylindoyl. In embodiments of formula XX-XXIII, R_{N} is R_{N}38 and R₁₂ is a thienyl, thienylmethyl, furyl, furylmethyl, indolyl or methylindoyl. In embodiments of formula XX-XXIII, R_{N} is R_{N}39 and R₁₂ is a thienyl, thienylmethyl, furyl, furylmethyl, indolyl or methylindoyl.

In embodiments of formula XX-XXIII, R₁₀ is —NHCOR₁₂. In embodiments of formula XX-XXIII, R₁₀ is —CONHR₁₂. In embodiments herein of formula XX-XXIII, R₁₀ is —CO—NH—R₁₂ and R_{N} is any one of R_{N}1-R_{N}25 and R₁₂ is any one of R12-1-R12-22. In embodiments herein of formula XX-XXIII, R₁₀ is —CO—NH—R₁₂ and R_{N} is any one of R_{N}1-R_{N}25 and R₁₂ is any one of R12-1-R12-69.

In further embodiments of the forgoing embodiments of formula XXI or XXIII, x is 1. In further embodiments of the forgoing embodiments of formula XXI or XXIII, x is 1 and Li is —(CH₂)ₙ—. In further embodiments of the forgoing embodiments of formula XXI or XXIII, x is 1 and L₁ is —(CH₂)ₙ—, where n is 1 or 2. In further embodiments of the forgoing embodiments of formula XXI or XXIII, x is 1 and Li is —(CH₂)ₙ—, where n is 1. In further embodiments of the forgoing embodiments of formula XXI or XXIII, x is 1 and Li is —(CH₂)—, and R₅ is an electronegative group. In further embodiments of the forgoing embodiments of formula XXI or XXIII, x is 1 and Li is —(CH₂)—, and R₅ is a halogen. In further embodiments of the forgoing embodiments of formula XXI or XXIII, x is 1 and Li is —(CH₂)—, and R₅ is a fluorine. In further embodiments of the forgoing embodiments of formula XXI or XXIII, x is 1 and L₁ is —(CH₂)—, R₅ is a fluorine and R₄ is a C1-C3 alkyl group. In further embodiments of the forgoing embodiments of formula XXI or XXIII, x is 1 and Li is —(CH₂)—, R₅ is a fluorine and R₄ is a methyl group.

In embodiments, the compound is of formula XXX:

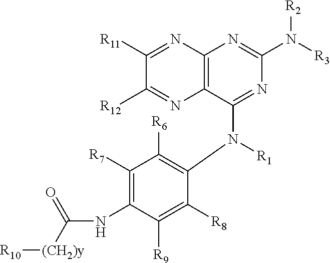

or salts (or solvates) thereof,

wherein:

R1 is selected from the group consisting of hydrogen, alkyl group. alkenyl group, cycloalkyl group, cycloalkenyl group, heterocyclyl group, or aryl group, each of which groups is optionally substituted (need to define substitution);

R₂ and R₃ together form an optionally substituted 5- or 6-member heterocyclic ring which can contain one or two double bonds or be aromatic;

R₆-R₉ are independently selected from hydrogen, halogen, alkyl group, alkenyl group, cycloalkyl group, cycloalkenyl group, or heterocyclyl group, each of which groups is optionally substituted;

Y is 0 or an integer ranging from 1-3 inclusive;

R₁₀ is selected from alkyl group. alkenyl group, cycloalkyl group, cycloalkenyl group, heterocyclyl group, or aryl group, each of which groups is optionally substituted; and

R11 and R₁₂ are independently selected from hydrogen, halogen, alkyl group, alkenyl group, cycloalkyl group, cycloalkenyl group, or heterocyclyl group, each of which groups is optionally substituted. In embodiments, R₁₀ is any one of RH1-RH12.

In specific embodiments, compounds useful in the methods herein include those of formula XXXI:

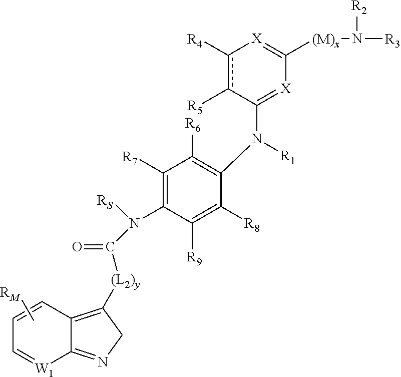

or salts or solvates thereof; where variables are as defined in formula I, R₆-R₉ are independently selected from hydrogen and R_{A} groups defined in formula I, R_{M} represents optional substitution on the fused ring and R_{M} takes the values of RA in formula I and W₁ is N or CH.

In embodiments, y is 1. In embodiments, y is 0. In embodiments, both X are nitrogens. In embodiments, x is 1 and M is —N—(CH₂)ₙ—, where n is 1, 2 or 3. In embodiments, x is 1 and M is —(CH₂)ₙ—, where n is 1, 2 or 3. In embodiments, x is 0. In embodiments, L₂ is —(CH₂)ₙ—, where n is 1, 2 or 3. In embodiments R₁ is hydrogen In embodiments, R₁ is hydrogen, methyl or trifluoromethyl. In embodiments, R₇-R₉ are independently selected from hydrogen, C1-C3 alkyl, optionally substituted C1-C3 alkyl, or aryl. In embodiments, R₇-R₉ are independently selected from hydrogen, halogen C1-C3 alkyl, C1-C3 alkoxyl, C1-C3 acyl, or C1-C3 haloalkyl. In embodiments, R₇-R₉ are all hydrogens. In embodiments, R₄ and R₅ are selected from hydrogen, halogen, C1-C3 alkyl, C1-C3 alkoxyl, or C1-C3 haloalkyl. In embodiments, R₄ and R₅ together form a 5- or 6-member carbocyclic or heterocyclic ring which is saturated, partially unsaturated or is heteroaromatic. In embodiments, R_{M} is one or more hydrogen, halogen, C1-C3 alkyl group or C1-C3 haloalkyl group. In embodiments, R_{M} is one or more hydrogen, halogen, particularly Br, methyl or trifluoromethyl. In embodiments, R_{M} is hydrogen.

In embodiments, compounds useful in the methods, pharmaceutical compositions and pharmaceutical combinations of this invention include compounds of formula XXXII:

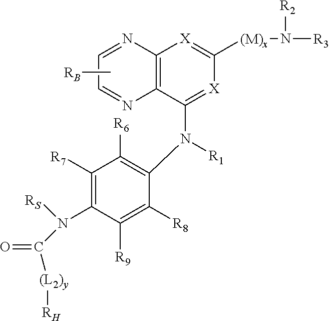

or salts or solvates thereof,

where variables are as defined in formula I, R_{B} represents optional substitution as defined in formula I and R₆—R₉ are hydrogen or take values of R_{A} from formula I.

In embodiments, y is 1. In embodiments, y is 0. In embodiments, both X are nitrogens. In embodiments, x is 1 and M is —N—(CH₂)ₙ—, where n is 1, 2 or 3. In embodiments, x is 1 and M is —(CH₂)ₙ—, where n is 1, 2 or 3. In embodiments, x is 0. In embodiments, L₂ is —(CH₂)ₙ—, where n is 1, 2 or 3. In embodiments R₁ is hydrogen In embodiments, R₁ is hydrogen, methyl or trifluoromethyl. In embodiments, R₆-R₉ are independently selected from hydrogen, C1-C3 alkyl, optionally substituted C1-C3 alkyl, or aryl. In embodiments, R₆-R₉ are independently selected from hydrogen, halogen C1-C3 alkyl, C1-C3 alkoxyl, C1-C3 acyl, or C1-C3 haloalkyl. In embodiments, R₇-R₉ are all hydrogens. In embodiments, R₄ and R₅ are selected from hydrogen, halogen, C1-C3 alkyl, C1-C3 alkoxyl, or C1-C3 haloalkyl. In embodiments, R_{B} is one or more hydrogen, halogen, C1-C3 alkyl group or C1-C3 haloalkyl group. In embodiments, R_{B} is one or more hydrogen, halogen, particularly Br, methyl or trifluoromethyl. In embodiments, R_{B} is hydrogen. In embodiments, R_{H} is a heterocyclyl or heteroaryl group. In embodiments, R_{H} is optionally substituted naphthyl, thiophene, indoyl, or pyridinopyrroyl.

Compounds of formulas XXXV-XLII are useful in the methods, pharmaceutical compositions and pharmaceutical combinations herein:

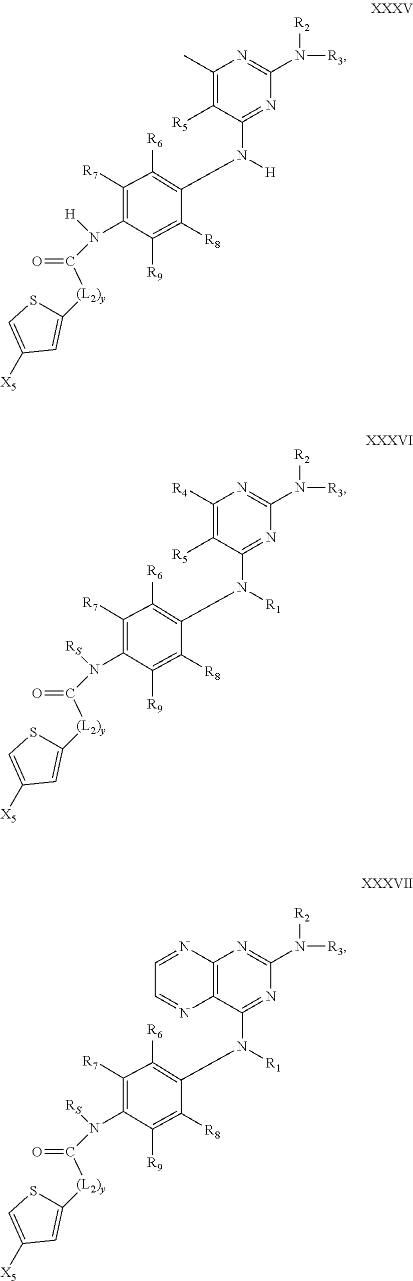

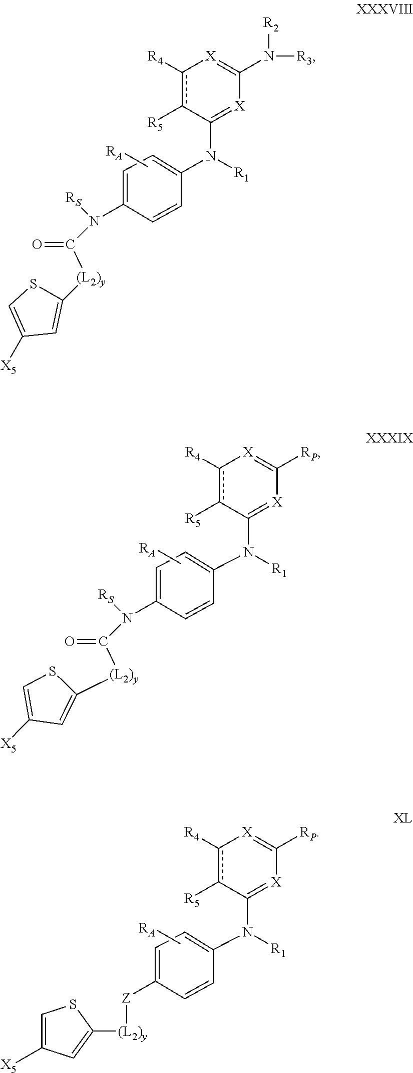

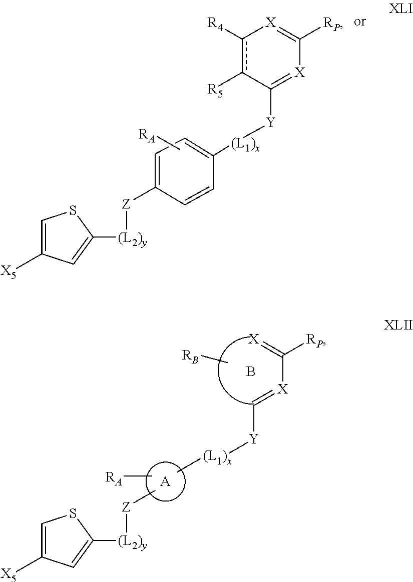

where variables are as defined in formulas I-XIX above and X₅ is a halogen, including F, Cl and Br and in a specific embodiment is Br. In specific embodiments of formulas XXXV-XLII, y is 0. In specific embodiments of formulas XXXV-XLII, y is 1 and L₂ is —(CH₂)ₙ— and n is 1, 2 or 3. In specific embodiments of formulas XXXV-XLII, the A ring is a phenyl ring where R_{A} is hydrogen. In specific embodiments R_{P} is a group selected from any one of R_{N}-1 to R_{N}-31. In specific embodiments, the B ring of formula XLII is that of formula RBI as shown in Scheme 4. In more specific embodiments, the B ring of formula XLII is that of RB2-RB5 of Scheme 4.

The invention provides salts, particularly pharmaceutically acceptable salts of each of the compounds of any of formulas I-IX, XI-XIX, XXX-XXXII, XXXV-XLII and formula XX below. The invention provides solvates and salts thereof, particularly pharmaceutically acceptable solvates and salts of each of the compounds of any of formulas I-XIX, XXX-XXXII, XXXV, XXXV-XLII and formula XX and XXI below. A preferred solvate is a hydrate. The invention provides pharmaceutical compositions comprising any compound of any one of the formulas herein.

In embodiments, compounds of formula XLV are useful in the methods, pharmaceutical compositions and pharmaceutical combinations herein:

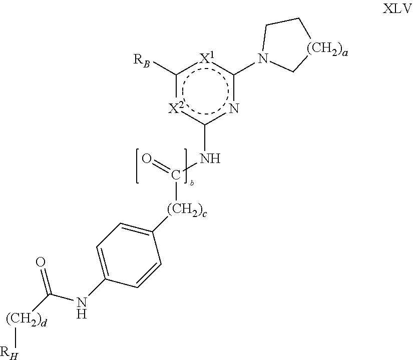

or salts or solvates thereof,

wherein:

X¹ and X² are independently CH or N;

R_{B} is hydrogen, C1-C3 alkyl or C1-C3 fluoroalkyl;

a, b, c or d are zero or integers, where a is 1 or 2, b is 0 or 1, c is 0 or 1, and d is 0 or 1; and

R_{H} is selected from any one of the moieties of Scheme 3, R12-1 to R12-84.

In embodiments of formula XLV:

X¹ is N and X² is CH or X¹ is CH and X² is N;

X¹ is N and X² is CH;

X¹ is CH and X² is N;

a is 1 and X¹ is N and X² is CH or X¹ is CH and X² is N;

a is 1 and X¹ is N and X² is CH;

a is 1 and X¹ is CH and X² is N;

a is 2 and X¹ is N and X² is CH or X¹ is CH and X² is N;

a is 2 and X¹ is N and X² is CH; or

a is 2 and X¹ is CH and X² is N.

In embodiments of formula XLV and each of the forgoing embodiments of X¹, X², and a therein:

b is 0 and c is 0, b is 0 and c is 1, b is 1 and c is 0, orb is 1 and c is 1;

b is 0 and c is 0;

b is 0 and cis 1;

b is 1 and c is 0; or

b is 1 and c is 1.

In embodiments of formula XLV and each of the forgoing embodiments of X¹, X², a, b and c

therein:

d is 0 or

d is 1.

In embodiments of formula XLV and each of the forgoing embodiments of X¹, X², a, b, c and d

therein:

R_{H} is one of moieties R12-1 to R12-84 of Scheme 3; or

R_{H} is one of moieties R12-5; R12-44; R12-45; R12-58; R12-62; R12-75, R12-79; or R12-80; or

RH is:

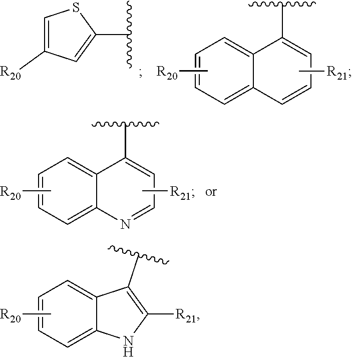

where:

R₂₀ and R₂₁ are independently, a hydrogen, a C1-C3 alkyl, a C1-C3 fluoroalkyl or a halogen on the indicated carbon or represents substitution on the indicated ring with one or more of the listed atoms or groups.

In embodiments of the foregoing embodiments of formula XLV, R₂₀ and R₂₁ are both hydrogens or represent hydrogens at all available ring positions.

In embodiments of the foregoing embodiments of formula XLV, R₂₀ and R₂₁ are independently a hydrogen, methyl, trifluormethyl or halogen on the indicated carbon or represents substitution on the indicated ring with one or more of the listed atoms or groups.

In embodiments of the foregoing embodiments of formula XLV, R₂₀ and R₂₁ are independently a methyl, trifluormethyl or halogen on the indicated carbon above or represents substitution on the indicated ring with one or more of the listed atoms or groups.

In embodiments of the foregoing embodiments of formula XLV, R₂₁ is hydrogen or represents hydrogen at all available positions on the indicated ring and R₂₀ is a methyl, trifluormethyl or halogen on the indicated carbon above or represents substitution on the indicated ring with one or more of the listed atoms or groups.

In embodiments of the foregoing embodiments of formula XLV, R₂₀ is hydrogen or represents hydrogen at all available positions on the indicated ring and R₂₁ is a methyl, trifluormethyl or halogen on the indicated carbon above or represents substitution on the indicated ring with one or more of the listed atoms or groups.

In embodiments of the foregoing embodiments of formula XLV, the halogen of R₂₀ or R₂₁ is independently fluorine, chlorine or bromine.

In embodiments of the foregoing embodiments of formula XLV, R_{H} is R12-79; R12-80; R12-44, wherein R′ represents hydrogens at all ring positions; R12-45, wherein R′ represents hydrogens at all ring positions; R12-58, wherein R′ represents hydrogens at all available ring positions; R12-62, wherein R′ represents hydrogens at all available ring positions; 2-haloquinolin-4-yl; 2-chloroquinolin-4-yl; R12-75, where both Rs are hydrogen and X is a halogen, R12-5, wherein R is hydrogen and R′ represents hydrogens on all ring positions; R12-5, where R is hydrogen and R′ represents a halogen at the 6-ring position; 6-chloroquinolin-4-yl, 2-C1-C3alkyl-1H-indol-3-yl or 2-methyl-1 H-indol-3-yl.

In embodiments of the foregoing embodiments of formula XLV, R_{H} is R12-80; R12-44, wherein R′ represents hydrogens at all ring positions; R12-58, wherein R′ represents hydrogens at all available ring positions; 2-haloquinolin-4-yl; 2-chloroquinolin-4-yl; R12-5, wherein R is hydrogen and R′ represents hydrogens on all ring positions; R12-5, where R is hydrogen and R′ represents a halogen at the 6-ring position; 6-chloroquinolin-4-yl, 2-C1-C3 alkyl-1 H-indol-3-yl or 2-methyl-1H-indol-3-yl.

In embodiments of the foregoing embodiments of formula XLV, R_{H} is naphth-1-yl.

In embodiments of the foregoing embodiments of formula XLV, R_{H} is 4-bromothiophen-2-yl.

In embodiments of the foregoing embodiments of formula XLV, R_{H} is thiophen-2-yl.

In embodiments of the foregoing embodiments of formula XLV, R_{H} is 1 H-indol-3-yl.

In embodiments of the forgoing embodiments of formula XLV, R_{H} is 6-chloro-1H-indol-3-yl.

In embodiments of the forgoing embodiments of formula XLV, R_{H} is 2-methyl-1H-indol-3-yl.

In embodiments of the foregoing embodiments of formula XLV, R_{H} is quinolin-4-yl.

In embodiments of the foregoing embodiments of formula XLV, R_{H} is 2-chloroquinolin-4-yl.

In embodiments of formula XLV, the compound is selected from compounds 28, 31, 52, 54, 57, 75, 118, 126, 131, 150, or 169. In more specific embodiments, the compound is selected from compounds 52, 118, 126, 131, 150, or 169. In embodiments of formula XLV, the compound is selected from compounds 28, 31, 54, 57, or 75. In embodiments of formula XLV, the compound is one or more of compounds 28, 31, 52, 54, 57, 75, 118, 126, 131, 150, or 169. In embodiments of formula XLV, the compound is one of compounds 28, 31, 52, 54, 57, 75, 118, 126, 131, 150, or 169.

In embodiments, the compounds of formula XLVI are useful in the method, pharmaceutical compositions and pharmaceutical combinations as described herein:

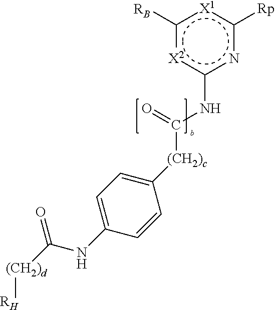

or salts or solvates thereof;

wherein variables are as defined for formula XLV and R_{H} and R_{P} are as defined in formula I and various embodiments thereof listed above. In embodiments, R_{F} is any of the moieties RN1-RN39,

In embodiment of formula XLVI, R_{P} is any of R_{N}1; R_{N}3; R_{N}2 or R_{N}4; R_{N}5 or R_{N}6; R_{N}7 or R_{N}8; R_{N}9; R_{N}10; R_{N}11; R_{N}12; R_{N}13; R_{N}14; R_{N}15; R_{N}16; R_{N}17 or R_{N}18; R_{N}19 or R_{N}20; R_{N}21; R_{N}22; R_{N}23 or R_{N}24; R_{N}25; R_{N}26-R_{N}29; R_{N}27-R_{N}32; R_{N}30; R_{N}31; R_{N}33-R_{N}36; R_{N}37; R_{N}38; R_{N}39; or

RN1, RN2, R_{N}3, R_{N}4, R_{N}11, R_{N}13, or R_{N}14; or

R_{N}1-R_{N}31 which is unsubstituted or

R_{N}32-R_{N}-39; or

RN37; or

R_{N}38 or R_{N}39.

An aliphatic compound is an organic compound containing carbon and hydrogen joined together in straight chains, branched chains, or non-aromatic rings and which may contain single, double, or triple bonds. Aliphatic compounds are distinguished from aromatic compounds. The term aliphatic group herein refers to a monovalent group containing carbon and hydrogen that is not aromatic. Aliphatic groups include alkyl, alkenyl, alkynyl, cycloalkyl, cycloalkenyl, and cycloalkynyl, as well as aliphatic groups substituted with other aliphatic groups, e.g., alkenyl groups substituted with alkyl groups, alkyl groups substituted with cycloalkyl groups.

The terms alkyl or alkyl group refer to a monoradical of a straight-chain or branched saturated hydrocarbon. Alkyl groups include straight-chain and branched alkyl groups. Unless otherwise indicated alkyl groups have 1-8 carbon atoms (C1-C8 alkyl groups) and preferred are those that contain 1-6 carbon atoms (C1-C6 alkyl groups) and more preferred are those that contain 1-3 carbon atoms (C1-C3 alkyl groups). Alkyl groups are optionally substituted with one or more non-hydrogen substituents as described herein. Exemplary alkyl groups include methyl, ethyl, n-propyl, iso-propyl, n-butyl, s-butyl, t-butyl, n-pentyl, various branched-pentyl, n-hexyl, various branched hexyl, all of which are optionally substituted, where substitution is defined elsewhere herein. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl.

Cycloalkyl groups are alkyl groups having at least one 3- or higher member carbon ring. Cycloalkyl groups include those having 3-12-member carbon rings. Cycloalkyl groups include those having 3-20 carbon atoms and those having 3-12 carbon atoms. More specifically, cycloalkyl groups can have at least one 3-10-member carbon ring. Cycloalkyl groups can have a single carbon ring having 3-10 carbons in the ring. Cycloalkyl groups are optionally substituted. Cycloalkyl groups can be bicyclic having 6-12 carbons. Exemplary cycloalkyl groups include among others, cyclopropyl, cyclobutyl, cyclopentyl, cyclohexyl, cycloheptyl, cyclooctyl, cyclononyl and cyclodecyl groups. Bicyclic alkyl groups include fused bicyclci grouos and bridged bicyclic groups. Exemplary bicycloalkyl groups include, among others, bicyclo[2.2.2]octyl, bicyclo[4.4.0] decyl (decalinyl), and bicyclo[2.2.2]heptyl (norbornyl).

Cycloalkylalkyl groups are alkyl groups as described herein which are substituted with a cycloalkyl group as described herein. More specifically, the alkyl group is a methyl or an ethyl group and the cycloalkyl group is a cyclopropyl, cyclobutyl, cyclopentyl, or cyclohexyl group. Cycloalkyl groups are optionally substituted. In specific embodiments, optional substitution includes substitution with one or more halogens, alkyl groups having 1-3 carbon atoms, alkoxy groups having 1-3 carbo atoms, hydroxyl and nitro groups

The term alkylene refers to a divalent radical of a straight-chain or branched saturated hydrocarbon. Alkylene groups can have 1-12 carbon atoms unless otherwise indicated. Alkylene groups include those having 2-12, 2-8, 2-6 or 2-4 carbon atoms. Linker groups (L1) herein include alkylene groups, particularly straight chain, unsubstituted alkylene groups, —(CH2)n-, where n is 1-12, n is 1-10, n is 1-9, n is 1-8, n is 1-7, n is 1-6, n is 1-5, n is 1-4, n is 1-3, n is 2-10, n is 2-9, n is 2-8, n is 2-7, n is 2-6, n is 2-5 or n is 2-4.

An alkoxy group is an alkyl group, as broadly discussed above, linked to oxygen (Ralkyl-O—). An alkoxy grou is monovalent.

An alkenylene group is a divalent radical of a straight-chain or branched alkylene group which has one or more carbon-carbon double bonds. In specific embodiments, the same carbon atom is not part of two double bonds. In an alkenylene group one or more CH2-CH2 moieties of the alkylene group are replaced with a carbon-carbon double bond. In specific embodiments, an alkenylene group contains 2-12 carbon atoms or more preferably 3-12 carbon atoms. In specific embodiments, an alkenylene group contains one or two double bonds. In specific embodiments, the alkenylene group contains one or two trans-double bonds. In specific embodiments, the alkenylene group contains one or two cis-double bonds. Exemplary alkenylene groups include:

—(CH₂)n-CH═CH—(CH₂)n-, where n is 1-4 and more preferably is 2; and

—(CH₂)n-CH═CH—CH═CH—(CH₂)n-, where n is 1-4 and more preferably is 1 or 2.

An alkoxyalkyl group is an alkyl group in which one or more of the non-adjacent internal —CH₂— groups are replaced with —O—, such a group may also be termed an ether group. The alkoxyalkyl group is monovalent. These groups may be straight-chain or branched, but straight-chain groups are preferred. Alkoxyalkyl groups include those having 2-12 carbon atoms and 1, 2, 3 or 4 oxygen atoms. More specifically, alkoxyalkyl groups include those having 3 or 4 carbons and 1 oxygen, or those having 4, 5 or 6 carbons and 2 oxygens. Each oxygen in the group is bonded to a carbon in the group. The group is bonded into a molecule via a bond to a carbon in the group.

An alkoxyalkylene group is a divalent alkoxyalkyl group. This group can be described as an alkylene group in which one or more of the internal —CH₂— groups are replaced with an oxygen. These groups may be straight-chain or branched, but straight-chain groups are preferred. Alkoxyalkylene groups include those having 2-12 carbon atoms and 1, 2, 3 or 4 oxygen atoms. More specifically, alkoxyalkylene groups include those having 3 or 4 carbons and 1 oxygen, or those having 4, 5 or 6 carbons and 2 oxygens. Each oxygen in the group is bonded to a carbon in the group. The group is bonded into a molecule via bonds to a carbon in the group. Linker groups (L1) herein include alkoxyalkylene groups, particularly straight chain, unsubstituted alkoxyalkylene groups. Specific alkoxyalkylene groups include, among others, —CH₂—O—CH₂—, —CH₂—CH₂—O—CH₂—CH₂—, —CH₂—CH₂—CH₂—O—CH₂—CH₂—CH₂—, —CH₂—CH₂—O—CH₂—, —CH₂—O—CH₂—CH₂—, —CH₂—CH₂—O—CH₂—CH₂—O—CH₂—, —CH₂—CH₂—O—CH₂—CH₂—O—CH₂—CH₂—, —CH₂—CH₂—CH₂—O—CH₂—CH₂—CH₂—O—CH₂—, and —CH₂—CH₂—CH₂—O—CH₂—CH₂—CH₂—O—CH₂—CH₂—.

The term acyl group refers to the group —CO—R where R is hydrogen, an alkyl or aryl group as described herein.

Aryl groups include monovalent groups having one or more 5- or 6-member aromatic rings. Aryl groups can contain one, two or three, 6-member aromatic rings. Aryl groups can contain two or more fused aromatic rings. Aryl groups can contain two or three fused aromatic rings. Aryl groups are optionally substituted with one or more non-hydrogen substituents. Substituted aryl groups include among others those which are substituted with alkyl or alkenyl groups, which groups in turn can be optionally substituted. Specific aryl groups include phenyl groups, biphenyl groups, and naphthyl groups, all of which are optionally substituted as described herein. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms.

Alkyl groups include arylalkyl groups in which an alkyl group is substituted with an aryl group. Arylalkyl groups include benzyl and phenethyl groups among others. Arylalkyl groups are optionally substituted as described herein. Substituted arylalkyl groups include those in which the aryl group is substituted with 1-5 non-hydrogen substituents and particularly those substituted with 1, 2 or 3 non-hydrogen substituents. Useful substituents include among others, methyl, methoxy, hydroxy, halogen, and nitro. Particularly useful substituents are one or more halogens. Specific substituents include F. Cl, and nitro.

An acyl group is an R—CO— groups where R is alkyl, cycloalkyl or aryl as defined herein each of which is optionally substituted.

An acyl oxy group is an R—COO— group where R is alkyl, cycloalkyl or aryl as defined herein each of which is optionally substituted.

An alkoxycarbonyl group is an RO—CO— group where R is an alkyl or cycloalkyl as defined herein each of which is optionally substituted.

A carboxyl group is a —COOH group which may be in the ionized form —COO—.

A heterocyclic group is a monovalent group having one or more saturated or unsaturated carbon rings and which contains one or more heteroatoms (e.g., N, O or S) per ring. In specific embodiments, a heterocyclic group contains one to six heteroatoms (e.g., N, O or S). In specific embodiments, a heterocyclic groups contains one to three heteroatoms. These groups optionally contain one, two or three double bonds. To satisfy valence requirements, a ring atom may be bonded to one or more hydrogens or be substituted as described herein. One or more carbons in the heterocyclic ring can be —CO— groups. The heteroatoms in the ring may be substituted with one or more substituents dependent upon valency or substituted with one or more oxygen atoms. Heterocyclic ring members can include, for example, —N═, —NH—, —NR—, —SO—, or —SO₂—. Heterocyclic groups include those having 3-12 carbon atoms, and 1-6, heteroatoms, wherein 1 or 2 carbon atoms are replaced with a —CO— group. Heterocyclic groups include those having 3-12 or 3-10 ring atoms of which up to three can be heteroatoms other than carbon. Heterocyclic groups can contain one or more rings each of which is saturated or unsaturated. Heterocyclic groups include bicyclic and tricyclic groups. Preferred heterocyclic groups have 5- or 6-member rings. Heterocyclic groups are optionally substituted as described herein. Specifically, heterocyclic groups can be substituted with one or more alkyl groups. Heterocyclic groups include those having 5- and 6- member rings with one or two nitrogens and one or two double bonds. Heterocyclic groups include those having 5- and 6-member rings with an oxygen or a sulfur and one or two double bonds. Heterocyclic group include those having 5- or 6-member rings and two different heteroatoms, e.g., N and 0, 0 and S or N and S. Specific heterocyclic groups include among others among others, pyrrolidinyl, piperidyl, piperazinyl, pyrrolyl, pyrrolinyl, furyl, thienyl, morpholinyl, oxazolyl, oxazolinyl, oxazolidinyl, indolyl, triazoly, triazinyl groups, sultam groups (e.g., 1,1-dioxidoisothiazolidin-2-yl, 1,1-dioxidothiazinan-2-yl) Heterocycylalky groups are alkyl groups substituted with one or more heterocycyl groups wherein the alkyl groups optionally carry additional substituents and the heterocycyl groups are optionally substituted. Specific groups are heterocycyl-substituted methyl or ethyl groups.

Heteroaryl groups are monovalent groups having one or more aromatic rings in which at least one ring contains a heteroatom (a non-carbon ring atom). Heteroaryl groups include those having one or two heteroaromatic rings carrying 1, 2 or 3 heteroatoms and optionally have one 6-member aromatic ring. Heteroaryl groups can contain 5-20, 5-12 or 5-10 ring atoms. Heteroaryl groups include those having one aromatic ring contains a heteroatom and one aromatic ring containing carbon ring atoms. Heteroaryl groups include those having one or more 5- or 6-member aromatic heteroaromatic rings and one or more 6-member carbon aromatic rings. Heteroaromatic rings can include one or more N, O, or S atoms in the ring. Heteroaromatic rings can include those with one, two or three N, those with one or two O, and those with one or two S, or combinations of one or two or three N, O or S. Specific heteroaryl groups include furyl, pyridinyl, pyrazinyl, pyrimidinyl, quinolinyl, purinyl, indolyl groups. In a specific embodiment, the heteroaryl group is an indolyl group and more specifically is an indol-3-yl group:

Heteroatoms include O, N, S, P or B. More specifically heteroatoms are N, O or S. In specific embodiments, one or more heteroatoms are substituted for carbons in aromatic or carbocyclic rings. To satisfy valence any heteroatoms in such aromatic or carbocyclic rings may be bonded to H or a substituent group, e.g., an alkyl group or other substituent.

Heteroarylalkyl groups are alkyl groups substituted with one or more heteroaryl groups wherein the alkyl groups optionally carry additional substituents and the aryl groups are optionally substituted. Specific alkyl groups are methyl and ethyl groups.

The term amino group refers to the species —N(H)₂. The term alkylamino refers to the species —NHR″ where R″ is an alkyl group, particularly an alkyl group having 1-3 carbon atoms. The term dialkylamino refers to the species —N(R″)² where each R″ is independently an alkyl group, particularly an alkyl group having 1-3 carbon atoms.

Groups herein are optionally substituted. Most generally any alky, cycloalkyl, aryl, heteroaryl and heterocyclic groups can be substituted with one or more halogen, hydroxyl group, nitro group, cyano group, isocyano group, oxo group, thioxo group, azide group, cyanate group, isocyanate group, acyl group, haloakyl group, alkyl group, alkenyl group or alkynyl group (particularly those having 1-4 carbons), a phenyl or benzyl group (including those that are halogen or alkyl substituted), alkoxy, alkylthio, or mercapto (HS—). In specific embodiments, optional substitution is substitution with 1-12 non-hydrogen substituents. In specific embodiments, optional substitution is substitution with 1-6 non-hydrogen substituents. In specific embodiments, optional substitution is substitution with 1-3 non-hydrogen substituents. In specific embodiments, optional substituents contain 6 or fewer carbon atoms. In specific embodiments, optional substitution is substitution by one or more halogen, hydroxy group, cyano group, oxo group, thioxo group, unsubstituted C1-C6 alkyl group or unsubstituted aryl group. The term oxo group and thioxo group refer to substitution of a carbon atom with a=O or a=S to form respectively —CO— (carbonyl) or —CS— (thiocarbonyl) groups.

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups, and methoxyphenyl groups, particularly 4-methoxyphenyl groups.

The term aromatic as applied to cyclic groups refers to ring structures which contain double bonds that are conjugated around the entire ring structure, possibly through one or more heteroatoms such as an oxygen atom, sulfur atom or a nitrogen atom. Aryl groups, and heteroaryl groups are examples of aromatic groups. The conjugated system of an aromatic group contains a characteristic number of electrons, for example, 6 or 10 electrons that occupy the electronic orbitals making up the conjugated system, which are typically un-hybridized p-orbitals.

The term carbocyclic refers to a monovalent group having a carbon ring or ring system which comprises 3 to 12 carbon atoms and may be monocyclic, bicyclic or tricyclic. The ring does not contain any heteroatoms. The ring may be unsaturated, partially unsaturated or saturated.

Compounds and substituent groups of formulas herein are optionally substituted. A substituent refers to a single atom (for example, a halogen atom) or a group of two or more atoms that are covalently bonded to each other, which are covalently bonded to an atom or atoms in a molecule to satisfy the valency requirements of the atom or atoms of the molecule, typically in place of a hydrogen atom. Examples of substituents include among others alkyl groups, hydroxyl groups, alkoxy groups, acyloxy groups, mercapto groups, and aryl groups. Substituent groups may themselves be substituted.

Substituted or substitution refer to replacement of a hydrogen atom of a molecule or of an chemical group or moiety with one or more additional substituents such as, but not limited to, halogen, alkyl, alkoxy, alkylthio, trifluoromethyl, acyloxy, hydroxy, mercapto, carboxy, aryloxy, aryl, arylalkyl, heteroaryl, amino, alkylamino, dialkylamino, morpholino, piperidino, pyrrolidin-1-yl, piperazin-1-yl, nitro, sulfato, or other R-groups.

Carbocyclic or heterocyclic rings are optionally substituted as described generally for other groups, such as alkyl and aryl groups herein. Substitution if present is typically on ring C, ring N or both. In addition, carbocyclic and heterocyclic ring can optionally contain a —CO—, —CO—O—, —CS— or —CS—O-moiety in the ring.

As to any of the chemical groups herein that are substituted, i.e., contain one or more non-hydrogen substituents, it is understood, that such groups do not contain any substitution or substitution patterns which are sterically impractical and/or synthetically non-feasible. In addition, the compounds of this invention include all stereochemical isomers arising from the substitution of these compounds.

Protected derivatives of the disclosed compounds also are contemplated. A variety of suitable protecting groups for use with the disclosed compounds are disclosed in Greene and Wuts, Protective Groups in Organic Synthesis; 3rd Ed.; John Wiley & Sons, New York, 1999. In general, protecting groups are removed under conditions which will not affect the remaining portion of the molecule. These methods are well known in the art and include acid hydrolysis, hydrogenolysis, and the like. One preferred method involves the removal of an ester, such as cleavage of a phosphonate ester using Lewis acidic conditions, such as in TMS-Br mediated ester cleavage to yield the free phosphonate. A second preferred method involves removal of a protecting group, such as removal of a benzyl group by hydrogenolysis utilizing palladium on carbon in a suitable solvent system such as an alcohol, acetic acid, and the like or mixtures thereof. A t-butoxy-based group, including t-butoxy carbonyl protecting groups can be removed utilizing an inorganic or organic acid, such as HCl or trifluoroacetic acid, in a suitable solvent system, such as water, dioxane and/or methylene chloride. Another exemplary protecting group, suitable for protecting amino and hydroxy functions amino is trityl. Other conventional protecting groups are known, and suitable protecting groups can be selected by those of skill in the art in consultation with Greene and Wuts, Protective Groups in Organic Synthesis; 3rd Ed.; John Wiley & Sons, New York, 1999. When an amine is deprotected, the resulting salt can readily be neutralized to yield the free amine. Similarly, when an acid moiety, such as a phosphonic acid moiety is unveiled, the compound may be isolated as the acid compound or as a salt thereof. Protected derivatives of compounds herein can, for example, be employed in the synthesis of structurally related compounds herein.

The present invention provides novel therapeutic strategies for targeting TCF-driven EMT, a process that promotes tumor cell heterogeneity, MDR, and metastasis. The inventors' structure-based drug design has produced novel potent CHD1L inhibitors which in an embodiment target TCF-driven EMT. Reversion of EMT by CHD1L inhibitors may be an effective treatment when used in combination with cytotoxic chemotherapy and targeted antitumor drugs as well as radiation therapy. These EMT-targeting agents may also sensitize both primary tumors and metastatic lesions to clinically relevant therapies, and potentially inhibit tumor cell metastasis.

Thus, one aspect of this invention are CHD1L inhibitors which can be used to treat or prevent metastasis of a wide variety of advanced solid tumors and blood cancers. Pharmaceutically acceptable salts, prodrugs, stereoisomers, and metabolites of all the CHD1L inhibitor compounds of this invention also are contemplated.

The invention expressly includes pharmaceutically usable solvates of compounds according to formulas herein. Specifically, useful solvates are hydrates. The compounds of formula I or salts thereof can be solvated (e.g., hydrated). The solvation can occur in the course of the manufacturing process or can take place (e.g., as a consequence of hygroscopic properties of an initially anhydrous compound of formulas herein (hydration)).

Compounds of the invention can have prodrug forms. Prodrugs of the compounds of the invention are useful in the methods of this invention. Any compound that will be converted in vivo to provide a biologically, pharmaceutically or therapeutically active form of a compound of the invention is a prodrug. Various examples and forms of prodrugs are well known in the art. A prodrug is an active or inactive compound that is modified chemically through in vivo physiological action, such as hydrolysis, metabolism and the like, into an active compound following administration of the prodrug to a subject. The term prodrug as used throughout this text means the pharmacologically acceptable derivatives such as esters, amides and phosphates, such that the resulting in vivo biotransformation product of the derivative is the active drug as defined in the compounds described herein. Prodrugs preferably have excellent aqueous solubility, increased bioavailability, and are readily metabolized into the active TOP2A inhibitors in vivo. Prodrugs of compounds described herein may be prepared by modifying functional groups present in the compound in such a way that the modifications are cleaved, either by routine manipulation or in vivo, to the parent compound. The suitability and techniques involved in making and using prodrugs are well known by those skilled in the art. Examples of prodrugs are found, inter alia, in Design of Prodrugs, edited by H. Bundgaard, (Elsevier, 1985), Methods in Enzymology, Vol. 42, at pp. 309-396, edited by K. Widder, et. al. (Academic Press, 1985); A Textbook of Drug Design and Development, edited by Krosgaard-Larsen and H. Bundgaard, Chapter 5, “Design and Application of Prodrugs,” by H. Bundgaard, at pp. 113-191, 1991); H. Bundgaard, Advanced Drug Delivery Reviews, Vol. 8, p. 1-38 (1992); H. Bundgaard, et al., Journal of Pharmaceutical Sciences, Vol. 77, p. 285 (1988); and Nogrady (1985) Medicinal Chemistry A Biochemical Approach, Oxford University Press, New York, pages 388-392).

Administration of and administering a compound or composition should be understood to mean providing a compound or salt thereof, a prodrug of a compound, or a pharmaceutical composition comprising a compound. The compound or composition can be administered by another person to the patient (e.g., intravenously) or it can be self-administered by the subject (e.g., tablets or capsules). The term “patient” refers to mammals (for example, humans and veterinary animals such as dogs, cats, pigs, horses, sheep, and cattle). Administration of CHD1L inhibitors herein in combination with other agents, such as alternative anti-cancer, antineoplastic or cancer cytotoxic agents is contemplated. Such combined administration includes administration of two or more active ingredients at the same time or at times separated by minutes, hours or days as is found to be effective and consistent with the administration of any known alternative treatments with which the CHD1L inhibitor is to be combined. Combined administration further includes administration by the same method and/or location of the patient's body or by different methods at different locations, again as is consistent with and consistent with the administration of known alternative treatments with which the CHD1L inhibitor is to be combined.

In embodiments, the CHD1L inhibitors are administered together with an alternative cancer cytotoxic or cancer cytotoxic or antineoplastic agent or antineoplastic procedure (e.g., radiation treatment) in one or more acceptable pharmaceutical dosage forms or are administered separately within a selected time period to provide synergistic effect.

In embodiments, the CHD1L inhibitor(s) is (are) administered by the same route as the alternative cancer cytotoxic or cancer cytotoxic or antineoplastic agent. In embodiments, the CHD1L inhibitor(s) is administered by a route different from the alternative cancer cytotoxic or antineoplastic agent. In embodiments, the CHD1L inhibitor(s) are administered orally or by injection. In embodiments, the alternative cancer cytotoxic or antineoplastic agent are administered orally or by injection. In embodiments, the CHD1L inhibitor(s) are administered locally to tumors or systemically or a combination of both forms of administration. In embodiments, the alternative neoplastic agent is administered locally to tumors or systemically or a combination of both forms of administration.

In embodiments, components of the pharmaceutical combination (one or more CHD1L with one alternative neoplastic agent) are administered to a subject in need thereof in a joint therapeutic amount to provide synergistic therapeutic effect. In embodiments, components of the pharmaceutical combination are administered by any appropriate mode of administration to a subject in need thereof in a joint therapeutic amount to provide synergistic therapeutic effect. In embodiments, components of the pharmaceutical combination are administered by local or systemic administration or by a combination of local and systemic administration to a subject in need thereof in a joint therapeutic amount to provide synergistic therapeutic effect.

If a patient is to receive or is receiving multiple pharmaceutically active compounds, the compounds can be administered simultaneously or sequentially. For example, in the case of tablets, the active compounds may be found in one tablet or in separate tablets, which are administered at once or sequentially in any order. In addition, it should be recognized that the compositions may be in different dosage forms. For example, one or more compounds may be delivered via a tablet, while another is administered via injection or orally as a syrup. All combinations, delivery methods and administration sequences are contemplated.

In embodiments, the combination therapy herein comprises administration of one or more CHD1L inhibitor and administration of one or more alternative cancer cytotoxic or antineoplastic agent to a patient in need of treatment. Administration includes any form or forms of administration which achieves synergistic therapeutic action of the CHD1L inhibitor(s) and the alternative cancer cytotoxic or antineoplastic agent. Administration includes simultaneous, concurrent, sequential, successive, alternate or separate administration of inhibitor(s) CHD1L with the alternative cancer cytotoxic or antineoplastic agent. In embodiments, oral administration of CHD1L inhibitor(s) may be combined with administration of the alternative cancer cytotoxic or antineoplastic agent orally or by injection. The order (sequence) and relative timing of administration of CHD1L inhibitor(s) and administration of the alternative cancer cytotoxic or antineoplastic agent is adjusted to achieve synergistic therapeutic action. In embodiments, administration of CHD1L inhibitor(s) is at the same time (i.e., within up to 2 hours of each other) as administration of alternative cancer cytotoxic or antineoplastic agent. In embodiments, administration of CHD1L inhibitor(s) is separate from administration of the alternative cancer cytotoxic or antineoplastic agent within a selected time period of more than 2 hours of each other. In embodiments, administration of CHD1L inhibitor(s) is separate from administration of the alternative cancer cytotoxic or antineoplastic agent, but within a selected time period of ±24 hours to 1 week.

In embodiments, the invention provides a pharmaceutical combination of one or more CHD1L inhibitor and one or more alternative cancer cytotoxic or antineoplastic agent. In embodiments, the components of the pharmaceutical combination can be together or separate. In embodiments, the pharmaceutical combination is a pharmaceutical compositions containing one or more CHDL1 inhibitor and one or more topoisomerase inhibitor, PARP inhibitor, or thymidylate synthase inhibitor. In embodiments, the pharmaceutical combination is two or more separate pharmaceutical compositions each containing different components of the pharmaceutical combination. In embodiments, the pharmaceutical combination is two separate pharmaceutical compositions, one containing one or more CHD1L inhibitors and one containing one or more topoisomerase inhibitor, one or more PARP inhibitor and/or one or more thymidylate synthase inhibitor. In embodiments, the pharmaceutical combination is a single pharmaceutical composition, containing one or more CHD1L inhibitors and one containing one or more inhibitor of PARP. In embodiments, the pharmaceutical combination is a single pharmaceutical composition, containing one or more CHD1L inhibitors and one containing one or more inhibitor of topoisomerase. In embodiments, the pharmaceutical combination is a single pharmaceutical composition, containing one or more CHD1L inhibitors and one containing one or more inhibitor of thymidylate synthase.

In embodiments, the components of the pharmaceutical combination are administered together in a single dosage form appropriate for the selected mode of administration, e.g., oral or by injection. In embodiments, where the pharmaceutical combination is a single dosage form, the relative amount of the one or more CHD1L inhibitor and one or more alternative cancer cytotoxic or antineoplastic agent in the dosage form is fixed. In embodiments, the pharmaceutical combination is administered as two separate pharmaceutical compositions or dosage forms, one containing one or more CHD1L inhibitors and one containing one or more alternative cancer cytotoxic or antineoplastic agent. Such separate administration may be in the same or different dosage form for appropriate for the selected mode of administration.

In embodiments, the components of the pharmaceutical combination are administered in one or more dosage form and may be administered at the same time or at different times. In embodiments, the components of the pharmaceutical combination can be administered simultaneously, concurrently or sequentially with or without specific time limits where such administration provides therapeutically effective combined amounts of the one or more CHD1L inhibitor and the one or more alternative cancer cytotoxic or antineoplastic agent. In embodiments, the combined therapeutically effective amount of the one or more CHD1L inhibitor and the one or more alternative cancer cytotoxic or antineoplastic agent exhibits greater than an additive therapeutic effect. In embodiments, the combined therapeutically effective amount of the one or more CHD1L inhibitor and the one or more alternative cancer cytotoxic or antineoplastic agent exhibits a synergistic therapeutic effect.

In embodiments, the one or more CHD1L inhibitor and the one or more alternative cancer cytotoxic or antineoplastic agent are formulated separately and sold separately, but administered to a subject in need thereof as a pharmaceutical combination. In embodiments, the one or more CHD1L inhibitor and the one or more alternative cancer cytotoxic or antineoplastic agent are administered for treatment of the same disorder or disease state. In specific embodiments, the disorder or disease state is a proliferative disorder and more specifically is cancer. In embodiments, the components of the pharmaceutical combination may be sold together or separately in the same or different dosage forms, in combination with instructions for simultaneous, concurrent or sequential administration of the components of the pharmaceutical combination.

Any forms of administration that achieve the desired combined therapeutic effect can be employed. For example, the combined administration can be local to the site of one or more tumors or can be systemically administered to the subject. In embodiments, one or more components of the pharmaceutical combination can be administered locally to one or more tumor site and one or more other components of the pharmaceutical combination can be administered systemically to the subject. Local or systemic administration can be by any appropriate mode of administration. Local administration can, for example, be by injection, infusion or by topical application. Systemic administration can, for example, be oral, topical or by injection.

One or more CHD1L inhibitors as described herein can be administered in combination with chemotherapy, radiotherapy, immunotherapy, surgery or any combination of such therapies. The combination therapy(ies) described herein can be administered in combination with chemotherapy, radiotherapy, immunotherapy, surgery or any combination of such therapies.

Pharmaceutical compositions herein comprise a named active ingredient or combination of named active ingredients in an amount effective for achieving the desired biological activity for a given form of administration to a given patient and optionally contain a pharmaceutically acceptable excipient or carrier. Pharmaceutical compositions can include an amount (for example, a unit dosage) of one or more of the disclosed compounds together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, Pa. (19th Edition).

In embodiments, pharmaceutical compositions herein comprise one or more compounds of any of formulas I-XIX, XXX-XXXII, XXXV, XXXV-XLII, XLV, XLVI and formula XX and XXI or pharmaceutically acceptable salts, or solvates thereof and a pharmaceutically acceptable excipient. The term “excipient” means any pharmaceutically acceptable additive, carrier, diluent, adjuvant, or other ingredient, other than the active pharmaceutical ingredient (API) or another clearly designated active pharmaceutical ingredient, which is typically included for formulation and/or administration to a patient.

Pharmaceutically acceptable carriers are those carriers that are compatible with the other ingredients in the formulation and are biologically acceptable. Carriers can be solid or liquid.

In some embodiments, carriers are solids, for example, in which oral dosage forms are pills. In some embodiments, carriers are liquids, for example, in which oral dosage forms are solutions or suspensions. Carriers can include one or more substances that can also act as solubilizers, suspending agents, fillers, glidants, compression aids, binders, tablet-disintegrating agents, or encapsulating materials. Liquid carriers can be used in preparing solutions, suspensions, emulsions, syrups and elixirs. The active ingredient can be dissolved or suspended in a pharmaceutically acceptable liquid carrier such as water (of appropriate purity, e.g., pyrogen-free, sterile, etc.), an organic solvent, a mixture of both, or a pharmaceutically acceptable oil or fat. The liquid carrier can contain other suitable pharmaceutical additives such as, for example, solubilizers, emulsifiers, buffers, preservatives, sweeteners, flavoring agents, suspending agents, thickening agents, colors, viscosity regulators, stabilizers or osmo-regulators. Compositions for oral administration can be in either liquid or solid form.

Suitable examples of liquid carriers for oral and parenteral administration include water of appropriate purity, aqueous solutions (particularly containing additives, e.g., cellulose derivatives, sodium carboxymethyl cellulose solution), alcohols (including monohydric alcohols and polyhydric alcohols e.g., glycols) and their derivatives, and oils. In specific examples, liquid carriers for oral administration include solutions of active ingredients (i.e., CHD1L inhibitors preferably dissolved or suspended in a liquid carrier. For parenteral administration, the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant. Liquid pharmaceutical compositions that are sterile solutions or suspensions can be administered by, for example, intramuscular, intraperitoneal or subcutaneous injection. Sterile solutions can also be administered intravenously. Compositions for oral administration can be in either liquid or solid form. The carrier can also be in the form of creams and ointments, pastes, and gels. The creams and ointments can be viscous liquid or semisolid emulsions of either the oil-in-water or water-in-oil type.

In embodiments, administration of CHD1L inhibitors employs dosage forms comprising pharmaceutically acceptable polyethylene glycol (PEG). In such embodiments, the pharmaceutically acceptable PEG may be combined with a pharmaceutically acceptable organic solvent, particularly a pharmaceutically acceptable polar, aprotic solvent. In embodiments, the organic solvent is pharmaceutically acceptable DMSO. In embodiments, oral administration employs oral dosage forms comprising low molecular weight polyethylene glycol having molecular weight of 600 g/mole or less. In more specific embodiments, oral administration employs PEG 400. In more specific embodiments, oral administration employs PEG 200. In embodiments, PEG is described by its average Mn (number average) molecular weight. PEG having Mn of 600 or less are suitable for use in formulations of CHD1L inhibitors herein. More specifically, PEG having Mn of 400 or 200 are suitable for formulations herein. In embodiments, administration employs oral formulations comprising PEG, preferably low molecular weight PEG and more specifically PEG having Mn of 600 or less. In embodiments, oral formulations comprise a therapeutically effective amount of a CHD1L inhibitor in combination with PEG, particularly where the CHD1L inhibitor suspended or dissolved in the PEG. In embodiments, a combination of PEG and an appropriate pharmaceutically acceptable polar aprotic solvent. In embodiments, the polar aprotic solvent is pharmaceutically acceptable DMSO. In specific embodiment, the oral formulation comprises PEG and DMSO. In embodiments, the solvent combination of PEG and DMSO is miscible. In embodiments, the combination of PEG and DMSO dissolves the therapeutically effective amount of the CHD1L inhibitor. In embodiments, the volume ratio of PEG to DMSO in oral formulations ranges from 100 to 4. More specifically, the volume ratio of PEG to DMSO ranges from 20 to 4, or 9 to 4 or 12 to 6 or 10 to 8. In specific embodiments, a solvent mixture of 90% by volume PEG, particularly low molecular weight PEG, and 10% by volume DMSO is employed in oral formulations.

A “therapeutically effective amount” of the disclosed compounds is a dosage of the compound that is sufficient to achieve a desired therapeutic effect, such as an anti-tumor or anti-metastatic effect. It will be understood that the therapeutically effective amount of a given compound depends upon the compound, the route of administration and the dosage form as well as the patient to be treated (age, weight, etc.). In some examples, a therapeutically effective amount is an amount sufficient to achieve tissue concentrations at the site of action that are similar to those that are shown to modulate TCF-transcription and/or epithelial-mesenchymal transition (EMT) in tissue culture, in vitro, or in vivo. For example, a therapeutically effective amount of a compound may be such that the subject receives a dosage of about 0.1 μg/kg body weight/day to about 1000 mg/kg body weight/day, for example, a dosage of about 1 μg/kg body weight/day to about 1000 μg/kg body weight/day, such as a dosage of about 5 μg/kg body weight/day to about 500 μg/kg body weight/day. In cases in which treatment using a CHD1L inhibitor of the invention is combined with treatment using another active ingredient or with another form of cancer treatment or therapy, the therapeutically effect amount of the CHD1L inhibitor may depend upon the active ingredient, treatment or therapy with which it is combined.

The term modulate refers to the ability of a disclosed compound to alter the amount, degree, or rate of a biological function, the progression of a disease, or amelioration of a condition. For example, modulating can refer to the ability of a compound to elicit an increase or decrease in angiogenesis, to inhibit TCF-transcription and/or EMT, or to inhibit tumor metastasis or tumorigenesis.

Treatment refers to a therapeutic intervention that ameliorates a sign or symptom of a disease or pathological condition after it has begun to develop. As used herein, the term ameliorating, with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. The phrase treating a disease is inclusive of inhibiting the full development of a disease or condition, for example, in a subject who is at risk for a disease, or who has a disease, such as cancer or a disease associated with a compromised immune system. Preventing a disease or condition refers to prophylactically administering a composition to a subject who does not exhibit signs of a disease or exhibits only early signs of the disease, for the purpose of decreasing the risk of developing a pathology or condition, or diminishing the severity of a pathology or condition.

In general the CHD1I inhibitors herein can be used to treat cancer alone or in combination therapies as described herein. Cancers which may generally be treated with compounds of the present invention include, without limitation, carcinomas such as cancer of the bladder, breast, colon, rectum, kidney, liver, lung (small cell lung cancer, and non-small-cell lung cancer), esophagus, gall-bladder, ovary, pancreas, stomach, cervix, thyroid, prostate, and skin (including squamous cell carcinoma); hematopoietic tumors of lymphoid lineage (including leukemia, acute lymphocytic leukemia, chronic myelogenous leukemia, acute lymphoblastic leukemia, B-cell lymphoma, T-cell-lymphoma, Hodgkin's lymphoma, non-Hodgkin's lymphoma, hairy cell lymphoma and Burkett's lymphoma); hematopoietic tumors of myeloid lineage (including acute and chronic myelogenous leukemias, myelodysplastic syndrome and promyelocytic leukemia); tumors of mesenchymal origin (including fibrosarcoma and rhabdomyosarcoma, and other sarcomas, e.g., soft tissue and bone); tumors of the central and peripheral nervous system (including astrocytoma, neuroblastoma, glioma and schwannomas); and other tumors (including melanoma, seminoma, teratocarcinoma, osteosarcoma, xenoderoma pigmentosum, keratoctanthoma, thyroid follicular cancer and Kaposi's sarcoma). Other cancers that can be treated with the compound of the present invention include endometrial cancer, head and neck cancer, glioblastoma, malignant ascites, and hematopoietic cancers.

All references throughout this application, for example patent documents including issued or granted patents or equivalents; patent application publications; and non-patent literature documents or other source material; are hereby incorporated by reference herein in their entireties, as though individually incorporated by reference. All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (e.g., to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

Esquer et al., 2021 and any supplementary information for that journal article are each incorporated by reference herein in its entirety for descriptions of biological and chemical methods useful in making and assessing the activities and properties of the CHD1L inhibitors herein.

Abbott et al., 2020 and the supplementary information for that journal article are each incorporated by reference herein in its entirety for descriptions of biological and chemical methods useful in making and assessing the activities and properties of the CHD1L inhibitors herein.

Esquer et al., 2020 and any supplementary information for that journal article are each incorporated by reference herein in its entirety for descriptions of biological and chemical methods useful in making and assessing the activities and properties of the CHD1L inhibitors herein.

Yang et al., 2020 and any supplementary information for that journal article are each incorporated by reference herein in its entirety for descriptions of biological and chemical methods useful in making and assessing the activities and properties of the CHD1L inhibitors herein.

Abraham et al., 2019 and any supplementary information for that journal article are each incorporated by reference herein in its entirety for descriptions of biological and chemical methods useful in making and assessing the activities and properties of the CHD1L inhibitors herein.

Zhou et al., 2020 and any supplementary information for that journal article are each incorporated by reference herein in its entirety for descriptions of biological and chemical methods useful in making and assessing the activities and properties of the CHD1L inhibitors herein.

PCT/US2021/023981, filed Mar. 24, 2021, U.S. provisional applications 62/994,259, filed Mar. 24, 2020 and 63/139,394, filed Jan. 20, 2021, are each incorporated by reference herein in its entirety.

Prigaro et al. 2022 and any supplementary information for that journal article are each incorporated by reference herein in its entirety for descriptions of biological and chemical methods useful in making compounds herein and assessing the activities and properties of the CHD1L inhibitors herein.

When a group of substituents is disclosed herein, it is understood that all individual members of the group and all subgroups, including any isomers and enantiomers of the group members, and classes of compounds that can be formed using the substituents are disclosed separately. When a compound is claimed, it should be understood that compounds known in the art including the compounds disclosed in the references disclosed herein are not intended to be included. When a Markush group or other grouping is used herein, all individual members of the group and all combinations and subcombinations possible of the group are intended to be individually included in the invention.

When a compound is described herein such that a particular isomer, enantiomer or diastereomer of the compound is not specified, for example, in a formula or in a chemical name, that description is intended to include each isomers and enantiomer (e.g., cis/trans isomers, R/S enantiomers) of the compound described individual or in any combination. Additionally, unless otherwise specified, all isotopic variants of compounds disclosed herein are intended to be encompassed by the invention. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Isotopic variants, including those carrying radioisotopes, may also be useful in diagnostic assays and in therapeutics. Methods for making such isotopic variants are known in the art.

Molecules disclosed herein may contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the invention herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

CHD1L inhibitors of this invention are commercially available or can be prepared without undue experimentation by the methods disclosed herein or by routine adaptation of such methods using starting materials and reagents which are commercially available or which can be made by known methods. It will be appreciated that it may be necessary, dependent upon the compound to be synthesized, to protect potentially reactive groups in starting materials from undesired conjugation. Useful protective groups, for various reactive groups are known in the art, for example as described in Wutts & Greene, 2007.

Compounds herein can be in the form of salts, for example ammonium salts, with a selected anion or quaternized ammonium salts. The salts can be formed as is known in the art by addition of an acid to the free base. Salts can be formed with inorganic acids such as hydrochloric acid, hydrobromic acid, sulfuric acid, nitric acid, phosphoric acid and the like, or organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like.

In specific embodiments, compounds of the invention can contain one or more negatively charged groups (free acids) which may be in the form of salts. Exemplary salts of free acids are formed with inorganic base include, but are not limited to, alkali metal salts (e.g., Li⁺, Na⁺, K⁺), alkaline earth metal salts (e.g., Ca²⁺, Mg²⁺), non-toxic heavy metal salts and ammonium (NH₄⁺) and substituted ammonium (N(R′)₄⁺ salts, where R′ is hydrogen, alkyl, or substituted alkyl, i.e., including, methyl, ethyl, or hydroxyethyl, specifically, trimethyl ammonium, triethyl ammonium, and triethanol ammonium salts), salts of cationic forms of lysine, arginine, N-ethylpiperidine, piperidine, and the like. Compounds of the invention can also be present in the form of zwitterions. Compound herein can be in the form of pharmaceutically acceptable salts, which refers to those salts which retain the biological effectiveness and properties of the free bases or free acids, and which are not biologically or otherwise undesirable.

The scope of the invention as described and claimed encompasses the racemic forms of the compounds as well as the individual enantiomers and non-racemic mixtures thereof. The compounds of the invention may contain one or more asymmetric carbon atoms, so that the compounds can exist in different stereoisomeric forms. The compounds can be, for example, racemates or optically active forms. The optically active forms can be obtained by resolution of the racemates or by asymmetric synthesis. In a preferred embodiment of the invention, enantiomers of the invention exhibit specific rotation that is +(positive). Preferably, the (+) enantiomers are substantially free of the corresponding (−) enantiomer. Thus, an enantiomer substantially free of the corresponding enantiomer refers to a compound which is isolated or separated via separation techniques or prepared free of the corresponding enantiomer. “Substantially free,” means that the compound is made up of a significantly greater proportion of one enantiomer. In preferred embodiments the compound is made up of at least about 90% by weight of a preferred enantiomer. In other embodiments of the invention, the compound is made up of at least about 99% by weight of a preferred enantiomer. Preferred enantiomers may be isolated from racemic mixtures by any method known to those skilled in the art, including high performance liquid chromatography (HPLC) and the formation and crystallization of chiral salts or prepared by methods described herein. [See, for example, Jacques et al., 1981; Wilen et al., 1977; Eliel, 1962; Wilen, 1972.]

Compounds of the invention, and salts thereof, may exist in their tautomeric form, in which hydrogen atoms are transposed to other parts of the molecules and the chemical bonds between the atoms of the molecules are consequently rearranged. It should be understood that all tautomeric forms, that may exist, are included within the invention.

Every formulation, compound or combination of components described or exemplified herein can be used to practice the invention, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomers and enantiomer of the compound described individual or in any combination.

It will be appreciated by one of ordinary skill in the art that chemical compounds can be named using various conventions and that even within a given convention chemical names for a given compound may vary, such that the same compound can be properly named in different ways. Where herein, there is an inconsistency between a compound name and a compound structure, if specifically provided, the compound structure is given precedence.

One of ordinary skill in the art will appreciate that methods, alternative therapies, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the invention.

The singular terms “a,” “an,” and “the” include plural referents unless context clearly indicates otherwise. Similarly, the word “or” is intended to include “and” unless the context clearly indicates otherwise. The term “comprises” means “includes.” Also, “comprising A or B” means including A or B, or A and B, unless the context clearly indicates otherwise. It is to be further understood that all molecular weight or molecular mass values given for compounds are approximate and are provided for description. Although methods and materials similar or equivalent to those described herein can be used in the practice or testing of this invention, suitable methods and materials are described below. In addition, the materials, methods, and examples are illustrative only and not intended to be limiting.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. More specifically, the term “consisting essentially of” is open to the listed component(s), excluding (1) active ingredients that do not function for the intended therapeutic application, and (2) other components that negatively affect the activity or combined activity of the listed components, but not excluding pharmaceutically acceptable excipients which do not negatively affect the activity or combined activity of the listed component(s). Any recitation herein of the term “comprising”, particularly in a description of components of a composition or in a description of elements of a device, is understood to encompass those compositions and methods consisting essentially of and consisting of the recited components or elements.

The invention illustratively described herein suitably may be practiced in the absence of any element or elements, limitation or limitations which is not specifically disclosed herein.

Without wishing to be bound by any particular theory, there can be discussion herein of beliefs or understandings of underlying principles relating to the invention. It is recognized that regardless of the ultimate correctness of any mechanistic explanation or hypothesis, an embodiment of the invention can nonetheless be operative and useful.

The terms and expressions that have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention.

### THE EXAMPLES

### Example 1: Clinicopathological Characterization of CHD1L in Patients with CRC

CHD1L expression is correlated with poor prognosis in several cancers, but only limited information about the pathology of CHD1L in CRC is known. This example describes the pathogenic characterization and mechanisms of pathology for CHD1L in CRC patients. The clinicopathological characteristics of 585 patients with CRC were analyzed from the Cartes d'ldentite des Tumeurs (CIT) program with respect to CHD1L expression (GEO: GSE39582). [Marisa et al., 2013] These characteristics are summarized in Abbott et al., 2020, supplementary information.

Additional data for this example are found in Abbott et al., 2020 and its supplementary information. Follow up information was available for all patients in the CIT cohort over a period of 15 years. For the entire patient cohort, high CHD1L expression is associated with lower OS (P=0.0167) and median survival (MS) of 8.8 years for high CHD1L patients. Median survival was not reached in the low CHD1L cohort as 72% (115/159) of patients were censored and 26% (42/159) were deceased. Patient data were evaluated using the TNM staging system. As Stage I and IV patients have a high likelihood of survival or death, respectively, survival of Stage II and III CRC patients was evaluated. High CHD1L expression was associated with a lower OS (P=0.0191) and MS of 11 years for Stage II and III CRC, again median survival was not reach in the low CHD1L cohort. Survival was also analyzed with respect to CHD1L expression for each stage of CRC. Stage II patients showed a significant difference in survival (P=00319) with a M.S. of 11 years, no significant difference was observed for Stage I, III or IV patients. Analysis of CHD1L expression indicated a significant difference in expression cancer stage. Patients with Stage I and II colorectal cancer versus patients with Stage III and IV were evaluated and showed a significant increase in CHD1L expression in the Stage III and IV versus early stage cohort (P=0.0051). Analysis of CHD1L expression with respect to lymph node metastasis suggests that CHD1L is overexpressed in patients with increased regional lymph node metastasis (N1 P=0.0128, N2 P=0.05 compared to NO). Although the trend of CHD1L expression was the same for the N3 cohort, no significance was determined due to the limited number of patient samples available. No significant difference in CHD1L expression with respect to tumor size, metastasis or location was found.

Evaluation of CHD1L in CRC Molecular Subtypes.

The association of CHD1L expression with six molecular subtypes of CRC [Marisa et al., 2013]: C1 (immune system down, n=116), C2 (deficient mismatch repair, n=104), C3 (KRAS mutant, n=75), C4 (CSC, n=59), C5 (activated WNT pathway, n=152), and C6 (chromosomal instability normal, n=60) was investigated. There is a significant difference of CHD1L expression among the six molecular subtypes (P<0.001). CHD1L expression was high in C5, C4, and C3, and low in C2 and C6. The C2 subtype is associated with a decrease in the WNT signaling pathway and deficient for mismatch repair. The C4 and C6 subtypes are associated with poorer relapse-free survival compared to other subtypes. The C4 subtype is associated with increased CSC stemness and the C5 subtype is associated with activated WNT signaling and deregulated EMT pathways. The lower CHD1L expression in the C2 (deficient mismatch repair) subtype is consistent with its known function in DNA damage response. [Ahel et al., 2009] Additionally, CHD1L expression was lower in patients with deficient mismatch repair than in patients without (P<0.001). CHD1L expression was also higher in patients with KRAS mutations (P=0.049). The expression of CHD1L in the C3, C4, and C5 molecular subtypes prompted a further investigation of the function of CHD1L expression in EMT, CSC stemness, and the WNT/TCF pathway.

CHD1L Expression Correlates with Wnt/TCF Associated Genes

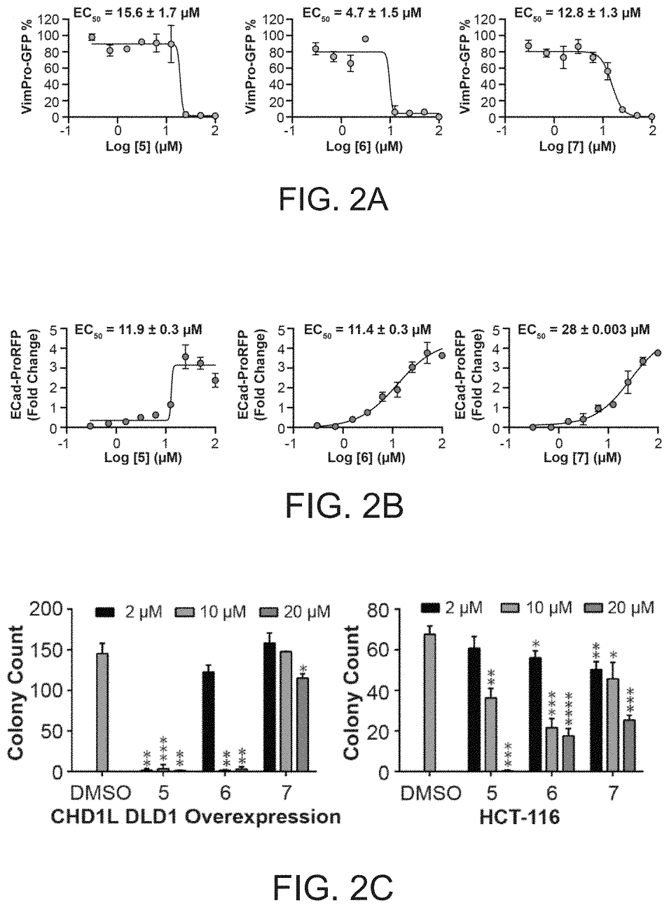

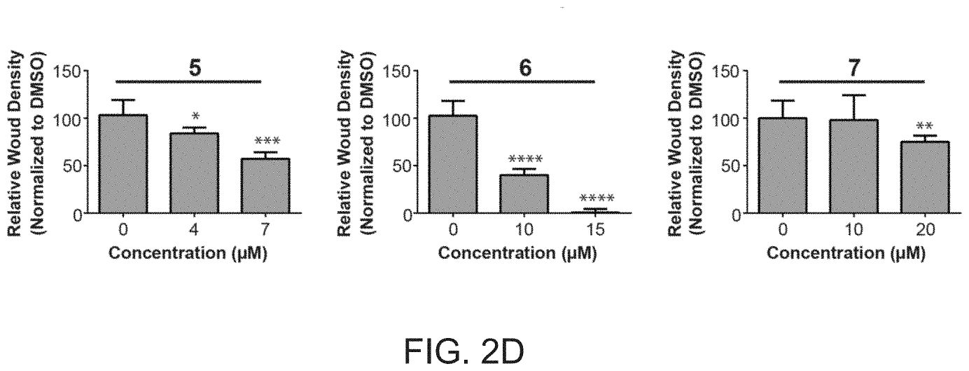

Utilizing a smaller cohort of CRC patients (n=26) from the UCCC GI tumor tissue bank, a similar trend was observed as with the larger CIT cohort CHD1L expression significantly correlated with late stage and metastatic CRC compared to early stage and primary CRC (Abbott et al., 2020, Supplementary information). The expression is quantified as FPKM (fragments per kilobase exon per million fragments mapped). Metastatic tumor samples had significantly more CHD1L than primary tumors. Additionally, CHD1L levels were higher in Stage IV compared to Stage II/III patient cohorts. When analyzing CHD1L expression with genes involved in KEGG WNT pathway, using Spearman's correlation a significant positive correlation with 65 of 125 genes was observed. Among these were well-established genes involved in TCF-mediated transcription such as topoisomerase IIα (TOP2A) (r=0.65, P=0.004 [Zhou et al., 2016; Abraham et al, 2019], and TCF4 (r=0.61, P=0.0012) (Abbott et al., Supplementary FIG. 2). Genes had P<0.05 correlation value. Transcript expression was Log₂ normalized and quantified by FPKM (fragments per kilobase exon per million fragments mapped).

A significant positive correlation was observed between known CSC markers CD44 (r=0.43, P=0.038), LGR5 (r=0.55, P=0.0075) and CHD1L. When comparing the CIT cohort to the UCCC cohort a significant correlation was observed for TOP2A (r=404 0.1275, P=0.0020) and TCF4 (r=0.1050, P=0.011). Consistent with this result, it has been shown that TOP2A is a required component of the TCF-complex, promoting EMT in CRC. [Zhou et al., 2016; Abraham et al., 2019] Hence, CHD1L appears to be involved in TCF-transcription and EMT in CRC patients.

### Example 2: CHD1L Mediates TCF-Transcription in CRC

Based on the correlation of CHD1L with TCF-complex members, CHD1L may have a mechanistic role in TCF-transcription. To assess this role, SW620 and DLD1 cell lines, which have high and low endogenous CHD1L expression, respectively, were utilized. Additional data for this example are found in Abbott et al, 2020, and its Supplemetary Information. Small hairpin RNA (shRNA) was used to knockdown CHD1L in SW620 cells (SW620^{CHD1L-KD}). CHD1L was overexpressed in DLD1 cells (DLD1^{CHD1L-OE}). Using the TOPflash luciferase reporter [Morin et al., 1997; Zhou et al., 2016] transfected into SW620^{CHD1L-KD} or DLD1^{CHD1L-OE} it was determined that overexpression of CHD1L produced a significant increase in TCF-transcription (P<0.0001) (Abbott et al., 2020). Conversely, SW620^{CHD1L-KD} cells displayed a significant decrease in TCF-transcription (P=0.0006). These results indicate that CHD1L is a potential factor directly involved in TCF-transcription. Each of Morin et al., 1997 and Zhou et al., 2016 is incorporated by reference herein in its entirety for descriptions of the TOPflash reporter and assays employing it.

CHD1L Directly Interacts with the TCF-Transcription Complex

Activation of TCF-transcription is a dynamic process that involves the shedding of co-repressor proteins, binding of co-activator proteins, and remodeling of the chromatin landscape. [Lorch et al. 2010, Shitashige et al., 2008] Co-immunoprecipitation (Co-IP) studies with TCF4 were performed, demonstrating that CHD1L directly binds to the TCF-complex [Abbott et al, 2020].

CHD1L has been well characterized as a binding partner with PARP1 in DNA damage response. [Pines, 2012; Ahel et al., 2009] PARP1 is also a component of the TCF-complex binding to TCF4 and β-catenin. [Idogawa et al. 2005] The results herein demonstrate that CHD1L binds to the TCF-complex, which is likely through interactions between TCF4 and PARP1.

To further characterize CHD1L as a component of the TCF-complex, chromatin immunoprecipitation (ChIP) of CHD1L to TCF-complex WNT response elements (WREs) was performed in SW620 cells. [Abbott et al., 2020] CHD1L was enriched at c-Myc, vimentin, slug, LEF1, and N-cadherin WREs, further supporting that CHD1L is functioning directly with the TCF-complex. Taken together, the data implicate CHD1L as a critical component of the TCF-transcription.

CHD1L Mediated TCF-Transcription Promotes EMT and CSC Stemness in CRC

Previously, TCF-transcription was characterized as a master regulator of EMT in CRC. [Zhou et al., 2016] In addition, CHD1L localizes at WREs of EMT effector genes. [Abbott et al, 2020] Therefore, biomarker expression in SW620^{CHD1L-KD} and DLD1^{CHD1L-OE} cells was measured to determine whether knockdown or overexpression of CHD1L modulates EMT. Knockdown of CHD1L induced reversion of EMT, decreasing vimentin and slug while increasing E-cadherin expression. [Abbott et al, 2020] Conversely, EMT was induced in DLD1^{CHD1L-OE} cells, evidenced by a decrease in E-cadherin and an increase in vimentin and slug expression. [Abbott et al, 2020] These results indicate that CHD1L is an EMT effector gene involved in promoting the mesenchymal phenotype in CRC. A hallmark of EMT is an increase in CSC stemness.

Clonogenic colony formation assays [Abbott et al, 2020] were performed to characterize the impact of CHD1L expression on stemness. [Franken et al., 2006] CSC stemness increased in DLD1^{CHD1L-OE} (P=0.0001) and decreased in SW620^{CHD1L-KD} (P=0.002) cells measured by colony formation.

### Example 3: Identification of Small Molecule Inhibitors of CHD1L

As established in Examples 1 and 2, herein, CHD1L is a driver of TCF-mediated EMT. Based on this, an assay to identify small molecule inhibitors of CHD1L is described herein. The drug discovery goal was to target CHD1L DNA translocation or interactions with DNA, which are dependent on CHD1L's catalytic domain ATPase activity. [Ryan & Owen-Hughes, 2011; Flaus et al., 2011]

CHD1L belongs to the SNF2 (sucrose non-fermenter 2) ATPase superfamily of chromatin remodelers that contains a two-lobe ATPase domain. [Abbott et al, 2020 and its Supplemental Information] CHD1L also has a macro domain that is unique relative to other chromatin remodelers, which promotes an auto-inhibited state through interactions between the macro and the ATPase domains. [Lehmann et al., 2017; Gottschalk et al., 2009] However, the macro domain binds to PARP1, the major activator of CHD1L, alleviating auto-inhibition. [Lehmann et al., 2017; Gottschalk et al., 2009]

Using the methodology of Lehmann et al., 2017, full-length CHD1L (fl-CHD1L) and the catalytic ATPase domain (cat-CHD1L) were purified. [Abbott et al, 2020 and its Supplementary Information] Protein constructs were used for recombinant expression and purification of CHD1L for in vitro HTS, as illustrated in Abbott et al., 2020. An SDS page gel showed purified cat-CHD1L (68 kD) and fl-CHD1L (101 kD). Enzyme kinetics of cat-CHD1I versus fl-CHD1L were compared. The cat-CHD1L provides for a more robust ATPase assay compared to fl-CHD1L, which is consistent with the report from Lehman et al., 2017. Therefore, to identify direct inhibitors of CHD1L ATPase, an exemplary High-through-put screening (HTS) assay in the context of TCF-transcription is described which includes: cat-CHD1L, c-Myc DNA, ATP, and phosphate-binding protein that fluoresces upon binding inorganic phosphate (Pi).

This assay was validated and pilot screening was preformed against clinically relevant kinase inhibitors. [Abbott et al, 2020 and its Supplemental Information]. The pilot screen found no hits, demonstrating that CHD1L is not a likely target for kinase inhibitors. Once validated, a primary HTS was preformed using 20,000 compounds from the Life Chemicals Diversity Set, which were screened at 20 μM in 1% DMSO with 10 mM EDTA as a positive control [Abbott et al, 2020 and its Supplementary Information] The screen provided robust statistics with an average Z′-factor value of 0.57±0.06 over 64 plates. The average compound activity was 92.3%±17.8. As a result, the hit limit was set to be 3 standard deviations from the mean at 39% ATPase activity. This stringent hit limit identified 64 hits, of which 53 hits were confirmed against recombinant CHD1L ATPase activity.

### Example 4: Exemplary Inhibitors

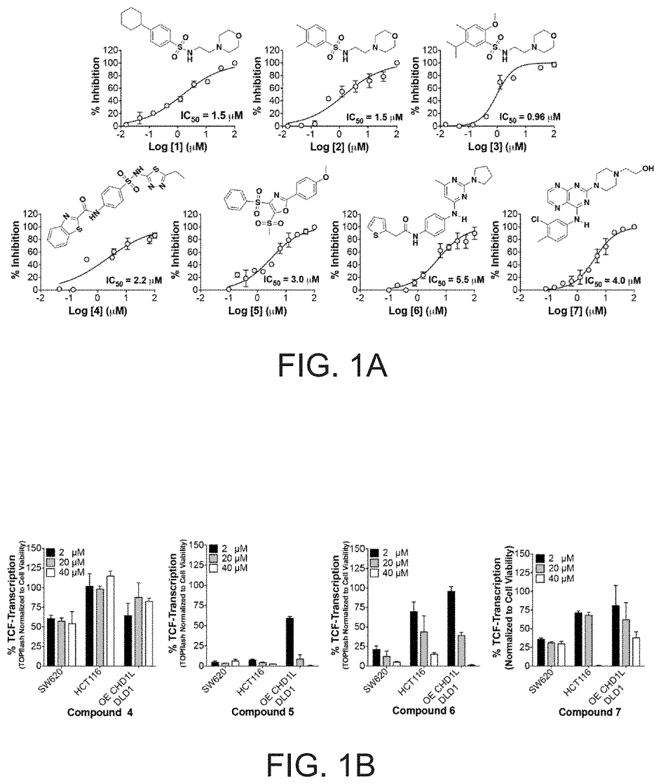

A subset of seven confirmed hits (compounds 1-7, see Scheme 1) were purchased, representing a range of pharmacophores with greater than 50% inhibition against cat-CHD1L ATPase. Compounds 1-7 were subjected to dose response studies against cat-CHD1L ATPase, which validated these hits as potent CHD1L inhibitors with activity between 900 nM to 5 μM (FIG. 1A). Structures of additional exemplary compounds 8-73 are provided in Scheme 1, where SEM represents the protecting group trimethylsilylethoxy methyl. Structures of additional exemplary compounds 74-116 are provided in Scheme 1. Note that in a number of cases in Scheme 1, an additional compound number is given in parenthesis which may be employed in Tables and Figures herein or in Abbott et al., 2020 and its Supplementary Information or Prigaro et al.

Compounds 1-7 were tested in HCT116, SW620, and DLD1^{CHD1L-OE} cells for their ability to inhibit TCF-transcription using the TOPflash reporter system (FIG. 1B). Compounds 1-3 were shown to have no significant activity in cells. Compound 4 was shown to have modest activity in cells with no dose dependent inhibition of TCF-activity. However, compounds 5-7 demonstrate superior dose dependent activity against TCF transcription in all three CRC cell lines. Notably, decreased inhibition of TCF-transcription was observed for 5-7 at the low 2 μM dose in DLD1^{CHD1L-OE} cells, which is evidence of cellular CHD1L target engagement.

CHD1L Inhibitors Reverse EMT and Malignant Properties in CRC.

After validating hits 5-7 against CHD1L mediated TCF transcription, the ability of these compounds to reverse EMT and other malignant properties in CRC were evaluated. E-cadherin and vimentin are putative biomarkers for the epithelial and mesenchymal phenotypes, respectively. [McDonald et al., 2015] Loss of E-cadherin and gain of vimentin are also clinical biomarkers of poor prognosis. [Yun et al., 2014; Richardson et al., 2012; Dhanasekaran et al., 2001; Kashiwagi et al., 2010; Toiyama et al., 2013] Accordingly, lentiviral promoter driven reporters for E-cadherin (pCDH1-EcadPro-RFP) and vimentin (pCDH1-VimPro-GFP) were developed, which faithfully report E-cadherin and vimentin protein expression, respectively. [Zhou et al., 2016; Abraham et al., 2019] SW620 cells transduced with either EcadPro-RFP or VimPro-GFP were cultured as tumor organoids for 72 h, reaching a diameter of 600 μm. Tumor organoids were treated with compounds 5-7 for an additional 72 h to determine the effective concentration 50 percent (EC₅₀) for modulating promoter activity. Changes in promoter expression was quantified using a 3D confocal image 507 based high-content analysis algorithm (FIG. 2A-2B). [Zhou et al., 2016; Abraham et al., 2019]

Compounds 5-7 effectively downregulated vimentin promoter activity with EC₅₀ values of 15.6±1.7 μM (5), 4.7±510 1.5 μM (6), and 12.8±1.3 μM (7). Conversely, E-cadherin promoter activity was upregulated with EC₅₀ values of 11.9±0.3 μM (5), 11.4±0.3 μM (6), and 28±0.003 μM (7). Representative images exhibiting reversion of EMT by compound 6 in SW620 tumor organoids measured by EMT reporter assays are shown in Abbott et al., 2020. These results indicate that small molecule inhibitors of CHD1L reverse TCF-driven EMT in CRC. To confirm that CHD1L inhibitors reverse EMT, protein expression of two additional putative biomarkers of EMT, slug (mesenchymal) and zona occludens-1 (ZO-1, epithelial) were evaluated. Changes in slug and ZO-1 are considered major criteria for EMT. [Zeisberg & Neilson, 2009] SW620 tumor organoids treated with CHD1L inhibitors downregulate slug and upregulate ZO-1, further indicating a reversion of EMT. Western blot analysis showing protein expression changes of additional EMT biomarkers slug and ZO1 is shown in Abbott et al., 2020.

A hallmark of EMT is an increase in CSC stemness and cell invasion. Therefore, the ability of compounds 5-7 to inhibit migration and invasion in HCT-116 and DLD1^{CHD1L-OE} cells was tested. All three compounds demonstrated a significant inhibition of CSC stemness (FIG. 2C). However, compounds 5 and 6.0 display more potent dose dependent inhibition. Note that DLD1^{CHD1L-OE} cells form two times more colonies than HCT-116 cells, which have moderate CHD1L expression. This observation is consistent with CHD1L's oncogenic and tumorigenic properties. Next, using HCT-116 cells with uniform scratch wounds imbedded in 50% Matrigel® matrix (Corning Life Sciences, Corning, N.Y.) cells were treated with CHD1L inhibitors at concentrations indicated and invasion was monitored over 72 h. Compounds 5-7 exhibited a dose dependent inhibition of invasion (FIG. 2D), with compound 6.0 displaying the most potent activity.

### Example 5: Inhibition of CHD1L Efficacy of DNA Damaging Drugs

CHD1L is known to function in PARP1 mediated DNA damage response repair, which is a mechanism of with increased drug resistance to DNA damaging chemotherapy [Li et al., 2019; Ahel et al., 2009; Gottschalk et al., 2009]. For example, drug resistance to cisplatin in lung cancer was observed in cells overexpressing CHD1L. The efficacy of cisplatin was restored after CHD1L knockdown. [Li Y., et al., 2019] In addition, knockdown of CHD1L alone does not increase DNA damage. [Ahel D, et al., 2009] In order to determine if CHD1L inhibitors could increase the efficacy of DNA damaging drugs against low CHD1L expressing DLD1 cells transduced with empty vector (DLD1CHD1L-EV) and overexpressing DLD1CHD1L-OE in CRC cells, compound 6 was evaluated alone as a single agent and in combination with SN-38 (active pharmacophore of prodrug irinotecan), oxaliplatin, and etoposide. To assess DNA damage, the phosphorylation of H2AX (γ-H2AX) by immunofluorescence, a biomarker for DNA damaging chemotherapy, [Ahel D, et al., 2009], was measured as shown in Abbott et al., 2020 and its Supplementary Information. Compound 6 alone showed no significant DNA damage when treating cells at 10 μM and measuring γ-H2AX activity, which is consistent with previously reported CHD1L knockdown studies. [Ahel D, et al., 2009]. However, combination treatments in DLD1CHD1L-OE cells with compound 6 synergized with etoposide (10 μM) and SN-38 (1 μM), significantly increasing DNA damage compared to etoposide and SN-38 alone. In DLD1CHD1L-EV cells only the combination of etoposide and compound 6 displayed significant synergy. Under the experimental conditions used we observed no synergy was observed with oxaliplatin. Nevertheless, SN-38 (i.e. irinotecan) combination therapy, known as FOLFIRI, is a standard of care in the treatment of CRC. Therefore, the enhanced DNA damage that occurs with compounds 6 in combination with SN-38 supports the hypothesis that CHD1L inhibitors can increase the efficacy of CRC standard of care DNA damaging chemotherapies.

### Example 6: CHD1L Inhibitors Reverse EMT Prior to the Induction of Cell Death

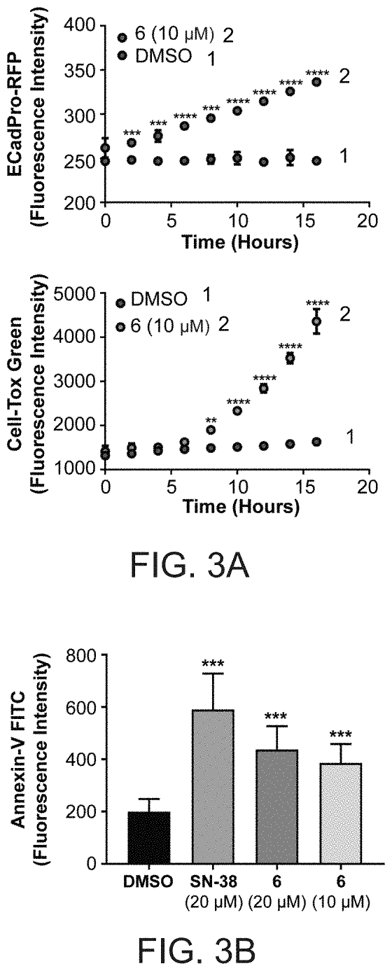

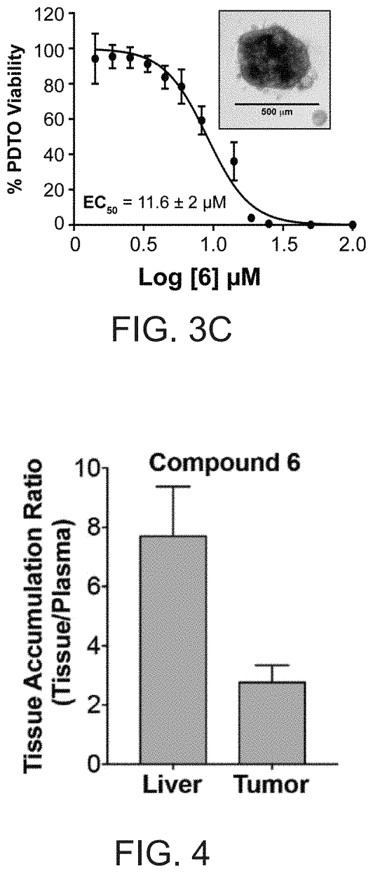

CHD1L has been reported to confer anti-apoptotic activity by inhibiting activation of caspase-dependent apoptosis. [Li et al., 2013; Sun et al., 2016] Additionally, reversal or inhibition of EMT is known to restore apoptotic activity of cancer cells. [Lu et al., 2014] To determine if CHD1L inhibitors reverse EMT prior to induction of cell death, E-cadherin expression by EcadPro-RFP reporter activity was monitored and cytotoxicity was measured using the CellTox™ Green assay. Cells were treated with CHD1L inhibitors for 72 h and imaged every 2 h. A significant increase in E-cadherin expression prior to induction of cytotoxicity for compound 6 relative to DMSO (FIG. 3A).

To determine if CHD1L inhibitors are able to induce apoptosis in CRC, western blots from SW620 tumor organoids were performed and it was observed that E-cadherin is cleaved after treatment with 5 and 6 [Abbott et al., 2020]. Cleavage of E-cadherin is a marker of apoptosis [Steinhusen et al., 2001]

The more potent CHD1L inhibitor 6, exhibited increases in cleaved PARP1, cleaved caspase 8, and cleaved caspase 3 relative to DMSO control [Abbott et al., 2020] These results indicate that compound 6 induces extrinsic apoptosis that is consistent with E-cadherin mediated apoptosis through death receptors. [Lu et al., 2014]

To further characterize the apoptotic activity of CHD1L inhibitors, annexin-V staining in SW620 cells over 12 h was examined. Compound 6.0 induced significant apoptosis compared to DMSO alone and had similar activity to the positive control SN-38, the active metabolite of irinotecan (FIG. 3B)

CHD1L inhibitors are effective against patient-derived tumor organoids (PDTOs). The use of PDTOs in preclinical drug development has been established as a predictive in vitro cell model for clinical efficacy. [Drost J & Clevers H, 2018] After establishing the ability of compound 6 to reverse EMT and induce apoptosis using cell line based models, the efficacy of compound 6 was evaluated in PDTOs produced from patient sample CRC102 obtained from the University of Colorado Cancer Center (UCCC) gastrointestinal (GI) tissue bank (FIG. 3C). Consistent with the results in CRC cell lines, compound 6.0 showed potent cytotoxicity in PDTOs with an EC50 of 11.6±2 μM

### Example 7: In Vitro and In Vivo PK, PD, and Liver Toxicity of Exemplary Inhibitor Compound 6

To assess the drug-like potential and properties of compound 6.0 in silico, in vitro, and in vivo PK studies were conducted assessing CLogP, aqueous solubility, stability in mouse liver microsomes, and PK in CD-1 mice.

Table 1 provides a summary of in vivo and in vitro pharmacokinetic parameters of compound 6. The consensus LogP (CLogP) values were obtained using the SwissADME web tools. [Daina et al., 2017] Compound 6 was administered by i.p. injection to athymic nude mice QD for 5 days to measure accumulation in SW620 xenograft tumors (FIG. 4) and to assess histopathology of liver toxicity. Representative H&E-stained photomicrograph sections (5× magnification) of liver in both vehicle and compound 6 treated animals are shown in Abbott et al., 2020. The images demonstrate normal hepatic cord and lobule architecture, with no evidence of hepatocyte degeneration, necrosis, hyperplasia, or parenchymal inflammation. Compound 6.0 has an excellent balance of lipophilicity (CLogP=3.2) and aqueous solubility that is relatively stable to liver metabolizing enzymes, and an excellent PK disposition when administered to CD-1 mice. Compound 6.0 reaches a high plasma drug concentration C_{Max} (˜30,000 ng/mL) and AUC (-80,000 ng/mL/h) with a relatively long half-life (T₁/2a) of 3 h after intraperitoneal (i.p.) administration.

In an initial study, compound 6, exhibited a half-life in liver microsomes of less than 20 minutes. In subsequent analogous in vitro liver microsome half-life experiments conducted with a different liver microsome preparation (data not shown), compound 6 exhibited a longer half-life of 67 minutes and compound 6.3 exhibited an improved (over 6) in vitro half-life of 98 minutes and compound 6.11 exhibited a further improved (over 6) in vitro half-life of 130 minutes. The initial half-life studies with compound 6 were conducted with a different liver microsome preparation and not comparable to later in vitro microsome half-live experiments. The results of the second series of in vitro and in vivo half-life measurements is provided in Table 2 which includes data for several additional compounds as indicated.

A second acute in vivo experiment was conducted using a maximum tolerated dose of 6.0 (50 mg/kg) administered to athymic nude mice by i.p. QD over five days. The goals of this experiment were to (1) determine if compound 6.0 causes acute toxicity to livers, (2) accumulates in VimPro-GFP SW620 xenograft tumors, and (3) to determine PD effects. Compound 6.0 accumulates in SW620 tumors at a concentration of 10,533±5,579 ng/mL (n=4). As expected, when comparing the ratio of compound 6.0 accumulation in tissue/plasma, 2.7 times more accumulation in liver compared to tumor was observed (FIG. 4). However, there was no apparent liver toxicity resulting from compound 6.0 at the dose and schedule administered (Table 3). Overall, there were no significant histological differences between the livers of vehicle or compound 6.0 treated mice. The primary histological changes observed were minimal fibrosis and inflammation of the hepatic capsule in both vehicle and compound 6.0 treated animals. This suggests a very low grade, sub-clinical peritonitis, and is consistent with being secondary to i.p. drug administration.

In accordance with accumulation of compound 6.0 in tumors, PD effects on tumor tissue were measured by Western blot analysis, indicating a significant downregulation of mesenchymal markers vimentin, vimentin reporter (VimPro-GFP), and slug [Abbott et al., 2020]. Although not statistically significant, upregulation of the epithelial marker ZO-1 and induction of cleaved caspase 3 (the putative biomarker of apoptosis) were also observed. Taken together, these observations of PD effects by compound 6.0 indicate the reversion of EMT and apoptosis in vivo that were consistent with in vitro cell-based antitumor activity of compound 6. Compound 6.0 displays good PK drug-like properties and the ability to alter EMT and induce apoptosis in vivo with no observed liver toxicity.

In contrast, compound 6.11 exhibits significantly longer half-life (T_{1/2λ}) compared to that of compound 6 of much greater than 6 h after intraperitoneal (i.p.) administration.

TABLE 1
PK Parameters Compound 6

| | | In Vitro | | | | In vivo | | |
|---|---|---|---|---|---|---|---|---|
| | | PBS | Microsomal | | | AUC0-t | | |
| PK Parameters | | Solubility | Half-life | C_{Max} | V₂ | (ng/mL × | CL | T1/2λ |
| Comp. | ClogP | (mg/mL) | (min) | (ng/mL) | (L/kg) | h) | (L/h/kg) | (h) |
| 6 | 3.2 | 0.70 | 17.0 | 29,900 | 2.7 | 80,333 | 0.62 | 2.97 |

TABLE 2
CHD1L Inhibitor Pharmacokinetics

| | Pharmacokinetics (PK) | |
|---|---|---|
| CHD1L | In Vitro Half- | In vivo Half- |
| Inhibitor | Life (Min) | Life (hr) |
| 6 | 67 | 3 |
| 6.1 | 34.9 | — |
| 6.2 | 26.1 | — |
| 6.3 | 98 | — |
| 6.4 | 31.4 | — |
| 6.9 | 295 | — |
| 6.10 | 16.7 | — |
| 6.11 | 130 | 8 |
| 6.12 | 9.3 | — |
| 6.13 | 43.1 | — |
| 6.14 | 70.4 | — |
| 6.15 | 22.7 | — |

TABLE 3
Histological evaluation raw scores of livers from athymic nude mice
treated with vehicle or compound 6.0 (50 mg/kg) QD for 5 days.

| Animal Groups | Organ | Assessment¹ | Inflammation score² |
|---|---|---|---|
| Vehicle -1 | Liver | N | — |
| Vehicle - 2 | Liver | A | 1 |
| Vehicle - 3 | Liver | N | — |
| Vehicle - 4 | Liver | A | 1 |
| Compound 6.0 - 1 | Liver | N | — |
| Compound 6.0 - 2 | Liver | A | 1 |
| Compound 6.0 - 3 | Liver | N | — |
| Compound 6.0 - 3 | Liver | A | 1 |

¹Assessment: N = normal background lesion for mouse strain; A = abnormal.
²Inflammation score (performed if abnormal tissue assessment): 0 = none, 1 = minimal, 2 = mild, 3 = moderate, 4 = severe

### Example 8: Biological Evaluation of Compound 8

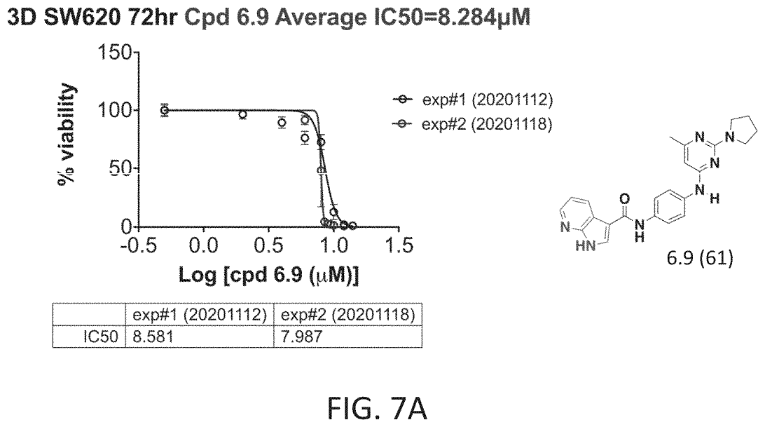

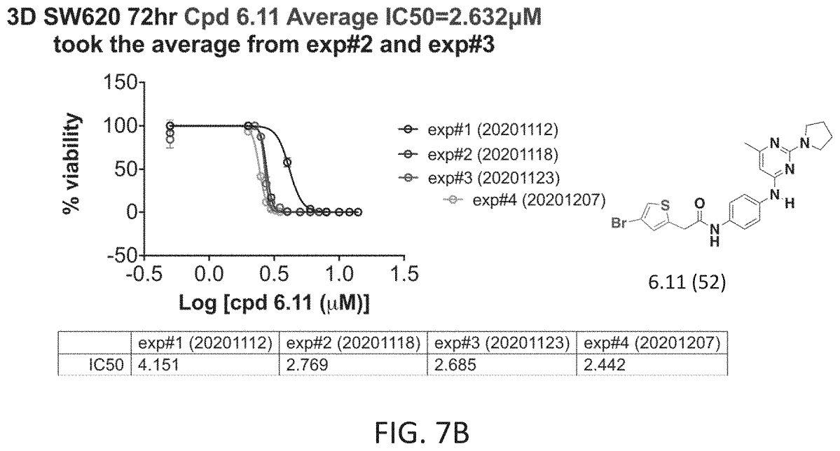

Compound 8 was evaluated in a number of biological assays described above. Results are presented in FIGS. 7A-E. Compound 8 displays more potent dose dependent inhibition of CHD1L-mediated TCF-transcription (FIG. 7A) compared to compound 6. Likewise, compound 8 reverses EMT, evidenced by the downregulation of vimentin and upregulation of E-cadherin promoter activity (FIGS. 7B and 7C, respectively). Compound 8 significantly inhibits clonogenic colony formation over 10 days (FIG. 7D). Compound 8 significantly inhibits HCT116 invasive potential over 48 h (FIG. 7E).

### Example 9: Methods Applied in Examples Herein

Additional Materials and Methods

Antibodies. Monoclonal mouse anti-TCF4 antibody was purchased from EMD Millipore (Billerica, Mass., USA) (catalog #05-511), a 1:1000 dilution was used for Western blot and 2 μg antibody per 300 μg of protein was used for IP. Monoclonal rabbit anti-CHD1L antibody was purchased from Abcam (Cambridge, Mass., USA) (catalog #ab197019), a 1:5000 dilution was used for Western blot, and 1.5 μg antibody per 300 μg of protein was used for IP. Monoclonal rabbit anti-Vimentin (catalog #5741), anti-Slug (catalog #9585), anti-E-cadherin (catalog #3195), anti-ZO-1 (catalog #8193), anti-Histone H3 (catalog #4620) were purchased from Cell Signaling (Danvers, Mass., USA) and mouse anti-α-tubulin (catalog #3873) were purchased from Cell Signaling and a 1:1000 dilution was used for Western blot. Monoclonal rabbit anti-β-catenin (catalog #9582) were purchased from Cell Signaling, a 1:1000 dilution was used for Western blot. Monoclonal rabbit anti-phospho-β-catenin was purchased from Cell Signaling (catalog #5651). Monoclonal rabbit anti-TCF4 (catalog #2569) and anti-Histone H3 (catalog #4620) were purchased from Cell Signaling and 2 μg antibody per 1 mg of protein was used for ChIP. Anti-rabbit IgG HRP-linked secondary antibody (catalog #7074) was purchased from Cell Signaling and a 1:3000 dilution was used for Western blot. Anti-goat and anti-mouse IgG HRP-linked secondary antibodies (catalog #805-035-180 and #115-035-003) were from Jackson ImmunoResearch (West Grove, Pa.), a 1:10,000 dilution was used for Western blot.

Clinicopathological Characterization of CHD1L

Transcriptome expression data of 585 CRC patients from the CIT cohort (GEO: GSE39582) were used for in silico validation (GSE39582). [Marisa et al., 2013] Gene expression analyses were performed by the Affymetrix GeneChip™ Human Genome U133 Plus 2.0 Array (Thermo Fisher Scientific, Waltham, Mass.). Robust Multi-Array Analysis (RMA) was used for data preprocessing and ComBat (empirical Bayes regression) for batch correction. Signal intensity was log 2 normalized. The CHD1L cutoff for CRC risk stratification based on disease specific survival was determined by the receiver operating characteristic (ROC) curve. Cutoff for CHD1L expression was set to 6.45. Differences in OS were estimated by the Kaplan-Meier method and compared using the log-rank test. The Fisher's exact test was used for the comparison of categorical variables. The Mann-Whitney U test was used for 2 groups of continuous variables. In case of more than two groups, data was analyzed by the Kruskal-Wallis test. For all 2-sided P-values, the unadjusted significance level of 0.05 was applied.

The CHD1L cutoff and clinicopathologic characteristics were evaluated by multiple cox regression analysis. Only variables that were significant in univariate analyses were integrated in the cox regression model using the Wald forward algorithm for significance determination. All variables including more than 2 groups were categorized and the stepwise entry criterion for covariates was P<0.05 and the removal criterion was P>0.1. Statistical analysis was performed using IBM® SPSS Statistics (IBM, Armonk, N.Y.), Prism8 (GraphPad Software, San Diego, Calif.), JMP® (SAS Institute, Cary, N.C.), and RStudio™IDE (RStudio Inc, Boston, Mass.).

UCCC Patient Sample RNA-Seq Analysis

RNA-seq data from CRC patient tumor xenograft explants were obtained from the UCCC (University of Colorado Cancer Center) GI tumor tissue bank, and analyzed as previously described. [Scott et al, 2017] Briefly, gene expression was Log2 normalized and measured by FPKM (Fragments Per Kilobase of transcript per Million mapped reads). The Wnt signaling pathway defined by the Kyoto Encyclopedia of Genes and Genomes (KEGG) was used as the gene set in this study. Samples with expression of CHD1L<1 FPKM were considered low expression and were removed from this study. Genes with significant Spearman's correlations (P<0.05) were displayed as heatmap using matrix2png (gene-wise Z-normalized) [See: Abbott et al, 2020 and its Supplementary Information]

CHD1L Overexpression and shRNA Knockdown

Full length CHD1L was synthesized in a pGEX-6P-1 plasmid (GenScript, Piscataway, N.J.). The CHD1L sequence flanked by EcoR/and Not/was digested out and ligated to a lentiviral backbone to create pCDH1-CMV-CHD1L-EF1-puro plasmid for overexpression of CHD1L in human CRC cells. Mission® shRNA (Sigma-Aldrich Co. LLC, St. Louis, Mo.) (scrambled) and TRCN0000013469 and TRCN0000013470 (sh69 and sh70) specific for CHD1L were purchased from Sigma-Aldrich (St. Louis, Mo.). Virus was produced in HEK293T cells using TransIT®-293 reagent (Mirus, Madison, Wis.), and plasmids pHRdelta8.9 and pVSV-G. CRC cells were transduced with overexpression or shRNA knockdown virus and selected with 2 μg/ml puromycin for 7 days.

Western Blots

CRC cell lines and homogenized tumor tissue samples from mice were resuspended in RIPA lysis buffer (20 mM Tris-HCl (pH 7.5), 150 mM NaCl, 1 mM Na₂EDTA, 1 mM EGTA, 1% NP-40, 1% sodium deoxycholate, 2.5 mM sodium pyrophosphate, 1 mM b-glycerophosphate, 1 mM Na₃VO₄, 0.1 mM PMSF. Protein concentration was determined using the Pierce™ BCA protein assay kit (ThermoFisher, Waltham, Mass.). Forty micrograms of sample were run on 10% Bis-Tris gels. Following electrophoresis, the proteins were transferred to a nitrocellulose membrane. The membranes were blocked at room temperature with 5% non-fat milk in TBS/Tween® 20 (TBST contains 20 mM Tris, 150 mM NaCl, and 0.1% Tween®20 (Croda International PLC, Snaith, UK) for 1 hour at room temperature. Membranes were washed three times with TBST. Blots were incubated with the appropriate primary antibody in 5% nonfat milk in TBST overnight at 4° C. Membranes were washed three times with TBST and then incubated with appropriate secondary antibody for one hour. Membranes were washed again with TBST three times. Blots were exposed using SuperSignal™West Pico PLUS Chemiluminescent Substrate (ThermoFisher, Waltham, Mass.) and imaged using a ChemiDoc imaging system (Bio-Rad, Hercules, Calif.). [See: Abbott et al., 2020 and its Supplementary Information for Western Blots]

TOPflash TCF-Transcriptional Reporter Assay

TOPflash assay (Millipore, Billerica, Mass.) was used to evaluate TCF transcriptional activity in CRC cells. A total of 20,000 cells per well were plated into 96-well white plates and transfected with TransIT®-LT1 transfection reagent (Mirus, Madison, Wis.). Cells were incubated with transfection mix for 24 h. Next, cells were washed with phosphate-buffered solution (PBS) and a 1:1 ratio of PBS:ONE-Glo™ luciferase reagent Promega (Madison, Wis.) was added and the luminescence was detected within 10 min. A duplicate experiment was conducted to measure cell viability using CellTiter-Glo® Luminescent Cell Viability Assay (Promega, Madison, Wis.), which was used to normalize TOPflash luminescence to obtain the fold change in TCF activity. Experiments were replicated 2× (n=3 for each experiment).

Co-ImmunoPrecipitation (Co-IP)

Nuclear cell lysates were generated 138 from untreated SW620 cells. For the input control, 100 μL of 1 mg/mL nuclear extract was saved and used as the input. ImmunoPreciptation (IP) was conducted with Dynabeads™ Protein A IP Kit (ThermoScientific, Waltham, Mass.). Briefly, 300 μg of lysate incubated with 2 μg of the anti-TCF4 and anti-CHD1L IP antibody, anti-rabbit IgG and anti-mouse IgG were used as nonspecific binding controls and were rotated at 4° C. for 2 h. After preincubation, 50 μL of beads were transferred to the preincubated antibody/lysate mixture followed by overnight incubation at 4° C. The flow through was collected and the beads were washed 3× with PBST. Proteins were eluted with 20 μL of 50 mM glycine (pH=2.8) at 70° C. for 10 min.

Chromatin Immunoprecipitation (ChIP)

Using detailed methods previously described [Zhou et al., 2016], cells were cross-linked with 1.42% formaldehyde for 15 min and quenching with 125 mM glycine for 5 min. Cells were lysed with Szak's RIPA (Radioimmunoprecipitation assay buffer) buffer and sonicated. The IP steps were conducted at 4° C. as follows: 50 μL of protein A/G agarose beads were prewashed with cold Szak's RIPA buffer and incubated with 1 mg of lysate for 2 h. 0.3 mg/mL of salmon sperm DNA was added and incubated for 2 h. Lysate (100 μL) was set aside as the input control. Anti-CHD1L (2 μg) was added to the remainder and incubated overnight. Beads were washed and the supernatant was aspirated to 100 μL followed by the addition of 200 μL of 1.5×-Talianidis elution buffer (70 mM Tris-CI pH 8.0, 1 mM EDTA pH 8.0, 1.5% w/v SDS). To elute immunocomplexes and reverse crosslink, 12 μL of 5M NaCl was added and the mixture was incubated at 65° C. for 16 h. The supernatant was mixed with 20 μg of proteinase K and incubated for 30 min at 37° C. DNA was extracted with phenol/chloroform and precipitated with ethanol. The IP product was amplified with PowerUp™ SYBR™ Green Master Mix (Applied Biosystems, Austin, Tex.) using known published primers. [Zhou et al., 2016]

Clonogenic Assay

Colony formation was assessed after CHD1L knockdown in SW620 cells or overexpression in DLD1 cells as previously described. [Zhou et al., 2016; Abraham et al., 2019] Cells were plated at 1,000 cells/well in six-well plates and medium was changed 2× per week over a 10-day time course. Colony formation analysis was also performed as previously described. [Zhou et al., 2016; Abraham et al., 2019]

To assess CHD1L inhibitors for their ability to suppress CSC stemness, HCT-116 or CHD1L overexpressing DLD1 cell lines were pre-treated in monolayer cultures for 24 h with vehicle control (0.5% DMSO) or CHD1L inhibitors at the concentrations indicated in FIG. 2C. Pretreated viable cells were plated at 1,000 cells/well in 6-well plates or 200 cells/well in a 24-well plates. Colonies were analyzed using the IncuCyte® S3 2018A (Sartorius, France) software (with the following parameters modified from default: (1) for HCT116 cells segmentation adjustment=0.6; Min area (μm2)=3×104; Max area (μm2)=1.6×106; Max eccentricity=0.9; (2) for DLD1CHD1L OE cells segmentation adjustment=1; Min area (μm2)=1×104; Max area was not constrained; Max eccentricity=0.95. Experiments were replicated 2× (n=2 for each experiment).

Tumor Organoid Culture

Cell lines were cultured [Zhou et al., 2016; Abraham et al., 2019] as tumor organoids using phenol red free RPMI-1640 containing 5% FBS and by seeding 5,000 cells/well into un-coated 96-well U-bottom Ultra Low Attachment Microplates (Perkin-Elmer, Hopkinton, Mass.) followed by centrifugation for 15 min at 1,000 rpm to promote cells aggregation. A final concentration of 2% Matrigel® matrix (Corning Incorporated, Corning, N.Y.) was added and tumor organoids were allowed to self-assemble over 72 h under incubation (5% CO₂, 37° C., humidity) before treatment, and maintained under standard cell culture conditions during treatment time courses.

VimPro-GFP and EcadPro-RFP Reporter 3D High-Content Imaging Assays

Stable VimPro-GFP or EcadPro-RFP SW620 reporter cells were generated using pCDH imPro-GFP-EF1-puro virus or pCDH-EcadPro-mCherry-EF1-puro virus as previously reported. [Zhou et al., 2016; Abraham et al., 2019] The stable fluorescently labeled reporter cells were used to generate tumor organoids as described herein. Tumor organoids were treated with CHD1L inhibitors at 10 μM for an additional 72 h. Following treatment, tumor organoids were stained with 16 μM of Hoechst 33342 for 1 h (nuclei stain). Images were taken with a 5× air objective. Z-stacks were set at 26.5 μm apart for a total of 15 optical slices. Imaging and high-content analysis were performed using an Opera Phenix™ and Harmony® software (PerkinElmer, Hopkinton, Mass.). Nuclei were identified within each layer and cells were found with either GFP or mCherry channel. The fluorescence intensities of each channel were calculated and thresholds were set based on the background intensities. Percentages of GFP or mCherry RFP positive cells were calculated and normalized to the DMSO treated group.

Tumor Organoid Cytotoxicity.

SW620 tumor organoids were cultured as described herein. CellTox™ Green cytotoxicity assay solution was prepared per manufacturer's protocol (Promega, Madison, Wis.). Briefly, tumor organoids were treated for 72 h with CellTox™ Green reagent (0.5×) and various doses of CHD1L inhibitors over a range of 0-to-100 μM. Organoids were imaged using the Opera Phenix™ 207 screening system (PerkinElmer Cellular Technologies, Hamburg, Germany) with excitation at 488 nm and emission at 500-550 nm. Mean intensity of the whole well was utilized for calculating cytotoxicity with Lysis Buffer (Promega, Madison, Wis.) as the 100% cytotoxicity control and 0.5% DMSO as the 0% cytotoxicity control. Intensity values were normalized to these controls using Prism8 (GraphPad, San Diego, Calif.).

Invasion Assays.

HCT116 cells were plated at 60,000 cells/well into an IncuCyte® ImageLock 96-well plate (Sartorius, France) and allowed to attach overnight. A wound was created in all wells using the IncuCyte® WoundMaker then washed 2× with PBS. The plate was brought to 4° C. using a Corning XT Cool Core to avoid polymerization of the Matrigel® matrix (Corning Life Sciences, Corning, N.Y.) during the preparation of the invasion conditions. Wells were coated with 50 μL of 50% Matrigel® matrix in RPMI-1640 media. Plates were centrifuged at 150 rpm at 4° C. for 3 min, using a swing bucket rotor to ensure even matrix coating with no air bubbles. Afterwards, plates were placed on a Corning XT CoolSink module prewarmed inside a cell culture incubator (5% CO₂, 37° C., humidity) for 10 min to evenly polymerize the matrix, followed by the addition of CHD1L inhibitors dissolved in 50 μL of RPMI-1640 media containing 5% FBS. Finally, the plate was placed in an IncuCyte® S3 live cell imager (Sartorius, France) for 48 h. The wound was imaged every hour using the phase contrast channel and 10× objective in wide mode.

Cloning and Purification of Recombinant Human CHD1L

Cat-CHD1L (residues 16-61) and fl-CHD1L (residues 16-879) constructs were a generous gift from Helena Berglund at the Karolinska Institute, Department of Medical Biochemistry and Biophysics. Proteins were expressed in Rosetta™ 2 (DE3) pLysS cells (Novagen available from Sigma-Aldrich, St. Louis, Mo.) in Terrific Broth (ThermoFischer, Waltham, Mass.). Cultures were induced with 0.2 mM IPTG at OD600=2.0 at 18° C. for 16 h. Cells were harvested and resuspended in buffer-A, containing 20 mM HEPES, pH 7.5, 500 mM NaCl, 50 mM KCl, 20 mM imidazole, 10 mM MgCl₂, 1 mM TCEP (tris(2-carboxyethyl)phosphine), 10% glycerol and 500 μM PMSF. Cells were lysed by sonication and cellular debris was removed by centrifugation. The supernatant was loaded onto a Ni-NTA resin column (Qiagen, Hilden, Germany). Protein bound to the column was washed with 1× with buffer-A, 1× with buffer-A containing 10 mM ATP, and washed an additional time with buffer-A. Proteins were eluted using buffer-B (buffer-A with 500 mM imidazole) with a gradient from 20 to 500 mM imidazole. Following affinity purification, cat-CHD1L was dialyzed overnight into 50 mM Tris, pH 7.5, 200 mM NaCl, and 1 mM DTT. Similarly, fl-CHD1L was dialyzed overnight into 20 mM MES, pH 6.0, 300 mM NaCl, 10% glycerol, and 1 mM DTT. Protein was then purified by ion-exchange chromatography. cat-CHD1L was bound to a Q-sepharose column (GE Healthcare, Chicago, Ill.) and fl-CHD1L was bound to a S-sepharose column (GE Healthcare, Chicago, Ill.), and proteins were eluted using a NaCl gradient of 0.2-1M for cat-CHD1L and 0.3-1M for fl-CHD1L. Pure fractions were pooled, concentrated, and further purified by size-exclusion chromatography using a Superdex™ 200 column (GE Healthcare, Chicago, Ill.) with 20 mM HEPES, pH 7.5, 100 mM NaCl, 1 mM TCEP, and 10% Glycerol. Protein purifications were conducted using an ÄCTA Start FPLC (GE Healthcare, Chicago, Ill.).

CHD1L ATPase Assay

All reactions were carried out using low volume non-binding surface 384-well plates (Corning Inc., Corning N.Y.). cat-CHD1L or fl-CHD1L (100 nM) and 200 nM c-Myc DNA or mononucelosome (Active Motif, Carlsbad, Calif.) were added to a buffer containing 50 mM Tris pH 7.5, 50 mM NaCl, 1 mM DTT, 5% glycerol, and the reaction was initiated by the addition of 10 μM ATP (New England Biolabs, Ipswich, Mass.) to a total volume of 10 μL and incubated at 37° C. for 1 h. ATPase activity was assayed by adding 500 nM of Phosphate Sensor (Life Technologies, Carlsbad, Calif.), containing labeled phosphate-binding protein, specifically labeled with the fluorophore MDCC, and measuring excitation (430 nm) and emission (450 nm) immediately on an EnVision® plate reader (PerkinElmer, Hopkinton, Mass.). An inorganic phosphate standard curve was used to convert the fluorescence to [Pi], and enzyme kinetics were determined using Prism8 (GraphPad Software, San Diego, Calif.).

HTS Drug Discovery for Inhibitors of CHD1L

Assay composition was the same as described above using cat-CHD1L, except that the reaction mixture volume was modified to accommodate addition of drug or DMSO. Using a Janus® liquid handler (PerkinElmer, Hopkinton, Mass.), a selected amount of compounds dissolved in 100% DMSO were mixed with 50 mM Tris pH 7.5, 50 mM NaCl, 1 mM DTT, 5% glycerol buffer to 200 μM in 10% DMSO. Next, 1 μL of each compound was added to the enzyme mixture to give a final concentration of 20 μM. The negative control used was 1% DMSO and 10 mM EDTA was used as a positive control. Reactions were initiated with the addition of 10 μM ATP and incubated at 37° C. for 1 h. ATPase activity was measured by fluorescence by adding 500 nM Phosphate Sensor. cat-CHD1L was screened against a 20,000-compound diversity set from Life Chemicals (Woodbridge, Conn.) and a Kinase Inhibitor library from Selleck Chemicals (Houston, Tex.). Both libraries were prescreened before purchase to remove Pan-assay interference compounds (PAINS) which tend to react nonspecifically with many biological targets rather than selectively with a desired target. [Baell & Nissink, 2018; Baell & Holloway, 2010]

Patient Derived Tumor Organoid (PDTO) Culture and Viability Assay

CRC patient tumor tissues were obtained from the UCCC GI tissue bank and expanded following established protocols. [Morin et al., 1997]. Briefly, cells were seeded at 5,000 cells per well in 96-well plates and cultured by established methods [Franken et al., 2006] allowing PDTO formation over 72 h. PDTOs were treated with DMSO (0.5%) or compound 6.0 with various concentrations for an additional 72 h to obtain a dose response. PDTO cell viability was measured using CellTiter-Blue® reagent (Promega, Madison, Wis.). Media (80 μL) was aspirated from wells and 80 μL of the reagent was added and incubated for 4 h and cell viability was measured by fluorescence intensity using excitation 560 excitation and 590 emission.

Evaluation of Apoptosis

SW620 cells were plated at 30,000 cells/well in 96-well plates. Cells were treated with DMSO (negative control), SN-38 (apoptosis positive control), and compound 6.0 at concentrations indicated for 12 h. Cells were then rinsed 2× with cold PBS, 1× with cold Annexin-V staining buffer (10 mM HEPES, pH 7.4, 140 mM NaCl, 2.5 mM CaCl₂), and then incubated with Annexin-V FITC at 1:100 for 30 min in the dark. Cells were then rinsed 2× with Annexin-V staining buffer and FITC intensity was measured using an EnVision® plate reader (PerkinElmer, Hopkinton, Mass.).

Evaluation of DNA Damage by γ-H2AX

DLD1^{CHD1L-OE} cells were seeded into a 96-well PerkinElmer Cell Carrier plate and allowed to adhere overnight. Cells were then treated with the appropriate compound at 10 μM (0.5% DMSO) or with CHD1L inhibitor in combination SN-38 (1 μM), oxaliplatin (10 μM), and etoposide (10 μM). Cells were treated for 6 h. Media was aspirated and cells were washed with cold PBS. Cells were then fixed with 3% paraformaldehyde for 15 min at room temperature, fixed cells were washed with PBS three times. Cells were blocked for 1 hour at room temperature in 5% BSA, 0.3% Triton X-100 in PBS. Cells were then immunostained with phospho-(S139)-g-H2AX rabbit mAb using a 1:800 dilution in 1% BSA, 0.3% Triton X-100 in PBS at 4° C. overnight. Primary antibody was aspirated and cells were washed with PBS. Cells were incubated for 2 h at room temperature with goat anti-rabbit Alexa Fluor Plus™ 647 fluorescent secondary antibody at a concentration of 5 μg/mL in 1% BSA, 0.3% Triton X-100 in PBS. Cells were then washed with PBS, Hoechst 33342 stain was diluted to a concentration of 1:1000 in PBS, and added to cells for 15 min at room temperature. Cells were then imaged using a 20× water objective on the Opera Phenix™ HCS imaging system (PerkinElmer). Synergy was determined using the coefficient of drug interaction (CDI) equation, CDI=(A+B)/(AB). Synergy was defined in these experiments with a CDI<0.8. Additivity was 0.8-1.2 and antagonism was defined by a CDI>1.2.

Aqueous Solubility and CLogP

Using a recently reported detailed method [Abraham et al., 2019], aqueous solubility was measured for compound 6. The PBS UV absorption spectra were compared to a fully saturated solution in 1-propanol and the solubility of compound 6.0 in 10% DMSO in PBS (pH 7.4) was determined using linear regression analysis. The measurement of solubility in PBS was conducted in duplicate experiments. The consensus LogP (CLogP) values were obtained using the SwissADME web tools. [Daina et al., 2017]

Microsome Stability Studies

The microsomal stability of compound 6.0 was determined using female CD-1 mouse microsomes (M1500) purchased from Sekisui XenoTech (Kansas City, Kans.), following the recently reported method. [Abraham et al., 2019] Samples were centrifuged at 20,000 g for 10 min and the supernatant was transferred to an autosampler vial for LCMS analysis. The following mass transition (m/z, amu) was monitored for compound 6 (molecular weight=393.5).

In Vivo Pharmacology All animal studies were conducted in accordance with the animal protocol procedures approved by the Institutional Animal Care and Use Committee (IACUC) at the University of Colorado Anschutz Medical Campus (Aurora, Colo.) and Colorado State University (Fort Collins, Colo.).

Pharmacokinetics

Nine-week old female CD-1 mice, purchased from Charles River (Wilmington, Mass.), were used for PK studies using recently reported methods [Abraham et al., 2019] Briefly, the PK studies were designed to cover a range of 0.25-to-24 h with 3 mice/time point for a total of 21 mice/compound 6. Each mouse was dosed with a single i.p. injection of compound 6.0 at 50 mg/kg prepared in 100% DMSO. Whole blood was harvested at specific time points and the separated plasma frozen at −80° C. for storage or used for LC-MS/MS analysis.

Pharmacodynamics and Liver Toxicity

Two million VimPro-GFP SW620 cells suspended in 100 μL of a 1:1 mixture of Matrigel® matrix (Corning Life Sciences, Corning, N.Y.) and RPMI 1640 were injected into the flanks of 9-week old female athymic nude mice (Nude-Foxn1nu (069)) (Envigo, Huntingdon, Cambridgeshire, UK). Growth was monitored with caliper measurements 3× per week. At four weeks, mice were randomized into 2 groups and treated with 50 mg/kg of compound 6.0 in 200 μL of vehicle (10% DMSO, 40% PEG 400, 50% PBS pH=7.4) or with vehicle control. Treatments were administered i.p. QD over five days. Mice were sacrificed 2 h after the final dose on day five of the treatment. Tumors and livers were collected for analysis of compound 6.0 accumulation measured by LCMS, Western blot analysis measuring effects on EMT and apoptosis, and liver toxicity.

Statistical Analysis

Data were subjected to unpaired two-tailed Student's t-test with Welch's correction statistical analysis or as otherwise stated using Prism8 (GraphPad, LaJolla, CA). All experiments were replicated 3× (n=3) or as described in the methods.

### Example 10: Additional Experimental Methods for Assessment of Compound Activities

Microsome stability. CD-1 mouse microsomes were commercially purchased and the reactions were performed as previously described. Briefly, a master mix was prepared as follows: Microsomes (0.5 mg/mL), 10 μM CHD1Li solubilized in DMSO (0.1%), 5 mM UDPGA, 25 μg alamethicin, and 1 mM MgCl2 in 100 mM phosphate buffer (pH 7.4). The master mix was pre-incubated at 37° C. for 5 min, then 1 mM NADPH was added to start the microsomal activity reaction and maintained at 37° C. throughout the time course. Reactions were stopped at 0, 5, 15, 30, 45, and 60 min by adding 200 μL acetonitrile and analyzed by mass spectrometry. The appropriate microsome controls were also performed in the same reaction conditions.

γ-H2AX DNA damage combination studies with irinotecan (SN38). CHD1L inhibitor 6 alone and in combination with SN38 was assessed for DNA damage as previously reported [Abbott et al., 2020; Abraham et al., 2019]. Using DLD1 colorectal cancer cells that have low CHD1L endogenous expression and DLD1 cells engineered to overexpress CHD1L, DNA damage studies were conducted measuring the immunofluorescence of γ-H2AX, a well-established biomarker of DNA damage [Ji et al., 2017; Ivashkevich et al., 2012]. Cells were seeded into a 96-well plate as monolayers and treated with compound 6.0 at 10 μM (0.5% DMSO) or SN-38 (1 M), or the combination of 6.0 and SN38 over 6 hours. Media was aspirated and cells were washed with cold PBS. Cells were then fixed with 3% paraformaldehyde for 15 min at room temperature and washed with PBS three times. Cells were blocked for 1 hour at room temperature in 5% BSA, 0.3% Triton X-100 in PBS. Cells were then immunostained with phospho-(S139)-γ-H2AX rabbit mAb using a 1:800 dilution in 1% BSA, 0.3% Triton X-100 in PBS at 4° C. overnight. Primary antibody was aspirated, and cells were washed with PBS. Cells were incubated for 2 hours at room temperature with goat anti-rabbit Alexa Fluor Plus™ 647 fluorescent secondary antibody at a concentration of 5 μg/mL in 1% BSA, 0.3% Triton X-100 in PBS. Cells were then washed with PBS; Hoechst 33342 stain was diluted to a concentration of 1:1000 in PBS and added to cells for 15 min at room temperature. Cells were then imaged using a 20× water objective on the PerkinElmer Phenix HCS imaging system. We observed synergy between compound 6.0 and SN38 in inducing damage in DLD1 cells that overexpress CHD1L, determined using the coefficient of drug interaction (CDI) equation. CDI=(A+B)/(AB), synergy was determined with a CDI<0.8, additivity was 0.8-1.2, and antagonism was defined by a CDI>1.2. Welch's t-test statistical analysis was used to determine significance, where **=P≤0.01.

Cell based cytotoxicity dose response and combination studies. CHD1L inhibitors and SN38 (the active pharmacophore of irinotecan) were assessed for antitumor activity against colorectal cancer cell lines alone or in combination. Cell lines were cultured as monolayers or 3D tumor organoids using RPMI-1640 containing 5% fetal bovine serum as previously reported [Abbott et al., 2020]. For 3D SW620 tumor organoid cytotoxicity studies, 2,000 cells in 100 μL were plated into each well of the 96-well U-bottom ultra-low attachment microplates (Corning Inc., Corning, N.Y., USA). Plates were centrifuged at 1,000 rpm for 15 minutes to promote cell aggregation. A final 2% of Matrigel concentration was reached by coating the centrifuged cells with 25 μL of 10% Matrigel per well. Plates were then incubated for 3 days before treatment. 3D organoids were treated with 25 μL of various concentrations of drugs. 3 days after treatment, organoids with 40 μL of medium were manually transferred to 96-well white solid bottom plates. An equal amount of Celltiter-glo 3D (Promega) was added, and the plates were kept on a plate shaker for 45 minutes at 400 rpm before luminescence was read with Envision plate reader (PerkinElmer). For combination studies, synergy scores were determined using Combenefit analysis [De Veroli et al., 2016].

In vivo studies. CHD1L inhibitors compound 6.0 and 6.11 were assessed pharmacokinetically to determine the plasma half-life in nine-week-old female CD-1 mice as previously reported [Abbott et al., 2020]. Compound 6 was further assessed for antitumor activity alone and in combination with irinotecan against SW620 tumor xenografts in athymic nude mice. Xenografts were generated using the methodology as previously reported [Zhou et al., 2016]. Briefly, compound 6 was administered at 5 mg/kg by intraperitoneal injection (i.p.) 2×/day 7 days/week for a total of 5 weeks. Irinotecan was administered i.p. at 60 mg/kg 1×/week for 3 weeks, starting after the first week of compound 6 treatment. Body weight and tumor volumes were monitored 2×/week. Mice were sacrificed and tissues collected when single tumors reached 2000 mm³ or the total tumor volume reached 3000 mm [Ji et al., 2017]. Compound 6.11 was analogously assessed for antitumor activity alone and in combination with irinotecan against SW620 tumor xenografts. It was recently reported [Esquer et al., 2021 and its Supplementary Information] that the CRC M-phenotype is significantly more tumourogenic than other CRC EMT-phenotypes and that the M-phenotype also has significantly higher TCF-transcription. The xenografts used in this study were generated using isolated dual-reporter mesenchymal cells (M-phenotype) as described in Esquer et al, 2021. The half-life of compound 6.11 is 8 hours in CD-1 mice, which is 2.7-fold more stable compared to compound 6 (half-life=3 hours). Thus, the number of treatments was reduced from 2×/day to 1×/day. In addition, irinotecan was administered i.p. at 50 mg/kg.

FIGS. 7A and 7B illustrate representative single agent cytotoxicity dose response studies in SW620 colorectal cancer (CRC) tumor organoids and provide IC₅₀ for exemplary compounds as indicated. Tables 4A and 4B below provides a summary of cytotoxicity data for exemplary compounds. Table 4A provides cytotoxicity data for representative single compounds in several different CRC tumor organaoids.

TABLE 4A
Tumor Organoid Cytotoxicity

| | Tumor Organoid Cytotoxicity IC₅₀ (μM) | | | |
|---|---|---|---|---|
| CHD1L | SW620 | HCT116 | CRC042 | CRC102 |
| Inhibitor | (μM) # | (μM) | (μM) | (μM) |
| 6 | 4.6 | 4.93 | 18.61 | 22.6 |
| 6.1 | >40 | >30 | — | — |
| 6.2 | >40 | — | — | — |
| 6.3 | 3.8 | 3.72 | — | — |
| 6.4 | 28.4 | >30 | — | — |
| 6.5 | 1.2 | 2.2 | — | — |
| 6.6 | >40 | >30 | — | — |
| 6.7 | 12.7 | 17.6 | — | — |
| 6.8 | 3.6 | 6.85 | — | — |
| 6.9 | 8.1 | 19.6 | — | — |
| 6.10 | 22.6 | >30 | — | — |
| 6.11 | 2.6 | 3.48 | 4.5 | 8.38 |
| 6.12 | >20 | >30 | — | — |
| 6.13 | >20 | >30 | — | — |
| 6.14 | 10.4 | 15.3 | — | — |
| 6.15 | 16.4 | 19.7 | — | — |
| 6.16 | 1.4 | 2.43 | — | — |
| 6.17 | 5.5 | 3.86 | — | — |
| 6.18 | 1.4 | 2.95 | — | — |
| 6.19 | 7.7 | >30 | — | — |
| 6.20 | 2.1 | — | — | — |
| 6.21 | 1.6 | — | — | — |
| 6.22 | 12.6 | — | — | — |
| 6.23 | 5.7 | — | — | — |
| 6.24 | 2.7 | — | — | — |
| 6.25 | 19.7 | — | — | — |
| 6.26 | 5.5 | — | — | — |
| 6.27 | 2.1 | — | — | — |
| 6.28 | >30 | — | — | — |
| 6.29 | 5.1 | — | — | — |
| 6.30 | 6.0 | — | — | — |
| 6.31 | 2.4 | — | — | — |
| 6.32 | 5.4 | — | — | — |

Table 4B provides results of combination treatments of the indicated representative CHD1L Inhibitors (CHD1Li) with SN38 or Olaparib. Treatments are performed in four different CRC tumor organoid types. The concentration of CHD1L inhibitor is varied as indicated. IC₅₀ for the combination treatment are generally decreased compared to SN38 and Olaparib alone. #updated experimental for improved comparison among compounds, data rounded to one significant digit after the decimal point.

TABLE 4B
Tumor Organoid Cytotoxicity Combination Treatments

| | Tumor Organoid Cyctotoxicity Combination Treatments | | | | | |
|---|---|---|---|---|---|---|
| | SW620 | HCT116 | CRCO42 | CRCO42 | CRC102 | CRC102 |
| CHD1L | CHDILi (μM) + | CHDILi (pWl) + | CHDILi (μM) + | CHDILi (μM) + | CHDILi (μM) + | CHDILi (μM) + |
| Inhibitor | SN38 (nM) | Olaparib μM | SN38 nM | Olaparib uM | SN38 nM | Olaparib μM |
| 6 | (0 μM)-356 nM; | (0 μM)-377.8 μM; | (0 μM)-52 nM; | (0 μM)-134 μM; | (0 uM)-340 nM; | (0 μM)-202 μM; |
| | (3 μM)-191 nM; | (5 μM)-114.4 μM; | (14 μM)-26 nM; | (22 μM)-116 μM; | (20 μM)-111 nM; | (22 μM)-73 μM; |
| | (4 μM)-42 nM; | (6 μM)-64.7 μM; | (18 μM)-23 nM; | (25 uM)-58 μM; | (22 μM)-56 nM; | (25 μM)-70 μM; |
| | (5 μM)-8 nM; | (7 μM)-17.4 μM; | (22 μM)-6 nM; | (28 μM)-42 μM; | (25 uM)-26 nM; | (28 μM)-62 μM; |
| 6.3 | (0 μM)-356 nM: | — | — | — | — | — |
| | (2 μM)-96 nM; | | | | | |
| | (3 μM)-6 nM; | | | | | |
| | (4 μM)-1 nM; | | | | | |
| 6.11 | (0 μM)-356 nM; | (0 μM)-261.6 μM; | (0 μM)-52 nM; | (0 μM)-134 μM; | (0 μM)-340 nM; | (0 μM)-202 μM; |
| | (2.5 μM)-9 nM: | (2.5 μM)-300.8 μM; | (3.5 μM)-43 nM: | (4 μM)-52 μM; | (6 μM)-305 nM; | (6 μM)-50 μM; |
| | (3 μM)-8 nM; | (3.5 μM)-16.59 μM; | (4.5 μM)-8 nM: | (5 μM)-23 μM; | (8 μM)-16 nM; | (8 μM)-37 μM; |
| | | (4.5 μM)-8.44 μM; | (5 μM)-1 nM; | (6 μM)-10 μM; | (10 μM)-6 nM; | (10 μM)-11 μM; |
| 6.15 | (0 μM)-356 nM; | — | — | — | — | — |
| | (8 μM)-228 nM; | | | | | |
| | (9 μM)-197 nM; | | | | | |
| | (10 μM)-224 nM; | | | | | |
| 6.16 | (0 μM)-356 nM: | — | — | — | — | — |
| | (1.25 μM)-205 nM; | | | | | |
| | (1.5 μM)-90 nM; | | | | | |
| | (1.75 μM)-5 nM; | | | | | |

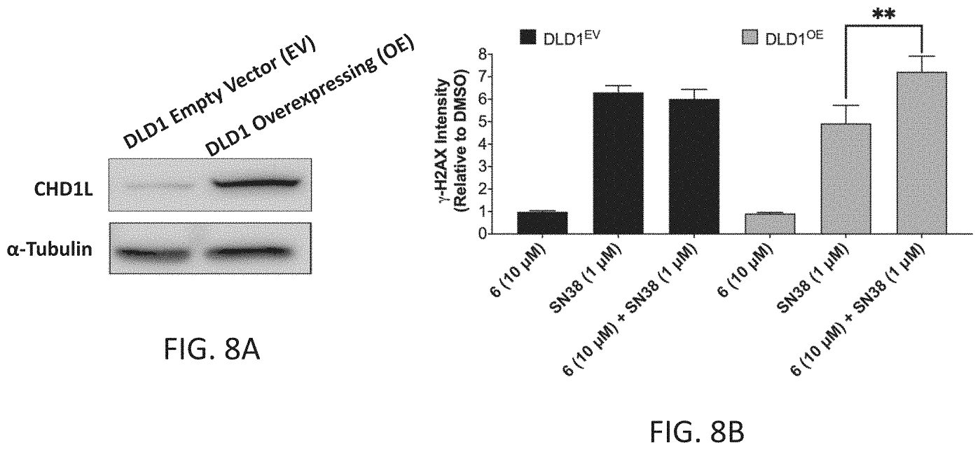

FIG. 8B presents a graph of γ-H2AX intensity (relative to DMSO) for compound 6 alone, irinotecan (SN38) alone, and a combination of the two in DLD1 empty vector (EV) cells and DLD1 (OE) overexpressing cells. FIG. 8A is a Western Blot showing relative expression of CHD1L in DLD1(EV) cells compared to DLD1(OE) cells compared to control expression of α-tubulin in these cells. CHD1L is known to be essential for PARP-1 Mediated DNA Repair, causing resistance to DNA damaging chemotherapy [Ahel et al., 2009; Tsuda et al., 2017]. Data in FIG. 8B demonstrate CHD1L inhibitor “on target” effects that synergize with SN38 inducing DNA damage.

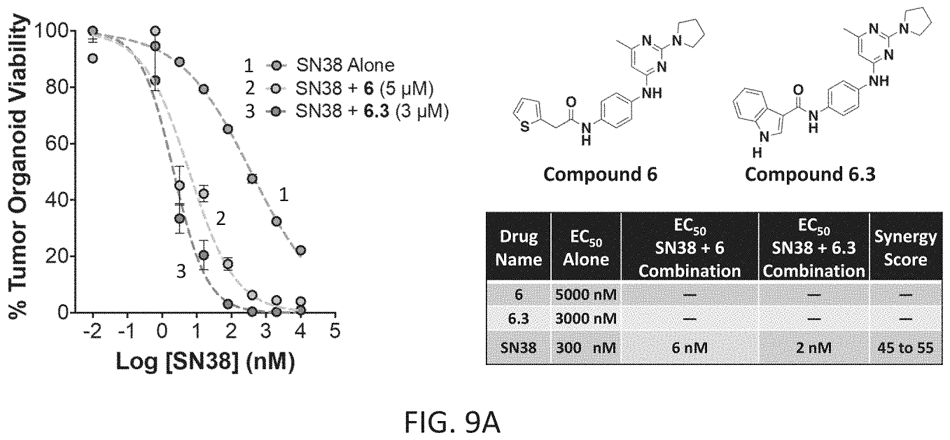

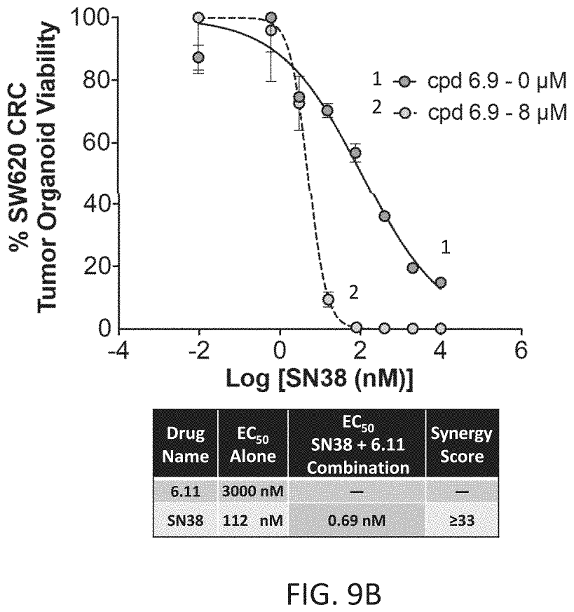

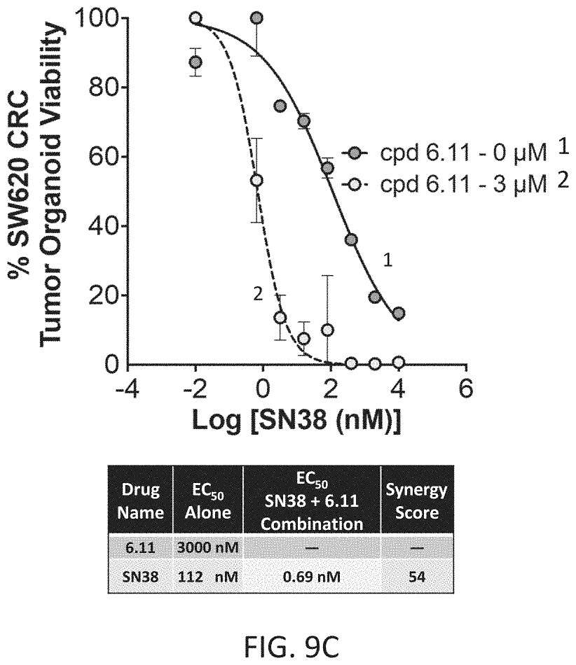

FIGS. 9A-9C illustrate the results of synergy studies with exemplary CHD1L Inhibitors 6, 6.3, 6.9 and 6.11 in SW620 Colorectal Cancer (CRC) Tumor Organoids. SN38 combinations with 6, and 6.3 are 50-fold, and 150-fold more potent, respectively, than SN38 alone in killing colon SW620 tumor organoids. SN38 combinations with 6.9 and 6.11 are both over 100-fold more potent than SN38 alone. Each of compounds 6, 6.3, 6.9 and 6.11 shows synergism with irinotecan (and SN38) for killing SW620 tumor organoids.

Synergy scores for exemplary CHD1L inhibitors where scores are determined as described in De Veroli et al. 2016 are provided in Table 5. For interpreting the value of synergy scores, as SynergyFinder has normalized input data as percentage inhibition, they can be directly interpreted as the proportion of cellular responses that can be attributed to the drug interactions. (e.g., synergy score 20 corresponds to 20% of response beyond expectation). According to our experience, the synergy scores near 0 gives limited confidence on synergy or antagonism.

When the synergy score is:
Less than −10: the interaction between two drugs is likely to be antagonism;
From −10 to 10: the interaction between two drugs is likely to be additivity;
Larger than 10: the interaction between two drugs is likely to be synergy.

TABLE 5
Exemplary Synergy Scores of SN38 with Representative Compounds
LOEWE Synergy Scores (Compound No.(IC₅₀ of Compound))

| SN38(nM) | Cpd 6 (5 μM) | Cpd 6.3 (3 μM) | Cpd 6.11 (3 μM) |
|---|---|---|---|
| 0.64 | −5 | 4 | 46 |
| 3.2 | 36 | 31 | 54 |
| 16 | 35 | 36 | 52 |
| 80 | 45 | 54 | 49 |
| 400 | 41 | 48 | 48 |
| 2000 | 28 | 35 | 36 |
| 10000 | 16 | 25 | 25 |

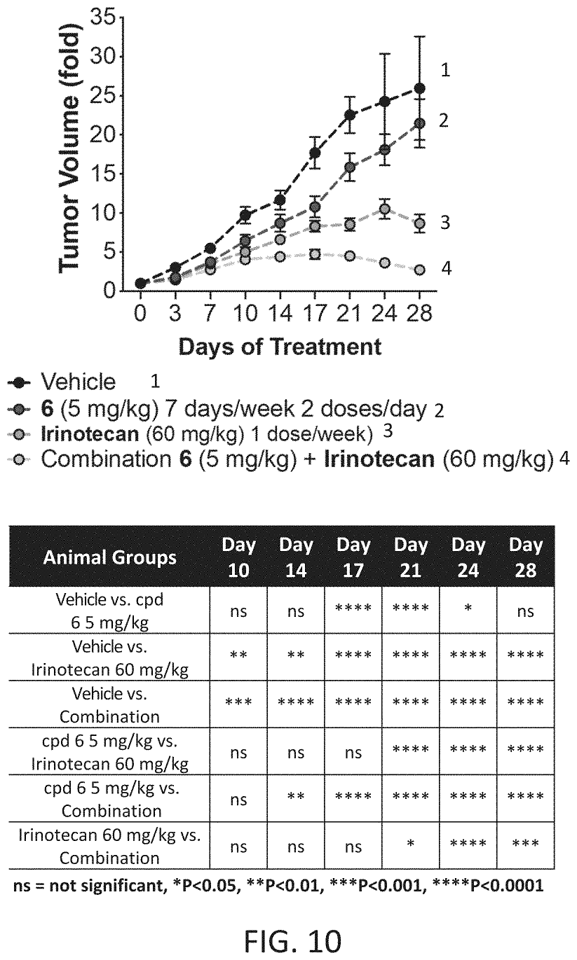

FIG. 10 includes a graph of tumor volume (fold) SW620 tumor xenografts as a function of days (up to 28 days) of treatment with Compound 6 alone, irinotecan alone or a combination thereof. The combination of irinotecan and Compound 6 significantly inhibit colon SW620 tumor xenografts to almost no tumor volume within 28 days of treatment compared to the single agent treatment groups or control.

FIG. 11 includes a graph of tumor volume (fold) SW620 tumor xenografts as a function of days (up to 28 days) of treatment with irinotecan alone (1) or a combination of Compound 6.0 and irinotecan (2). The combination of irinotecan and Compound 6 significantly inhibits colon SW620 tumors to almost no tumor volume beyond the last treatment compared to irinotecan alone. Within 2-weeks of the last treatment of irinotecan alone tumor volume rose to above the volume of the last treatment, signifying tumor recurrence. In contrast the combination maintained a lower tumor volume.

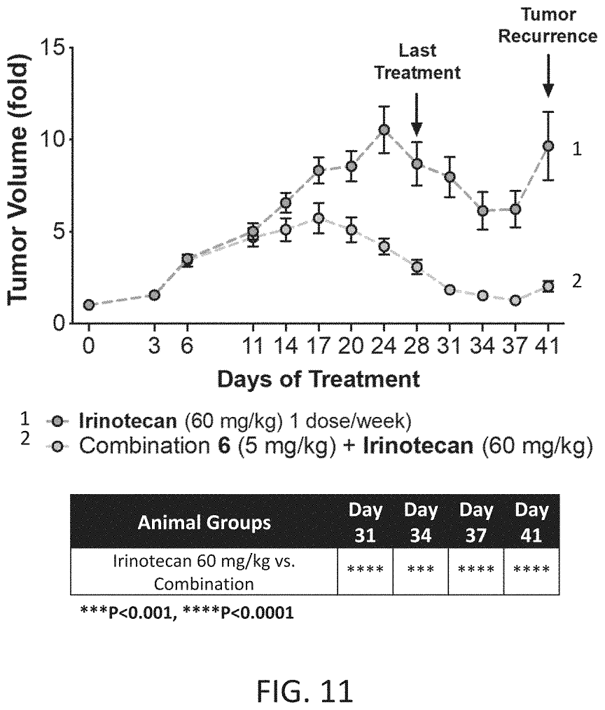

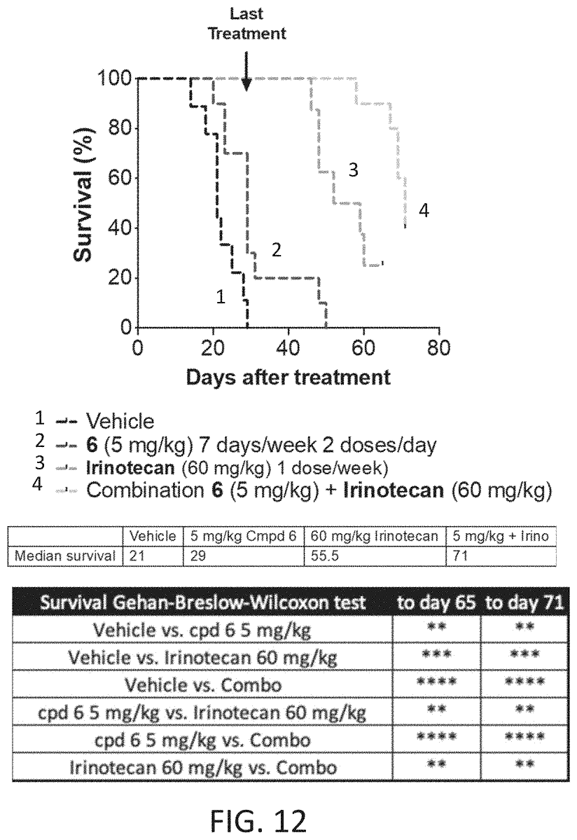

FIG. 12 shows that Compound 6 alone and in combination with irinotecan (4) significantly increases the survival of CRC-tumor-bearing mice compared to vehicle (1), Compound 6 alone (2) and irinotecan alone (3).

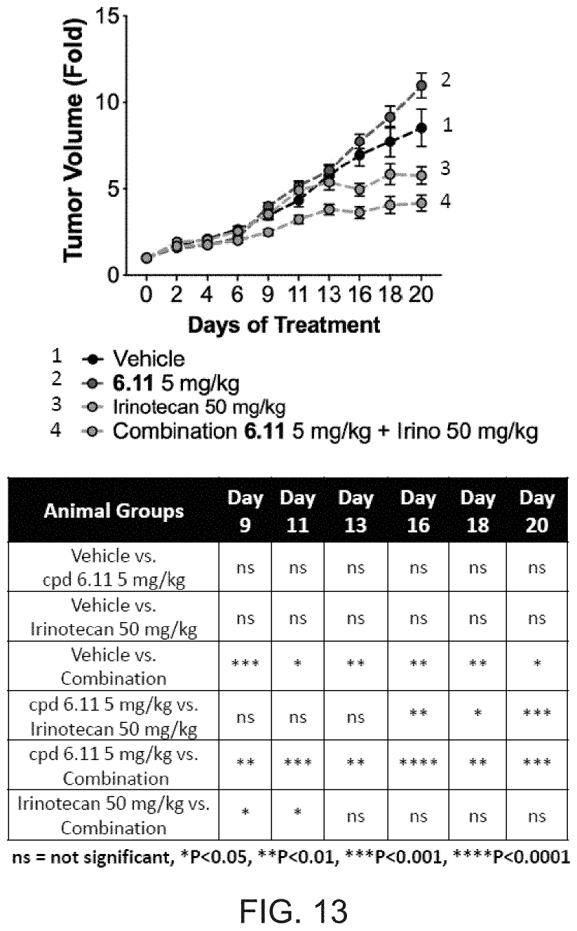

FIG. 13 includes a graph of tumor volume (fold) SW620 tumor xenografts as a function of days (up to 20 days) of treatment with Compound 6.11 alone, irinotecan alone or a combination thereof. The combination of irinotecan and Compound 6.11 significantly inhibits colorectal cancer SW620 tumor xenografts compared to irinotecan alone or control.

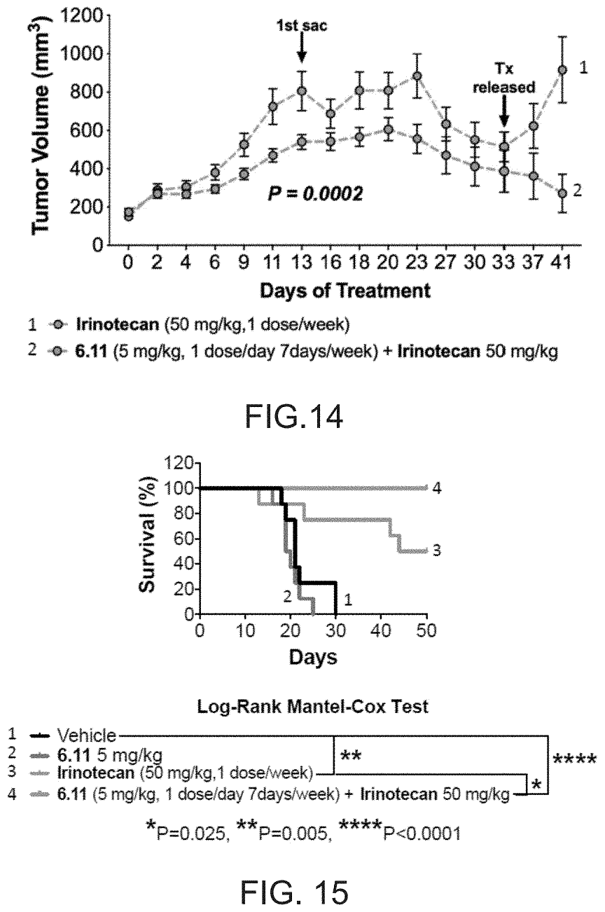

FIG. 14 includes a graph of tumor volume (fold) SW620 tumor xenografts as a function of days (up to 33 days) of treatment with irinotecan alone or a combination of compound 6.11 with irinotecan. The combination of irinotecan and compound 6.11 significantly inhibits colorectal SW620 tumors beyond the last treatment (day 33) compared to irinotecan alone. Eight days post treatment (Tx Released), tumor volume with irinotecan treatment alone rose ˜3-fold, signifying tumor recurrence. Conversely, tumor volume with treatment of the combination of 6.11 and irinotecan continued to drop (by ˜1.5-fold) post treatment. The difference in tumor volume between treatment with irinotecan alone and treatment with the combination of 6.11 and irinotecan 8 days post treatment is 3.4-fold.

FIG. 15 shows that Compound 6.11 in combination with irinotecan significantly increases the survival of CRC-tumor-bearing mice compared to irinotecan alone and control.

### Example 11: Summary of Currently Preferred Structure Activity Relationships for Inhibitors

The currently preferred structure activity relationship based on formula I for CHD1L Inhibitors of this invention is as follows:

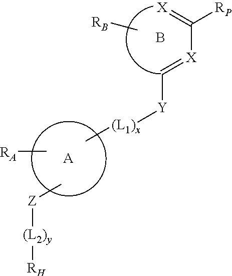

For the B ring, it is currently preferred the ring is a 6-member aromatic or fused 6,6-member aromatic ring and that both X are N. The B ring optionally contains a fused ring, which if present, can contain one or two additional N. Preferred R_{B} (B ring substitution), if present, include hydrogen, alkyl and fluoroalkyl groups. In certain embodiments, where x is 1 and L₁ is present, and preferably L₁ is —CH₂—, R_{B} can be an electronegative group, such as a halogen and particularly F or a haloalkyl, particularly CF₃—. Preferred R_{B} are hydrogen or C1-C3 alkyl. The preferred A ring is optionally substituted phenyl, with unsubstituted phenyl (where R_{A} is hydrogen) more preferred. The R_{P} group is believed to be associated with water solubility, with —N(R₂)(R₃) groups generally preferred and more particularly preferred optionally substituted N-containing heterocycles, where R₂ and R₃ together with the N to which they are attached form a 5- to 8-member ring which may contain one or more additional heteroatoms and which may be saturated (no double bond) or contain one or more double bonds. R_{H} is believed associated with activity and potency as well as metabolic stability. R_{H} is preferably an aromatic group and more particularly a heteroaromatic group with ring substitution that stabilizes the aromatic or heteroaromatic ring. Preferred Y is NR with R that is hydrogen more preferred. Preferably x is 0 except as noted above. Preferred Z is —CO—NH—. Preferred L₂ is —CH₂— or —CH₂—CH₂—. Preferred L₁, when present is —CH₂—.

In an embodiment, HTS screening for CHD1L identified a phenylamino pyrimidine pharmacophore illustrated in formula XX:

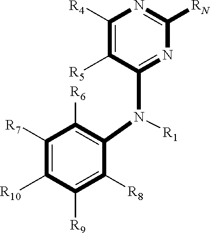

and salts thereof, where R₁-R₉ represent hydrogen or optional substituents, R₁₀ is a moiety believed to be associated with potency; and R_{N} is a moiety believed to be associated with physicochemical properties such as solubility. In embodiments, R₅ is a substituent other than hydrogen which is believed to be associated with metabolic stability. In specific embodiments, R₅ is a halogen, particularly F or C1, a C1-C3 alkyl group, particularly a methyl group. In embodiments, R₄ is a substituent other than hydrogen and in particular is a C1-C3 alkyl group, and more particularly is a methyl group. In a specific embodiment, R₅ is F and R₄ is methyl. In embodiments, R₆-R₉ are selected from hydrogen, C1-C3-alkyl, halogen, hydroxyl, C1-C3 alkoxy, formyl, or C1-C3 acyl. In embodiments, one or two of R₆-R₉ are moieties other than hydrogen. In an embodiment, one of R₆-R₉ is a halogen, particularly fluorine. In specific embodiments, all of R₆-R₉ are hydrogen. In embodiments, R_{N} is an amino moiety —N(R₂)(R₃). In specific embodiments, R_{N} is an optionally substituted heterocyclic group having a 5- to 7- member ring optionally containing a second heteroatoms (N, S or O). In embodiments, R_{N} is optionally substituted pyrrolidin-1-yl, piperidin-1-yl, azepan-1-yl, piperazin-1-yl, or morpholino. In R_{N} is substituted with one substituent selected from C1-C3 alkyl, formyl, C1-C3 acyl (particularly acetyl), hydroxyl, halogen (particularly F or Cl), hydroxyC1-C3 alkyl (particularly —CH₂—CH₂—OH). In embodiments, R_{N} is unsubstituted pyrrolidin-1-yl, piperidin-1-yl, azepan-1-yl, piperazin-1-yl, or morpholino.

In embodiments, R₁₀ is —NRy-CO-(L₂)y-R₁₂ or —CO—NRy--(L₂)y-R₁₂, where y is 0 or 1 to indicate the absence of presence of L₂ which is an optional 1-6 carbon atom linker group which linker is optionally substituted and wherein one or two, carbons of the linker are optionally replaced with O, NH, NRy or S, where Ry is hydrogen or a 1-3 carbon alkyl, and R₁₂ is an aryl group, cycloalkyl group, heterocyclic group, or heteroaryl group, each of which is optionally substituted. I_{N} embodiments, y is 1. L₂ is —(CH₂)p-, where p is 0-3. In embodiments, R₁₂ is thiophen-2-yl, thiophen-3-yl, 4-bromothiophen-2-yl, furany-2-yl, furan-3-yl, pyrrol-2-yl, pyrrol-3-yl, oxazol-4-yl, oxazol-5-yl, oxazol-2-yl, indol-2-yl, indol-3-yl, benzofuran-2-yl, benzofuran-3-yl, benzo[b]thiophen-2-yl, benzo[b]thiophen-3-yl, isobenzofuran-1-yl, isoindol-1-yl, or benzo[c]thiophen-1-yl. In embodiments, R₁ is hydrogen or methyl. In embodiments, R₁₂ is thiophen-2-yl, furany-2-yl, pyrrol-2-yl, oxazol-4-yl, indol-2-yl, benzofuran-2-yl, or benzo[b]thiophen-2-yl. In embodiments, R₁₂ is thiophen-2-yl or indol-2-yl. In embodiments, R₁ is hydrogen or methyl.

Exemplary compounds of the invention are illustrated in Scheme 1. In Scheme 1, X is halogen and preferably Cl or Br. In Scheme 1, R is C1-C5 alkyl or cycloalkyl, and preferably a C1-C3 alkyl or cyclopropyl and more specifically, methyl, ethyl, n-propyl or cyclopropyl.

Exemplary R_{P} and —N(R₂)(R₃) groups for any formulas herein are illustrated in Scheme 2.

Exemplary R₁₂ and R_{H} groups for any formulas herein are illustrated in Scheme 3.

Exemplary B rings for formula I are illustrated in Scheme 4.

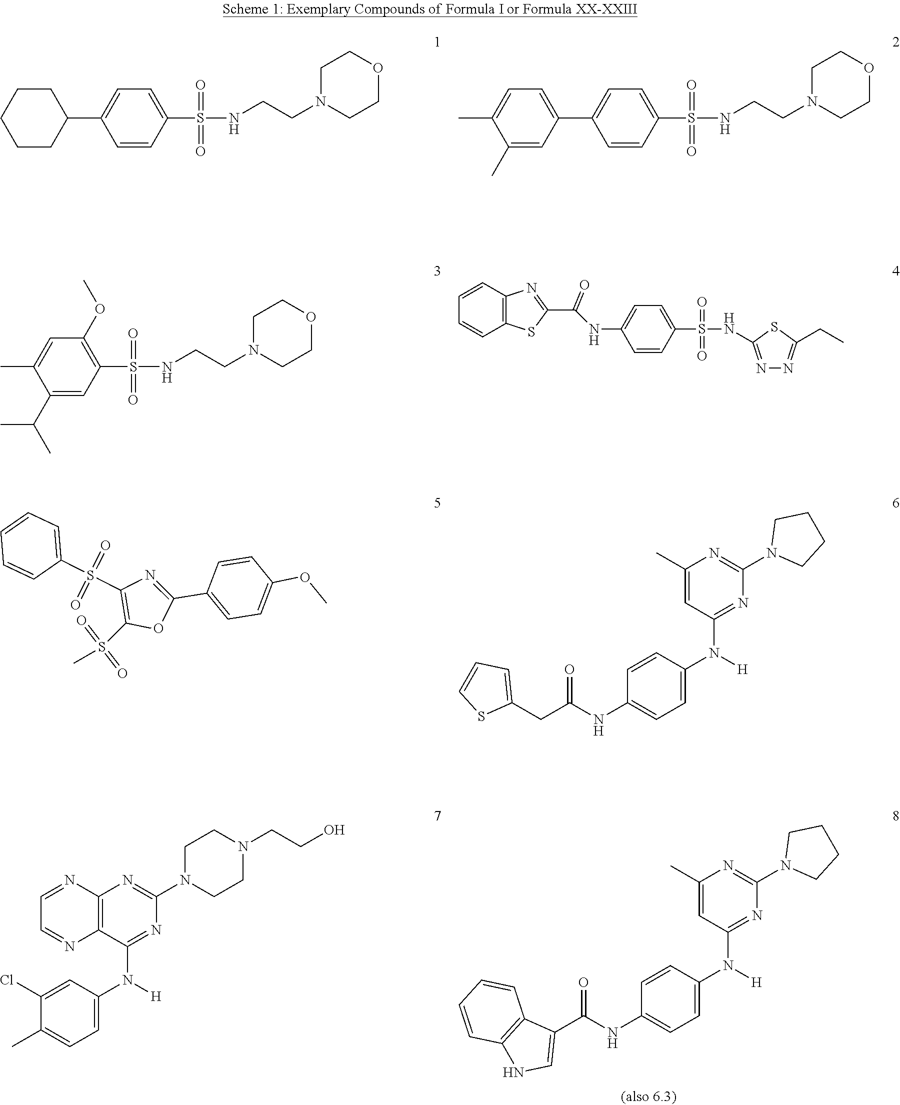

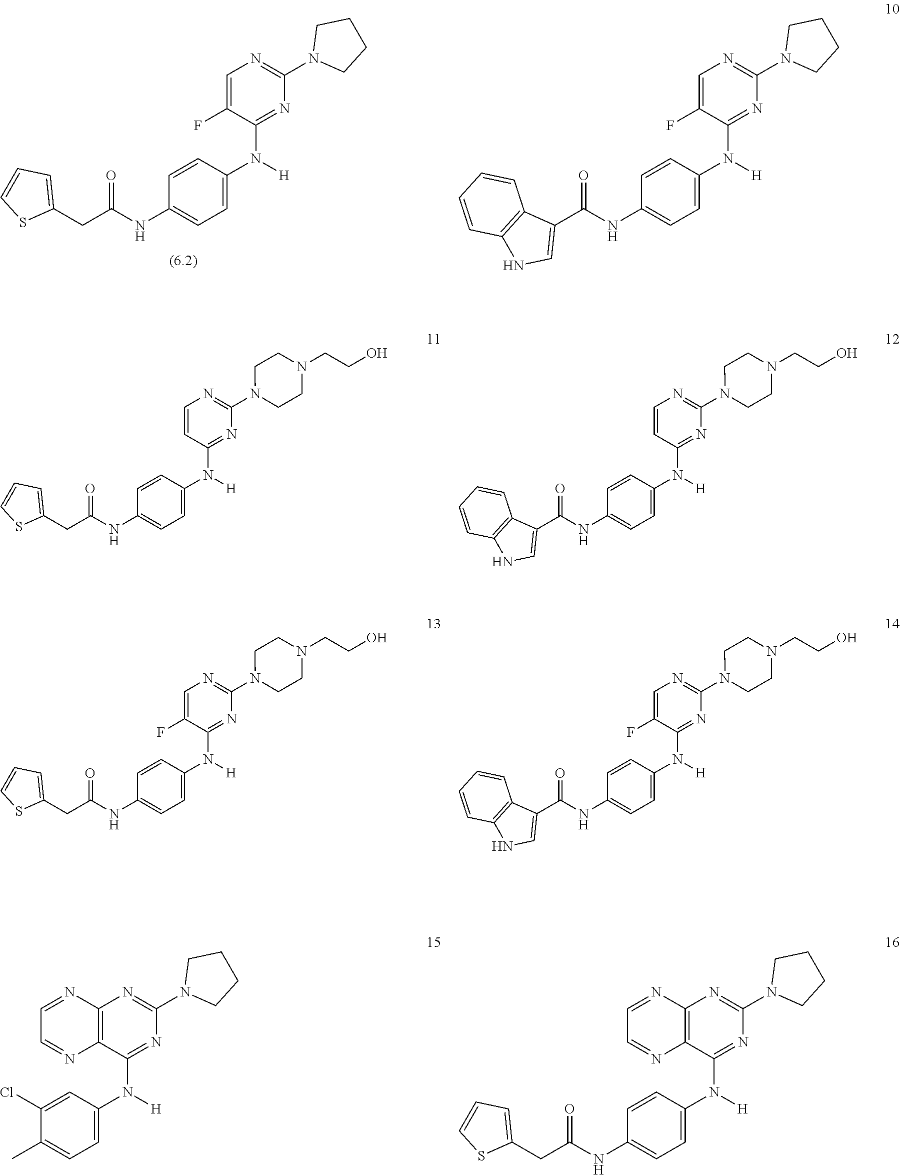

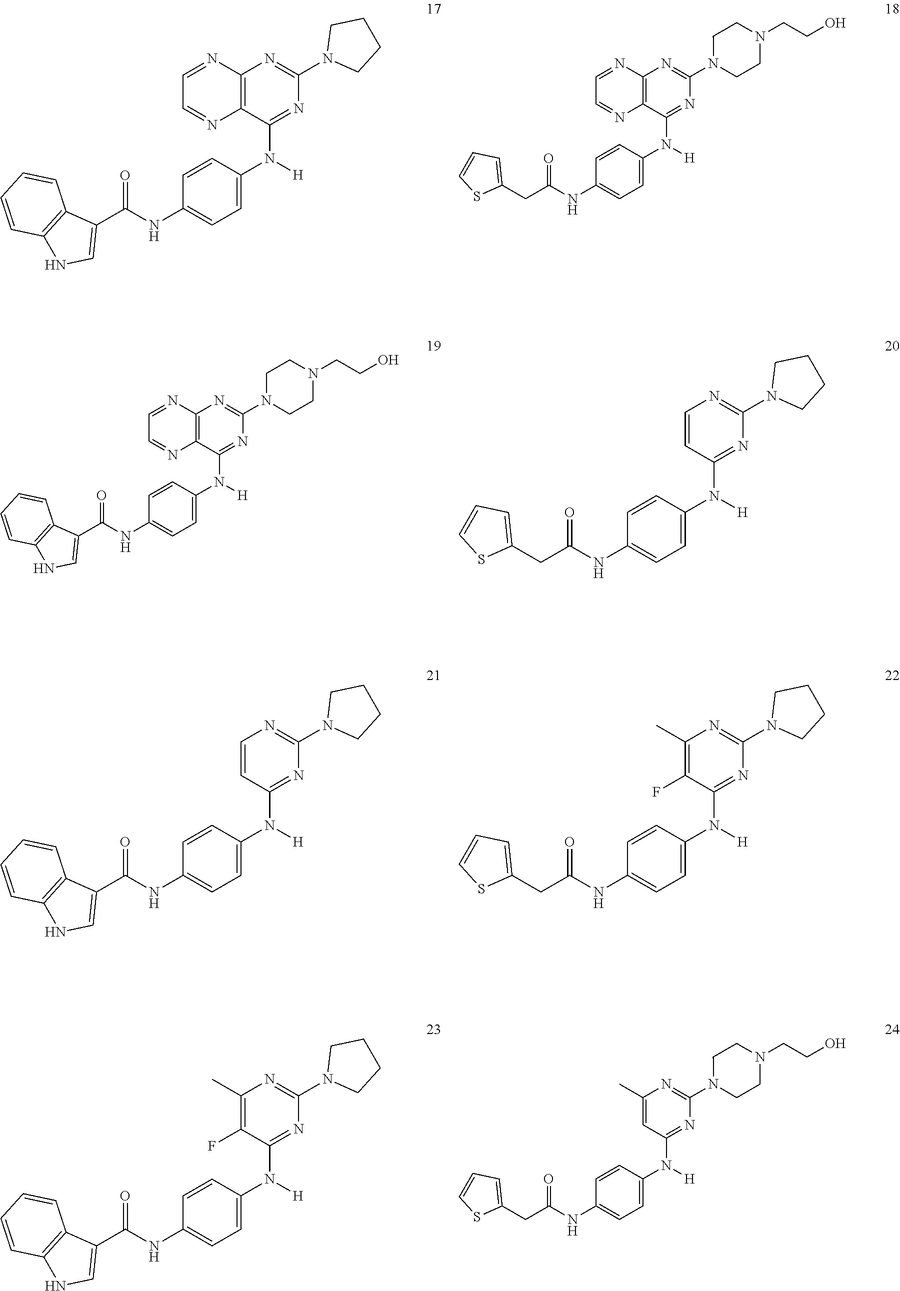

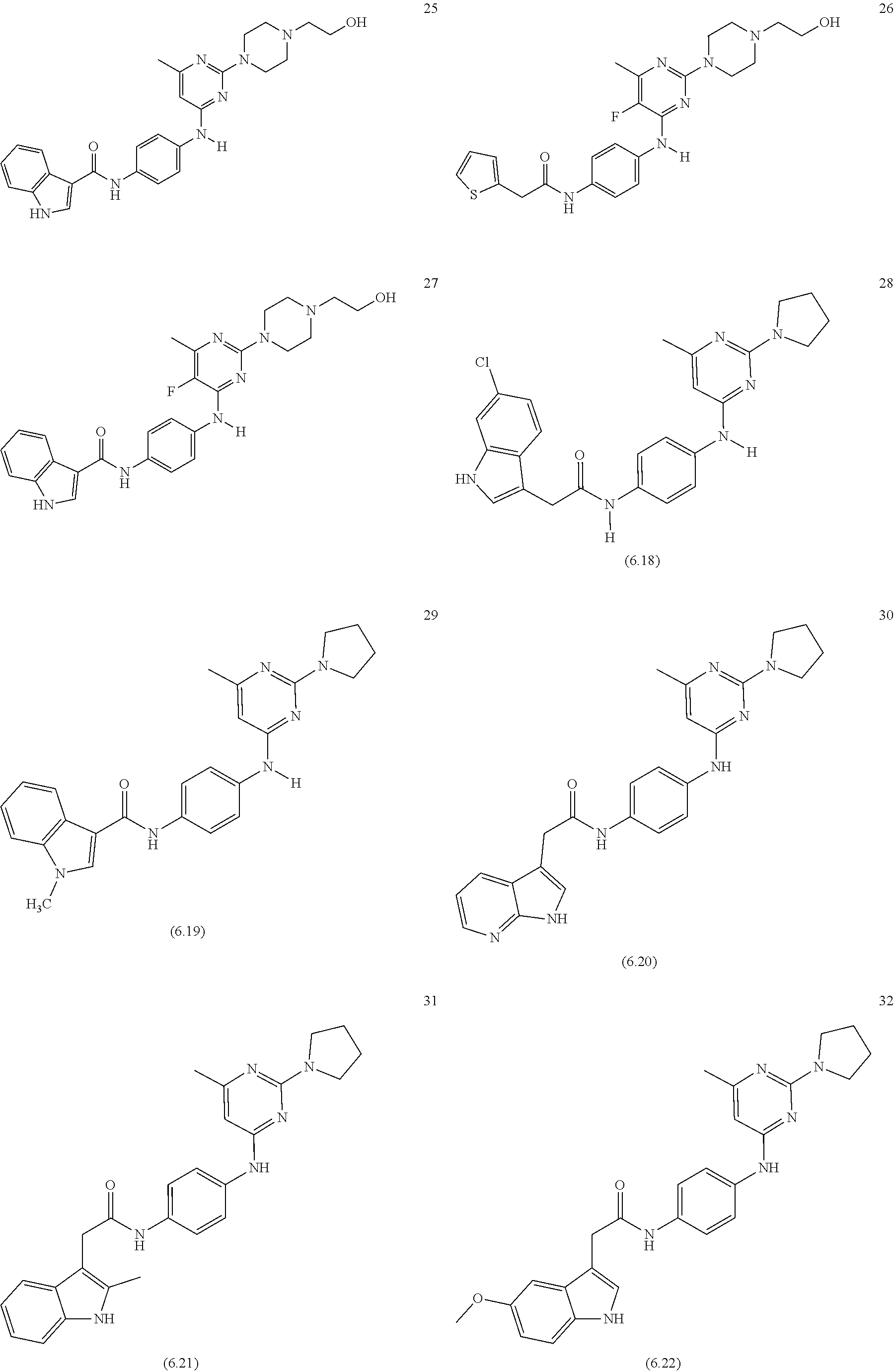

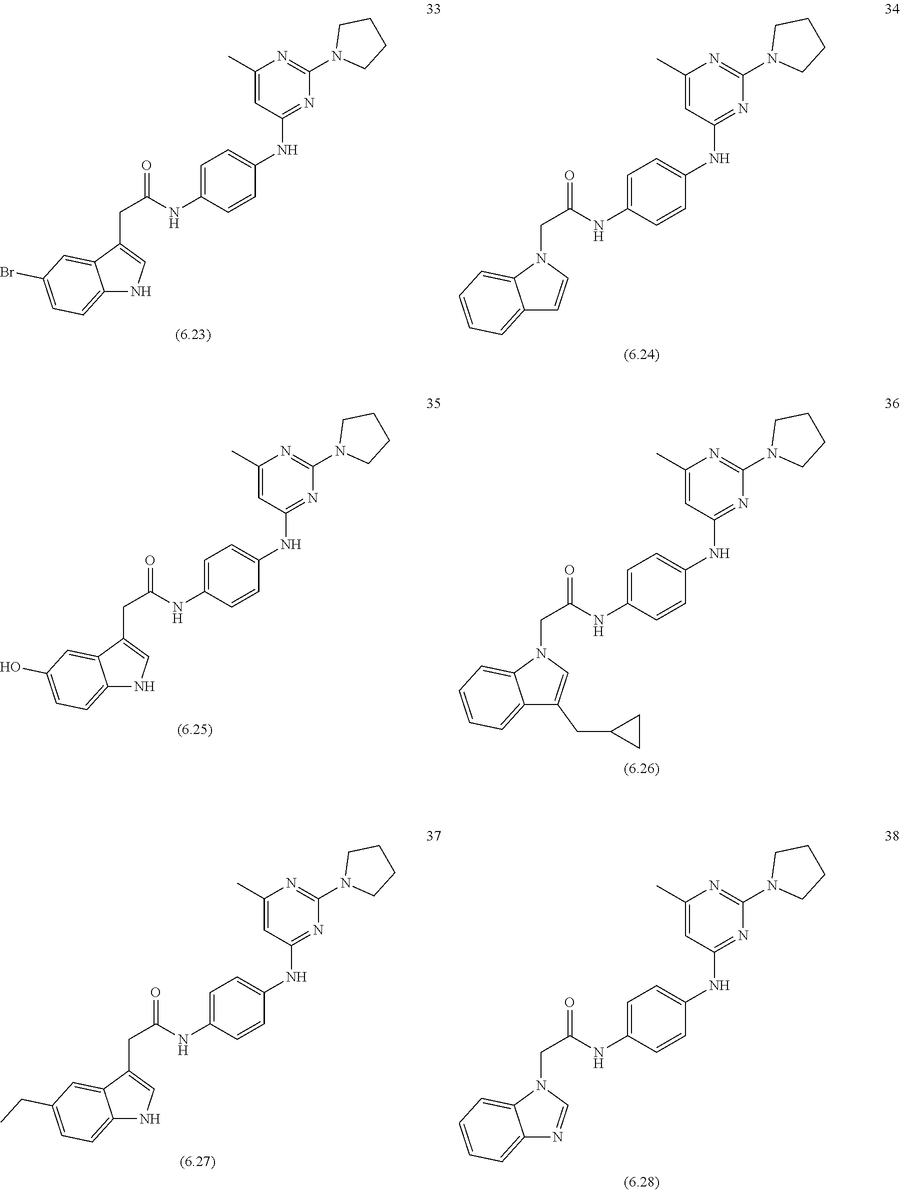 [SMILES: Cc1cc(Nc2ccc(NC(=O)Cc3c[nH]c4ccc(Br)cc34)cc2)nc(N2CCCC2)n1 ; Cc1cc(Nc2ccc(NC(=O)Cn3ccc4ccccc43)cc2)nc(N2CCCC2)n1 ; Cc1cc(Nc2ccc(NC(=O)Cc3c[nH]c4ccc(O)cc34)cc2)nc(N2CCCC2)n1 ; Cc1cc(Nc2ccc(NC(=O)Cn3cc(CC4CC4)c4ccccc43)cc2)nc(N2CCCC2)n1 ; CCc1ccc2[nH]cc(CC(=O)Nc3ccc(Nc4cc(C)nc(N5CCCC5)n4)cc3)c2c1 ; Cc1cc(Nc2ccc(NC(=O)Cn3cnc4ccccc43)cc2)nc(N2CCCC2)n1]

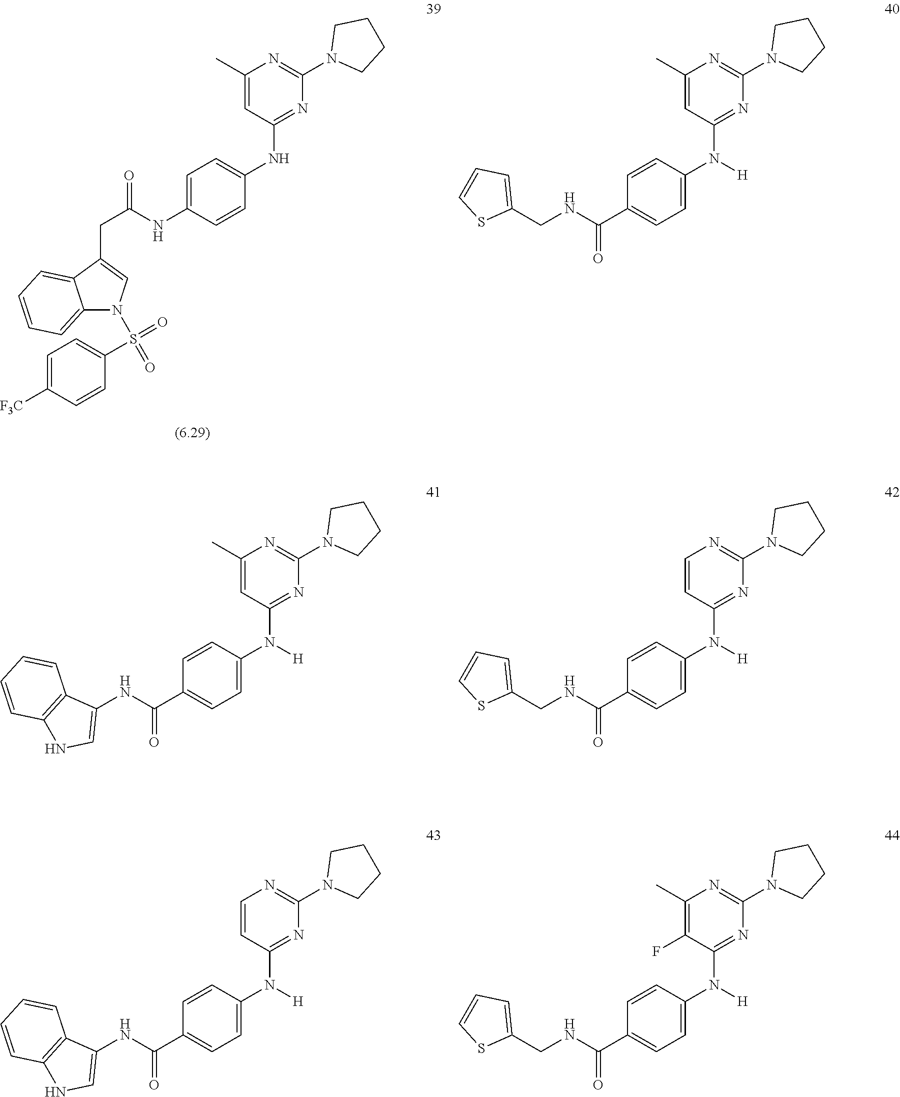 [SMILES: Cc1cc(Nc2ccc(NC(=O)Cc3cn(S(=O)(=O)c4ccc(C(F)(F)F)cc4)c4ccccc34)cc2)nc(N2CCCC2)n1 ; Cc1cc(Nc2ccc(C(=O)NCc3cccs3)cc2)nc(N2CCCC2)n1 ; Cc1cc(Nc2ccc(C(=O)Nc3c[nH]c4ccccc34)cc2)nc(N2CCCC2)n1 ; O=C(NCc1cccs1)c1ccc(Nc2ccnc(N3CCCC3)n2)cc1 ; O=C(Nc1c[nH]c2ccccc12)c1ccc(Nc2ccnc(N3CCCC3)n2)cc1 ; Cc1nc(N2CCCC2)nc(Nc2ccc(C(=O)NCc3cccs3)cc2)c1F]

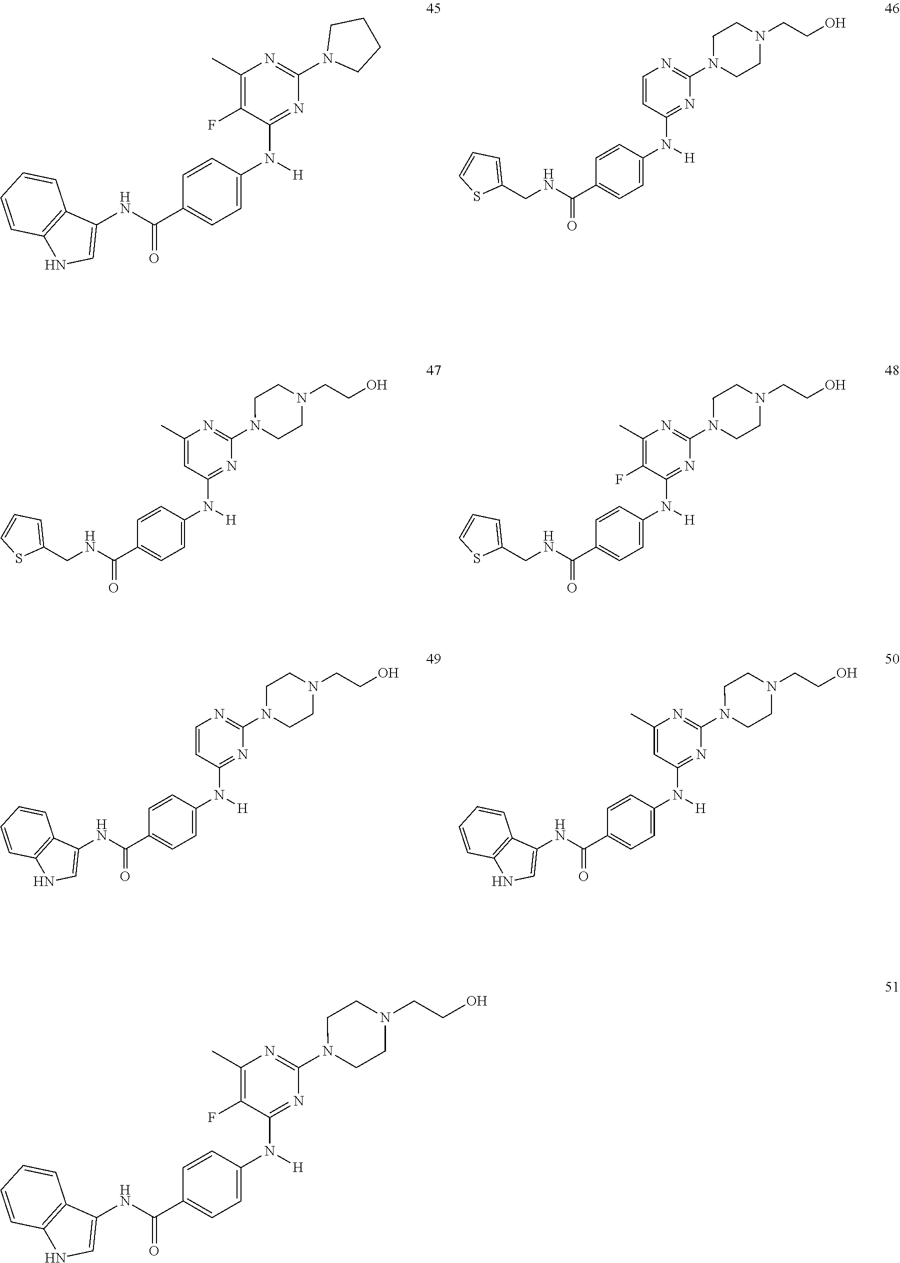

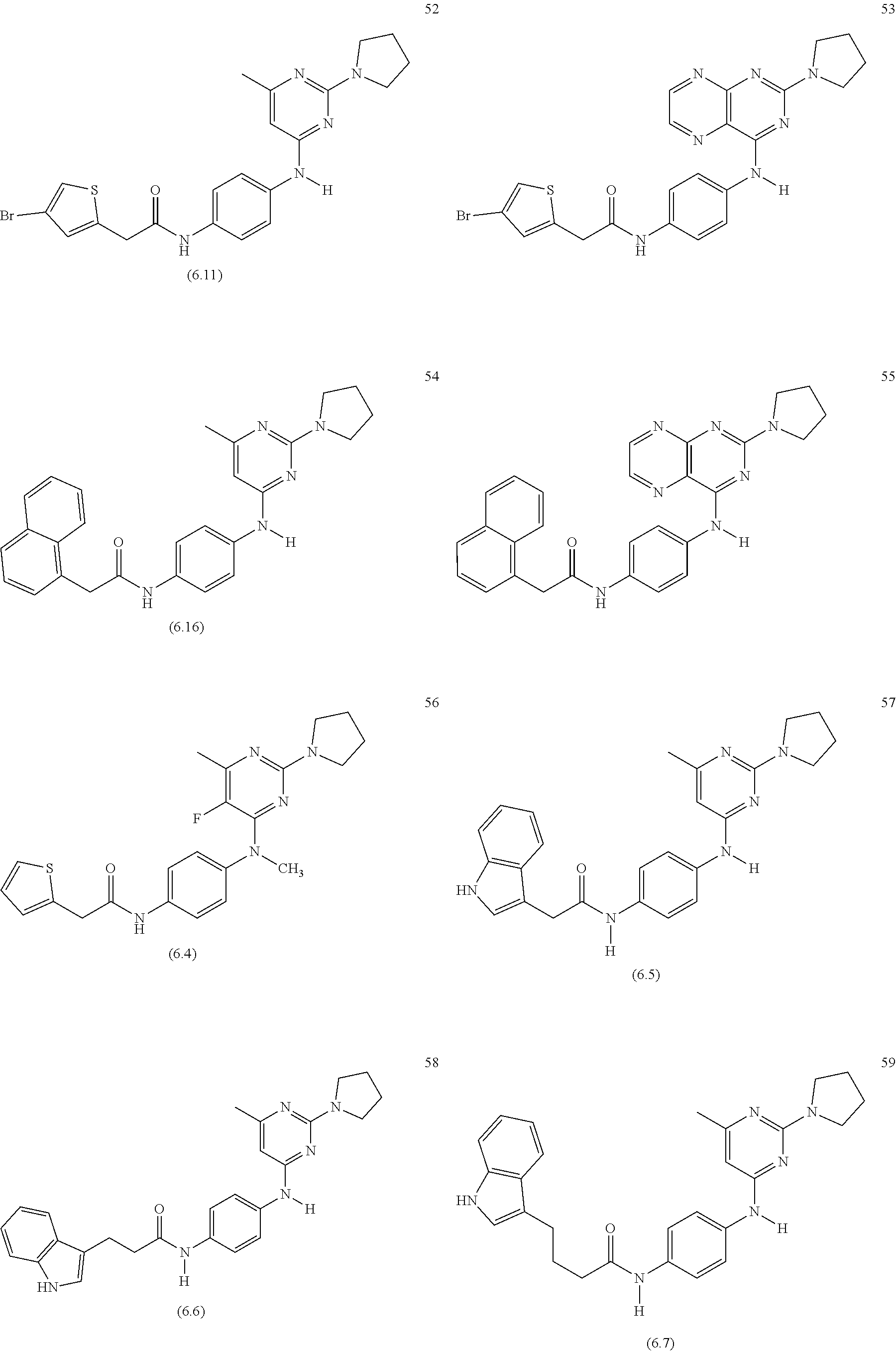

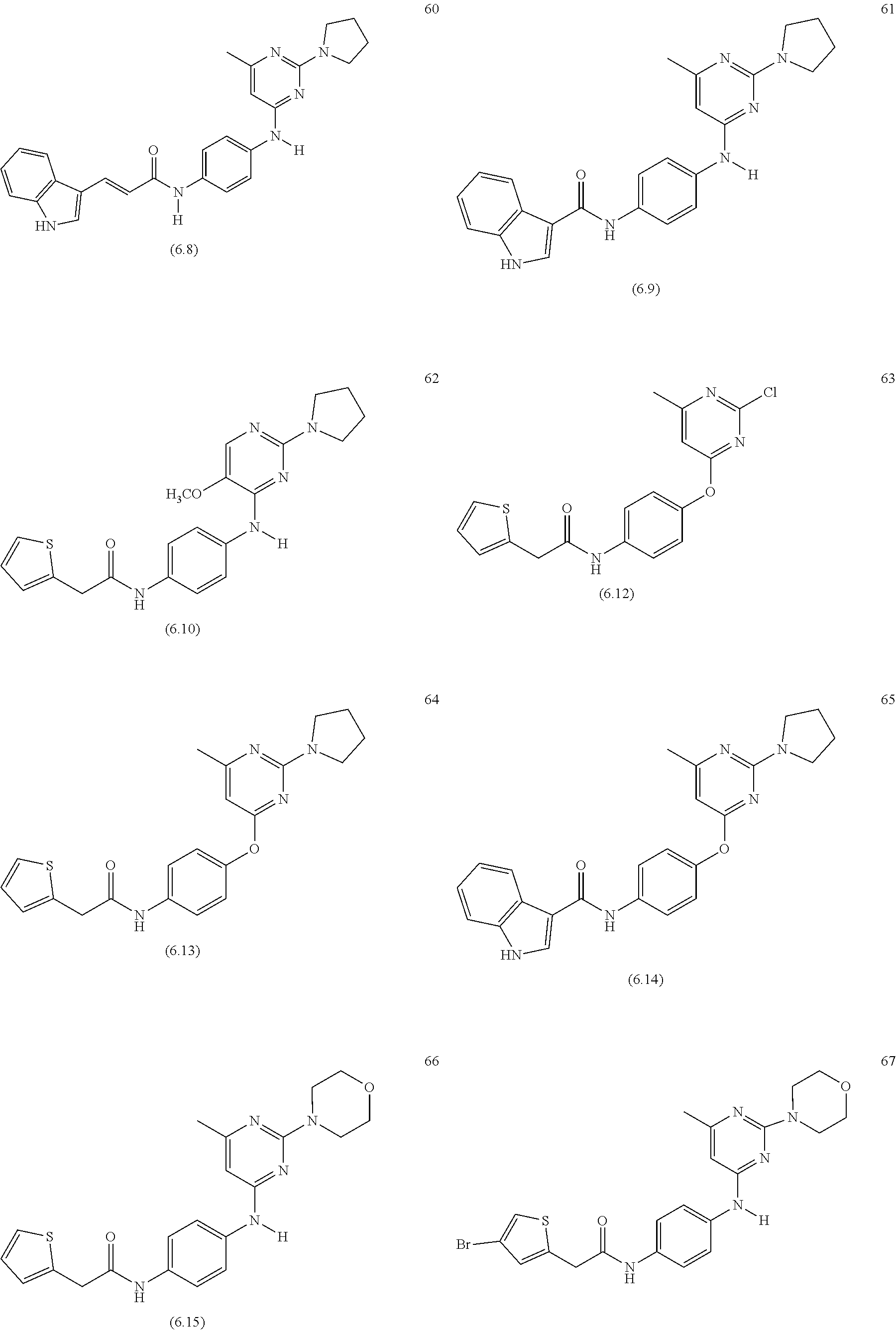

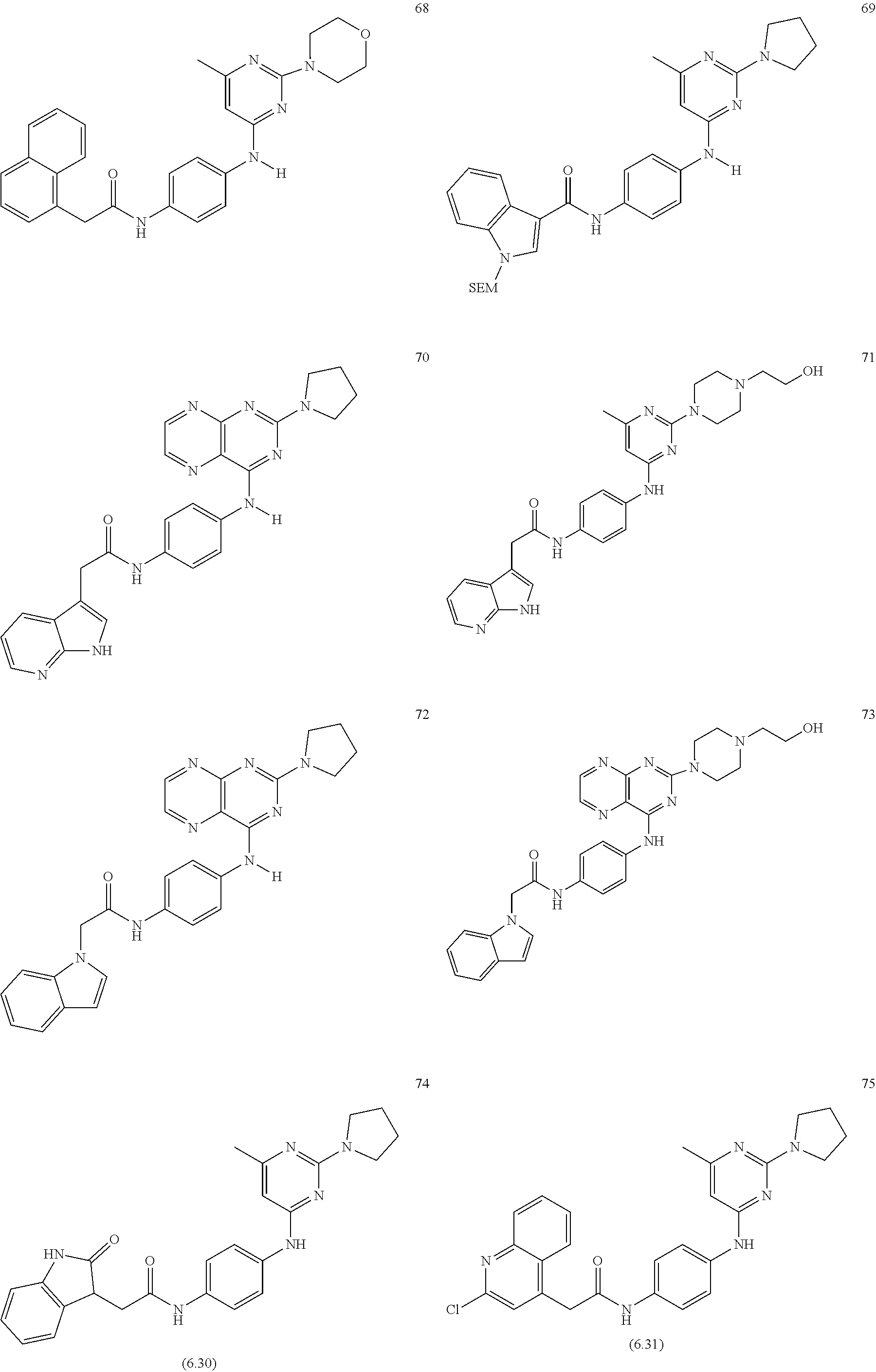

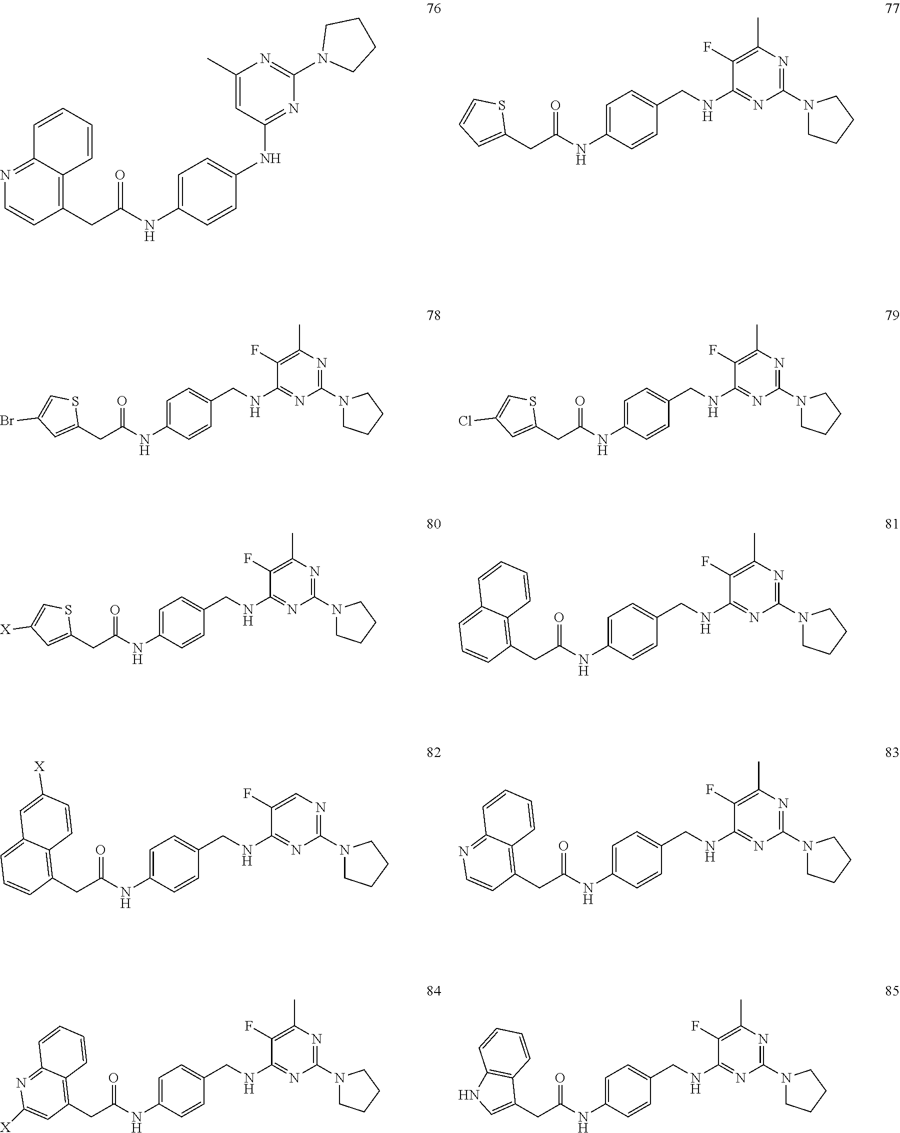

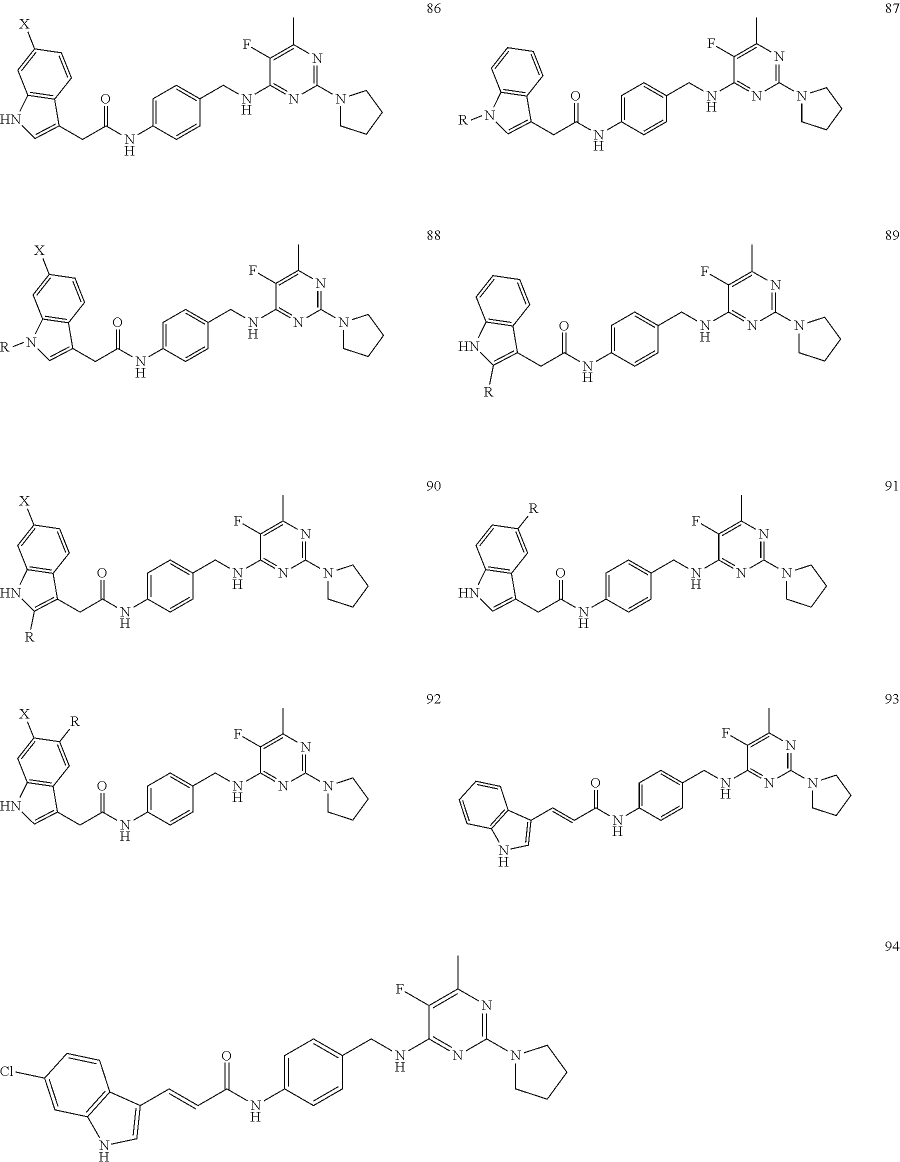

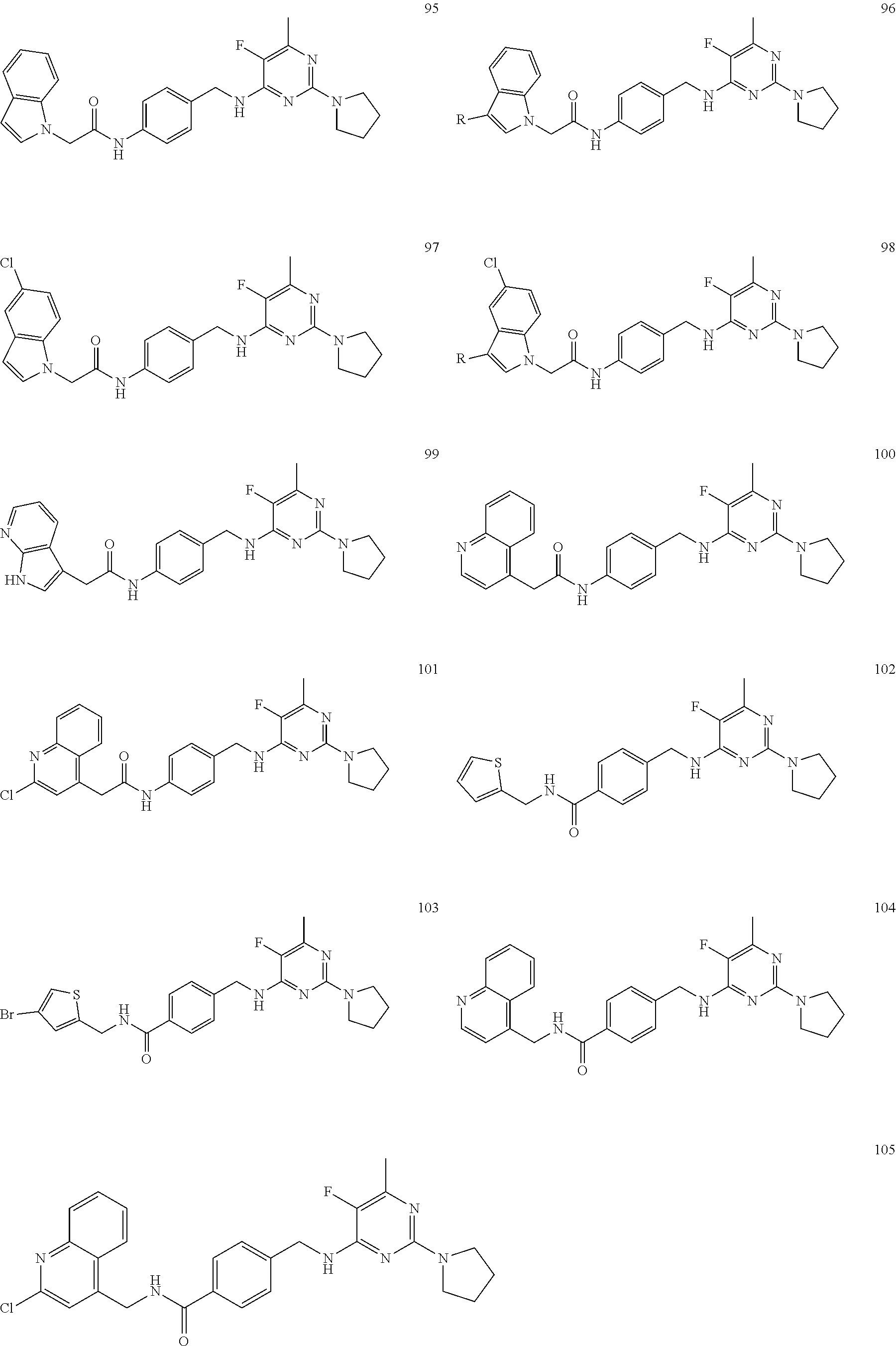

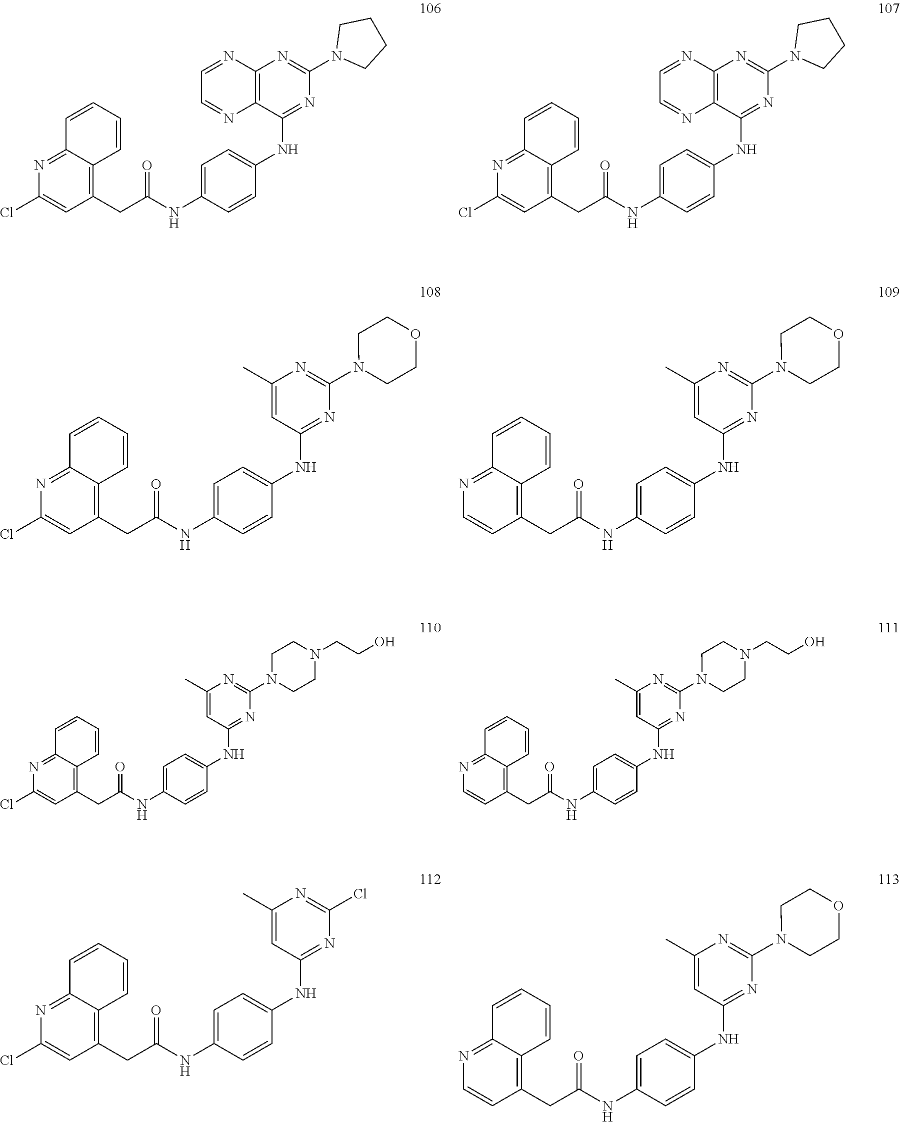

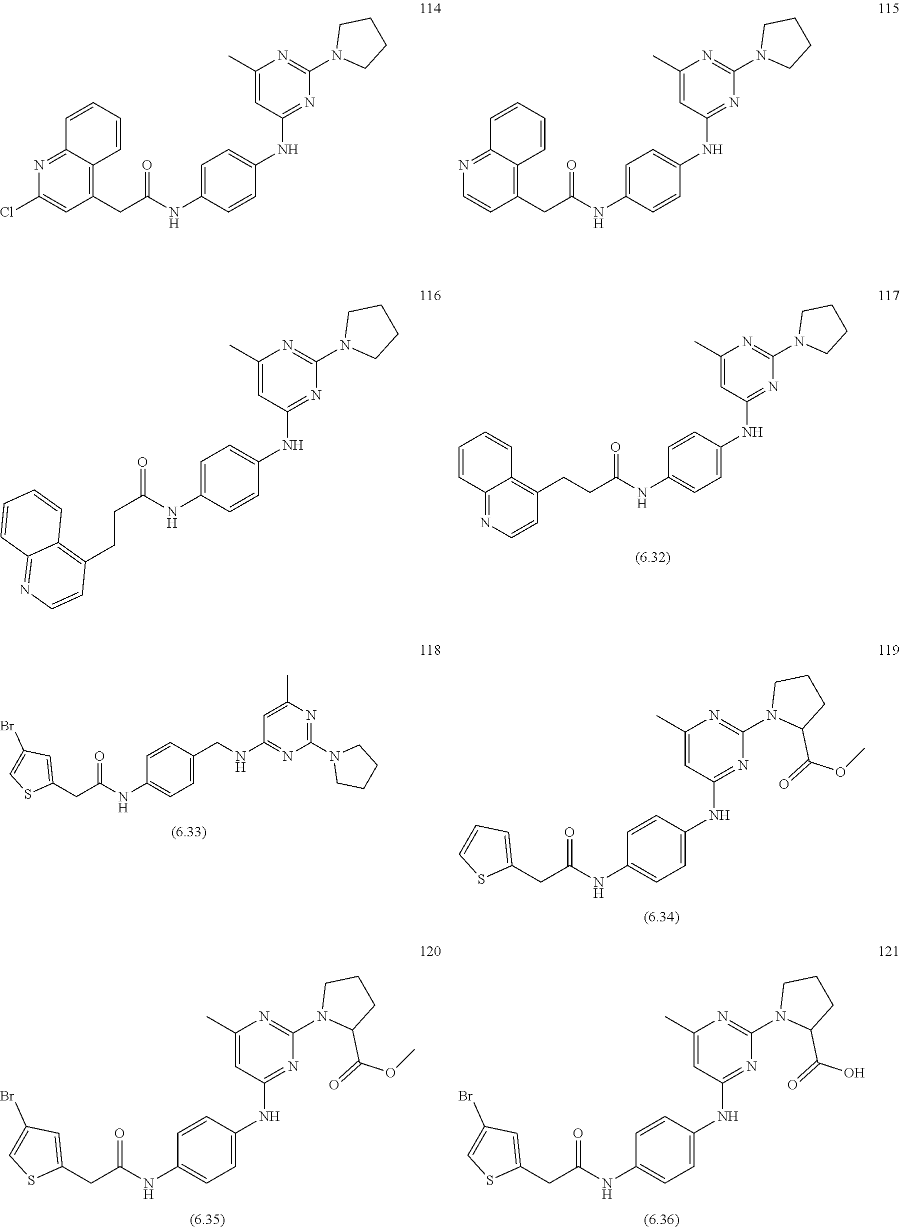

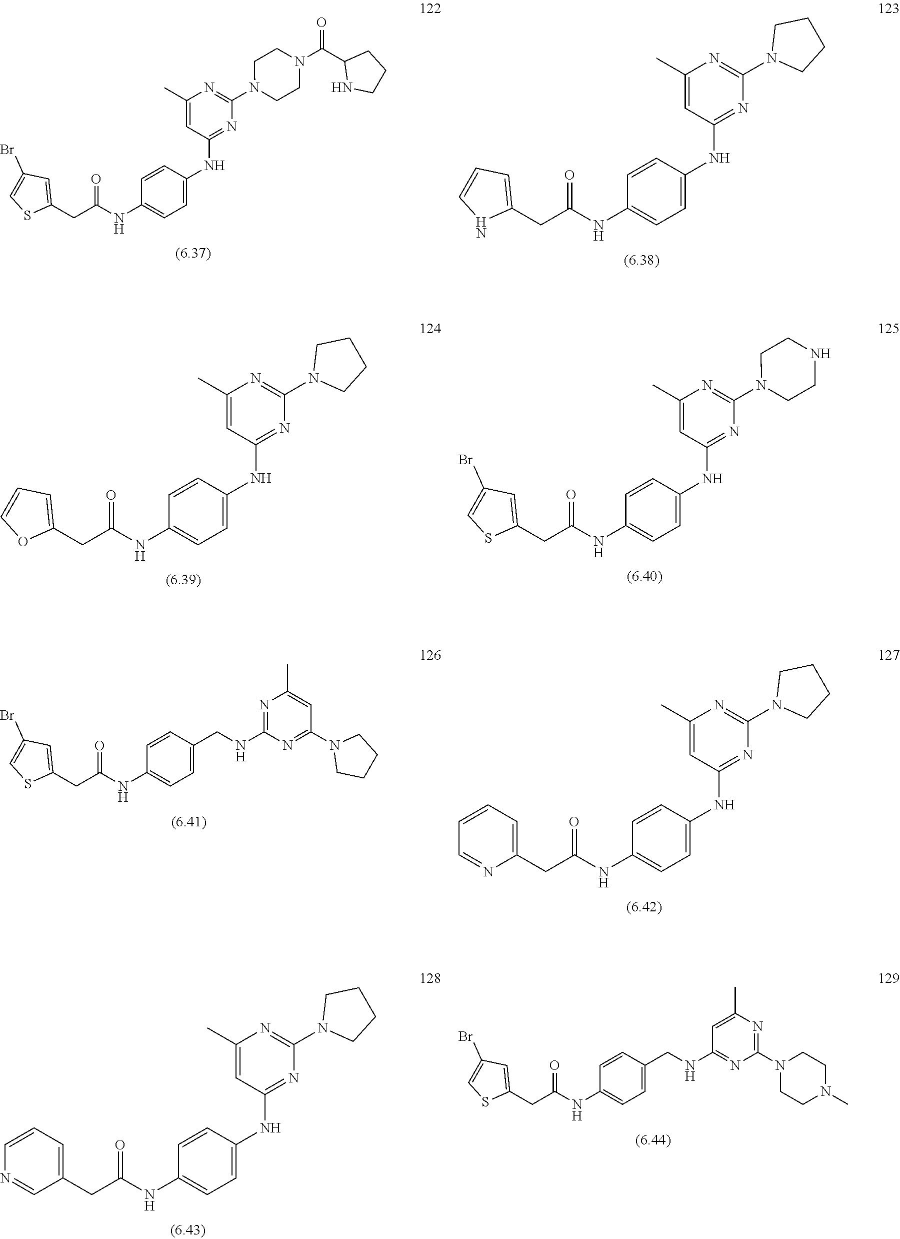

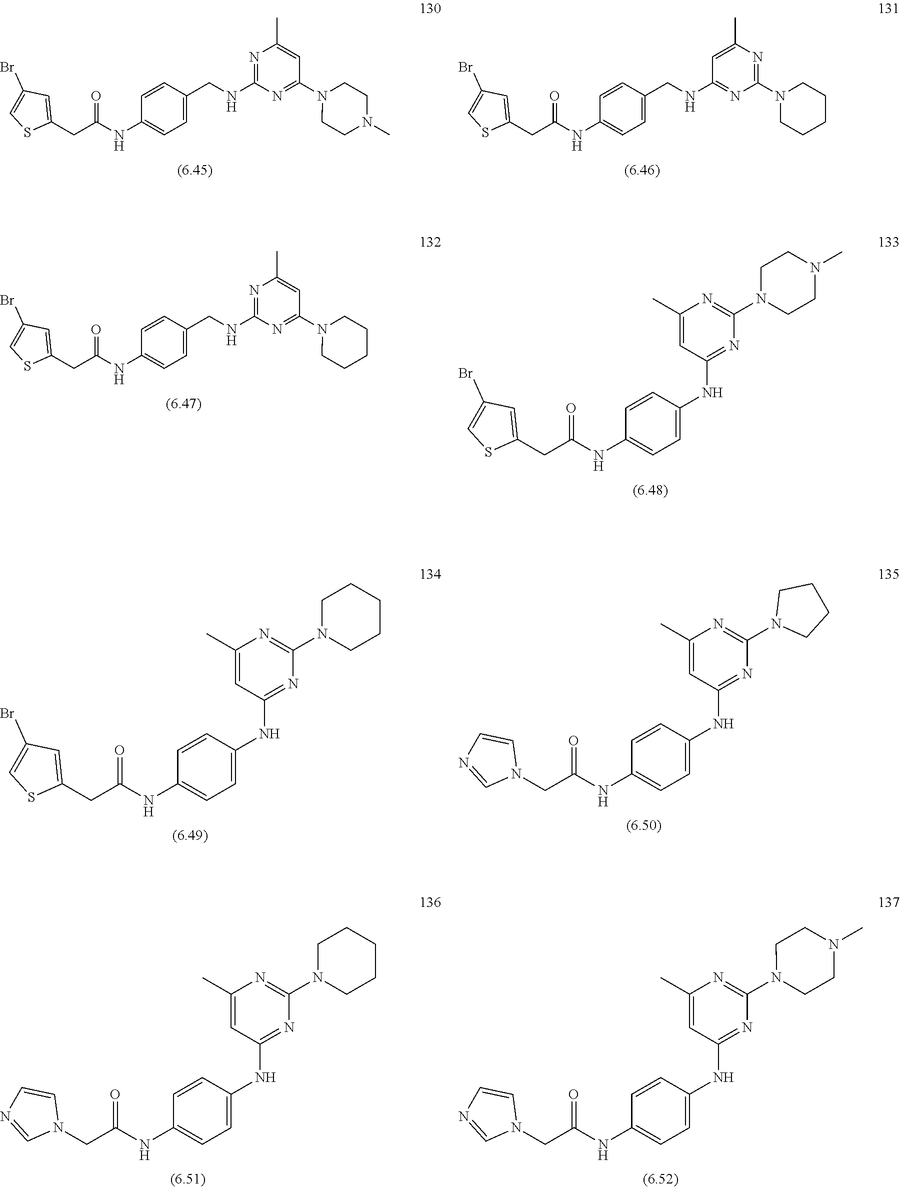

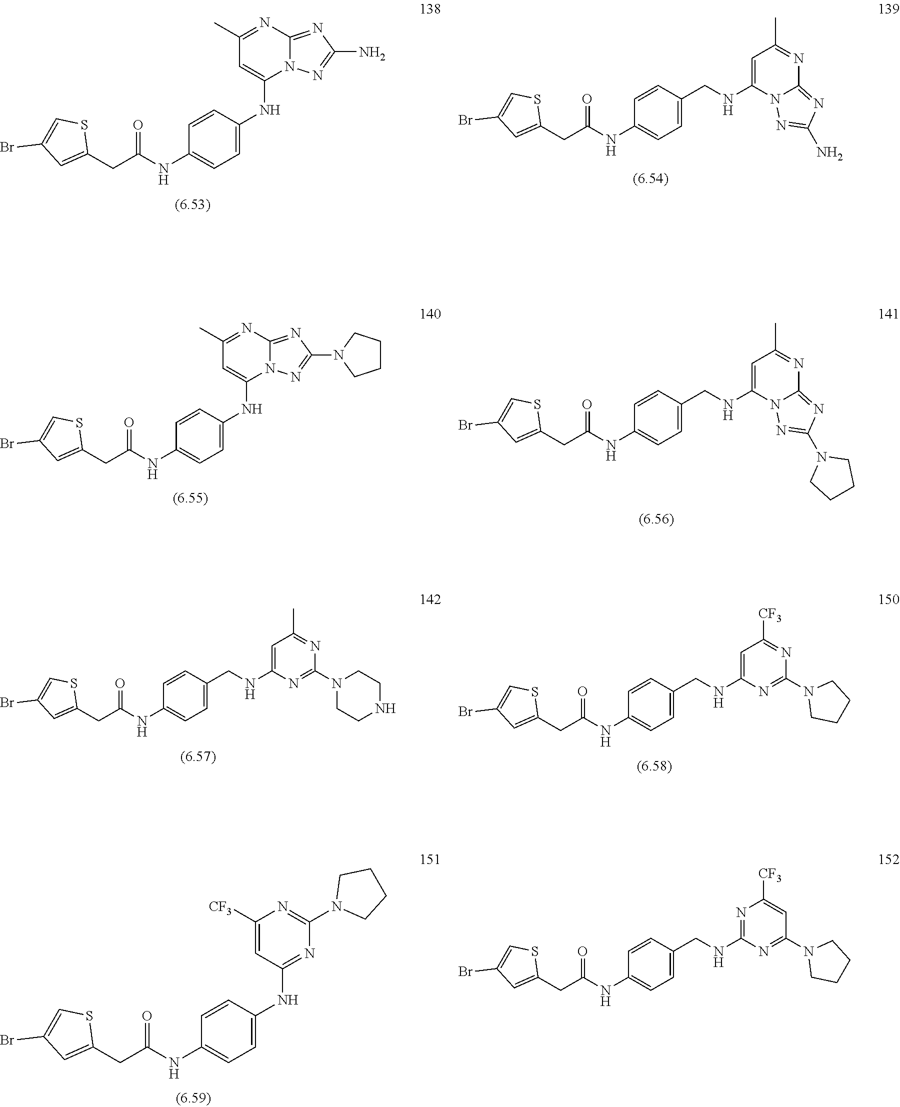

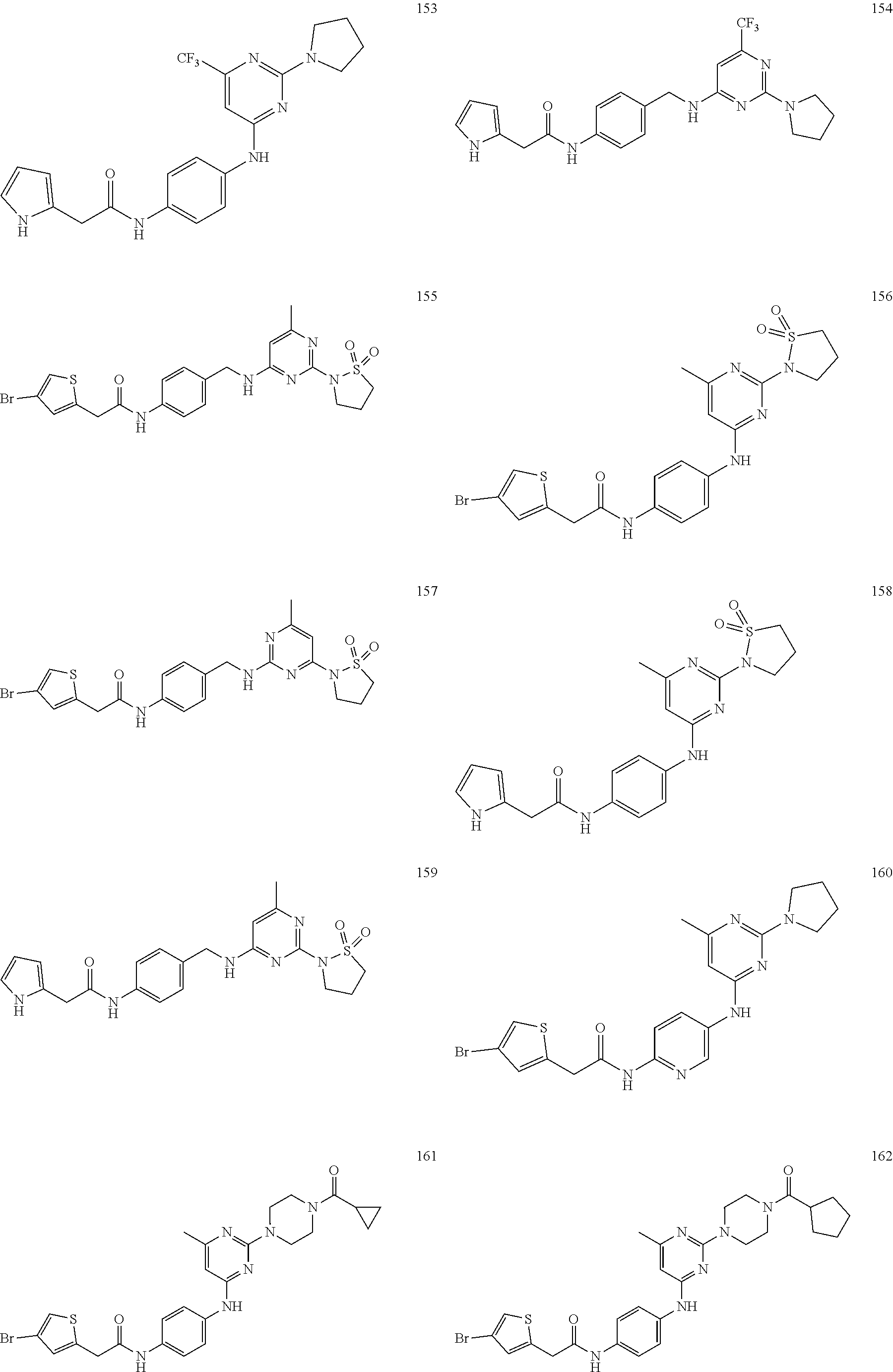

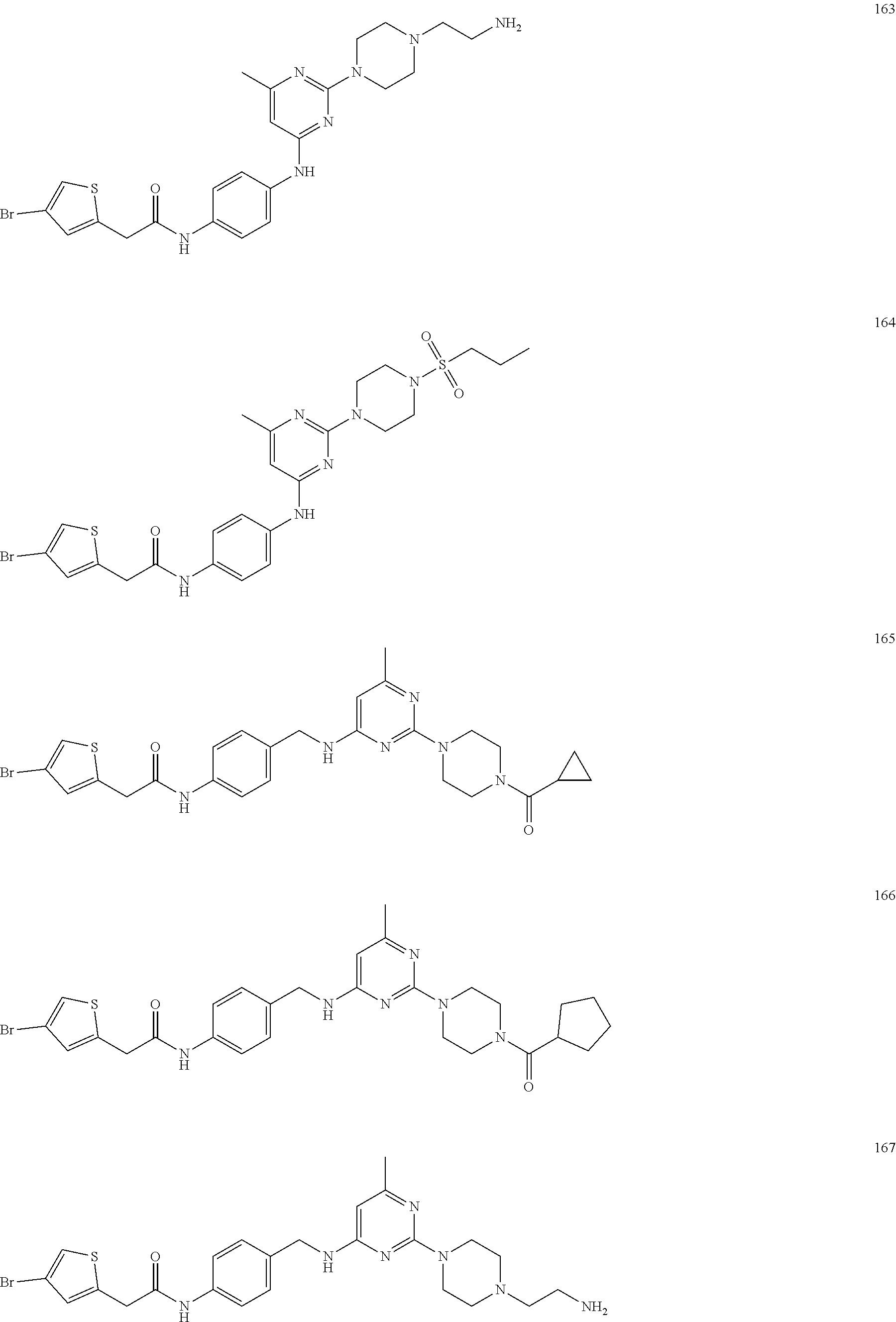 [SMILES: Cc1cc(Nc2ccc(NC(=O)Cc3cc(Br)cs3)cc2)nc(N2CCN(CCN)CC2)n1 ; CCCS(=O)(=O)N1CCN(c2nc(C)cc(Nc3ccc(NC(=O)Cc4cc(Br)cs4)cc3)n2)CC1 ; Cc1cc(NCc2ccc(NC(=O)Cc3cc(Br)cs3)cc2)nc(N2CCN(C(=O)C3CC3)CC2)n1 ; Cc1cc(NCc2ccc(NC(=O)Cc3cc(Br)cs3)cc2)nc(N2CCN(C(=O)C3CCCC3)CC2)n1 ; Cc1cc(NCc2ccc(NC(=O)Cc3cc(Br)cs3)cc2)nc(N2CCN(CCN)CC2)n1]

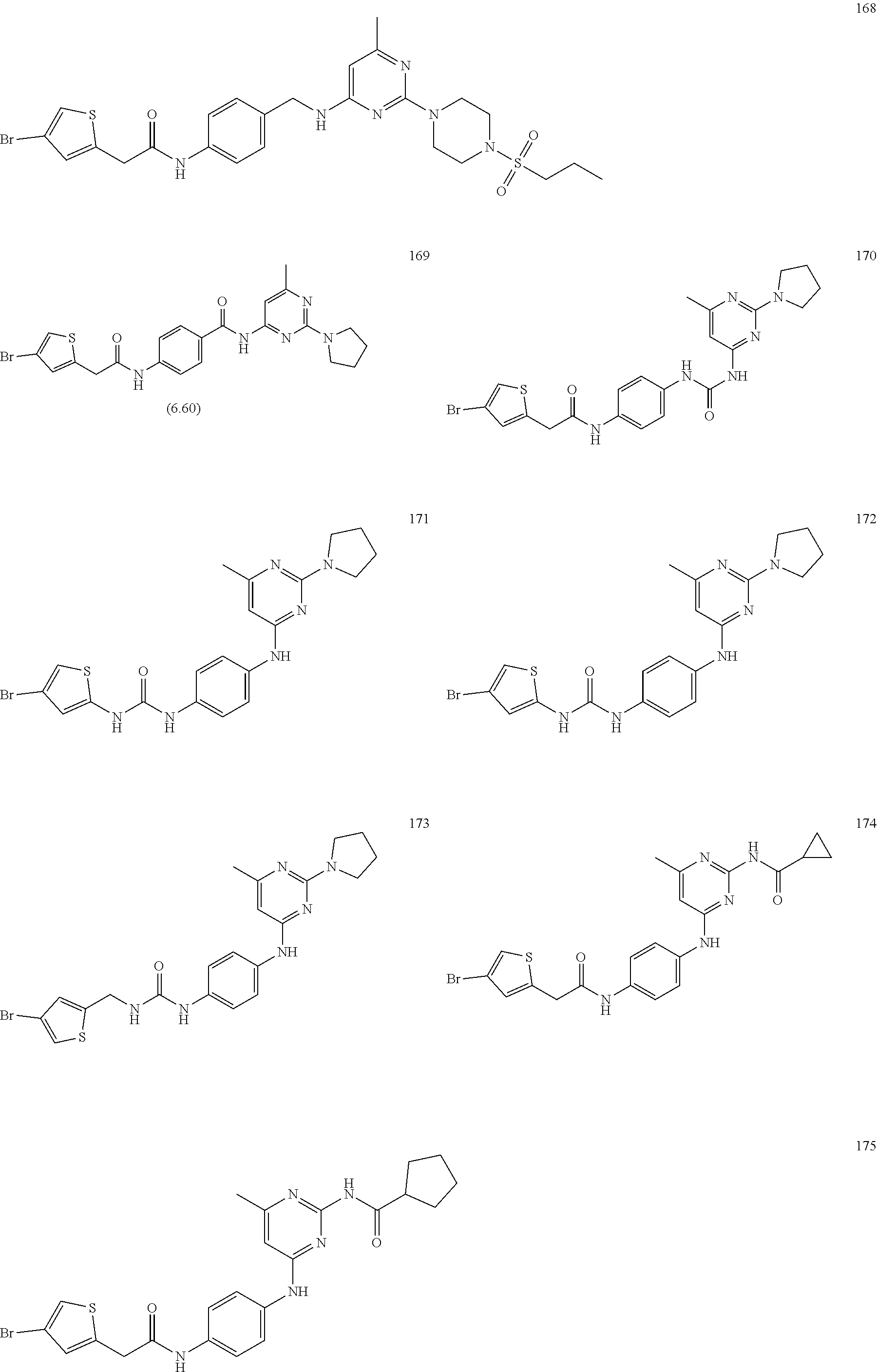

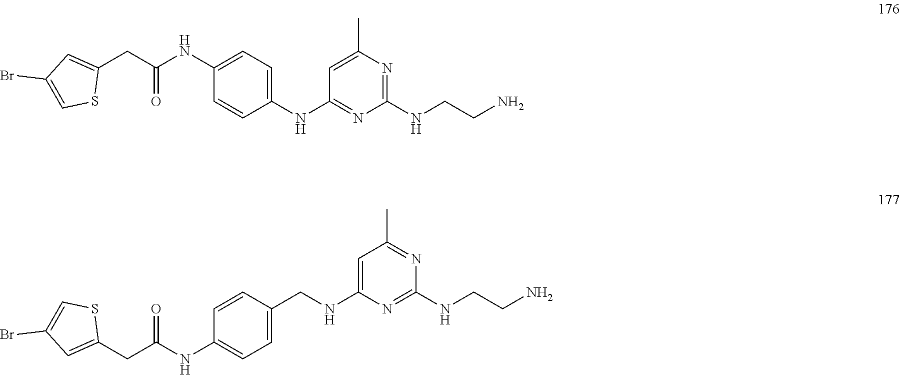 [SMILES: Cc1cc(Nc2ccc(NC(=O)Cc3cc(Br)cs3)cc2)nc(NCCN)n1 ; Cc1cc(NCc2ccc(NC(=O)Cc3cc(Br)cs3)cc2)nc(NCCN)n1]

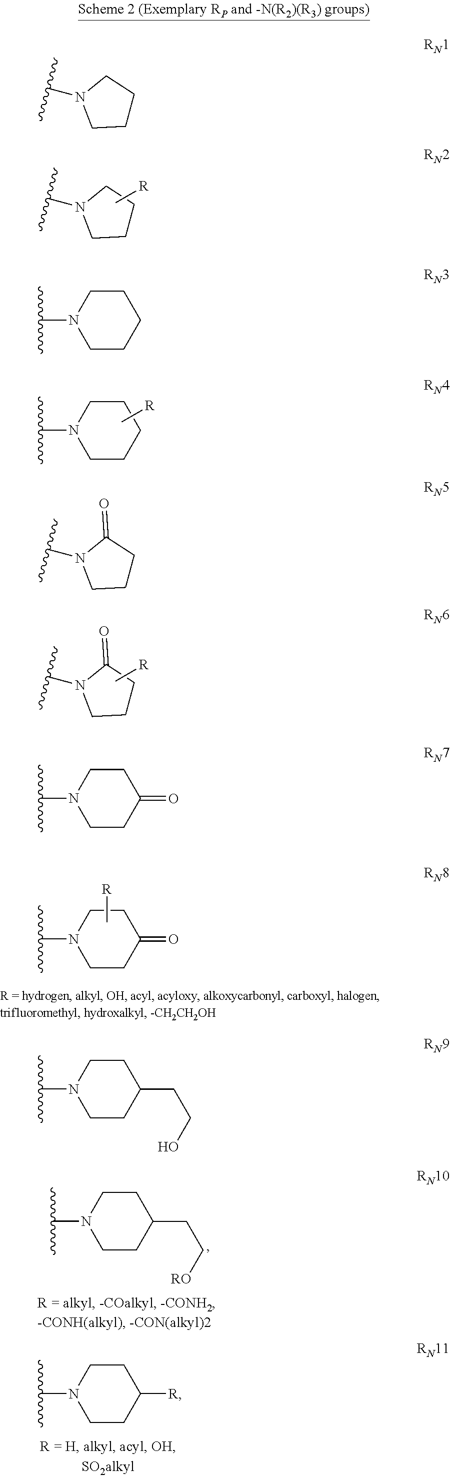

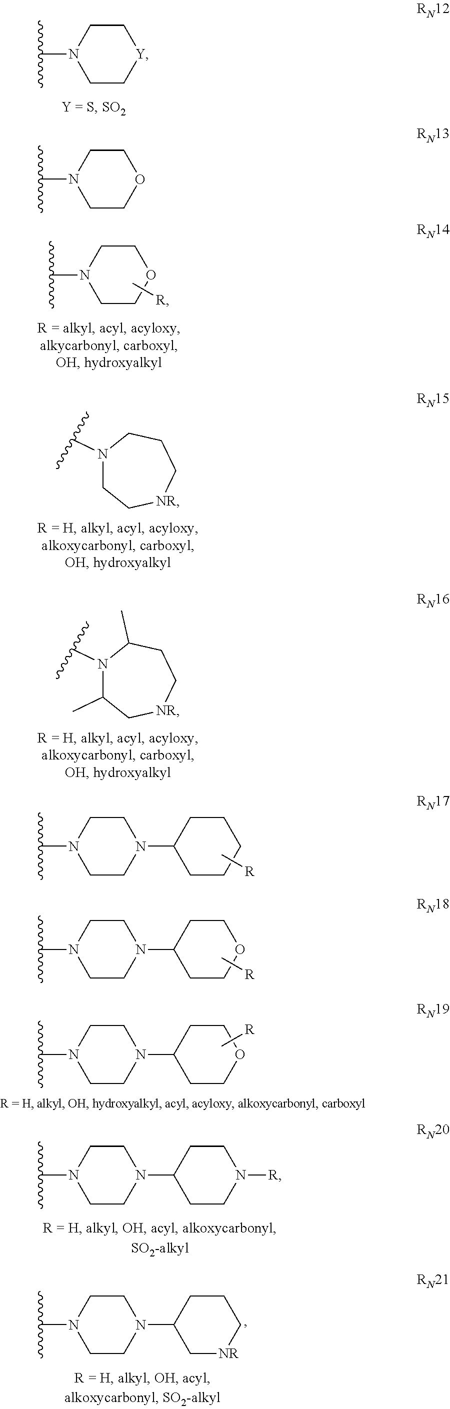

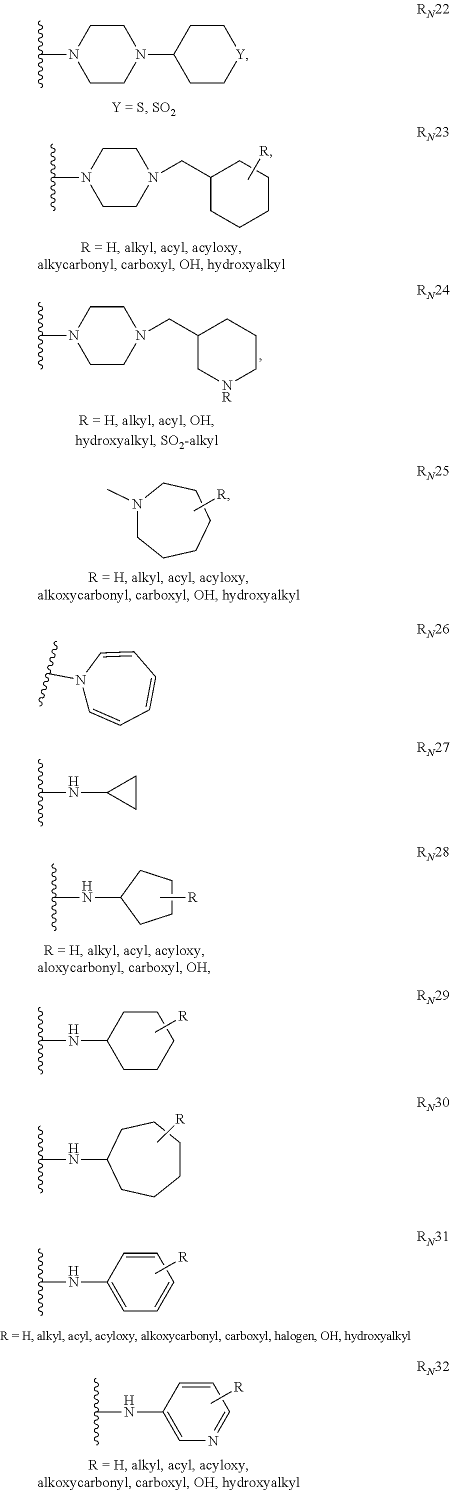

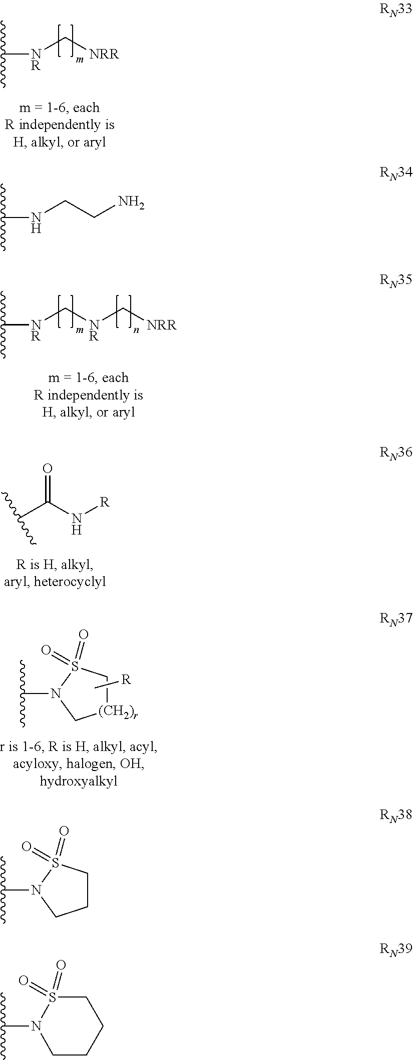

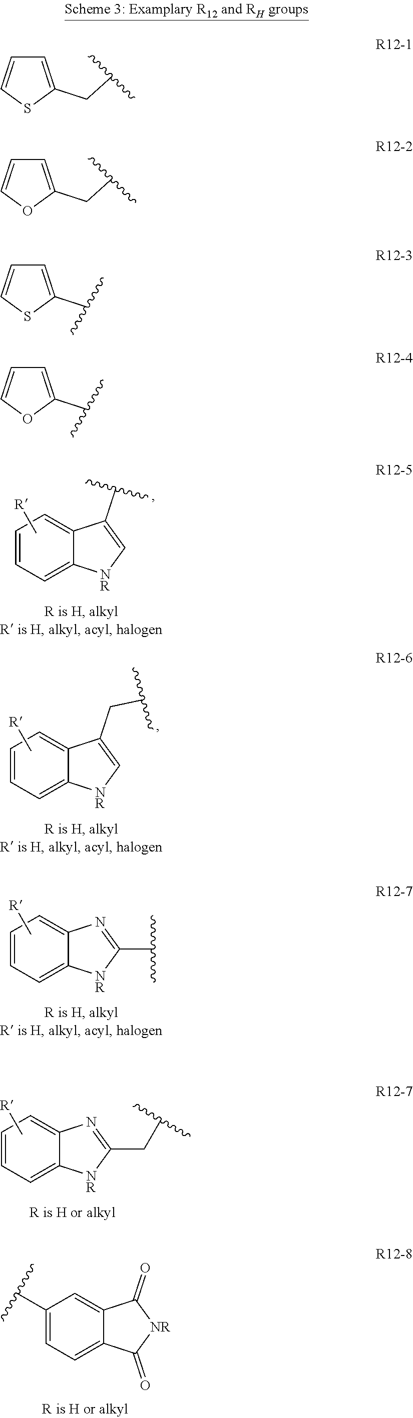

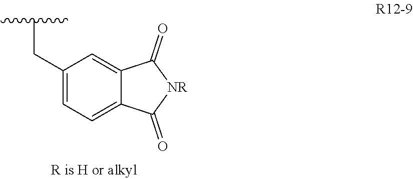

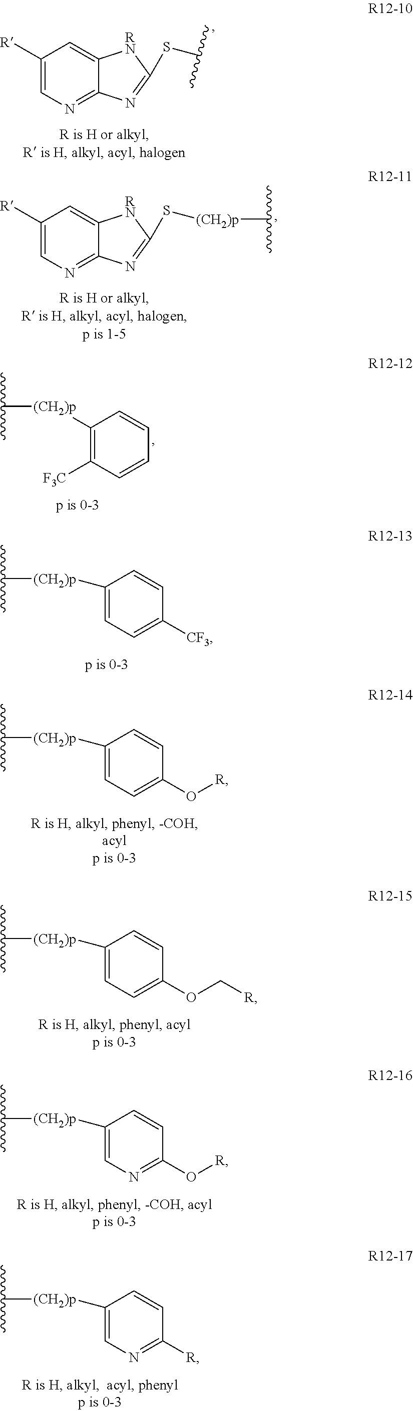

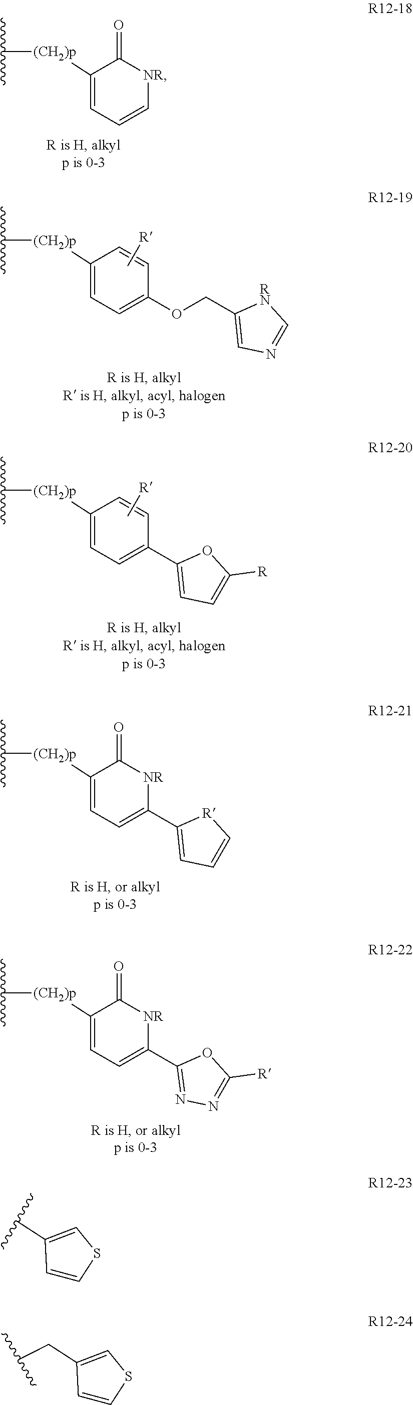

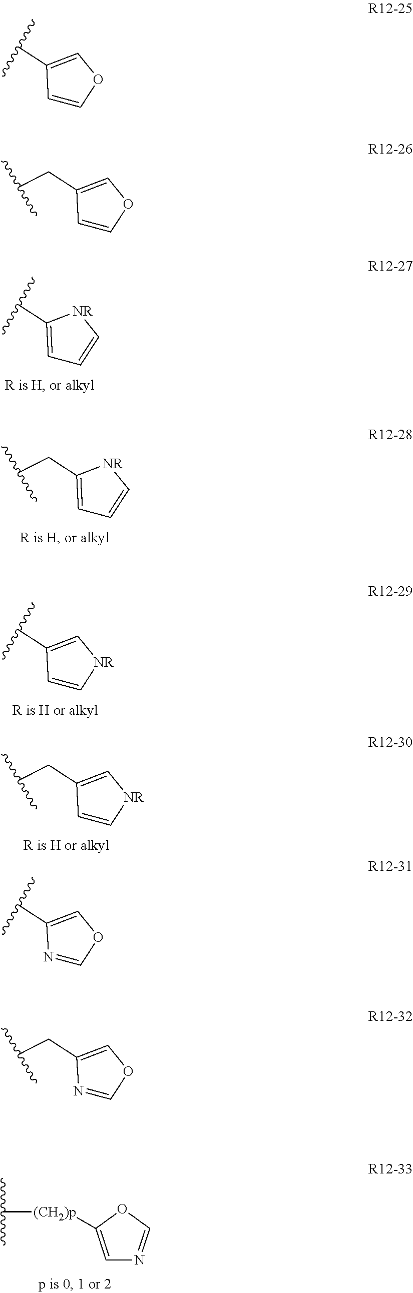

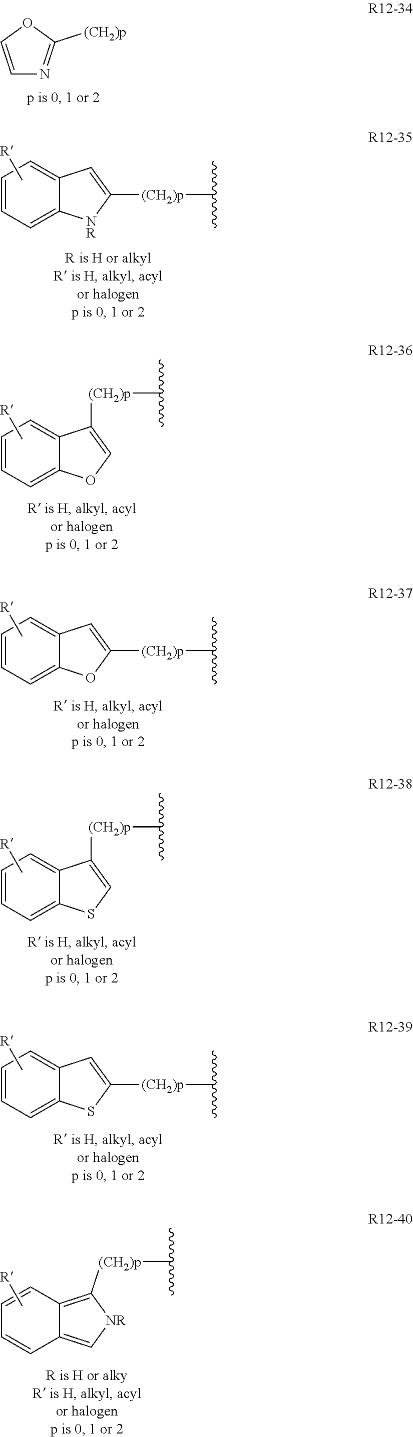

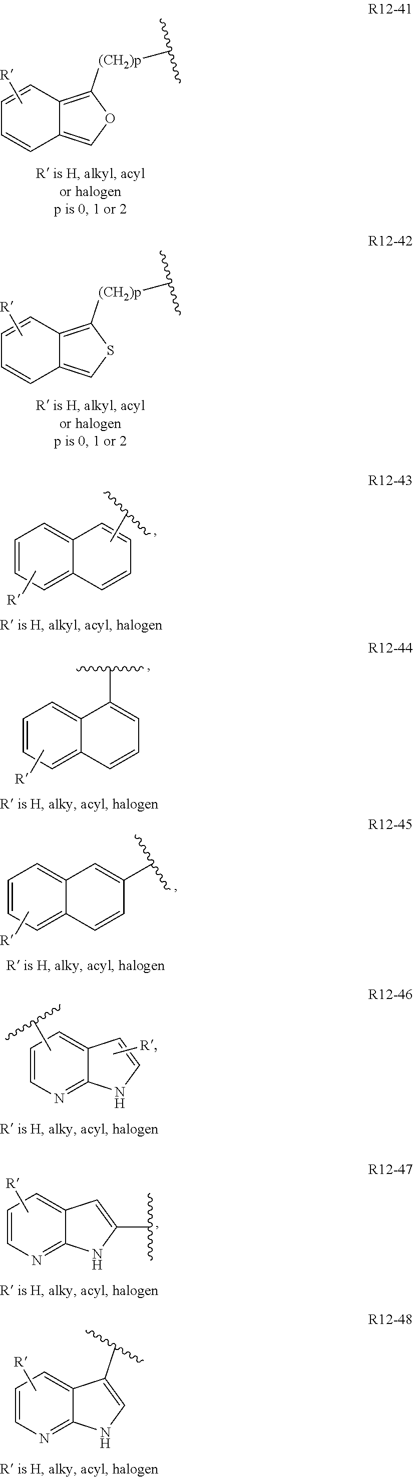

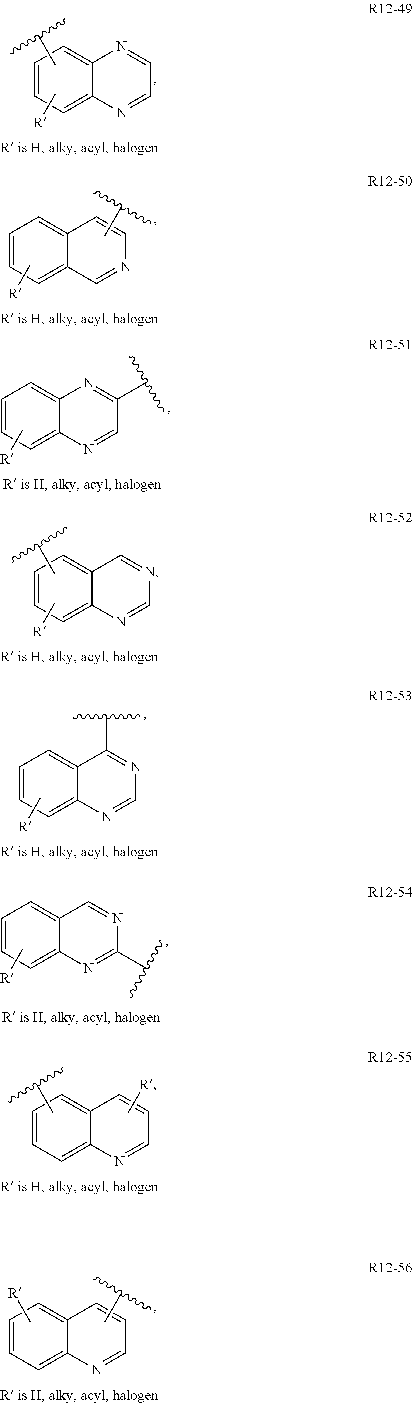

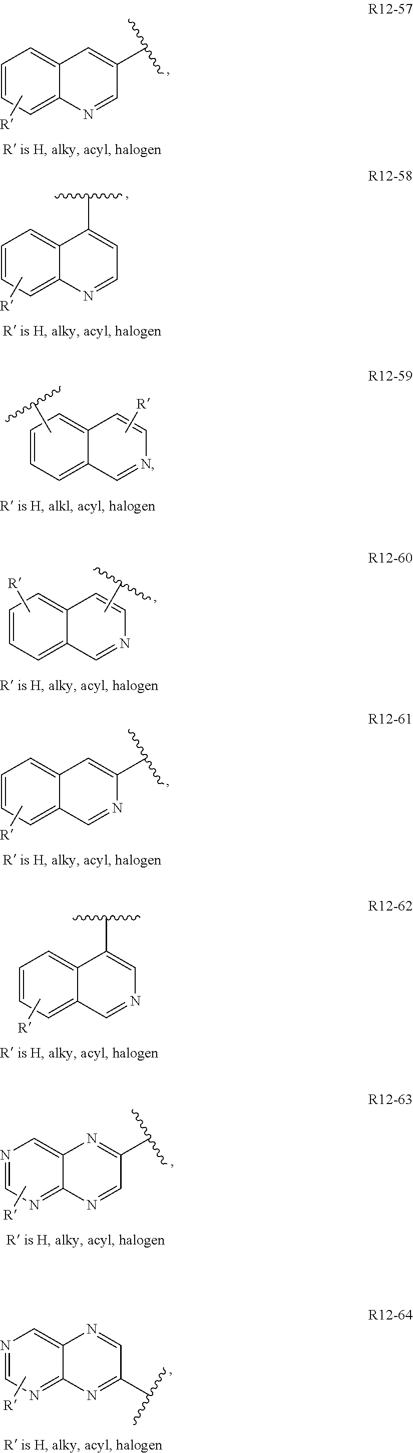

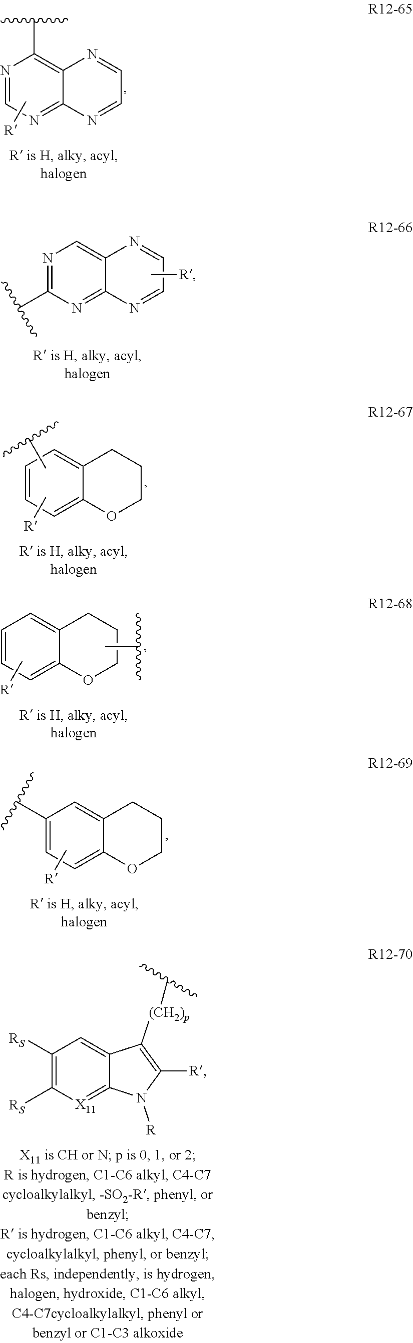

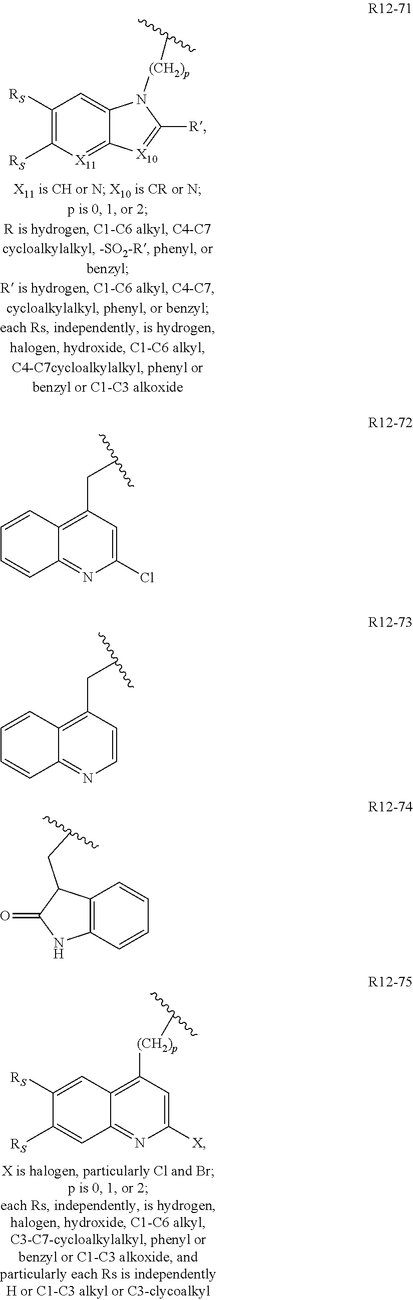

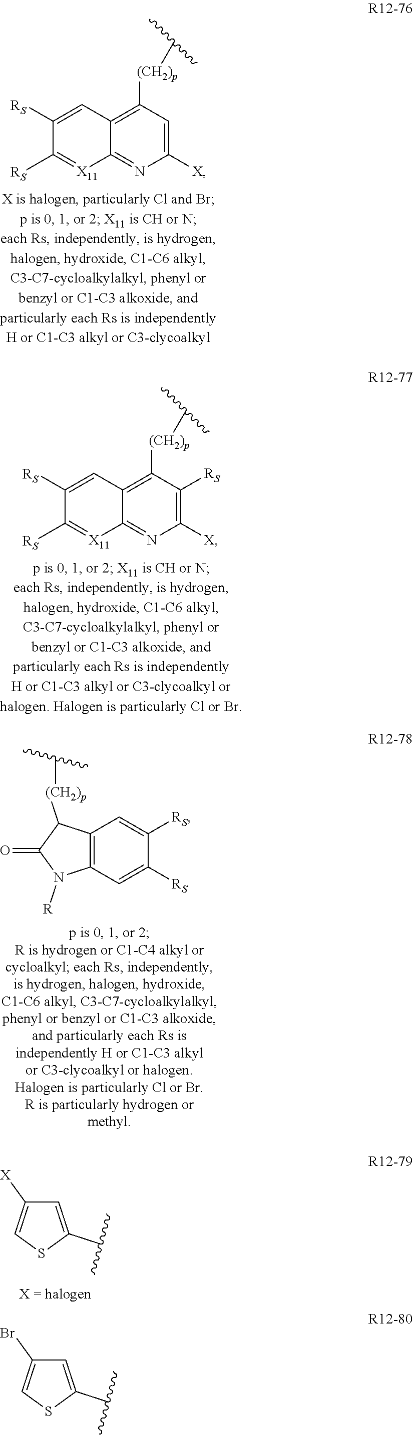

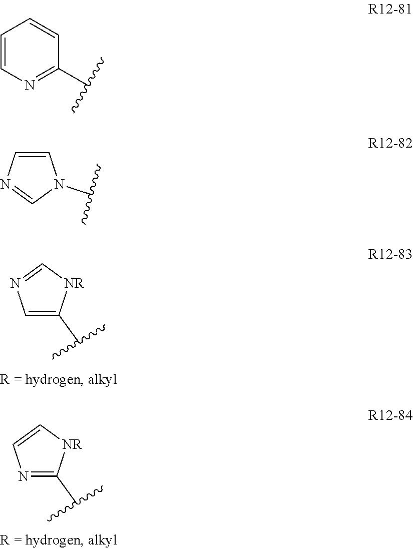

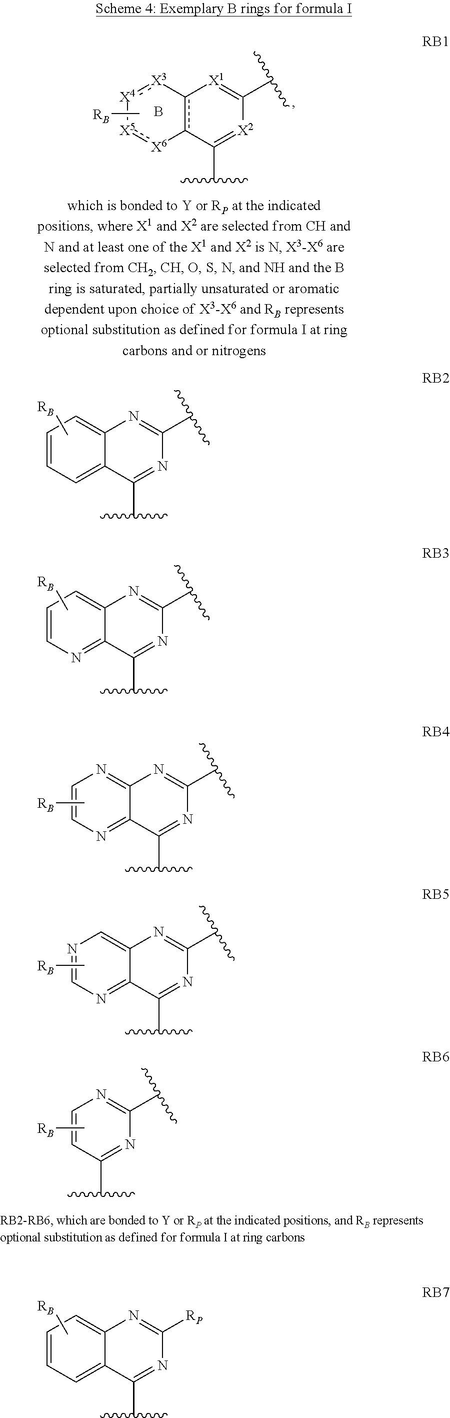

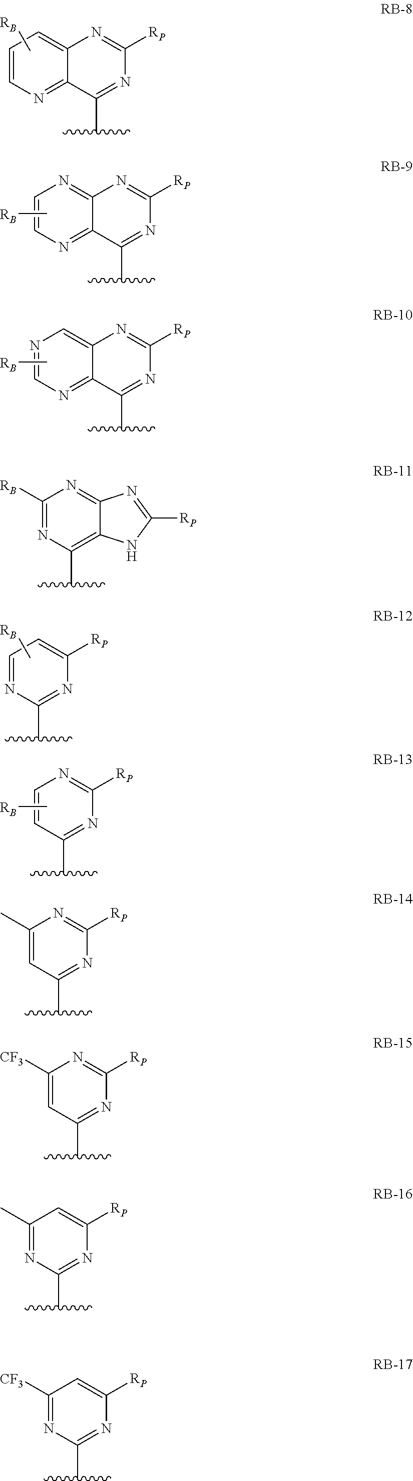

RB7-RB17, which are bonded to Y at the indicated position, and R_{B} represents optional substitution as defined for formula I at ring carbons or at specifically indicated carbons

### Example 12: Exemplary Synthetic Methods

Compounds of Formula XX-XXIII as well as many other compounds of this invention are prepared, for example, by the method illustrated in Scheme 5 where variables are as defined above. This three-step synthesis starts with selective aromatic nucleophilic substitution on the 4-position of a 2,4-dichloro-pyrimidine A (e.g., 2,4-dichloro-6-methylpyrimidine, where R₄ is methyl or 2,4-dichloro-5-fluoropyrimidine, where R₅ is fluorine) with a p-phenylenediamine B to form the intermediate C. Exemplary reaction conditions are shown in Scheme 5 where reactants are added with trimethylamine to ice cold ethanol and stirred at rt for 15 h. [Kumar et al., 2014; Odingo et al., 2014]. Chlorinated intermediate C is then reacted with any amine HNR₂R₃ D by amination to generate intermediate E. Exemplary amination conditions are shown in Scheme 5, where reactants are reacted in DMF in the presence of K₂CO₃ at elevated temperature. Step three couples the R₁₀ group employing acid F to intermediate E. Various known synthetic methods can be employed to introduce a selected R₁₀ group, for example, cross coupling, click chemistry or substation reactions (e.g., SN2, aromatic, electrophilic) [Li et al., 2014a; Li et al., 2014b; LaBarbera et al., 2007]. Scheme 5 illustrates coupling of the amine group of E with a selected carboxylic acid F to form R₁₀ which is —NH—CO—R₁₂ in compound G. Exemplary R₁₂ are aryl, aryl-substituted alkyl, heteroaryl and heteroaryl-substituted alkyl. Exemplary coupling conditions are illustrated in Scheme 5, where coupling proceeds in the presence of propylphosphonic anhydride (T3P) and triethyamine at room temperature to form the desired compound G. The illustrated method has been employed, for example to prepare compound 6, and compound 8 (see, Scheme 6).

Various substituted starting materials A, B, D and F are commercially available or can be prepared using known methods. In embodiments, aniline derivatives already substituted with R₁₀ (B′) can be used in place of p-phenylenediamine derivatives B to form a corresponding R₁₀-substituted intermediate C′. Carrying out step 2 of the illustrated reaction, by reacting intermediate C′ with D will result in desired corresponding compound G′ (where R₁₀ replaces R₁₂—CO—NH—). As will be appreciated by one of ordinary skill in the art, it may be useful or necessary to protect certain groups in the starting materials or intermediates during reactions shown to prevent undesired side-reactions. For example, ring N in reactants F may be protected with appropriate amine protecting groups. Use of appropriate protecting groups is generally routine in the art. A variety of primary or secondary amines (D) are commercially available or can be prepared by well-known methods. Alternatively, chlorinated intermediate C can be reacted with an appropriate nucleophile to add a selected —NR₂R₃ group at the 4-chloro position. For example, D can be a cyclic amine such as pyrrolidine. As another possible alternative, Suzuki coupling may be used to install an amine containing group by C—C bond formation [Li et al., 2014a]. As another possible alternative, Buchwald-Hartwig cross coupling can be used to form carbon and amine bonds in such intermediates.

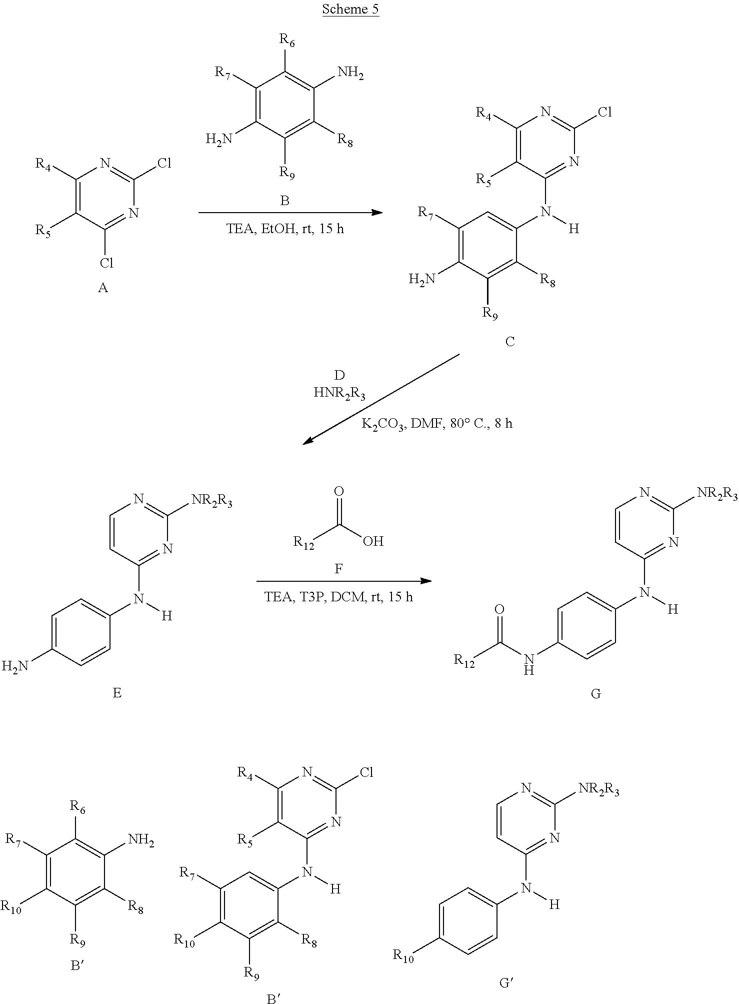

Detailed Synthesis of Compounds 6 and 8 (Scheme 6)

N-(4-aminophenyl)-2-chloro-6-methyl-pyrimidin-4-amine (102). To 0.5 g (3.06 mmol) of 2,4-dichloro-6-methylpyrmidine (100) dissolved in 10 mL of ethanol at 0° C. were added 513.7 μL (1.2 equivalents, 372.5 mg, 3.68 mmol) of triethyl amine (TEA), and 330.5 mg (3.06 mmol) of p-phenylenediamine (101). The reaction mixture was warmed to room temperature and stirred at that temperature overnight. The solvents were removed in vacuo and the resulting residue was chromatographed on silica gel using 40% hexane in ethyl acetate as the eluent to afford 500 mg (70% yield) of the pure product 102). ¹H NMR: (400 MHz, CDCl₃): δ 7.19 (broad s, 1H), 7.02 (d, J=8.8 Hz, 2H), 6.70 (d, J=8.8 Hz, 2H), 6.18 (s, 1H), 3.77 (broad s, 2H), 2.26 (s, 3H).

N-(4-aminophenyl)-6-methyl-2-(pyrolidin-1-yl)pyrimidin-4-amine (104). To 1.2 g of N-(4-aminophenyl)-2-chloro-6-methyl-pyrimidin-4-amine (102) dissolved in 120 mL of DMF were added 777.3 mg (5.62 mmol) of potassium carbonate and 3.63 mg (4.12 mL, 51.1 mmol) of pyrrolidine (103) at room temperature. The reaction mixture was heated 80° C. for 8 h. The reaction was cooled to room temperature and diluted with water. The product was extracted with ethyl acetate (3×100 mL). The organic layers were combined and washed with brine, followed by drying over Na₂SO₄, filtered and concentrated to give an oily crude product that was chromatographed on silica gel using 10% methanol in DCM (with drops of TEA) to give 1.31 g (96% yield) of the pure product (104). ¹H NMR: (400 MHz, CDCl3): δ 7.11 (d, J=8.4 Hz, 2H), 6.67 (d, J=8.4 Hz, 2H), 6.25 (broad s, 1H), 5.68 (s, 1H), 3.63 (broad s, 2H), 3.56 (t, J=6.8 Hz, 4H), 2.19 (s, 3H), 1.93 (t, J=6.8 Hz, 4H).

N-(4-((6-methyl-2-(pyrrolidin-1-yl)pyrimidin-4-yl)amino)phenyl)-2-(thiophen-2-yl)acetamide (6). To 700 mg (2.60 mmol) of N-(4-aminophenyl)-6-methyl-2-(pyrolidin-1-yl)pyrimidin-4-amine (104) in 15 mL were added 406 mg (2.86 mmol) of 2-thiopheneacetic acid (105), 906.8 μL (657.5 mg, 6.50 mmol) of TEA, and 3.06 mL (1.65 mg, 5.20 mmol) of T3P (50% weight solution in ethyl acetate) at 0° C. The mixture was warmed to room temperature and stirred for 15 h. The reaction was quenched by gradual addition of water, and the product was extracted with DCM (3×150 mL), followed by washing with brine. The organic layers were combined, dried over Na₂SO₄, filtered and concentrated under reduced pressure to give a crude product that was purified using silica gel and 10% methanol in DCM to give 864.5 mg (84% yield) of the pure product (6). ¹H NMR: (400 MHz, DMSO-d6): δ 10.07 (s, 1H), 9.06 (broad s, 1H), 7.64 (d, J=8.8 Hz, 2H), 7.49 (d, J=9.2 Hz, 2H), 7.39-7.37 (m, 1H), 6.98-6.96 (m, 2H), 3.84 (s, 2H), 3.47 (s, 4H), 2.13 (s, 3H), 1.89 (t, J=6.6 Hz, 4H). HPLC: 98% pure.

Boc-protected indole-3-caboxylic acid 106-boc was used in a peptide coupling methodology with compound 104 in the presence of T3P and TEA to achieve the synthesis of boc-protected indole derivative 8-boc, which was converted to the indole derivative 8 in good yield via TFA deprotection of the boc-protecting group. Note that the boc protecting group is —COO-t-butylK2Co3,KI, EtOH

N-(4-{[6-Methyl-2-(1-pyrrolidinyl)-4-pyrimidinyl]amino}phenyl)-1-{[(2-Methyl-2-propanyl)oxy]carbonyl}-1H-indole-3-carboxamide (8-boc). To 80 mg (0.297 mmol) of compound 104 in 8 mL DCM, were added 85.36 mg (0.3267 mmol of boc-protected indole-3-carboxylic acid (106-boc), 103.6 μL (75.13 mg, 0.742 mmol) of TEA, 350 μL (189 mg, 0.594 mmol) of T3P at 0° C. The mixture was warmed to room temperature and stirred for 20 h. The reaction was quenched by gradual addition of water, and the product was extracted with DCM (3×50 mL), followed by washing with brine. The organic layers were combined, dried over Na₂SO₄, filtered and concentrated under reduced pressure to give a crude product that was purified using silica gel and 10% methanol in DCM to give 76 mg (50% yield) of the pure product (8-boc). ¹H NMR: (400 MHz, CDCl₃): δ 8.28 (broad s, 1H), 8.24-8.13 (m, 3H), 7.57 (q, J=4.8, 8.8 Hz, 4H), 7.40-7.31 m, 3H), 5.92 (s, 1H), 3.54 (s, 4H), 2.23 (s, 3H), 1.86 (s, 4H), 1.66 (s, 9H).

N-(4-{[6-Methyl-2-(1-pyrroliidnyl)-4-pyrimidinyl]amino}phenyl)-1H-indole-3-carboxamide (8). 70 mg (0.136 mmol) of N-(4-{[6-Methyl-2-(1-pyrrolidinyl)-4-pyrimidinyl]amino}phenyl)-1H-indole-3-carboxamide (8-boc) was dissolved in 25% TFA in DCM (5 mL). The solution was stirred for 3 h at room temperature. The solvents were removed under reduced pressure and the crude product was purified using silica gel and 10% methanol in DCM to give 43.78 mg (78% yield) of the pure product. ¹H NMR: (400 MHz, DMSO-d6): δ 11.7 (s, 1H), 9.65 (s, 1H), 9.09 (s, 1H), 8.28 (broad s, 1H), 8.20 (d, J=7.6 Hz, 1H), 7.67 (s, 4H), 7.47 (d, J=8.0 Hz, 1H), 7.20-7.12 (m, 2H), 5.88 (s, 1H), 3.50 (s, 4H), 2.14 (s, 3H), 1.91 (s, 4H).

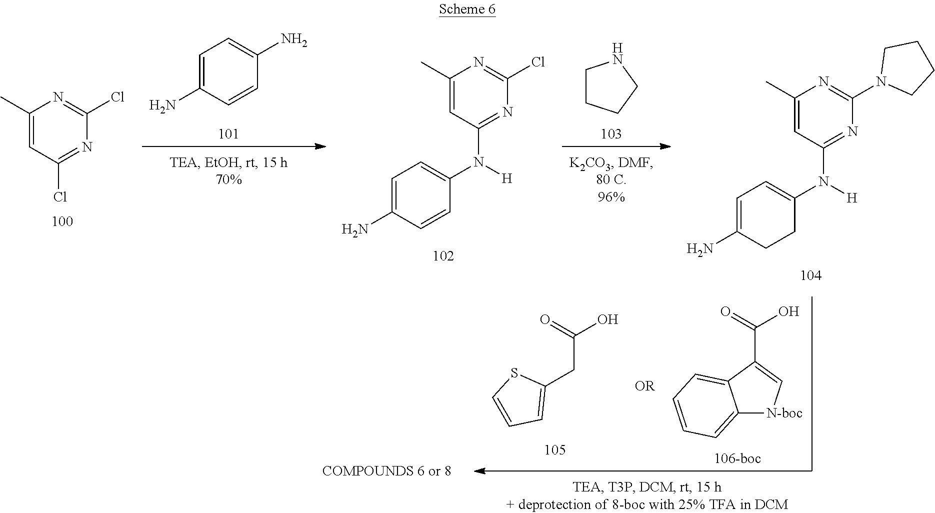

Scheme 7 illustrates an alternative method of synthesis optimized for yield of compound 6. In this method, a t-butyl protected carbamate, for example, compound 35 is reacted with a selected aromatic carboxylic acid, for example, compound 36 to form a protected carbamate intermediate, for example, compound 37. The intermediate is deprotected as known in the art, for example with trifluoroacetic acid (TFA) and the deprotected carbamate is reacted with a chlorinated heterocyclic group carrying a primary or secondary amine group (e.g., a pyrrolidinyl group), for example, compound 38 to form the desired compound of Formula XX, for example, compound 6. This method can also be employed to prepare various compounds of formula XX by selection of starting aromatic carboxylic acids and chlorinated heterocyclic compound carrying a primary of secondary amine group.

In Scheme 7, reagents employed for synthesis of compound 6 are shown, where in the first reaction DCC is N,N′-dicyclohexylcarbondiimide, DMAP is dimethylaminopyridine and the solvent is DCM dichloromethane. In the second reaction, after TFA deprotection, potassium carbonate, and potassium iodide in ethanol is employed. One of ordinary skill in the art can readily adapt the reagents and reaction conditions employed to prepare desired compounds of formula XX.

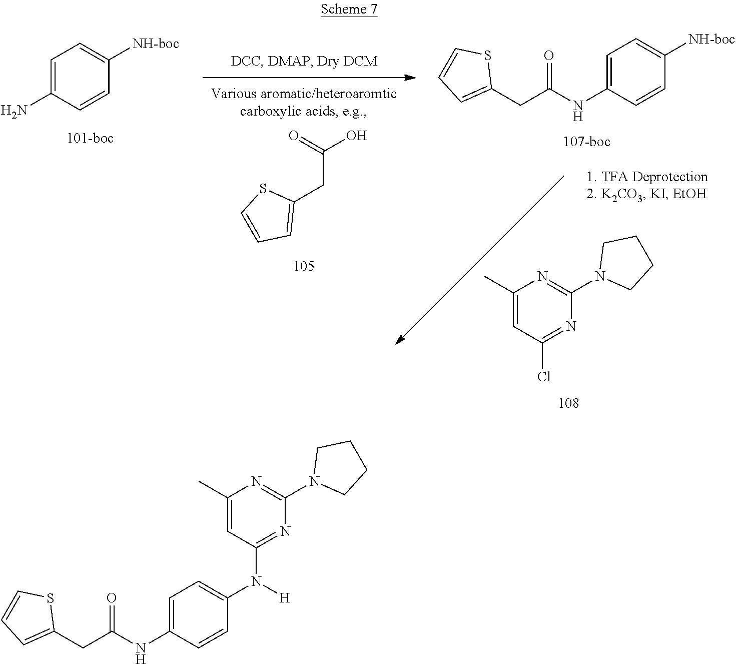

### Example 13: Biological Evaluation and Comparison of Inhibitors of Oncogenic CHD1L

CHD1L is unique from other chromatin remodelers and has a diverse repertoire of cellular functions. (Xiong et al., 2021) CHD1L is essential for PARP-mediated DNA repair and knockdown of CHD1L sensitizes tumor cells to DNA damaging agents. (Ahel et al., 2009) Two recent reports validate CHD1L as significant factor promoting drug resistance to PARP inhibitors via CHD1L mediated nucleosome sliding, alleviating PARP trapping. (Verma et al., 2021; Juhasz et al., 2020) Knockout of CHD1L is reported to sensitize BRCA1/2 mutant HR-deficient tumor cells to PARP inhibition causing cell death in vitro and loss of tumor growth with increased survival in vivo. (Verma et al., 2021; Juhasz et al., 2020)

In an aspect herein, we show that CHD1L is a required component of the TCF/LEF-transcription factor complex (denoted henceforth as TCF-transcription) (see also, Abbott et al., 2020), which is linked as a driver of GI cancers and many other cancers. (van de Wetering, et al., 2002; Ram Makena et al., 2019; Batlle et al., 2002; He et al., 2020; Polakis, 2012b; Clevers et al., 2006) We have determined this complex to be a master regulator of epithelial-mesenchymal transition (EMT) that promotes epithelial-mesenchymal plasticity (EMP). (Esquer et al., 2021; Zhou et al., 2016) Others have confirmed this. (Yang et al., 2020; Sánchez-Tilló et al., 2011; Kroger et al., 2019) In particular, we demonstrated the TCF-transcription is upregulated in isolated quasi-mesenchymal cell phenotypes compared to other EMT phenotypes, promoting increased cancer stem cell (CSC) stemness and invasiveness. (Esquer et al., 2021). Our work suggested that targeted small molecule inhibitors of CHD1L can provide an effective therapeutic strategy to treat CRC and other cancers.

Herein, we describe the high-throughput screen (HTS) drug discovery and hit-to-lead validation of the first-in-class CHD1L inhibitors (CHD1Li). (See also, Abbott et al., 2020) In this example, we provide additional description of the medicinal chemistry optimization of compound 6.0, its biological evaluation, and structure activity relationship (SAR) of certain the CHD1L inhibitors structurally related to compound 6.0. In addition, we demonstrate that analog 6.11 displays improved pharmacokinetics compared to 6.0, including oral bioavailability and in vivo antitumor efficacy against CRC HCT116 tumor xenografts.

In this example, we describe the synthesis of compound 6.0 and optimize the chemistry to efficiently prepare analogs for ligand-based drug design. The synthesis began employing commercially available starting material including p-phenylenediamine (1) and dichloropyrimidine analogs (2.1-2.3) (Scheme 8). Compounds 1 and 2 (Scheme 8) were reacted in the presence of triethylamine to obtain a selective nucleophilic aromatic substitution, providing intermediates 3.1-3.3 (Scheme 8) in good yield ranging from 70-83%. A second nucleophilic aromatic substitution with pyrrolidine afforded the core pyrimidine pharmacophore of 6.0. Next, using propanephosphonic acid anhydride (T3P), commercially available thiophene (5) (Scheme 8) was coupled to provide 6.0, 6.1, and 6.2 with yields ranging from 77-84%. Analog 6.1 was reacted with methyl iodide to provide analog 6.4. (Chemical structures of compounds 6.0-6.4, and 6.11-6.14 are found in Scheme 1.) This chemistry provided several CHD1L inhibitor analogs to investigate the structure activity relationship (SAR) around the pyrimidine ring. Initially, we also utilized this synthetic approach to modify the thiophene aromatic ring coupled as amides to the phenylenediamine ring.

The synthetic approach to produce 6.3 as shown in Scheme 8 gave low yields and difficulty in purification. Therefore, we further optimized the synthesis starting with a tert-butoxycarbonyl (BOC) protected phenylenediamine, which resulted in a significant increase in purity of the desired substitution at the 4-position of the pyrimidine ring, facilitating the pyrrolidine substitution in the 2-position. Unfortunately, after BOC deprotection the challenges persisted with peptide coupling of the 3-indole carboxylic acid to produce 6.3. However, increasing the carbon spacer between the aromatic rings and the carboxylic acid functionality allowed for efficient peptide coupling, leading to the optimized syntheses of CHD1Li (Schemes 9A and 9B). In Schemes 9A and 9B, we utilized BOC protecting groups to facilitate derivatization of the R₄ group therein with various aromatic groups, including thiophene, indole, azaindole, benzimidazole, and quinoline rings (Scheme 9A). In addition, we substituted pyrrolidine for morpholine amine rings in the R₃ group therein. Finally, to investigate the necessity of the aniline linkage of 6.0, we generated ether linked analogs of 6.0 (Scheme 9B). The methods of Scheme 9A and 9B produced analogs of 6.0, including among others 6.5-6.33 (see Scheme 1).

### Example 13: Synthetic Examples (Compound Number in the Following Paragraphs Refer to Schemes 8, 9A and 9B)

N-(4-aminophenyl)-2-chloro-6-methyl-pyrimidin-4-amine (3.1). To 0.5 g (3.06 mmol) of 2,4-dichloro-6-methylpyrmidine (2) dissolved in 10 mL of ethanol at 0° C. were added 513.7 μL (1.2 equivalents, 372.5 mg, 3.68 mmol) of triethyl amine, and 330.5 mg (3.06 mmol) of p-phenylenediamine (1). The reaction mixture was warm to room temperature and stirred overnight. The solvents were removed under reduced pressure and the resulting residue was chromatographed on silica gel using 40% hexane in ethyl acetate as the eluent to afford 500 mg (70% yield) of the 3.1. R_{f}=0.40; m.p. 157-159° C.; ¹H NMR (400 MHz, CDCl₃) δ 7.507 (s, N—H), 7.003-7.025 (d, J=8.6 Hz, 2H), 6.670-6.691 (d, J=8.6 Hz, 2H), 6.162 (s, 1H), 3.777 (s, N—H, 2H), 2.239 (s, 3H); ¹³C-NMR (100 MHz, CDCl₃); 168.384, 164.430, 160.118, 145.450, 127.560, 126.906, 115.827, 100.182, 23.936; IR (neat) vₘₐₓ 33214.14, 1590.07, 1506.88, 1424.41, 1214.54, 1028.66, 970.00, 905.78, 826.69, 757.43, 547.51, 510.10; ESI-H RMS [M+H]⁺ calculated for C₁₁H₁₁ClN₄, 234.07, found 235.0735.

N-(2-chloro-5-fluoro-6-methylpyrimidin-4-yl)benzene-1,4-diamine (3.2). 2,4-dichloro-5-fluoro-6-methylpyrimidine (2.2) (250 mg, 1.381 mmol, 1.0 equiv) was dissolved in ethanol (10 mL) and cooled in an ice bath. Triethyl amine (231 μL, 1.657 mmol, 1.2 equiv) and p-phenylenediamine (1) (324.1 mg, 1.381 mmol, 1.0 equiv) were added and the reaction was allowed to warm to RT and stir for 15h. The solvent was removed under reduced pressure and the crude mixture was purified via column chromatography using 60% ethyl acetate in Hexanes to provide 3.2 (290 mg, 83% yield) as a dark yellow solid. TLC (60% ethyl acetate in hexanes), R_{f}=0.40; m.p. 157-159° C.; ¹H NMR (400 MHz, CDCl₃) δ 7.313-7.335 (d, J=8.7 Hz, 2H), 6.746 (s, 1H), 6.674-6.695 (d, J=8.7 Hz, 2H), 3.667 (s, N—H, 2H), 2.360-2.376 (d, J=3.0 Hz, 3H); ¹³C-NMR (100 MHz, CDCl₃); 153.422, 151.036, 150.891, 150.742, 144.413, 143.984, 141.895, 128.176, 123.111, 115.638, 17.025; IR (neat) vₘₐₓ 3328.86, 1616.04, 1507.65, 1281.35, 829.94, 830.88, 624.12, 562.34, 511.93; ESI-HRMS [M+H]⁺ calculated for C₁₁H₁₃ClFN₄, 252.06, found 253.0640.

N-(2-chloro-5-fluoropyrimidin-4-yl)benzene-1,4-diamine (3.3). 2,4-dichloro-5-fluoropyrimidine (2.3) (500 mg, 2.995 mmol, 1.0 equiv) was dissolved in EtOH (20 mL) and cooled in an ice bath. Triethyl amine (501.57 μL, 3.593 mmol, 1.2 equiv) and p-phenylenediamine (1) (323.88 mg, 2.995 mmol, 1.0 equiv) were added and the reaction was allowed to warm to RT and stir for 8h. The solvent was removed under reduced pressure and the crude mixture was purified via column chromatography using 60% ethyl acetate in Hexanes to provide 3.3 (544 mg, 76% yield) as a tan solid. TLC (60% ethyl acetate in hexanes), R_{f}=0.36; m.p. 155-157° C.; ¹H NMR (400 MHz, CDCl₃) δ 7.981-7.988 (d, J=2.7 Hz, 1H), 7.340-7.361 (d, J=8.7 Hz, 2H), 6.801 (s, N—H), 6.692-6.714 (d, J=8.7 Hz, 2H), 3.691 (s, N—H, 2H); ¹³C-NMR (100 MHz, CDCl₃)154.642, 151.535, 151.433, 146.62, 144.223, 143.900, 140.533, 140.331, 127.753, 123.174, 115.620; IR (neat) vₘₐₓ 3014.43, 1627.78, 1580.69, 1506.69, 1323.90, 1235.19, 946.92, 816.77, 746.87, 690.44, 641.61, 592.67, 514.39, 430.10; ESI-HRMS [M+H]⁺ calculated for C₁₀H₈ClFN₄, 238.04, found 239.0484.

N-(6-methyl-2-(pyrrolidin-1-yl)pyrimidin-4-yl)benzene-1,4-diamine (4.1). 3.1 was dissolved in 5 mL of DCM and treated with 5 mL of TFA at 0° C., resulting in a red colored solution. The reaction was warmed to RT and allowed to stir for 3h. The reaction was concentrated and redissolved in 10% methanol and DCM, then washed with bicarb and water. The organic later was dried over sodium sulfate and concentrated, purified via column chromatography using 10% methanol in DCM to produce 4.1 (2.09 g, 67% over two steps) as an orange solid. TLC (5% methanol/dichloromethane), R_{f}=0.18; m.p. 190-192° C.; ¹H NMR (400 MHz, CDCl₃) δ 7.105-7.127 (d, J=8.6 Hz, 2H), 6.666-6.687 (d, J=8.6 Hz, 2H), 6.190 (s, N—H), 5.680 (s, 1H), 3.542-3.575 (m, 4H), 2.188 (s, 3H), 1.920-1.953 (m, 4H); ¹³C-NMR (100 MHz, CDCl₃) 192.975, 162.718, 160.872, 143.621, 130.275, 125.357, 115.779, 91.608, 46.674, 25.689, 24.546; IR (neat) vₘₐₓ 1755.53, 1658.71, 1612.71, 1548.12, 1504.15, 1403.49, 1251.05, 1138.16, 890.44, 696.38, 517.52; ESI-HRMS [M+H]⁺ calculated for C₁₅H₁₉N5, 269.13, found 270.1700.

N-(2-chloro-5-fluoro-6-methylpyrimidin-4-yl)benzene-1,4-diamine (4.2). 3.2 (260 mg, 1.029 mmol, 1.0 equiv) was dissolved in DMF (29 mL) and treated with potassium carbonate (156.4 mg, 1.132 mmol, 1.1 equiv) and pyrrolidine (422.5 μL, 5.145 mmol, 5.0 equiv). The reaction was heated to 80° C. for 8h then diluted with ethyl acetate and washed with water and a 5% lithium chloride solution. The organic layer was dried over sodium sulfate, concentrated under reduced pressure, then purified via column chromatography using 10% methanol in ethyl acetate, to provide 4.2 (243 mg, 82% yield) as a brown solid. TLC (10% methanol/ethyl acetate), R_{f}=0.57; m.p. 180-182° C.; ¹H NMR (400 MHz, CDCl₃) δ 7.456-7.478 (d, J=8.6 Hz, 2H), 6.663-6.685 (d, J=8.6 Hz, 2H), 6.436 (s, N—H), 3.497-3.530 (m, 4H), 2.267-2.274 (d, J=2.9 Hz, 3H), 1.914-1.947 (m, 4H); ¹³C-NMR (100 MHz, CDCl₃)156.073, 149.566, 149.460, 149.193, 149.062, 142.081, 139.428, 139.056, 130.876, 121.623, 115.600, 47.019, 25.812, 17.361; IR (neat) vₘₐₓ 3185.05, 1600.39, 1506.17, 1444.25, 1238.75, 826.91, 762.06, 509.83; ESI-HRMS [M+H]⁺ calculated for C₁₅H₁₈FN₅, 287.15, found 288.1606.

N-(5-fluoro-2-(pyrrolidin-1-yl)pyrimidin-4-yl)benzene-1,4-diamine (4.3). 3.3 (310 mg, 1.30 mmol, 1.0 equiv) was dissolved in DMF (36 mL) and treated with potassium carbonate (197.6 mg, 1.43 mmol, 1.1 equiv) and pyrrolidine (533.8 μL, 6.5 mmol, 5.0 equiv). The reaction was heated to 80° C. for 8h then diluted with ethyl acetate and washed with water and brine. The organic layer was dried over Na₂SO₄ and concentrated under reduced pressure to provide 4.3 as a dark yellow solid, which was carried on crude. TLC (10% methanol/dichloromethane), R_{f}=0.59; m.p. 179-181° C.; ¹H NMR (400 MHz, CDCl₃) δ 7.846-7.855 (d, J=3.7 Hz, 1H), 7.458-7.479 (d, J=8.7 Hz, 2H), 6.671-6.693 (d, J=8.7 Hz, 2H), 6.494 (s, N—H), 3.497-3.530 (m, 4H), 1.934-1.967 (m, 4H); ¹³C-NMR (100 MHz, CDCl₃)156.889, 149.984, 149.886, 142.406, 141.214, 139.910, 139.717, 138.808, 130.346, 122.189, 121.827, 115.572, 47.026, 37.755, 25.808; IR (neat) vₘₐₓ 3388.87, 1598.56, 1568.68, 1500.63, 1447.20, 1227.27, 930.93, 831.66, 763.87, 496.87; ESI-HRMS [M+H]⁺ calculated for C₁₄H₁₆FN₅, 273.14, found 274.1450.

N-(4-((6-methyl-2-(1-pyrrolidinyl)-4-pyrimidinyl)amino)phenyl)-2-(2-thienyl)acetamide (6.0) 4.1 (262.0 mg, 0.973 mmol, 1.0 equiv) was dissolved in DCM (40 mL, anhydrous) then treated with 5.1 (145.3 mg, 1.02 mmol, 1.05 equiv), DMAP (118.9 mg, 0.973 mmol, 1.0 equiv), and then DCC (251 mg, 1.22 mmol, 1.25 equiv) under nitrogen. The reaction was allowed to stir for 8h then the material was concentrated onto silica gel and purified via column chromatography using 1:1 ethyl acetate:dichloromethane and 3% methanol to provide compound 6.0 (302.8 mg, 79% yield) as a yellow solid). TLC (10% methanol/ethyl acetate), R_{f}=0.49; m.p. 186-188° C.; ¹H NMR (400 MHz, CDCl₃) δ 7.611 (s, N—H), 7.389 (s, 4H), 7.273-7.289 (dd, 1H), 7.012-7.032 (m, 2H), 6.600 (s, N—H), 5.762 (s, 1H), 3.919 (s, 2H), 3.537-3.570 (m, 4H), 2.204 (s, 3H), 1.904-1.938 (s, 4H); ¹³C-NMR (100 MHz, CDCl₃); 167.993, 166.584, 161.249, 160.656, 136.446, 135.883, 132.826, 127.768, 127.625, 126.049, 121.643, 120.996, 92.885, 46.727, 38.504, 25.626, 24.380; IR (neat) vₘₐₓ 2862.14, 1572.75, 1500.37, 1398.29, 1330.32, 1226.59, 1169.02, 830.35, 782.81, 681.54, 513.32; ESI-HRMS [M+H]⁺ calculated for C₂₁H₂₃N₅OS, 393.16, found 394.1680.

N-(4-((5-fluoro-6-methyl-2-(pyrrolidin-1-yl)pyrimidin-4-yl)amino)phenyl)-2-(thiophen-2-yl)acetamide (6.1). 4.2 (450 mg, 1.566 mmol, 1.0 equiv) was dissolved in DCM (15.0 mL, anhydrous) and treated with 2-thiopheneacetic acid (5.1) (244.9 mg, 1.723 mmol, 1.1 equiv), and triethylamine (546.4 μL, 3.915 mmol, 2.5 equiv). The reaction was allowed to stir for 5 min. then propanephosphonic acid anhydride (1.85 mL, 3.132 mmol, 2.0 equiv) was added and the reaction was allowed to stir for 15h. The reaction was then quenched with ice water and extracted with DCM (×3). The organic layer was dried over sodium sulfate and concentrated under reduced pressure and purified via column chromatography using 10% methanol in ethyl acetate to provide 6.1 as a yellow solid (529 mg, 82% yield). TLC (10% methanol/ethyl acetate), R_{f}=0.42; m.p. 245-247° C.; ¹H NMR (400 MHz, DMSO-d⁶) δ 10.093 (s, N—H), 8.950 (s, N—H), 7.752-7.774 (d, J=8.9 Hz, 2H), 7.482-7.504 (d, J=8.9 Hz, 2H), 7.375-7.391 (dd, 1H), 6.963-6.982 (m, 2H) 3.844 (s, 2H), 3.391-3.423 (m, 4H), 2.183-2.190 (d, J=2.9 Hz, 3H), 1.860-1.892 (m, 4H); ¹³C-NMR (100 MHz, DMSO-d⁶); 167.579, 155.240, 148.710, 149.054, 138.597, 137.281, 136.205, 135.432, 133.499, 126.622, 126.211, 124.986, 120.444, 119.297, 46.517, 37.493, 25.128, 17.109; IR (neat) vₘₐₓ 3256.79, 1654.91, 1621.65, 1587.57, 1501.62, 1417.54, 1292.95, 1226.40, 826.57, 689.26, 548.39, 512.40; ESI-HRMS [M+H]⁺ calculated for C₂₁H₂₂FN₅OS, 411.5, found 412.1587.

N-(4-((5-fluoro-2-(pyrrolidin-1-yl)pyrimidin-4-yl)amino)phenyl)-2-(thiophen-2-yl)acetamide (6.2). 4.1 (50.0 mg, 0.183 mmol, 1.0 equiv) was dissolved in DCM (15.0 mL, anhydrous) and treated with 2-thiopheneacetic acid (5.1) (28.6 mg, 0.201 mmol, 1.1 equiv), and triethylamine (63.9 μL, 0.458 mmol, 2.5 equiv). The reaction was allowed to stir for 5 min., then propanephosphonic acid anhydride (0.194 mL, 0.366 mmol, 2.0 equiv) was added and the reaction was allowed to stir for 15h. The reaction was then quenched with ice water and extracted with DCM (×3). The organic layer was dried over sodium sulfate and concentrated under reduced pressure and purified via column chromatography using 10% methanol in ethyl acetate to provide 6.1 as a yellow solid (56 mg, 77% yield). TLC (10% methanol/ethyl acetate), R_{f}=0.61; m.p. 245-247° C.; ¹H NMR (400 MHz, CD₃OD) δ 10.112 (s, N—H), 9.110 (s, N—H), 7.950-7.960 (d, J=3.9 Hz, 1H), 7.766-7.789 (d, J=8.9 Hz, 2H), 7.499-7.522 (d, J=8.9 Hz, 2H), 7.377-7.393 (dd, 1H), 6.964-6.983 (m, 2H) 3.848 (s, 2H), 3.402-3.434 (m, 4H), 1.874-1.907 (m, 4H); ¹³C-NMR (100 MHz, CD₃OD); 167.617, 156.244, 149.151, 149.046, 140.693, 140.541, 140.350, 138.276, 137.262, 135.08, 133.735, 126.627, 126.223, 124.994, 120.588, 119.306, 46.568, 37.498, 25.115; IR (neat) vₘₐₓ 3256.79, 1654.91, 1621.65, 1587.57, 1501.62, 1417.54, 1292.95, 1226.40, 826.57, 689.26, 548.39, 512.40; ESI-HRMS [M+H]⁺ calculated for C₂₀H₂₀FN₅OS, 397.47, found 398.1431.

N-(4-((5-fluoro-6-methyl-2-(pyrrolidin-1-yl)pyrimidin-4-yl)(methyl)amino)phenyl)-2-(thiophen-2-yl)acetamide (6.3). 6.1 (13 mg, 0.0316 mmol, 1.0 equiv) was dissolved in THF (1.0 mL, anhydrous) and treated with sodium hydride (1.52 mg, 0.0379 mmol, 1.2 equiv) at 0° C. under nitrogen. The reaction was allowed to stir for 10 min. then iodomethane (3.0 μL, 0.047 mmol, 1.5 equiv) was added and the reaction was allowed to stir for 15h. The reaction was then quenched with ice water and extracted with ethyl acetate (×3). The organic layer was dried over sodium sulfate and concentrated under reduced pressure and purified via 1000 mm prep plate using 3% methanol in dichloromethane to provide 6.3 as a yellow oil (6.8 mg, 44% yield). TLC (5% methanol/dichloromethane), R_{f}=0.62; m.p. 178-180° C.; ¹H NMR (400 MHz, CDCl₃) δ 7.783-7.806 (d, J=8.7 Hz, 2H), 7.154-7.166 (dd, 1H), 7.118-7.139 (d, J=8.7 Hz, 2H), 6.875-6.897 (m, 1H), 6.715-6.736 (m, 1H), 3.674 (s, 2H), 3.547-3.580 (m, 4H), 3.284 (s, 3H), 2.311 (s. 3H), 1.967-1.991 (m, 4H); ¹³C-NMR (100 MHz, CDCl₃); 170.285, 155.868, 148.927, 139.213, 138.053, 137.039, 127.982, 126.533, 126.362, 126.270, 125.259, 124.801, 120.274, 47.142, 37.865, 35.280, 25.822, 17.541; IR (neat) vₘₐₓ 1583.85, 1508.03, 1441.51, 1231.31, 910.69, 729.28; ESI-HRMS [M+H]⁺ calculated for C₂₂H₂₄FN₅OS, 425.17, found 426.1741.

N-(4-((6-methyl-2-(pyrrolidin-1-yl)pyrimidin-4-yl)amino)phenyl)-1H-indole-3-carboxamide (6.3). Indole-3-carboxylic acid (11.6 mg, 0.0722 mmol, 1.2 equiv) was slurried with DIPEA (12.6 μL, 0.0722 mmol, 1.2 equiv) in DMF (anhydrous, 0.5 mL) and was treated with Hbtu (27.4 mg, 0.0722 mmol, 1.2 equiv) in DMF (anhydrous, 0.2 mL). The reaction was allowed to stir for 15 min at RT then 4.1 (16.2 mg, 0.0602 mmol, 1.0 equiv) in DMF (anhydrous, 0.2 mL) was added dropwise and the reaction was allowed to continue stirring for another 8h at RT. The reaction was diluted with DCM and washed with water and brine. The organic layer was dried over sodium sulfate and concentrated onto silica gel. Purification via column chromatography using 1:1 ethyl acetate/dichloromethane and 3% methanol produced the desired product 6.3 as a white solid (7.5 mg, 25% yield). TLC (3% methanol/dichloromethane), R_{f}=0.20; m.p. 182-184° C.; 1H NMR (400 MHz, CDCl₃) δ 8.171-8.193 (d, J=7.7 Hz, 1H), 7.973 (s, 1H), 7.896 (s, N—H), 7.662 (s, 4H), 7.440-7.460 (d, J=7.7 Hz, 1H), 7.155-7.238 (m, 2H), 5.963 (s, 1H), 3.574-3.594 (m, 4H), 2.274 (s, 3H), 2.022-2.050 (m, 4H); ¹³C-NMR (100 MHz, CDCl₃); 166.616, 164.856, 162.165, 138.153, 136.427, 135.951, 129.349, 127.596, 123.682, 122.418, 122.314, 122.137, 122.074, 112.812, 112.005, 97.046, 79.467, 36.944, 31.641, 26.260, 20.453; IR (neat) vₘₐₓ1505.51, 1232.99, 1176.73, 833.69, 743.65, 552.85; ESI-HRMS [M+H]⁺ calculated for C₂₄H₂₄N₆O, 412.2, found 413.2066.

tert-butyl (4-((2-chloro-6-methylpyrimidin-4-yl)amino)phenyl)carbamate (8). 2,4-dichloro-6-methylpyrimidine (2.1) (2.36 g, 0.0145 mol, 1.05 equiv) was dissolved in 30 mL of absolute ethanol and cooled with an ice bath and triethyl amine (2.5 mL, 0.0179 mol, 1.3 equiv) was added. tert-butyl (4-aminophenyl)carbamate (2.87 g, 0.0138 mol, 1.0 equiv) was dissolved in 15 mL of absolute ethanol and transferred to an additional funnel. The aniline was added dropwise and the reaction was allowed to warm to RT. After 24h, the reaction was heated to 40° C. until the reaction was complete. The solvent was removed under reduced pressure and purified via column chromatography using 5-50% ethyl acetate in hexanes to produce 8 (3.88 g, 80%) as an orange solid. TLC (20% ethyl acetate/dichloromethane), R_{f}=0.49; m.p. 109-111° C.; ¹H NMR (400 MHz, CD₃OD) δ 7.371-7.437 (m, 4H), 6.446 (s, 1H), 2.281 (s, 3H), 1.513 (s, 9H); ¹³C-NMR (100 MHz, CD₃OD) 168.015, 163.907, 160.978, 155.334, 137.049, 134.613, 123.140, 120.421, 80.832, 54.787, 28.713, 23.136; IR (neat) vₘₐₓ 1723.38, 1591.78, 1518.26, 1398.75, 1310.28, 1221.08, 1152.21, 1024.67, 970.18, 910.69, 835.64, 735.57, 515.54; ESI-HRMS [M+H]⁺ calculated for C₁₆H₁₉ClN₄O₂, 334.12, found 335.1257.

tert-butyl (4-((6-methyl-2-(pyrrolidin-1-yl)pyrimidin-4-yl) amino)phenyl)carbamate (9.1). 8 (3.88 g, 0.0116 mol, 1.0 equiv) was dissolved in 25 mL anhydrous DMF. Potassium Carbonate (2.08 g, 0.015 mol, 1.3 equiv) was added followed pyrrolidine (4.85 mL, 0.0581 mol, 5.0 equiv) and the reaction was heated to 80° C. for 8h. The reaction was then diluted with ethyl acetate and washed with water then brine. The organic layer was dried over sodium sulfate and concentrated under reduced vacuum, resulting in an orange solid. The crude material was run through a plug of silica gel with 5% methanol in DCM, and concentrated to produce carbamate 9.1 as a yellow solid. TLC (3% methanol/dichloromethane), R_{f}=0.28; m.p. 118-120° C.; ¹H NMR (400 MHz, CDCl₃) δ 7.303-7.352 (m, 4H), 6.428 (s, N—H), 6.294 (s, N—H), 5.767 (s, 1H), 3.532-3.609 (m, 4H), 2.219 (s, 3H), 1.928-1.961 (m, 4H), 1.520 (s, 9H); ¹³C-NMR (100 MHz, CDCl₃) 166.567, 161.606, 160.709, 153.112, 134.881, 134.083, 122.437, 119.567, 92.520, 80.474, 46.660, 28.447, 25.602, 24.318; IR (neat) vₘₐₓ 2971.67, 1718.69, 1569.84, 1505.67, 1399.06, 1227.56, 1155.05, 1050.05, 750.41, 515.91; ESI-HRMS [M+H]⁺ calculated for C₂₀H₂₇H₅O₂, 369.22, found 370.2225.

tert-butyl (4-((6-methyl-2-morpholinopyrimidin-4-yl)amino)phenyl)carbamate (9.2). 8 (199.0 mg, 0.594 mmol, 1.0 equiv) was dissolved in acetone (3.4 mL) and cooled with an ice bath. Sodium carbonate (69.3 mg, 0.653 mmol, 1.1 equiv) was added followed by morpholine (53.0 μL, 0.612 mmol, 1.03 equiv) in 1.0 mL of acetone, dropwise. The ice bath was removed and the reaction was heated to 80° C. for 8h. The reaction was diluted with ethyl acetate, then washed with water and brine. The organic layer was dried over sodium sulfate, concentrated under reduced pressure, then purified via column chromatography using 1% methanol in dichloromethane to produce 9.2 (122.88 mg, 54% yield) as a white solid. TLC (5% acetone in dichloromethane), R_{f}=0.18; m.p. 226-228° C.; ¹H NMR (400 MHz, CDCl₃) δ 7.317-7.349 (m, 2H), 7.243-7.264 (m, 2H), 6.465 (s, N—H), 6.314 (s, N—H), 5.816 (s, 1H), 3.742-3.765 (m, 8H), 2.203 (s, 3H), 1.520 (s, 9H); ¹³C-NMR (100 MHz, CDCl₃) 167.048, 162.072, 161.944, 153.257, 134.747, 134.264, 123.308, 119.610, 93.338, 80.766, 67.148, 44.511, 28.500, 24.474; IR (neat) vₘₐₓ 2947.41, 1702.51, 1579.06, 1489.70, 1357.90, 1230.06, 1155.92, 1004.35, 811.14, 746.29, 516.15; ESI-HRMS [M+H]⁺ calculated for C₂₀H₂₇N₅O₃, 385.21, found 386.2170.

2-(1H-indol-3-yl)-N-(4-((6-methyl-2-(pyrrolidin-1-yl)pyrimidin-4-yl)amino)phenyl)acetamide (6.5). 4.1 (70.0 mg, 0.260 mmol, 1.0 equiv) was dissolved in anhydrous DCM (1.5 mL) then treated with DMAP (31.8 mg, 0.260 mmol, 1.0 equiv), DCC (67.1 mg, 0.325 mmol, 1.25 equiv), and 2-(1H-indol-3-yl)acetic acid (47.8 mg, 0.273 mmol, 1.05 equiv). The reaction was allowed to stir for 12h at room temperature. Upon completion, the reaction was filtered through a pad of celite and concentrated on to silica gel. Purification via column chromatography using 1:1 ethyl acetate/dichloromethane and 3% methanol produced the desired product 6.5 (92.0 mg, 83% yield) as a white solid. TLC (3% methanol/dichloromethane), R_{f}=0.24; m.p. 197-199° C.; ¹H NMR (400 MHz, CD₃OD) δ 7.894 (s, N—H), 7.603-7.638 (m, 3H), 7.417-7.440 (d, J=8.9 Hz, 2H), 7.347-7.367 (d, J=8.2 Hz, 1H), 7.226 (s, 1H), 7.109 (t, 1H), 7.028 (t, 1H), 5.831 (s, 1H), 3.807 (s, 2H), 3.527-3.560 (m, 4H), 2.187 (s, 3H), 1.969-1.978 (m, 4H); ¹³C-NMR (100 MHz, CD₃OD) 172.991, 162.612, 161.617, 138.384, 138.140, 134.053, 128.607, 124.802, 122.560, 121.985, 121.160, 119.975, 119.411, 112.328, 109.563, 95.337, 79.466, 47.804, 34.895, 26.455, 23.433; IR (neat) vₘₐₓ 1503.05, 1401.52, 1202.57, 828.19, 738.45, 512.00; ESI-HRMS [M+H]⁺ calculated for C₂₅H₂₆N₆O, 426.22, found 427.2220.

3-(1H-indol-3-yl)-N-(4-((6-methyl-2-(pyrrolidin-1-yl)pyrimidin-4-yl)amino)phenyl) propanamide (6.6). 4.1 (30.1 mg, 0.112 mmol, 1.0 equiv) was dissolved in anhydrous DCM (1.5 mL) then treated with DMAP (13.7 mg, 0.112 mmol, 1.0 equiv), DCC (28.9 mg, 0.14 mmol, 1.25 equiv), and 3-(1H-indol-3-yl)propanoic acid (23.3 mg, 0.123 mmol, 1.1 equiv). The reaction was allowed to stir for 12h at room temperature. Upon completion, the reaction was filtered through a pad of celite and concentrated on to silica gel. Purification via column chromatography using 1:1 ethyl acetate/dichloromethane and 3% methanol produced the desired product 6.6 (38.3 mg, 78% yield) as a light orange solid. TLC (3% methanol/dichloromethane), R_{f}=0.20; m.p. 89-90° C.; ¹H NMR (400 MHz, CD₃OD) δ 7.888 (s, N—H), 7.558-7.629 (m, 3H), 7.388-7.410 (d, J=8.9 Hz, 2H), 7.306-7.327 (d, J=8.0 Hz, 1H), 7.061-7.097 (m, 2H), 6.979-7.015 (t, 1H), 5.834 (s, 1H), 3.529-3.562 (m, 4H), 3.127-3.165 (m, 2H), 2.700-2.738 (t, 2H), 2.190 (s, 3H), 1.947-1.980 (m, 4H); ¹³C-NMR (100 MHz, CD₃OD) 174.149, 166.029, 162.608, 161.578, 138.254, 138.174, 134.099, 128.543, 122.992, 122.286, 121.974, 121.142, 119.538, 119.330, 115.073, 112.169, 95.353, 79.460, 47.808, 39.114, 26.452, 23.426, 22.627; IR (neat) vₘₐₓ 1570.80, 1503.13, 1399.39, 1227.17, 791.94, 738.86, 514.31; ESI-HRMS [M+H]⁺ calculated for C₂₆H₂₈N₆O, 440.23, found 441.2382.

4-(1H-indol-3-yl)-N-(4-((6-methyl-2-(pyrrolidin-1-yl)pyrimidin-4-yl)amino)phenyl)butanamide (6.7). 4.1 (26.7 mg, 0.0992 mmol, 1.0 equiv) was dissolved in anhydrous DCM (1.5 mL) then treated with DMAP (12.1 mg, 0.0992 mmol, 1.0 equiv), DCC (25.6 mg, 0.124 mmol, 1.25 equiv), and 4-(1H-indol-3-yl)butanoic acid (20.2 mg, 0.109 mmol, 1.1 equiv). The reaction was allowed to stir for 12h at room temperature. Upon completion, the reaction was filtered through a pad of celite and concentrated on to silica gel. Purification via column chromatography using 1:1 ethyl acetate/dichloromethane and 3% methanol produced the desired product 6.7 (43 mg, 95% yield) as a light orange solid. TLC (5% methanol/dichloromethane), R_{f}=0.33; m.p. 157-159° C.; ¹H NMR (400 MHz, CD₃OD) δ 7.656 (s, N—H), 7.404-7.426 (d, J=8.9 Hz, 2H), 7.314-7.334 (d, J=7.9 Hz, 1H), 7.212-7.234 (d, J=8.9 Hz, 2H), 7.090-7.110 (d, J=8.0 Hz, 1H), 6.750-6.875 (m, 3H), 5.609 (s, 1H), 3.250-3.350 (m, 4H), 2.616 (t, 2H), 2.188 (t, 2H), 1.967 (s, 3H), 1.877 (m, 2H), 1.660-1.750 (m, 4H); ¹³C-NMR (100 MHz, CD₃OD) 174.403, 165.762, 162.519, 161.371, 138.179, 138.136, 134.163, 128.769, 122.968, 122.176, 121.839, 121.154, 119.419, 119.384, 115.626, 112.132, 95.418, 79.435, 47.780, 37.599, 28.738, 27.761, 26.403, 25.708, 23.363; IR (neat) vₘₐₓ 2861.61, 1570.45, 1503.18, 1398.69, 1229.00, 785.54, 738.42, 511.98; ESI-HRMS [M+H]⁺ calculated for C₂₇H₃₀N₆O, 454.25, found 455.2534.

(E)-3-(1H-indol-3-yl)-N-(4-((6-methyl-2-(pyrrolidin-1-yl)pyrimidin-4-yl)amino)phenyl)-acrylamide (6.8). 4.1 (19.0 mg, 0.0706 mmol, 1.0 equiv) was dissolved in anhydrous DCM (1.5 mL) then treated with (E)-3-(1H-indol-3-yl)acrylic acid (13.2 mg, 0.0706 mmol, 1.0 equiv), HBTU (34.8 mg, 0.0918 mmol, 1.3 equiv), and DIPEA (25.0 mL, 0.141 mmol, 1.1 equiv). The reaction was allowed to stir for 12h at room temperature. Upon completion, the reaction was diluted with ethyl acetate and washed with water and brine. The organic layer was dried over sodium sulfate and concentrated, then purified via column chromatography using 0-10% methanol in dichloromethane to produce the desired product 6.8 (20 mg, 63% yield) as a yellow oil. TLC (3% methanol/dichloromethane, run twice), R_{f}=0.33; m.p. 188-190° C.; ¹H NMR (400 MHz, CD₃OD) δ 7.918-7.978 (d, J=7.9 Hz, 1H), 7.860-7.899 (d, J=15.6 Hz, 1H), 7.669 (s, 4H), 7.626 (s, 1H), 7.436-7.455 (d, J=7.9 Hz, 1H), 7.176-7.252 (m, 2H), 6.726-6.765 (d, J=15.6 Hz, 1H), 5.986 (s, 1H), 3.539-3.630 (bs, 4H), 2.296 (s, 3H), 2.072 (bs, 4H); ¹³C-NMR (100 MHz, CD₃OD) 168.323, 162.306, 139.243, 137.042, 135.967, 135.664, 131.257, 126.624, 123.692, 121.838, 121.798, 121.453, 121.117, 116.081, 114.168, 113.084, 96.286, 79.465, 36.938, 31.638, 26.347, 21.908; IR (neat) vₘₐₓ 3107.31, 1581.55, 1508.82, 1403.71, 1343.98, 1241.42, 1180.48, 829.45, 743.93; ESI-HRMS [M+H]⁺ calculated for C₂₆H₂₆N₆O, 438.22, found 439.2223.

1H-pyrrolo[2,3-b]pyridine-3-carboxylic acid N-(4-((6-methyl-2-(pyrrolidin-1-yl)pyrimidin-4-yl)amino)phenyl)-1H-pyrrolo[2,3-b]pyridine-3-carboxamide (6.9). 4.1 (13.2 mg, 0.049 mmol, 1.0 equiv) was dissolved in anhydrous DFM (0.5 mL) then treated with 1H-pyrrolo[2,3-b]pyridine-3-carboxylic acid (8.0 mg, 0.049 mmol, 1.0 equiv), HBTU (21.4 mg, 0.056 mmol, 1.15 equiv), and DIPEA (9.8 mL, 0.056 mmol, 1.15 equiv). The reaction was allowed to stir for 12h at room temperature. Upon completion, the reaction was diluted with ethyl acetate and washed with water and brine. The organic layer was dried over sodium sulfate and concentrated, then purified via column chromatography using 0-10% methanol in dichloromethane to produce the desired product 6.9 (17.4 mg, 86% yield) as a yellow solid. TLC (3% methanol/dichloro-methane), R_{f}=0.24 (3% methanol/dicloromethane); m.p. 295-297° C.; ¹H NMR (400 MHz, CD₃OD) δ 2.234 (s, N—H), 8.482-8.505 (m, 1H), 8.440 (s, 1H), 8.296-8.311 (m, 1H), 7.692 (s, 4H), 7-195-7.226 (dd, 1H), 5.926 (s, 1H), 3.509 (bs, 4H), 2.177 (s, 3H), 1.924 (bs, 4H); ¹³C-NMR (100 MHz, CDCl₃) 162.263, 160.458, 148.461, 143.662, 129.324, 128.763, 128.564, 120.305, 120.199, 120.197, 119.935, 118.752, 117.094, 109.395, 99.522, 46.569, 24.964; IR (neat) vₘₐₓ 3297.80, 1661.79, 1576.88, 1501.18, 1450.36, 1338.97, 1221.50, 1012.59, 825.14, 689.68, 596.71, 510.91; ESI-HRMS [M+H]⁺ calculated for C₂₃H₂₃N₇O, 413.20, found 414.2022.

N-(4-((5-methoxy-2-(pyrrolidin-1-yl)pyrimidin-4-yl)amino)phenyl)-2-(thiophen-2-yl)-acetamide (6.10). The synthesis of 6.10 utilized Scheme 8 methodology and the following starting materials. 2,4-dichloro-5-methoxypyrimidine (13) (116.7 mg, 0.652 mmol, 1.0 equiv) was dissolved in ethanol (3 mL) and cooled to 0° C., then treated with triethyl amine (109.1 μL, 0.782 mmol, 1.2 equiv) and 1 (70.5 mg, 0.652 mmol, 1.0 equiv). The reaction was allowed to warm to RT and stir for 8h then concentrated and purified via column chromatography using 5% methanol in dichloromethane to provide (14) (154.0 mg, 94% yield) as a white solid. TLC (7% methanol/dichloromethane), R_{f}=0.19; m.p. 167-169° C.; ¹H NMR (400 MHz, CDCl₃) δ 7.644 (s, 1H), 7.412-7.434 (d, J=8.7 Hz, 2H), 7.089 (s, N—H), 6.691-6.713 (d, J=8.7 Hz, 2H), 3.944 (s, 3H), 3.631 (s, N—H, 2H); ¹³C-NMR (100 MHz, CDCl₃) 152.720, 151.597, 143.403, 139.311, 133.572, 129.027, 122.385, 115.666, 56.372; IR (neat) vₘₐₓ 3332.41, 1608.05, 1575.91, 1509.54, 1461.05, 1335.49, 1254.70, 1003.45, 961.94, 833.83, 756.01, 634.82, 553.64, 515.78, 459.09; ESI-HRMS [M+H]⁺ calculated for C₁₁H₁₁ClN₄O, 250.06, found 251.0685.

2-(4-bromothiophen-2-yl)-N-(4-((6-methyl-2-(pyrrolidin-1-yl)pyrimidin-4-yl)amino)phenyl)-acetamide (6.11). 4.1 (2.33 g, 0.00866 mol, 1.0 equiv) was dissolved in anhydrous DCM (100 mL) then treated with DMAP (1.06 g, 0.00866 mol, 1.0 equiv), DCC (2.23 g, 0.0108 mol, 1.25 equiv), and 2-(4-bromothiophen-2-yl)acetic acid (2.11 g, 0.00952 mol, 1.1 equiv). The reaction was allowed to stir for 24h at room temperature. Upon completion, the reaction was filtered through a pad of celite and concentrated on to silica gel. Purification via column chromatography using 1:1 ethyl acetate/dichloromethane and 3% methanol produced the desired product 6.11 (3.76 mg, brown solid) in 92% yield. TLC (8% methanol/dichloromethane), R_{f}=0.42; m.p. 195-197° C.; ¹H NMR (400 MHz, CDCl₃) δ 7.386-7.434 (m, 4H), 7.325 (s, N—H), 7.197 (s, 1H), 6.957 (s, 1H), 6.370 (s, N—H), 5.777 (s, 1H), 3.885 (s, 2H), 3.547-3.580 (m, 4H), 2.224 (s, 3H), 1.925-1.959 (m, 4H); ¹³C-NMR (100 MHz, CDCl₃) 166.809, 161.075, 137.195, 136.470, 132.471, 130,052, 123.159, 121.889, 121.607, 120.978, 109.899, 99.980, 92.628, 46.618, 38.423, 25.531, 24.357; IR (neat) vₘₐₓ 2862.68, 1568.07, 1501.64, 1400.13, 1333.38, 1231.08, 785.83, 563.89; ESI-HRMS [M+H]⁺ calculated for C₂₁H₂₂BrN₅OS, 471.07, found 472.0787.

N-(4-((2-chloro-6-methylpyrimidin-4-yl)oxy)phenyl)-2-(thiophen-2-yl)acetamide (6.12). 11.1 (63.0 mg, 0.27 mool, 1.0 equiv) was dissolved in ethanol (absolute, 3 mL) then treated with 2.1 (44.1 mg, 0.270 mmol, 1.0 equiv), potassium carbonate (44.8 mg, 0.324 mmol, 1.2 equiv) then a crystal of KI. The reaction was allowed to stir at RT for 8h, which was then concentrated onto silica gel and purified via column chromatography using 0-3% methanol in dichloromethane to produce 6.12 (92.8 mg, 96%) was found as an off-white solid. TLC (3% methanol/dichloromethane), R_{f}=0.45; m.p. 176-177° C.; ¹H NMR (400 MHz, CDCl₃) □□7.566 (s, N—H), 7.476-7.512 (d, J=8.9 Hz, 2H), 7.287-7.304 (dd, 1H), 7.018-7.063 (m, 4H), 6.564 (s, 1H), 3.937 (s, 2H), 2.453 (s, 3H); ¹³C-NMR (100 MHz, CDCl₃) 171.760, 170.920, 168.118, 160.102, 148.396, 135.564, 135.517, 127.912, 127.728, 126.211, 122.103, 121.936, 121.570, 121.238, 115.629, 105.169, 38.549, 24.044; IR (neat) vₘₐₓ 1655.62, 1578.47, 1507.89, 1323.18, 1207.23, 846.34, 793.77, 690.66, 481.83; ESI-HRMS [M+H]⁺ calculated for C₁₇H₁₄ClN₃O₂S, 359.05, found 360.0552.

N-(4-((6-methyl-2-(pyrrolidin-1-yl)pyrimidin-4-yl)oxy)phenyl)-2-(thiophen-2-yl)acetamide (6.13). 6.12 (44.3 mg, 0.123 mmol, 1.0 equiv) was dissolved in DMF (anhydrous, 1 mL) and then treated with potassium carbonate (20.3 mg, 0.148 mmol, 1.2 equiv) and pyrrolidine (20.6 mL, 0.247 mmol, 2.0 equiv). The mixture was heated to 80° C. for 8h, then diluted in dichloromethane and washed with water then brine. The organic layer was dried over sodium sulfate, concentrated then purified via column chromatography using 30-60% ethyl acetate in hexanes to provide 6.13 (8.5 mg, 18% yield) as a white solid. TLC (3% methanol/dichloromethane), R_{f}=0.43; m.p. 187-189° C.; ¹H NMR (400 MHz, CDCl₃) δ 7.438-7.460 (d, J=8.9 Hz, 2H), 7.310-7.350 (m, 1H), 7.049-7.102 (m, 3H), 5.727 (s, 1H), 3.960 (s, 2H), 3.484 (bs, 4H), 2.258 (s, 3H), 1.882-1.936 (m, 4H); ¹³C-NMR (100 MHz, CDCl₃) 170.292, 169.595, 167.758, 160.509, 149.532, 135.530, 134.331, 127.895, 127.678, 126.174, 122.203, 120.974, 93.152, 46.590, 38.484, 25.418, 24.404; IR (neat) vₘₐₓ 1655.18, 1575.53, 1503.62, 1330.12, 1204.97, 961.26, 844.36, 792.73, 688.69, 567.34, 519.75, 480.84; ESI-HRMS [M+H]⁺ calculated for C₁₇H₁₄ClN₃O₂S, 394.15, found 395.1522.

N-(4-((6-methyl-2-(pyrrolidin-1-yl)pyrimidin-4-yl)oxy)phenyl)-1H-indole-3-carboxamide (6.14). 11.2 (9.0 mg, 0.024 mmol, 1.0 equiv) was dissolved in 1.0 mL anhydrous DMF. Potassium Carbonate (4.3 mg, 0.0312 mol, 1.3 equiv) was added followed pyrroldine (10 mL, 0.119 mmol, 5.0 equiv) and the reaction was heated to 80° C. for 8h. The reaction was then diluted with ethyl acetate and washed with water then brine. The organic layer was dried over sodium sulfate and concentrated under reduced vacuum, resulting in an orange solid. The crude material was run through a plug of silica gel with 5% methanol in dichloromethane and concentrated to produce 6.14 as a yellow solid. TLC (3% methanol/dichloromethane), R_{f}=0.20; m.p. 172-174° C.; ¹H NMR (400 MHz, CDCl₃) δ 8.710 (s, N—H), 8.054-8.094 (m, 1H), 7.882-7.889 (d, J=4.0 Hz, 1H), 7.733 (s, N—H), 7.667-7.689 (d, J=8.9 Hz, 2H), 7.457-7.500 (m, 1H), 7.300-7.343 (m, 2H), 7.162-7.184 (d, J=8.9 Hz, 2H), 5.778 (s, 1H), 3.522 (bs, 4H), 2.281 (s, 3H), 1.909-1.943 (m, 4H); ¹³C-NMR (100 MHz, CDCl₃) 170.631, 169.798, 163.563, 149.229, 136.539, 135.440, 128.389, 124.854, 123.429, 122.408, 122.143, 121.465, 120.113, 112.729, 112.252, 93.360, 60.557, 46.804, 25.581, 24.551, 21.214, 14.348; IR (neat) vₘₐₓ 3325.82, 1575.09, 1506.16, 1425.63, 1248.26, 1094.00, 949.73, 808.19, 775.56, 487.07; ESI-HRMS [M+H]⁺ calculated for C₂₄H₂₃N₅O₂, 413.19, found 414.1908.

N-(4-((6-methyl-2-morpholinopyrimidin-4-yl)amino)phenyl)-2-(thiophen-2-yl)acetamide (6.15). 4.1 (17.5 mg, 0.061 mmol, 1.0 equiv) was dissolved in anhydrous DCM (1.5 mL) then treated with DMAP (7.5 mg, 0.061 mmol, 1.0 equiv), DCC (19.9 mg, 0.0763 mmol, 1.25 equiv), and 5.1 (9.1 mg, 0.064 mmol, 1.05 equiv). The reaction was allowed to stir for 12h at room temperature. Upon completion, the reaction was filtered through a pad of celite and concentrated on to silica gel. Purification via column chromatography using 1:1 ethyl acetate/dichloromethane and 3% methanol produced the desired product 6.15 (14 mg, 56% yield) as a dark solid. TLC (5% Methanol/dichloromethane), R_{f}=0.60; m.p. 196-198° C.; ¹H NMR (400 MHz, CD₃OD) δ 7.454-7.522 (q, 4H), 7.263-7.277 (d, J=5.7 Hz, 1H), 6.995 (bs, 1H), 6.952-6.974 (t, 1H), 5.907 (s, 1H), 3.870 (s, 2H), 3.723 (s, 8H), 2.190 (s, 3H); ¹³C-NMR (100 MHz, CD₃OD) 170.716, 166.644, 163.165, 162.779, 137.897, 134.192, 127.716, 127.482, 125.742, 121.808, 121.653, 96.230, 67.886, 45.856, 38.672, 23.833; IR (neat) vₘₐₓ 2842.93, 1654.21, 1579.80, 1545.32, 1486.92, 1439.58, 1398.31, 1354.51, 1232.28, 1104.29, 993.25, 830.14, 786.59, 696.36, 483.49; ESI-HRMS [M+H]⁺ calculated for C₂₁H₂₃N₅O₂S, 409.16, found 410.1630.

N-(4-((6-methyl-2-(pyrrolidin-1-yl)pyrimidin-4-yl)amino)phenyl)-2-(naphthalen-1-yl)acetamide (6.16). 4.1 (33.6 mg, 0.125 mmol, 1.0 equiv) was dissolved in anhydrous DCM (1.5 mL) then treated with DMAP (15.3 mg, 0.125 mmol, 1.0 equiv), DCC (32.2 mg, 0.156 mmol, 1.25 equiv), and 2-(naphthalen-1-yl)acetic acid (25.6 mg, 0.137 mmol, 1.1 equiv). The reaction was allowed to stir for 12h at room temperature. Upon completion, the reaction was filtered through a pad of celite and concentrated on to silica gel. Purification via column chromatography using 1:1 ethyl acetate/dichloromethane and 3% methanol produced the desired product 6.16 (22 mg, 40% yield) as an off white solid. TLC (3% methanol/dichloromethane, run twice), R_{f}=0.5; m.p. 275-278° C.; ¹H NMR (400 MHz, DMSO-d⁶) δ 10.168 (s, N—H, 1H), 8.946 (s, N—H, 1H), 8.134-8.154 (d, J=8.1 Hz, 1H), 7.926-7.948 (d, J=7.9 Hz, 1H), 7.830-7.855 (dd, 1H), 7.609-7.631 (d, J=8.9 Hz 1H), 7.457-7.582 (m, 6H), 5.824 (s, 1H), 4.121 (s, 2H), 3.435-3.467 (m, 4H), 2.113 (s, 3H), 1.864-1.896 (m, 4H); ¹³C NMR (100 MHz, DMSO-d⁶) 168.533, 164.598, 160.633, 159.945, 136.439, 133.358, 132.978, 132.628, 131.997, 128.411, 127.776, 127.161, 126.065, 125.671, 125.539, 124.213, 119.633, 119.483, 93.799, 46.185, 40.620, 25.019, 23.938; IR (neat) vₘₐₓ 1660.02, 1575.10, 1503.95, 1401.14, 1227.15, 787.35, 558.68; ESI-HRMS [M+H]⁺ calculated for C₂₇H₂₇N₅O, 437.22, found 438.2273.

2-(6-chloro-1H-indol-3-yl)-N-(4-((6-methyl-2-(pyrrolidin-1-yl)pyrimidin-4-yl)amino)phenyl)acetamide (6.18). 4.1 (25.8 mg, 0.096 mmol, 1.0 equiv) was dissolved in dichloromethane, anhydrous (1.5 mL) and treated with DMAP (12.8 mg, 0.105 mmol, 1.1 equiv), DCC (24.8 mg, 0.12 mmol, 1.25 equiv) and 2-(6-chloro-1H-indol-3-yl)acetic acid (22.1 mg, 0.105 mmol, 1.0 equiv). The vial was purged with nitrogen and allowed to stir for 24h at RT. The solution was filtered through a pad of celite and concentrated on to silica gel. Purification via column chromatography using 1:1 ethyl acetate/dichloromethane and 3% methanol provided 6.18 (25.4 mg, 57% yield) as an off white solid. TLC (2% Methanol, 20% acetone, 78% dichloromethane), R_{f}=0.57; m.p. 146-148° C.; ¹H NMR (400 MHz, CD₃OD) δ 7.613-7.651 (d, J=8.9 Hz, 2H), 7.557-7.578 (d, J=8.5 Hz, 1H), 7.427-7.449 (d, J=8.9 Hz, 2H), 7.348-7.360 (d, J=2.0 Hz, 1H), 7.242 (s, 1H), 6.994-7.019 (dd, 1H), 5.825 (s, 1H), 3.784 (s, 2H), 3.529-3.563 (m, 4H), 2.189 (s, 3H), 1.949-1.983 (m, 4H); ¹³C-NMR (100 MHz, CD₃OD) 172.615, 162.616, 159.239, 138.467, 138.412, 134.068, 128.481, 127.362, 125.759, 121.970, 121.184, 120.604, 120.468, 112.127, 110.057, 95.356, 76.019, 47.820, 34.666, 26.458, 23.418; IR (neat) vₘₐₓ 1660.51, 1571.67, 1505.47, 1399.30, 795.55; ESI-HRMS [M+H]⁺ calculated for C₂₅H₂₅ClN₆O, 460.18, found 461.1834.

1-methyl-N-(4-((6-methyl-2-(pyrrolidin-1-yl)pyrimidin-4-yl)amino)phenyl)-1H-indole-3-carboxamide (6.19). 4.1 (60 mg, 0.223 mmol, 1.0 equiv) was dissolved in dichloromethane, anhydrous (1.5 mL) and treated with DMAP (30 mg, 0.245 mmol, 1.1 equiv), DCC (57.5 mg, 0.279 mmol, 1.25 equiv) and 1-methyl-1H-indole-3-carboxylic acid (39 mg, 0.223 mmol, 1.0 equiv). The vial was purged with nitrogen and allowed to stir for 24h at RT. The solution was filtered through a pad of celite and concentrated on to silica gel. Purification via column chromatography using 1:1 ethyl acetate/dichloromethane and 3% methanol provided 6.19 (23.6 mg, 25% yield) as a light yellow solid. TLC (5% methanol/dichloromethane, run twice), R_{f}=0.58; m.p. 262-264° C.; ¹H NMR (400 MHz, CD₃OD) δ 8.176-8.196 (d, J=8.1 Hz, 1H), 7.945 (s, 1H), 7.668-7.690 (d, J=8.9 Hz, 2H), 7.560-7.582 (d, J=8.9 Hz, 2H), 7.425-7.445 (d, J=8.1 Hz, 1H), 7.250-7.291 (t, 1H), 7.187-7.227 (t, 1H), 5.857 (s, 1H), 3.855 (s, 3H), 3.544-3.577 (m, 4H), 2.201 (s, 3H), 1.958-1.991 (m, 4H); ¹³C-NMR (100 MHz, CD₃OD) 166.950, 165.212, 162.641, 161.568, 138.775, 138.012, 134.530, 133.132, 128.188, 123.729, 122.537, 122.328, 121.277, 111.161, 110.925, 95.376, 79.464, 47.819, 36.937, 33.480, 26.463, 23.422; IR (neat) vₘₐₓ 3307.96, 1571.45, 1502.60, 1399.70, 1227.73, 1100.50, 741.44; ESI-HRMS [M+H]⁺ calculated for C₂₅H₂₆N₆O, 426.22, found 427.2227.

N-(4-((6-methyl-2-(pyrrolidin-1-yl)pyrimidin-4-yl)amino)phenyl)-2-(1H-pyrrolo[2,3-b]pyridin-3-yl)acetamide (6.20) 4.1 (25.2 mg, 0.0936 mmol, 1.0 equiv) was dissolved in dichloroemethane, anhydrous (1.5 mL) and treated with DMAP (12.6 mg, 0.103 mmol, 1.1 equiv), DCC (24.1 mg, 0.117 mmol, 1.25 equiv) and 2-(1H-pyrrolo[2,3-b]pyridin-3-yl)acetic acid (39 mg, 0.223 mmol, 1.0 equiv). The vial was purged with nitrogen and allowed to stir for 24h at RT. The solution was filtered through a pad of celite and concentrated on to silica gel. Purification via column chromatography using 1:1 ethyl acetate/dichloromethane and 3% methanol provided 6.20 (36.0 mg, 90% yield) as a white solid. TLC (20% acetone, 2% methanol, 78% dichloromethane, run twice), R_{f}=0.35; m.p. 236-238° C.; ¹H NMR (400 MHz, CD30D) δ 8.175-8.191 (d, J=5.1 Hz, 1H), 8.080-8.104 (d, J=7.9 Hz, 1H), 7.831 (s, 1H), 7.610-7.648 (d, J=9.0 Hz, 2H), 7.441-7.479 (d, J=9.0 Hz, 2H), 7.092-7.124 (dd, 1H), 5.849 (s, 1H), 3.818 (s, 2H), 3.545-3.578 (m, 4H), 2.204 (s, 3H), 1.965-1.999 (m, 4H); ¹³C-NMR (100 MHz, CD₃OD) 172.217, 162.578, 149.416, 143.291, 138.215, 134.262, 129.094, 125.774, 121.924, 121.851, 121.317, 116.466, 109.195, 101.393, 95.543, 47.878, 34.712, 26.440, 23.082; IR (neat) vₘₐₓ 2864.93, 1572.18, 1502.48, 1398.08, 1229.57, 753.09, 515.55; ESI-HRMS [M+H]⁺ calculated for C₂₄H₂₅N70, 427.21, found 428.2178.

2-(2-methyl-1H-indol-3-yl)-N-(4-((6-methyl-2-(pyrrolidin-1-yl)pyrimidin-4-yl)amino)phenyl)acetamide (6.21). 4.1 (20.8 mg, 0.0773 mmol, 1.0 equiv) was dissolved in dichloromethane, anhydrous (1.5 mL) and treated with DMAP (9.44 mg, 0.0773 mmol, 1.1 equiv), DCC (19.9 mg, 0.0966 mmol, 1.25 equiv) and 2-(2-methyl-1H-indol-3-yl)acetic acid (15.4 mg, 0.0773 mmol, 1.0 equiv). The vial was purged with nitrogen and allowed to stir for 24h at RT. The solution was filtered through a pad of celite and concentrated on to silica gel. Purification via column chromatography using 1:1 ethyl acetate/dichloromethane and 3% methanol provided 6.21 (24.1 mg, 73.3% yield) as a white solid. TLC (3% methanol/dichloromethane), R_{f}=0.42; m.p. 142-144° C.; ¹H NMR (400 MHz, CD₃OD) δ 7.592-7.614 (d, J=8.9 Hz, 2H), 7.471-7.491 (d, J=7.6 Hz, 1H), 7.383-7.405 (d, J=8.9 Hz, 2H), 7.240-7.258 (d, J=7.6 Hz, 1H), 6.947-7.038 (m, 2H), 5.802 (s, 1H), 3.739 (s, 2H), 3.492-3.525 (m, 4H), 2.417 (s, 3H), 2.168 (s, 3H), 1.908-1.941 (m, 4H); ¹³C-NMR (100 MHz, CD₃OD). 173.092, 165.696, 162.506, 161.309, 138.322, 137.089, 134.671, 133.965, 129.856, 122.078, 121.634, 121.134, 119.853, 118.485, 111.417, 105.226, 95.441, 47.791, 33.567, 26.409, 23.316, 11.539; IR (neat) vₘₐₓ 1571.52, 1505.98, 1399.07, 741.35; ESI-HRMS [M+H]⁺ calculated for C₂₆H₂₈N₆O, 440.23, found 441.2381.

2-(5-methoxy-1H-indol-3-yl)-N-(4-((6-methyl-2-(pyrrolidin-1-yl)pyrimidin-4-yl)amino)phenyl) acetamide (6.22). 4.1 (21.6 mg, 0.0802 mmol, 1.0 equiv) was dissolved in dichloromethane, anhydrous (1.5 mL) and treated with DMAP (9.80 mg, 0.0802 mmol, 1.1 equiv), DCC (20.7 mg, 0.100 mmol, 1.25 equiv) and 2-(5-methoxy-1H-indol-3-yl)acetic acid (17.3 mg, 0.0843 mmol, 1.0 equiv). The vial was purged with nitrogen and allowed to stir for 24h at RT. The solution was filtered through a pad of celite and concentrated on to silica gel. Purification via column chromatography using 1:1 ethyl acetate/dichloromethane and 3% methanol provided 6.22 (15.5 mg, 44% yield) as a white solid. TLC (3% methanol/dichloromethane), R_{f}=0.55; m.p. 167-169° C.; ¹H NMR (400 MHz, CD₃OD) δ 7.621-7.643 (d, J=8.9 Hz, 2H), 7.429-7.451 (d, J=8.9 Hz, 2H), 7.237-7.259 (d, J=8.7 Hz, 1H), 7.199 (s, 1H), 7.118-7.124 (d, J=2.0 Hz 1H), 6.760-6.788 (dd, 1H), 5.844 (s, 1H), 3.802 (s, 3H), 3.775 (s, 2H), 3.552 (bm, 4H), 2.195 (s, 3H), 1.974 (bm, 4H). ¹³C-NMR (100 MHz, CD₃OD); 173.054, 162.612, 155.223, 137.780, 134.158, 133.356, 128.899, 125.518, 121.988, 121.257, 113.029, 112.906, 109.361, 101.367, 95.418, 79.464, 56.255, 47.828, 35.031, 26.454, 23.724, 20.049; IR (neat) vₘₐₓ 2864.55, 1573.52, 1504.15, 1398.88, 1224.02, 790.75, 513.02; ESI-HRMS [M+H]⁺ calculated for C₂₆H₂₈N₆O₂, 456.23, found 457.2329.

2-(5-bromo-1H-indol-3-yl)-N-(4-((6-methyl-2-(pyrrolidin-1-yl)pyrimidin-4-yl)amino)phenyl) acetamide (6.23). 4.1 (17.0 mg, 0.0632 mmol, 1.0 equiv) was dissolved in dichloromethane, anhydrous (1.5 mL) and treated with DMAP (7.72 mg, 0.0632 mmol, 1.1 equiv), DCC (16.3 mg, 0.079 mmol, 1.25 equiv) and 2-(5-bromo-1H-indol-3-yl)acetic acid (17.7 mg, 0.0695 mmol, 1.0 equiv). The vial was purged with nitrogen and allowed to stir for 24h at RT. The solution was filtered through a pad of celite and concentrated on to silica gel. Purification via column chromatography using 1:1 ethyl acetate/dichloromethane and 3% methanol provided 6.23 (30.7 mg, 99% yield) as a white solid. TLC (5% methanol/dichloromethane), R_{f}=0.47; m.p. 151-153° C.; 1H NMR (400 MHz, CD₃OD) δ 7.899 (s, 1H), 7.784-7.788 (d, J=1.9 Hz, 1H), 7.630-7.652 (d, J=8.9 Hz, 2H), 7.436-7.459 (d, J=8.9 Hz 2H), 7.264-7.272 (m, 2H), 7.183-7.210 (dd, 1H), 5.843 (s, 1H), 3.769 (s, 2H), 3.518-3.598 (m, 4H), 2.193 (s, 3H), 1.948-2.003 (m, 4H); ¹³C-NMR (100 MHz, CD₃OD) 172.565, 162.617, 161.530, 155.127, 138.382, 136.716, 134.086, 130.445, 126.290, 125.261, 122.170, 122.017, 121.233, 113.973, 113.149, 109.537, 95.377, 47.823, 34.618, 26.455, 23.383; IR (neat) vₘₐₓ 2862.30, 1570.94, 1504.59, 1399.41, 1225.39, 789.49, 513.22; ESI-HRMS [M+H]⁺ calculated for C₂₅H₂₅BrN₆O, 504.13, found 505.1327.

2-(1H-indol-1-yl)-N-(4-((6-methyl-2-(pyrrolidin-1-yl)pyrimidin-4-yl)amino)phenyl)acetamide (6.24). 4.1 (16.6 mg, 0.0617 mmol, 1.0 equiv) was dissolved in dichloromethane, anhydrous (1.5 mL) and treated with DMAP (7.5 mg, 0.0617 mmol, 1.1 equiv), DCC (16.0 mg, 0.077 mmol, 1.25 equiv) and 2-(1H-indol-1-yl)acetic acid (11.3 mg, 0.0648 mmol, 1.0 equiv). The vial was purged with nitrogen and allowed to stir for 24h at RT. The solution was filtered through a pad of celite and concentrated on to silica gel. Purification via column chromatography using 1:1 ethyl acetate/dichloromethane and 3% methanol provided 6.24 (12.0 mg, 47% yield) as an off white solid. TLC (3% methanol/dichloromethane), R_{f}=0.32; m.p. 275-277° C.; ¹H NMR (400 MHz, CD₃OD) δ 7.111-7.130 (d, J=7.8 Hz, 1H), 6.875-6.899 (d, J=8.9 Hz, 2H), 6.756-6.825 (m, 4H), 6.708 (t, 1H), 6.599-6.650 (m, 2H), 6.083-6.091 (d, J=3.2 Hz, 1H), 5.230 (s, 1H), 4.381 (s, 2H), 2.960-2.993 (m, 3H), 1.642 (s, 3H), 1.377-1.410 (m, 4H); ¹³C-NMR (100 MHz, CD₃OD) 173.133, 162.616, 138.337, 136.690, 136.060, 134.116, 128.780, 124.921, 123.207, 122.022, 121.234, 117.835, 112.158, 109.187, 95.39434.965, 30.173, 26.451, 23.333, 17.153; IR (neat) vₘₐₓ 1668.49, 1576.69, 1502.37, 1400.50, 1330.94, 1227.86, 794.97, 735.87, 568.62, 509.47; ESI-HRMS [M+H]⁺ calculated for C₂₅H₂₆N₆O, 426.22, found 427.2224.

2-(5-hydroxy-1H-indol-3-yl)-N-(4-((6-methyl-2-(pyrrolidin-1-yl)pyrimidin-4-yl)amino)phenyl) acetamide (6.25). 4.1 (44.2 mg, 0.164 mmol, 1.0 equiv) was dissolved in dichloromethane, anhydrous (1.5 mL) and treated with DMAP (20.0 mg, 0.164 mmol, 1.0 equiv), DCC (42.3 mg, 0.205 mmol, 1.25 equiv) and 2-(5-hydroxy-1H-indol-3-yl)acetic acid (33.0 mg, 0.173 mmol, 1.0 equiv). The vial was purged with nitrogen and allowed to stir for 24h at RT. The solution was filtered through a pad of celite and concentrated on to silica gel. Purification via column chromatography using 1:1 ethyl acetate/dichloromethane and 3% methanol provided 6.25 (15.5 mg, 21% yield) as a white solid. TLC (5% methanol/dichloromethane), R_{f}=0.28; m.p. 196-198° C.; ¹H NMR (400 MHz, CD₃OD) δ 7.614-7.636 (d, J=8.9 Hz, 2H), 7.445-7.467 (d, J=8.9 Hz, 2H), 7.173-7.203 (m, 2H), 6.983-6.988 (d, J=3.2 Hz, 1H), 6.675-6.703 (dd, 1H), 5.877 (s, 1H), 3.746 (s, 2H), 3.547-3.581 (m, 4H), 2.221 (s, 3H), 1.961-2.025 (m, 4H); ¹³C-NMR (100 MHz, CD₃OD) 173.106, 162.529, 151.451, 151.370, 134.650, 133.095, 129.301, 125.657, 125.397, 121.978, 121.555, 112.809, 112.696, 108.727, 103.674, 103.594, 95.786, 52.325, 35.009, 31.977, 26.407; IR (neat) vₘₐₓ 3258.66, 1578.16, 1506.67, 1397.36, 1228.60, 793.25; ESI-HRMS [M+H]⁺ calculated for C₂₅H₂₆N₆O₂, 442.21, found 443.2172.

2-(3-(cyclopropylmethyl)-1H-indol-1-yl)-N-(4-((6-methyl-2-(pyrrolidin-1-yl)pyrimidin-4-yl)amino)phenyl)acetamide (6.26). 4.1 (17.0 mg, 0.0632 mmol, 1.0 equiv) was dissolved in dichloromethane, anhydrous (1.5 mL) and treated with DMAP (7.72 mg, 0.0632 mmol, 1.1 equiv), DCC (16.3 mg, 0.079 mmol, 1.25 equiv) and 2-(3-(cyclopropylmethyl)-1H-indol-1-yl)acetic acid (16.1 mg, 0.0663 mmol, 1.0 equiv). The vial was purged with nitrogen and allowed to stir for 24h at RT. The solution was filtered through a pad of celite and concentrated on to silica gel. Purification via column chromatography using 1:1 ethyl acetate/dichloromethane and 3% methanol provided 6.26 (23.7 mg, 78% yield) as an off white solid. TLC (3% methanol/dichloromethane), R_{f}=0.27; m.p. 275-277° C.; ¹H NMR (400 MHz, CD₃OD) δ 8.311 (s, 1H), 8.270-8.289 (d, J=7.8 Hz, 1H), 7.780 (s, 1H), 7.625-7.647 (d, J=8.9 Hz, 2H), 7.517-7.539 (d, J=8.9 Hz, 2H), 7.425-7.444 (d, J=7.8 Hz, 1H), 7.218-7.305 (m, 2H), 5.918 (s, 1H), 5.109 (s, 2H), 3.554-3.586 (m, 4H), 2.605-2.667 (m, 1H), 2.250 (s, 3H), 1.991-2.024 (m, 4H), 1.956 (s, 2H), 1.121-1.158 (m, 2H), 0.949-0.995 (m, 2H); ¹³C-NMR (100 MHz, CD₃OD) 197.917, 166.991, 162.055, 139.009, 138.658, 138.587, 137.137, 134.355, 127.271, 124.469, 123.493, 123.177, 121.899, 121.535, 118.579, 110.702, 96.389, 49.285, 50.561, 26.188, 22.943, 21.585, 18.662, 10.523; IR (neat) vₘₐₓ 2009.39, 1508.43, 1386.73, 1167.26, 1065.42, 946.58, 744.69; ESI-HRMS [M+H]⁺ calculated for C₂₉H₃₂N₆O₂, 494.20, found 495.2484.

2-(5-ethyl-1H-indol-3-yl)-N-(4-((6-methyl-2-(pyrrolidin-1-yl)pyrimidin-4-yl)amino)phenyl) acetamide (6.27). 4.1 (15.0 mg, 0.056 mmol, 1.0 equiv) was dissolved in dichloromethane, anhydrous (1.5 mL) and treated with DMAP (6.84 mg, 0.056 mmol, 1.1 equiv), DCC (14.4 mg, 0.070 mmol, 1.25 equiv) and 2-(5-ethyl-1H-indol-3-yl)acetic acid (11.9 mg, 0.0585 mmol, 1.0 equiv). The vial was purged with nitrogen and allowed to stir for 24h at RT. The solution was filtered through a pad of celite and concentrated on to silica gel. Purification via column chromatography using 1:1 ethyl acetate/dichloromethane and 3% methanol provided 6.27 (24.8 mg, 98% yield) as an off white solid. TLC (3% methanol/dichloromethane), R_{f}=0.30; m.p. 177-179° C.; ¹H NMR (400 MHz, CD₃OD) δ 7.617-7.639 (d, J=8.9 Hz, 2H), 7.417-7.439 (m, 3H), 7.257-7.278 (d, J=8.9 Hz, 1H), 7.189 (s, 1H), 6.972-6.996 (d, J=8.4 Hz 1H), 5.838 (s, 1H), 3.789 (s, 2H), 3.530-3.563 (m, 4H), 2.685-2.742 (q, 2H), 2.191 (s, 3H), 1.950-1.984 (m, 4H), 1.253 (t, 3H); ¹³C-NMR (100 MHz, CD₃OD) 173.133, 162.616, 138.337, 138.332, 136.690, 136.060, 134.116, 128.780, 124.921, 123.207, 122.022, 121.234, 117.835, 112.158, 109.187, 95.394, 47.829, 34.965, 30.173, 26.452, 23.333, 17.153; IR (neat) vₘₐₓ 2957.96, 1504.91, 1399.77, 1253.22, 803.62; ESI-HRMS [M+H]⁺ calculated for C₂₇H₃₀N₆O, 454.25, found 455.2535.

2-(1H-benzo[d]imidazole-1-yl)-N-(4-((6-methyl-2-(pyrrolidin-1-yl)pyrimidin-4-yl)amino)phenyl) acetamide (6.28). 4.1 (15.6 mg, 0.0580 mmol, 1.0 equiv) was dissolved in dichloromethane, anhydrous (1.5 mL) and treated with DMAP (7.1 mg, 0.0580 mmol, 1.1 equiv), DCC (15.0 mg, 0.0725 mmol, 1.25 equiv) and 2-(1H-benzo[d]imidazol-1-yl)acetic acid (10.7 mg, 0.0609 mmol, 1.0 equiv). The vial was purged with nitrogen and allowed to stir for 24h at RT. The solution was filtered through a pad of celite and concentrated on to silica gel. Purification via column chromatography using 1:1 ethyl acetate/dichloromethane and 3% methanol provided 6.28 (16.9 mg, 68% yld) as a white solid. TLC (3% methanol/dichloromethane), R_{f}=0.27; m.p. 175-177° C.; ¹H NMR (400 MHz, DMSO-d⁶) δ 10.368 (s, 1H), 8.229 (s, 1H), 7.641-7.678 (m, 3H), 7.486-7.542 (m, 3H), 7.190-7.270 (m, 2H), 5.857 (s, 1H), 5.148 (s, 2H), 3.446-3.505 (m, 4H), 2.135 (s, 3H), 1.885-1.916 (m, 4H); ¹³C-NMR (100 MHz, DMSO-d⁶) 179.572, 164.927, 144.989, 143.167, 134.379, 122.336, 121.465, 119.673, 119.323, 110.280, 72.465, 63.055, 48.568, 47.275, 46.295, 24.955; IR (neat) vₘₐₓ 3093.18, 1581.23, 1502.31, 1403.46, 1298.11, 1230.31, 1175.88, 835.05, 738.59; ESI-HRMS [M+H]⁺ calculated for C₂₄H₂₅N₇O, 427.21, found 428.2177.

N-(4-((6-methyl-2-(pyrrolidin-1-yl)pyrimidin-4-yl)amino)phenyl)-2-(1-((4-(trifluoromethyl) phenyl)sulfonyl)-1H-indol-3-yl)acetamide (6.29). 4.1 (15.6 mg, 0.0580 mmol, 1.0 equiv) was dissolved in dichloromethane, anhydrous (1.5 mL) and treated with DMAP (7.1 mg, 0.0580 mmol, 1.1 equiv), DCC (15.0 mg, 0.0725 mmol, 1.25 equiv) and 2-(1-((4-(trifluoromethyl)phenyl)sulfonyl-H-indol-3-yl) acetic acid (117, 1.0 equiv). The vial was purged with nitrogen and allowed to stir for 24h at RT. The solution was filtered through a pad of celite and concentrated on to silica gel. Purification via column chromatography using 1:1 ethyl acetate/dichloromethane and 3% methanol provided 6.29 (16.9 mg, 68% yield) as a white solid. TLC (3% methanol/dichloromethane), R_{f}=0.47; m.p. 230-232° C.; ¹H NMR (400 MHz, CD₃OD) δ 8.102-8.123 (d, J=8.4 Hz, 2H), 7.989-8.010 (d, J=8.4 Hz, 1H), 7.793-7.814 (d, J=8.4 Hz, 1H), 7.612-7.676 (m, 4H), 7.449-7.471 (d, J=8.9 Hz, 2H), 7.344-7.383 (t, 1H), 7.264-7.302 (t, 1H), 5.878 (s, 1H), 3.787 (s, 2H), 3.554-3.586 (m, 4H), 2.219 (s, 3H), 1.978-2.010 (m, 4H); ¹³C-NMR (100 MHz, CD₃OD) 170.699, 162.550, 142.731, 136.537, 136.446, 136.116, 132.265, 128.837, 127.658, 126.295, 126.000, 124.954, 121.830, 121.473, 121.061, 119.402, 114.738, 101.399, 79.474, 47.965, 33.994, 26.429; IR (neat) vₘₐₓ 1649.02, 1577.42, 1505.48, 1400.80, 1319.87, 1169.71, 1126.77, 1059.29, 830.21, 784.74, 713.73, 607.93, 558.38, 427.28; ESI-HRMS [M+H]⁺ calculated for C₃₂H₂₉F₃N₆O₃S, 634.2, found 635.2022.

Synthesis of intermediate 2-(1-((4-(trifluoromethyl)phenyl)sulfonyl-H-indol-3-yl) acetic acid (117) is shown in Scheme 10.

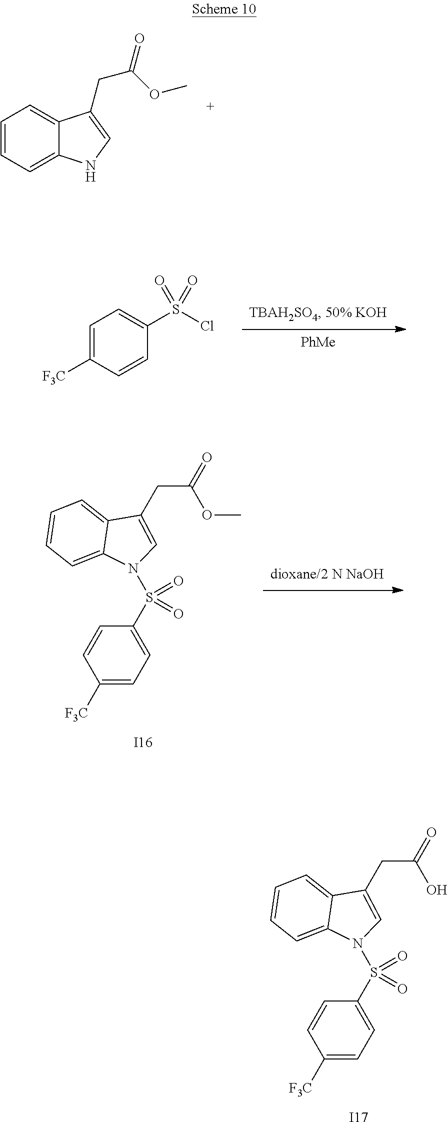

Methyl 2-(1-((4-(trifluoromethyl)phenyl)sulfonyl)-1 H-indol-3-yl)acetate (116) 37.3 mg, 0.0939 mmol) was dissolved in dioxane (1 mL)/water (0.5 mL) then treated with 2N NaOH aqueous solution (0.5 mL) and the reaction was allowed to stir for 0.5 h. The reaction was quenched with 1N HCl until the pH was around 4. The aqueous layer was extracted with ethyl acetate, dried over sodium sulfate, to obtain 117 as an off white solid (31.3 mg, 87% yield). TLC (3% methanol/dichloromethane), R_{f}=0.69; m.p. 196-198° C.; ¹H NMR (400 MHz, CD₃OD) δ 8.092 (d, J=8.4 Hz, 2H), 7.964-7.984 (d, J=8.4 Hz, 1H), 7.782-7.803 (d, J=8.4 Hz, 2H), 7.640 (s, 1H), 7.542-7.562 (d, J=7.8 Hz, 1H), 7.315-7.355 (t, 1H), 7.232-7.271 (t, 1H), 3.682 (s, 2H); ¹³C-NMR (100 MHz, CD₃OD) 175.390, 142.801, 136.448, 136.373, 136.044, 132.443, 128.792, 127.601, 126.092, 125.838, 124.780, 121.131, 119.441, 114.592, 32.205; IR (neat) vₘₐₓ 1690.56, 1272.57, 1318.67, 1170.49, 1121.30, 1057.78, 979.30, 837.70, 749.36, 711.52, 640.54, 603.04, 557.08, 426.66; ESI-HRMS [M+H]⁺ calculated for C17H12F3NO4S, 383.3412, found 384.0497.

Synthesis of intermediate Methyl 2-(1-((4-(trifluoromethyl)phenyl)sulfonyl)-1H-indol-3-yl)acetate (116). Methyl 2-(1Hindol-3-yl)acetate (54.8 mg, 0.290 mmol, 1.0 equiv), 4-(trifluoromethyl)benzenesulfonyl chloride (85.1 mg, 0.348 mmol, 1.2 equiv), and tetrabutylammonium hydrogen sulfate (9.85 mg, 0.029 mmol, 0.1 equiv) were dissolved in toluene (2 mL) and cooled in an ice bath (Scheme 10). A 50% potassium hydroxide solution (400 mL) was added dropwise and the reaction was allowed to stir for 8h with vigorous stirring. The reaction was diluted with ethyl acetate and washed with water and brine. The organic layer was dried over sodium sulfate, concentrated under reduced pressure, and purified via column chromatography using 5% acetone in dichloromethane to produce 16 (37.3 mg, 32% yield) as a clear oil. TLC (5% acetone/dichloromethane), R_{f}=0.62; m.p. 94-96° C.; ¹HNMR (400 MHz, CDCl₃) δ 7.976-8.011 (m, 3H), 7.689 (d, J=8.5 Hz, 2H), 7.579 (s, 1H), 7.502-7.512 (d, J=7.8 Hz, 1H), 7.338-7.380 (m, 1H), 7.265-7.305 (m, 1H), 3.714 (s, 3H); 13C-NMR (100 MHz, CDCl₃) 170.919, 141.610, 135.971, 135.309, 135.086, 130.706, 127.445, 126.620, 125.465, 124.601, 123.953, 119.910, 116.313, 113.709, 52.364, 30.816; 19F-NMR (100 MHz, CDCl₃)-63.358; IR (neat) vₘₐₓ 1442.28, 1737.13, 373.50, 1316.00, 1251.99, 1168.40, 1123.27, 1058.93, 1010.33, 976.94, 839.25, 749.05, 708.96, 605.14, 557.41, 425.79; ESI-HRMS [M+H]⁺ calculated for C₁₈H₁₄F₃NO₄S, 397.3682, found 398.0655.

N-(4-((6-methyl-2-(pyrrolidin-1-yl)pyrimidin-4-yl)amino)phenyl)-2-(2-oxoindolin-3-yl) acetamide (6.30). 4.1 (22.0 mg, 0.0817 mmol, 1.0 equiv) was dissolved in dichloromethane, anhydrous (1.5 mL) and treated with HBTU (40.3 mg, 0.106 mmol, 1. equiv), 2-(2-oxoindolin-3-yl)acetic acid (17.2 mg, 0.090 mmol, 1.1 equiv) and then DIPEA (28.3 mL, 0.163 mmol, 1.0 equiv). The vial was purged with nitrogen and allowed to stir for 24h at RT. The solution was concentrated on to silica gel and purified via column chromatography using 5-20% methanol in dichloromethane to provide 6.30 (16.0 mg, 45% yield) as a white solid. TLC (3% methanol/dichloromethane), R_{f}=0.27; m.p. 170-172° C.; ¹H NMR (400 MHz, CD₃OD) δ 7.626-7.648 (d, J=8.9 Hz, 2H), 7.484-7.506 (d, J=8.9 Hz, 2H), 7.192-7.252 (q, 2H), 6.961-6.999 (t, 1H), 6.907-6.926 (d, J=7.8 Hz, 1H), 5.952 (s, 1H), 3.882-3.915 (m, 1H), 3.581-3.654 (m, 4H), 3.060-3.111 (dd, 1H), 2.746-2.818 (m, 1H), 2.277 (s, 3H), 2.036 (m, 4H); ¹³C-NMR (100 MHz, CD₃OD) 181.633, 170.864, 162.145, 143.659, 136.567, 135.491, 130.594, 129.266, 125.084, 123.288, 122.212, 121.651, 110.921, 96.940, 55.830, 38.068, 36.945, 31.639, 26.245, 20.701, 13.165; IR (neat) vₘₐₓ 3323.06, 1571.06, 1502.57, 1335.88, 1231.30, 816.45, 663.70, 520.32; ESI-HRMS [M+H]⁺ calculated for C₂₅H₂₆N₆O₂, 442.21, found 443.2175.

N-(4-((6-methyl-2-(pyrrolidin-1-yl)pyrimidin-4-yl)amino)phenyl)-2-(1-((4-(trifluoromethyl) phenyl)sulfonyl)-1H-indol-3-yl)acetamide (6.31). 4.1 (62.0 mg, 0.230 mmol, 1.0 equiv) was dissolved in dichloromethane, anhydrous (6.0 mL) and treated with DMAP (28.1 mg, 0.230 mmol, 1.1 equiv), DCC (59.3 mg, 0.288 mmol, 1.25 equiv) and 2-(2-chloroquinolin-4-yl)acetic acid (56.0 mg, 0.253 mmol, 1.0 equiv). The vial was purged with nitrogen and allowed to stir for 24h at RT. The solution was filtered through a pad of celite and concentrated on to silica gel. Purification via column chromatography using 1:1 ethyl acetate/dichloromethane and 3% methanol provided 6.31 (13.5 mg, 57% yield) as a white solid. TLC (3% methanol/dichloromethane), R_{f}=0.58; m.p. 260-262° C.; ¹H NMR (400 MHz, CDCl₃) δ 8.199-8.220 (d, J=8.5 Hz, 1H), 7.976-7.997 (d, J=8.5 Hz, 1H), 7.798-7.839 (t, 1H), 7.658-7.710 (m, 3H), 7.553 (s, 1H), 7.475-7.497 (d, J=8.9 Hz, 2H), 5.859 (s, 1H), 4.243 (s, 2H), 3.558-3.591 (m, 4H), 2.214 (s, 3H), 1.976-2.009 (m, 4H); ¹³C-NMR (100 MHz, CDCl₃) 167.354, 165.894, 161.314, 160.216, 150.517, 147.781, 145.224, 136.193, 133.217, 130.757, 128.675, 127.402, 126.491, 124.017, 123.331, 121.492, 120.797, 116.000, 93.281, 46.653, 40.236, 25.483, 23.457; IR (neat) vₘₐₓ 1755.53, 1658.71, 1612.71, 1548.21, 1504.15, 1403.49, 1251.05, 1138.16, 890.44, 696.38, 517.52 ESI-HRMS [M+H]⁺ calculated for C₂₆H₂₅ClN₆O, 472.18, found 473.1834.

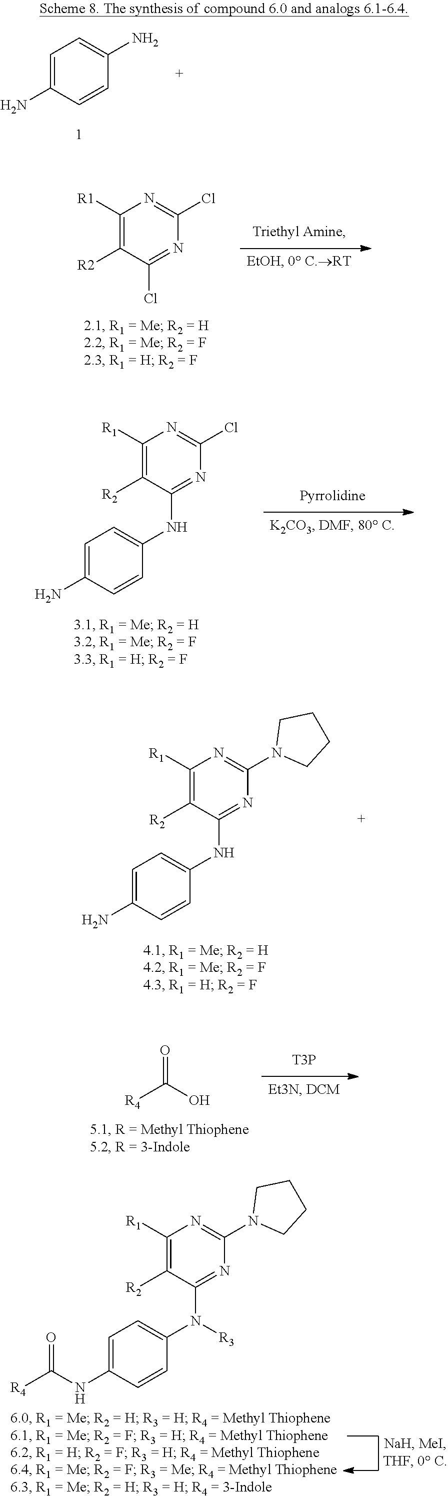

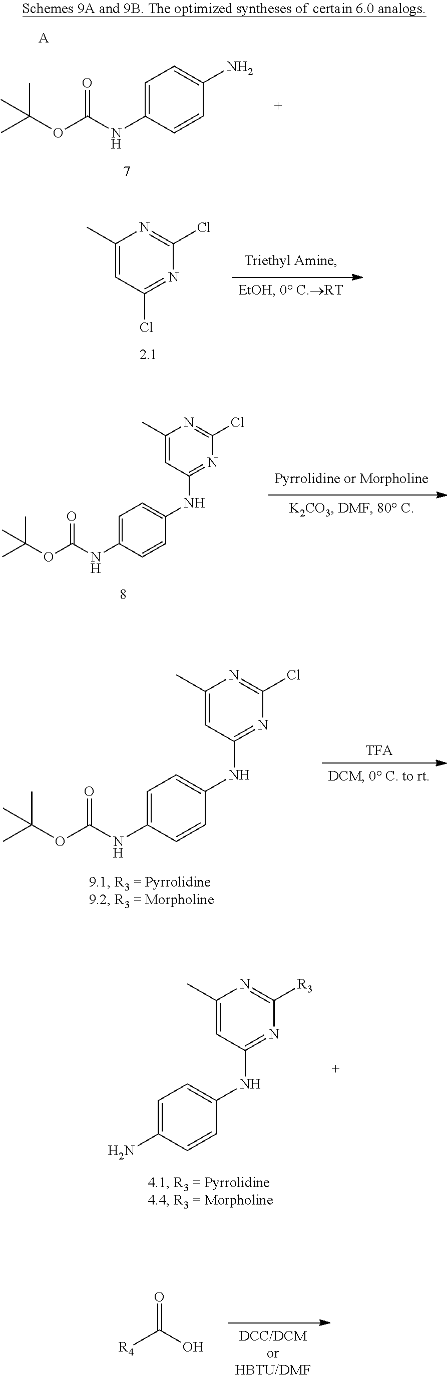

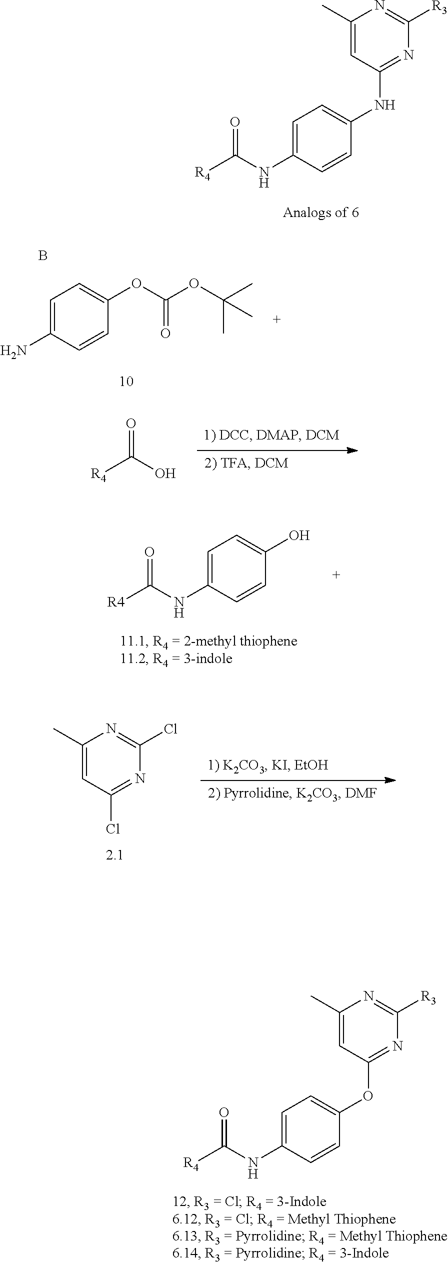

### Example 14: Additional Synthetic Examples

### Exemplary Synthesis of Benzyl Analogues of Compound 6

Scheme 11 illustrated an exemplary synthesis of benzyl analogues of compound 6. The compound numbers below refer to the compound numbers in Scheme 10.

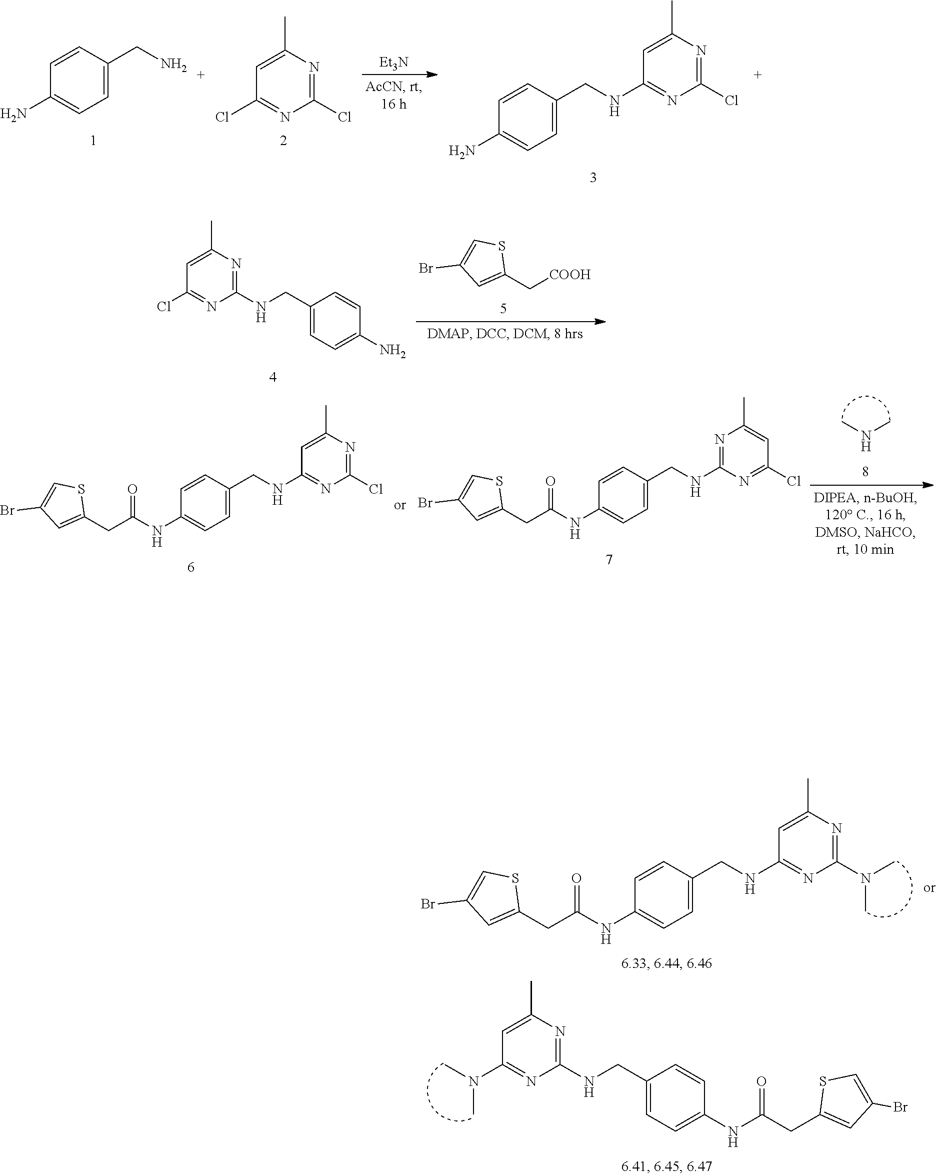

Synthesis of Intermediates 3 and 4 (Scheme 11)

1.5 g of 4-aminobenzylamine, 1, (12.28 mmol) was added to 25 mL acetonitrile solution of 2,4-dichloro-6-methylpyrimidine, 2, (2.0 g, 1 eq) and triethylamine (3.43 mL, 2 eq). The resulting solution was stirred at room temperature overnight, then diluted with water and saturated sodium bicarbonate solution followed by extraction with ethyl acetate. The combined organic layers were dried over anhydrous sodium sulfate, concentrated onto silica gel and purified with Teledyne Combiflash chromatography system to afford the 4-position isomer, 3, (orange yellow solid, 1.84 g, 60.3%) and 2-position isomer, 4, (pale yellow solid, 0.66 g, 5.4%).

Synthesis of intermediates 6 and 7 (Scheme 11)

To a slurry of 3 (500 mg) and DMAP (1.1 eq) in freshly dried DCM (50 mL), was added DCC (1.2 eq) and 4-bromothiophene acetic acid, 5, (1.2.eq). After stirring at room temperature for 8 h, the reaction mixture was filtered through a celite pad and concentrated on silica gel. Purification via silica gel flash chromatography gave 6 as brown solid in quantitative yield. The reaction protocol was repeated to obtain 7 as beige solid in quantitative yield.

Synthesis of CHD1Li 6.33, 6.40, 6.41, 6.44-6.47 and 6.57 (Scheme 11)

6 or 7 (150 mg) were each treated with 3 mL n-BuOH and DIPEA (3 eq) then stirred at room temperature for few minutes. The corresponding cyclic 2° amine was added and the reaction mixture was refluxed at 120° C. overnight, then concentrated in vacuo to dryness. The residue was dissolved in ethyl acetate and basified with saturated sodium bicarbonate solution. The combined ethyl acetate extracts were dried over anhydrous sodium sulfate, concentrated on silica gel and purified via silica gel flash chromatography. Subsequently, the brown solid products were dissolved in DMSO and treated with saturated sodium bicarbonate solution to give an off white precipitate. Which was collected by filtration to afford the desired CHD1Li.

### Exemplary Synthesis of Triazolopyridine Analogues of Compound 6.11

Scheme 12 illustrates an exemplary synthesis of triazolopyrimidine analogues of compound 6.11. Compound numbers referenced below refer to those in Scheme 12.

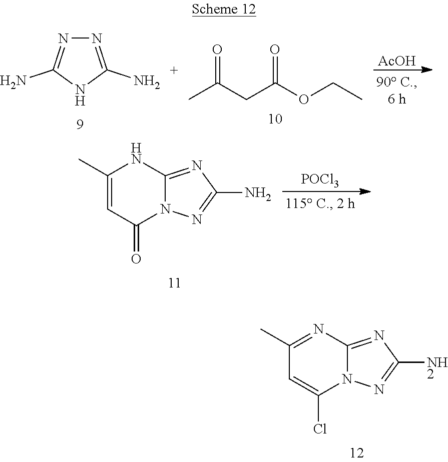

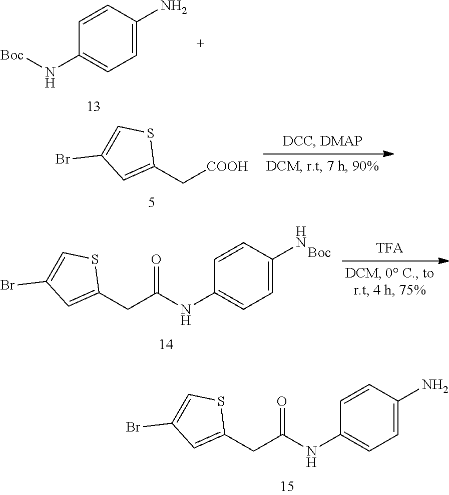 [SMILES: CC(C)(C)OC(=O)Nc1ccc(N)cc1 ; O=C(O)Cc1cc(Br)cs1 ; Nc1ccc(NC(=O)Cc2cc(Br)cs2)cc1 ; Nc1ccc(NC(=O)Cc2cc(Br)cs2)cc1]

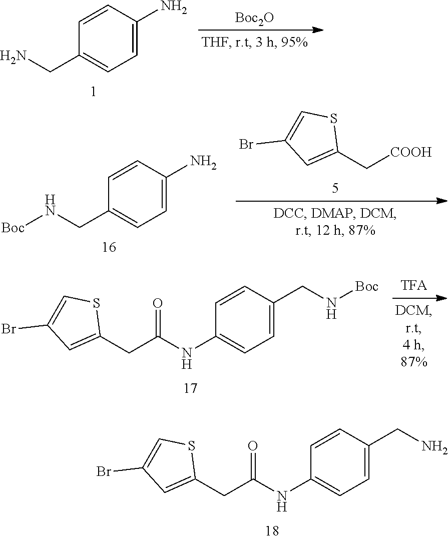 [SMILES: "Boc2O" ; NCc1ccc(N)cc1 ; CC(C)(C)OC(=O)NCc1ccc(N)cc1 ; O=C(O)Cc1cc(Br)cs1 ; CC(C)(C)OC(=O)NCc1ccc(NC(=O)Cc2cc(Br)cs2)cc1 ; NCc1ccc(NC(=O)Cc2cc(Br)cs2)cc1]

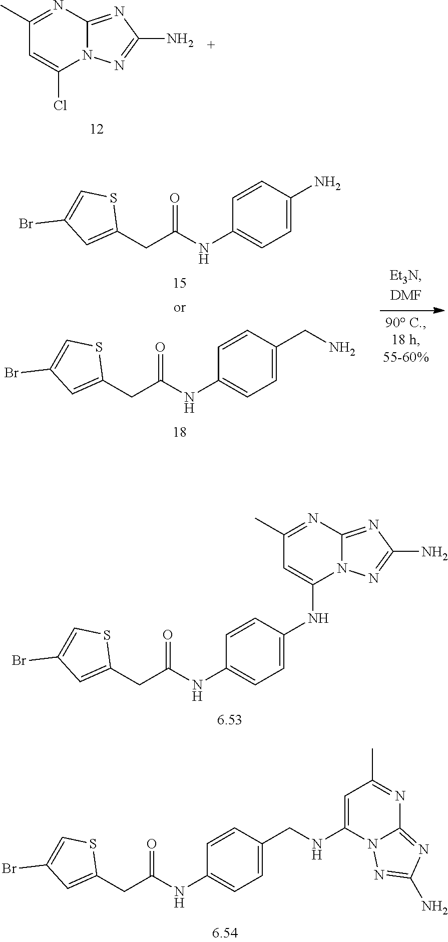 [SMILES: Cc1cc(Cl)n2nc(N)nc2n1 ; Nc1ccc(NC(=O)Cc2cc(Br)cs2)cc1 ; NCc1ccc(NC(=O)Cc2cc(Br)cs2)cc1 ; Cc1cc(Nc2ccc(NC(=O)Cc3cc(Br)cs3)cc2)n2nc(N)nc2n1 ; Cc1cc(NCc2ccc(NC(=O)Cc3cc(Br)cs3)cc2)n2nc(N)nc2n1]

Synthesis of Intermediate 12 (Scheme 12)

1.0 g of triazole 9 and 1.28 mL of ethyl acetoacetate 10 were added to 30 mL acetic acid and refluxed at 130° C. for 6 h. The reaction mixture was diluted with ethanol and kept at −20° C. overnight. The resulting white precipitate was filtered in vacuo to afford 11 in quantitative yield. Subsequently, 11 (0.7 g) was added in portions to 5 mL POCl₃ at 0° C. After stirring at this temperature for few minutes, the mixture was refluxed at 115° C. for 2 h then concentrated in vacuo to dryness. The brick red residue was re-dissolved in DCM, basified with saturated NaHCO₃ and filtered in vacuo to give 0.51 g of 12 as an orange solid.

Synthesis of intermediate 15 (Scheme 12)

The required acetamide intermediate 14 was synthesized in 90% yield as a beige solid following the synthetic procedure for 7. Subsequent boc-deprotection with DCM-TFA (1:1) solution afforded 15 in 75% yield as a brown solid.

Synthesis of intermediate 18 (Scheme 12)

A solution of 1 (1.0 mL) in dry THF was treated with dropwise addition of boc-anhydride (1 eq) pre-dissolved in dry THF. The reaction mixture was stirred under nitrogen for 2 h then concentrated to dryness under vacuum. The resulting yellow solid was washed with CHCl₃-hexane (1:2) to give 16 in quantitative yield. Analogous amide coupling and boc-deprotection as in 15 above afforded 18 in 87% yield as an off-white solid.

Synthesis of 6.53 and 6.54 (Scheme 12)

70 mg of 12 was added to a DMF solution (5 mL) of 15 or 16 (1 eq) containing triethylamine (2 eq). The reaction mixture was heated at 90° C. for 18 h then poured onto crushed ice, stirred and filtered in vacuo to obtain crude products as brown solids. Purification with silica gel Combiflash chromatography afforded pure 6.53 and 6.54 as off-white solids in 55 and 60% yields, respectively.

SCHEME 13 illustrates exemplary synthesis of compounds 6.55 and 6.56.

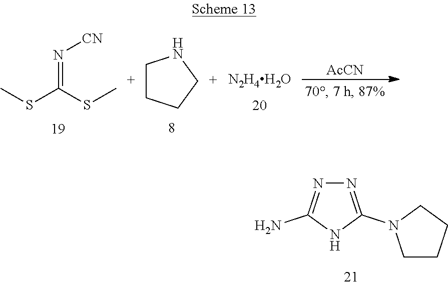

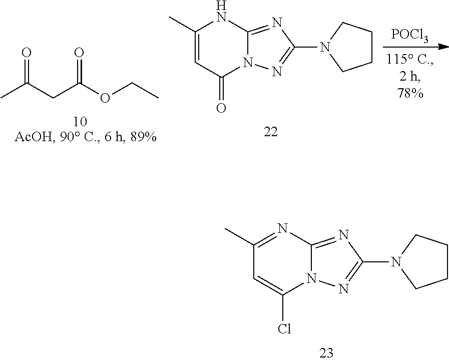

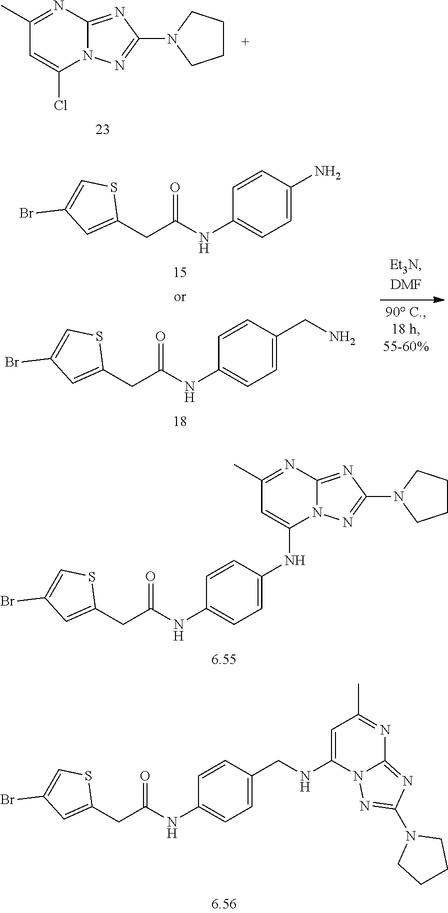 [SMILES: Cc1cc(Cl)n2nc(N3CCCC3)nc2n1 ; Nc1ccc(NC(=O)Cc2cc(Br)cs2)cc1 ; NCc1ccc(NC(=O)Cc2cc(Br)cs2)cc1 ; Cc1cc(Nc2ccc(NC(=O)Cc3cc(Br)cs3)cc2)n2nc(N3CCCC3)nc2n1 ; Cc1cc(NCc2ccc(NC(=O)Cc3cc(Br)cs3)cc2)n2nc(N3CCCC3)nc2n1]

In the following description, compound numbers refer to Scheme 13.

Synthesis of Intermediate 22

A solution of 1.024 g (7 mmol) of dimethyl cyanocarbonimidodithioate 10 and pyrrolidine (1.0 eq) in acetonitrile (5 mL) were refluxed at 85° C. for 2 h. Then, hydrazine monohydrate (1.5 eq) was added and refluxing continued for 5 h. After cooling to room temperature, the resulting pale pink precipitate was filtered in vacuo while washing with cold diethyl ether to obtain 21 in quantitative yield. Subsequently, 500 mg of 21 in acetic acid (7 mL) was treated with 10 (1 eq) and refluxed at 90° C. for 6 h. The resulting precipitate was filtered while washing with cold diethyl ether to give 22 as an off-white solid in 89% yield. The conversion of 22 to 23 achieved in 78% yield using the same procedure as in 12 above.

Synthesis of 6.55 and 6.56

6.55 and 6.56 were synthesized according the method described for 6.53 and 6.54 above.

### I. Synthesis of 1,3-propanesultam analogue (Scheme 14)

In the following description, compound numbers refer to Scheme 14. To a 5 mL acetonitrile solution of boc-protected p-phenylenediamine (250 mg, 1.2 mmol) and Et₃N (2 eq), 2,4-dichloro-6-methylpyrimidine was added at RT and the reaction mixture was stirred for 12 h. Routine workup with water, saturated NaHCO₃, and extraction with DCM, followed by purification with flash column chromatography using EtOAc-hexane afforded intermediate 1 as an off-white solid in excellent yield (90%). Subsequently, 1 (200 mg, 0.597 mmol) was dissolved in 5 mL dioxane and treated with CSCO₃ (3 eq), Pd₂(dba)₃ (0.5 eq) and xantphos (1.5 eq) at RT. After refluxing at 110° C. for 12 h, the reaction mixture was filtered through a pad of celite and concentrated on silica gel for flash chromatography using EtOAc-hexane. The boc-protected product was redissolved in 5 mL DCM, treated with 5 mL TFA and stirred at RT for 3 h. Routine workup with EtOAc and purification via flash chromatography using EtOAc-hexane gave intermediate 2 as a beige solid in 40% yield over 2 steps. Finally, to a solution of 2 (50 mg, 0.157 mmol) and DMAP (1.2 eq) in freshly dried DCM, DCC (1.2 eq) and 4-bromothiophene acetic (1.2 eq) were added simultaneously. After stirring at RT for 12 h, the reaction mixture was filtered through a pad of celite and concentrated on silica gel. Flash chromatography using methanol-chloroform afforded the desired CHD1Li (3/158) in 60% yield as an off-white solid.

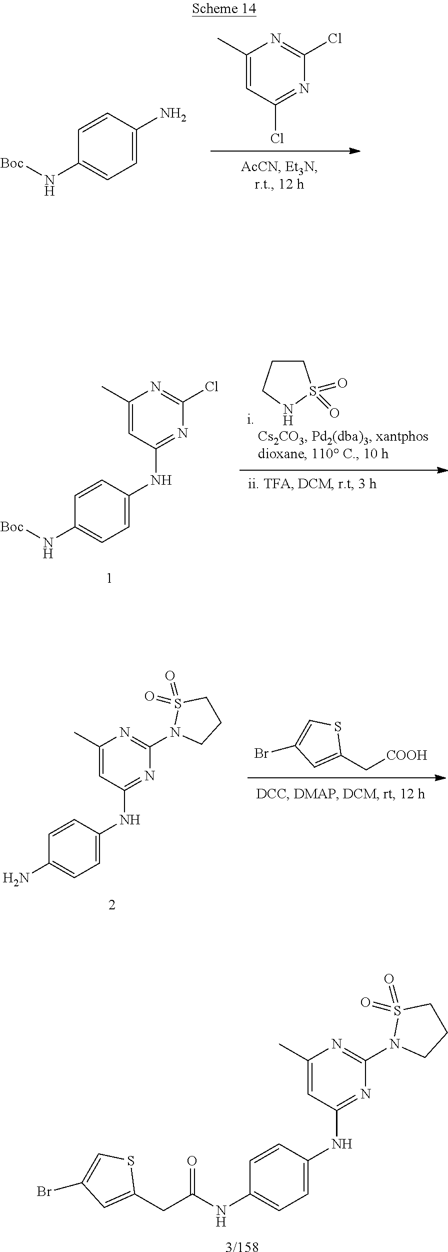

### Exemplary Synthesis of amide and urea analogues (Scheme 15)

In the following, compound numbers are referenced to Scheme 15.

Compound 6

2-chloro-6-methylpyrimidin-4-amine (500 mg, 3.48 mmol) and pyrrolidine (3 eq) were added simultaneously to a flask containing K₂CO₃ (1.05 eq) and DMF (2 mL). After refluxing at 75° C. for 8 h, the reaction mixture was poured into crushed ice and stirred vigorously. The resulting precipitate was collected via vacuum filtration to give intermediate 4 as an off-white solid in 76% yield. Thereafter, to a stirring solution of 4 (150 mg, 0.842 mmol), triethylamine (1.2 eq) and DMAP (1.0 eq) in freshly dried DCM at RT, 4-nitrobenzoyl chloride (1.0 eq) was added and stirring continued for 16 h. The mixture was then poured into water and basified with saturated NaHCO₃ solution. The aqueous layer was extracted with DCM and combined organic extracts were dried over anhydrous Na₂SO₄ and concentrated on silica gel. Flash chromatography with EtOAc-hexane gave the nitro precursor of 5 as a yellow solid. 100 mg (0.306 mmol) of this nitro precursor in 8 mL EtOH was added to a mixture of Fe powder (7 eq) and NH₄Cl (3.5 eq) in 2 mL water. The reaction mixture was refluxed at 80° C. for 3 h, filtered through a pad of celite and concentrated to dryness in vacuo. The residue was suspended in ethyl acetate and extracted with water. The ethyl acetate layer was dried over anhydrous Na₂SO₄ and concentrated in vacuo to afford crude intermediate 5 as a beige solid. 5 was treated with 4-bromothiophene acetic akin to 3 above to afford 6 as an off-white solid.

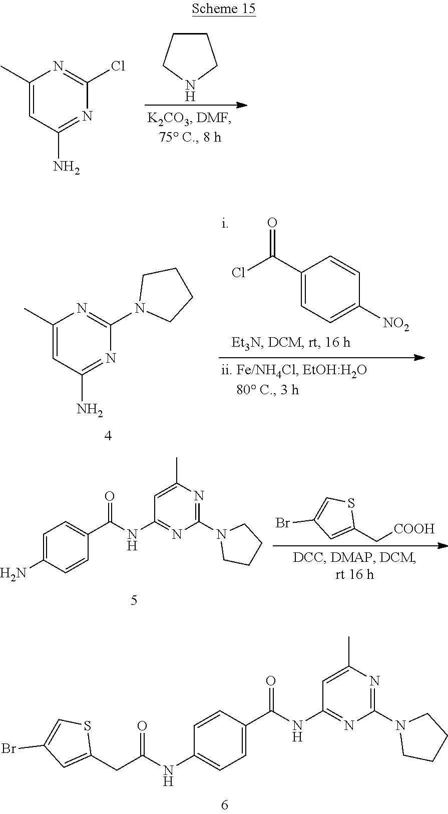

Compound 8 (Scheme 16)

In the following description, compound numbers refer to Scheme 16. Compound 8 was synthesized using the protocol for compound 6 synthesis. The required acid chloride was prepared by refluxing 250 mg of 4-nitrophenyl acetic acid in excess thionyl chloride.

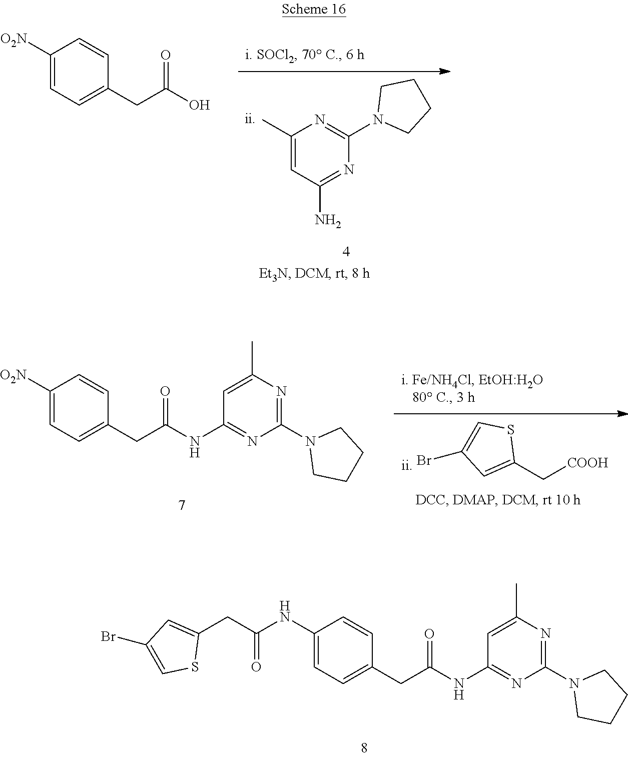

Compound 10 (Scheme 17)

In the following description, compound numbers refer to Scheme 17. Compound 10 was synthesized as an off-white solid using the protocol for compound 6 synthesis. The required nitro precursor of 9 was prepared as follows; intermediate 4 (150 mg, 0.842 mmol), 4-nitrophenyl isocyanate (1.0 eq) and triethylamine (3 eq) were refluxed in 5 mL dioxane at 110° C. for 16 h. After cooling to RT, the resulting precipitate was filtered while washing with 5 mL cold diethyl ether to afford the desired nitro precursor as a yellow solid.

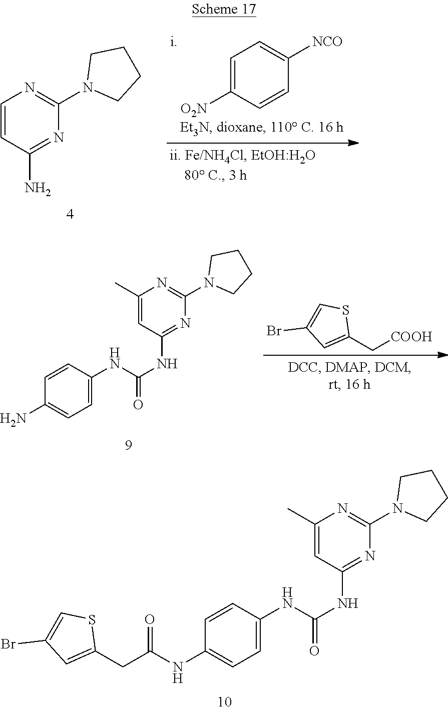

Compound 13 (Scheme 18)

The urea intermediate 11 was prepared as described for 9 while its conversion to 12 was achieved using the procedure for intermediate 4. Thereafter, 80 mg of 12 (0.177 mmol) in 3 mL n-BuOH was treated with pyrrolidine (3 eq) and refluxed at 120° C. for 16 h. The reaction mixture was concentrated in vacuo, redissolved in ethyl acetate and basified with saturated NaHCO₃ solution. The aqueous layer was extracted with EtOAc and the combined organic extracts were dried over anhydrous Na₂SO₄. Flash chromatography purification afforded 13 as beige solid.

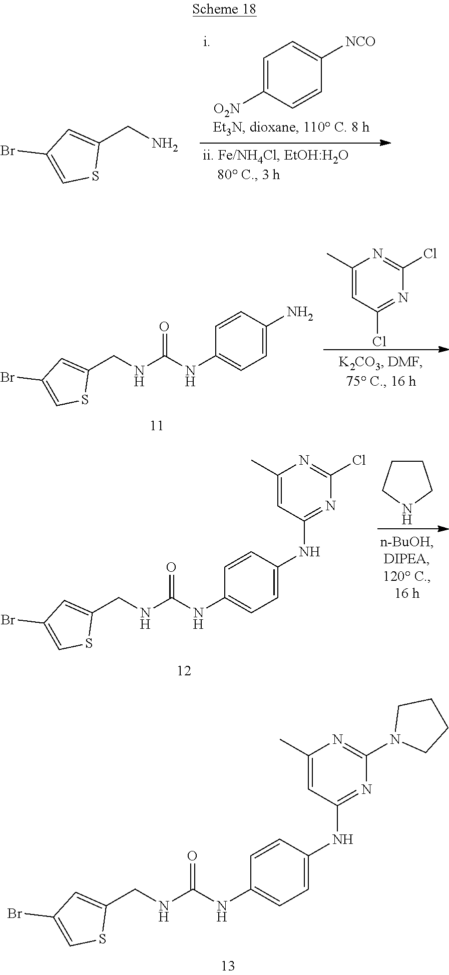

### Example 15: In Vitro Biological Evaluation of CHD1L Inhibitors

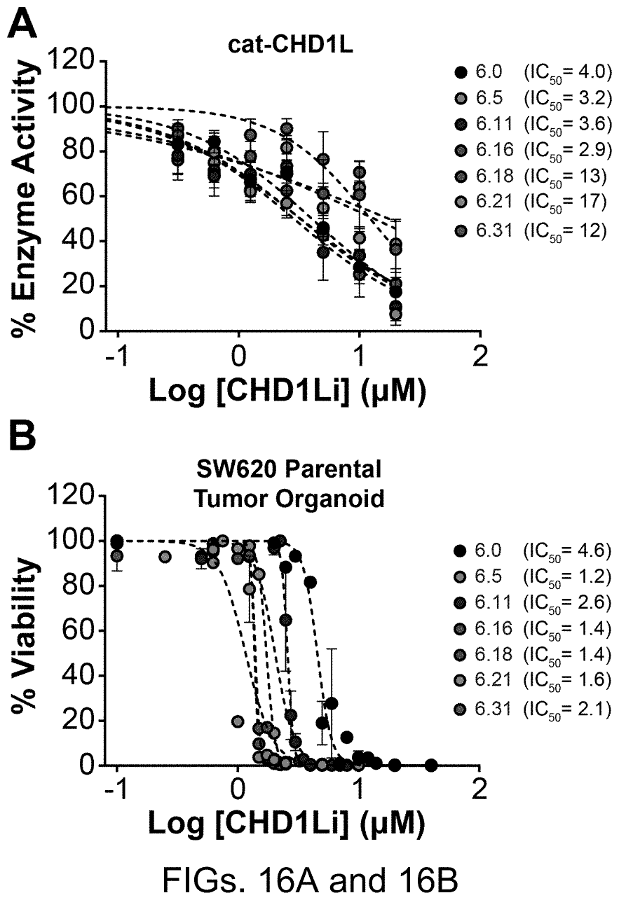

CHD1L inhibitors were assessed for their ability to inhibit the recombinant catalytic domain of CHD1L (cat-CHD1L). (See Abbott et al., 2020) The results of these studies demonstrate feasibility of designing drugs based on the pharmacophore of compound 6.0 (FIGS. 16A and 16B). Notably, the more potent CHD1L inhibitors were compounds 6.5, 6.11, 6.16, 6.18, 6.21, and 6.31 (FIG. 16A), which also displayed similar increases in cytotoxic potency in SW620 parental tumor organoids compared to 6.0 (FIG. 1B). Structures of compounds 6.5, 6.11, 6.16, 6.18, 6.21, and 6.31 are found in Scheme 1.

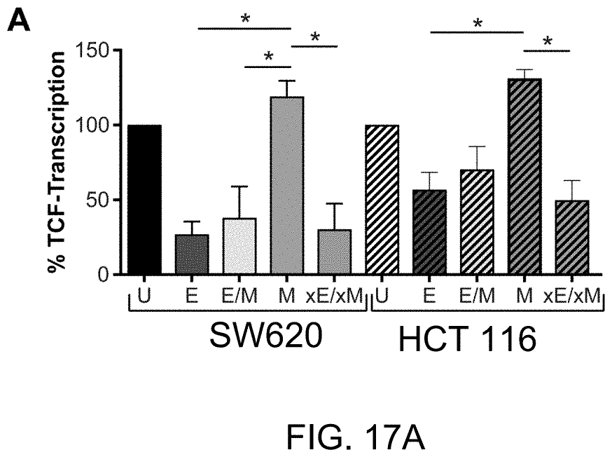

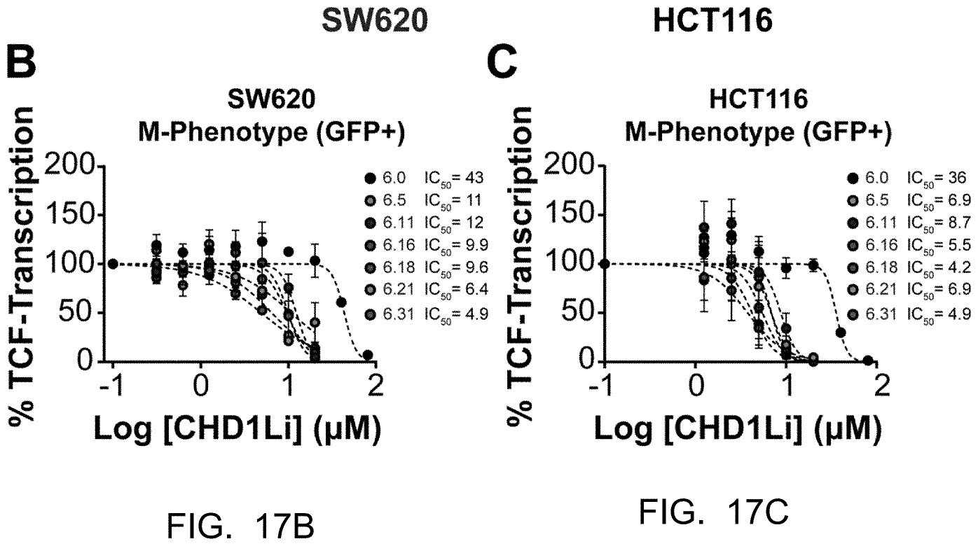

A novel fluorescent EMT dual-reporter suitable for 2D and 3D high-content imaging has been developed that is an effective tool to measure EMP in real time while simultaneously tracking the spectrum of EMT cellular phenotypes. (See also Zhou et al., 2016) EMT phenotypes can be isolated by FACS based on dual-reporter fluorescence and interrogated as individual cell populations in long-term culture, including stable xE/xM (RFP−/GFP−), and quasi-EMT populations E (RFP+), E/M (RFP+/GFP+), and M (GFP+). Isolated EMT phenotypes display unequivocal differences morphologically and metabolically, and these phenotypic differences are driven by TCF-transcription. The correlation between cytotoxicity and CHD1L inhibition are consistent with the inhibition of CHD1L mediated TCF-transcription in CRC. (Esquer et al., 2021; Abbott et al., 2020) In particular, we demonstrate that the more tumorigenic CSC quasi-M-phenotype has upregulated TCF-transcription compared to the other EMT phenotypes (FIG. 17A). Thus, we treated M-phenotype SW620 and HCT116 cells with CHD1L inhibitors and observed a dose-dependent decrease in TCF-transcription with all listed compounds with inhibition concentration 50% (IC₅₀) values in the low micromolar concentration (FIGS. 17B and 17C, respectively).

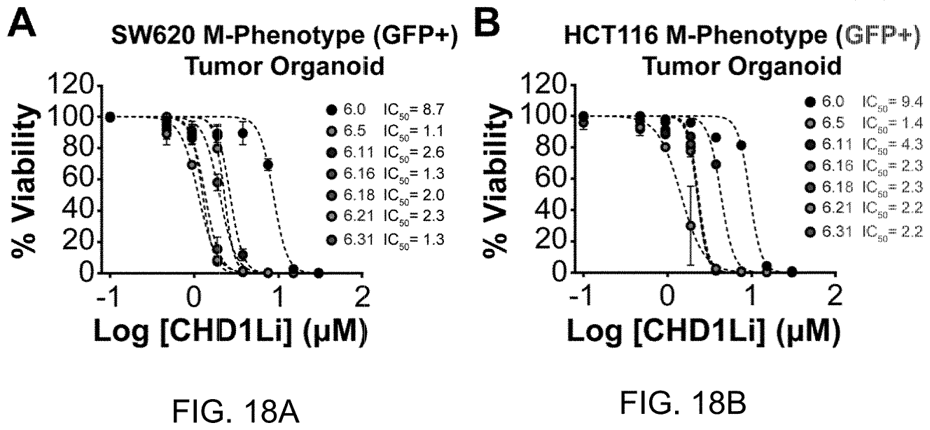

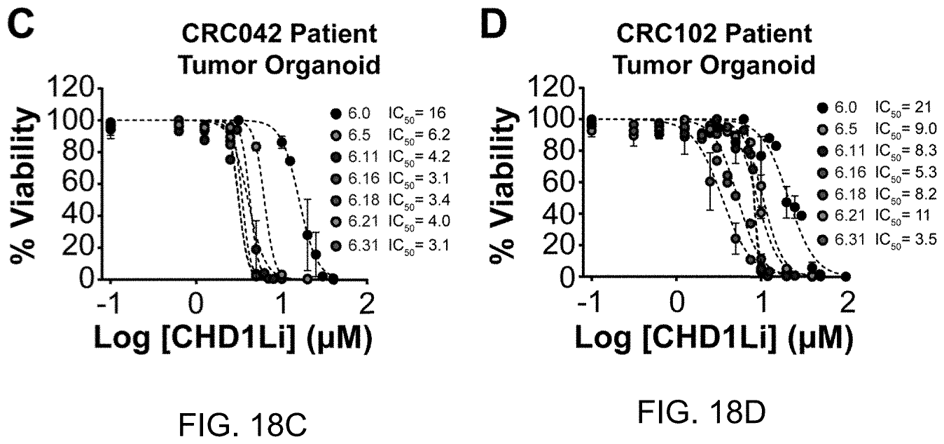

CHD1Li cytotoxicity was then measured in both cell line (SW620 and HCT116) and patient cell derived (CRC042 and CRC102) tumor organoids (FIGS. 18A-18D). CHD1L inhibitors displayed potent antitumor activity in SW620 and HCT116 M-Phenotype tumor organoids, inhibiting cell viability at low micromolar IC₅₀ values (FIGS. 18A and 18B). Likewise, CHD1Li had potent cytotoxicity against CRC042 and CRC102 patient tumor organoids (FIGS. 18C and 18D). These results underscore the potent antitumor activity of CHD1Li in a variety of CRC cell models, including CRC patient tumor organoids.

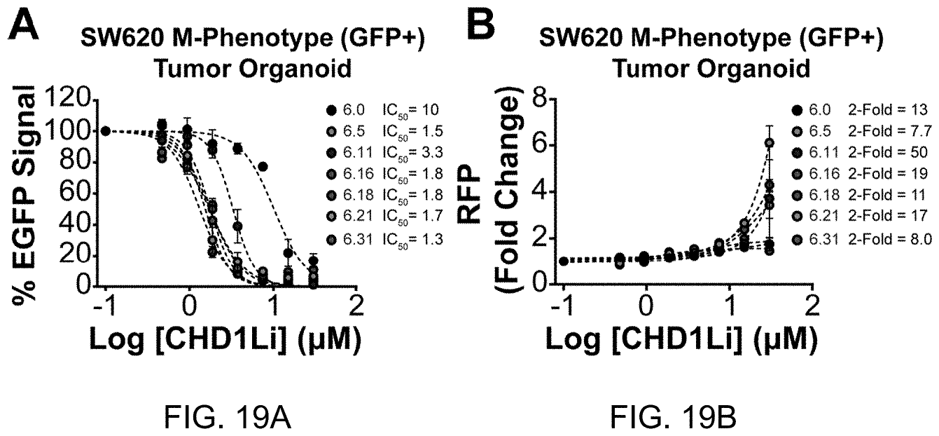

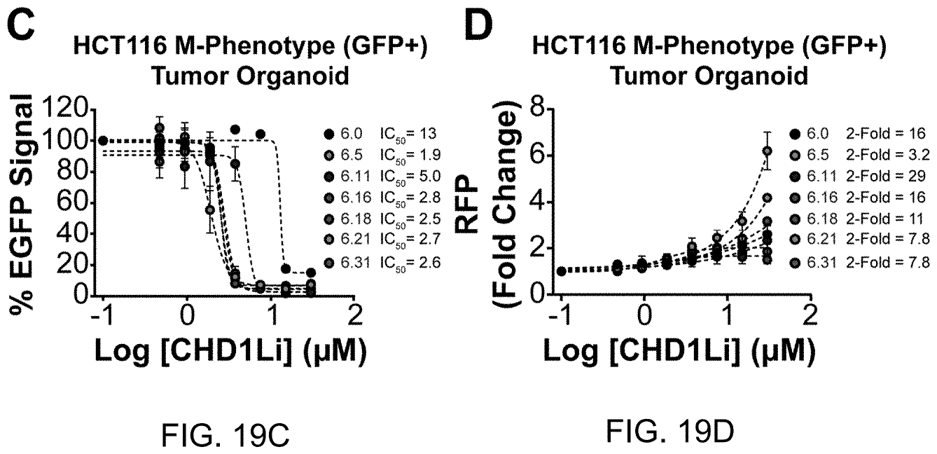

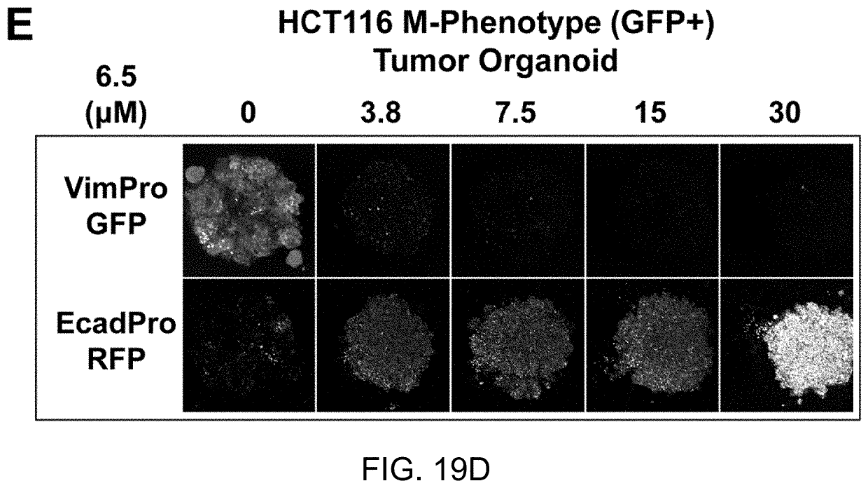

CHD1L inhibitors were then evaluated for their ability to inhibit EMT and/or induce mesenchymal-epithelial transition (MET, i.e. reverse EMT) by simultaneously measuring the fluorescent signal of the EMT dual-reporter (VimPro-GFP and EcadPro-RFP), using the high-content imaging methodology previously described. (Zhou et al., 2016) Indeed, CHD1L inhibitors prevent EMT and induce MET in SW620 and HCT116 M-Phenotype tumor organoids characterized by dose dependent downregulation of vimentin with concomitant upregulation of E-cadherin expression (FIGS. 19A-19E). To quantify E-cadherin upregulation, resulting from CHD1Li, we generated a non-linear regression model to determine the dose at which a 2-fold increase of RFP fluorescent signal occurs. Representative dual-reporter M-phenotype HCT116 tumor organoid images treated with compound 6.5 are shown (FIG. 19E). A marked down-regulation of the VimPro-GFP is observed, while an upregulation of EcadPro-RFP is noted as the treatment dose increases and is consistent with our previous results of CHD1Li 6.0 induced MET. (See Abbott et al., 2020)

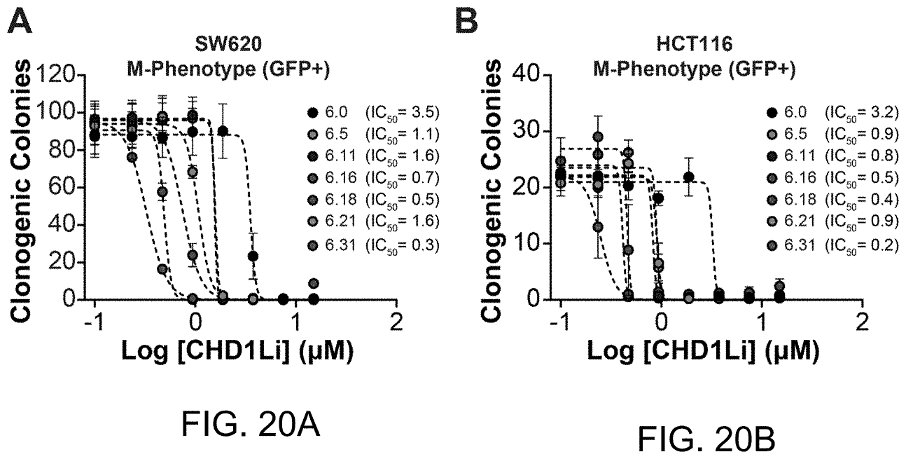

TCF-driven EMT is linked as a mechanism enabling mesenchymal cells with increased CSC traits including self-renewal, resistance to apoptosis, and increased metastatic potential. (Scheel et al., 2012; Chaffer et al., 2016) This fact is consistent with our results that isolated M-phenotype tumor cells have significant increased CSC stemness. (See also Esquer et al., 2021) We have shown that compound 6.0 significantly inhibits CSC stemness (See also Abbott et al., 2020) based on the clonogenic colony formation assay. (Esquer et al., 2020; Franken et al., 2006) Thus, we evaluated CHD1L inhibitors for their ability to inhibit CSC stemness in SW620 and HCT116 M-Phenotype cells, using a high-content imaging. (Esquer et al., 2020) CHD1L inhibitors effectively inhibited colony formation over a low M to nM range (FIGS. 20A and 20B). CHD1L inhibitor 6.31 was the most potent of the compounds assessed with an IC₅₀ value of 300 nM in SW620 cells and 200 nM in HCT116 cells. CHD1L inhibitors prove to be effective antitumor agents that prevent CHD1L-mediated TCF-transcription that in turn inhibits EMT and induces MET, resulting in loss of CSC stemness while promoting cytotoxicity to tumor cells.

### Example 16: In Vivo Biological Evaluation of CHD1L Inhibitors

We described that CHD1Li 6.0 has a good in vivo disposition, including a plasma half-life of 3 h in mice. (See above, also Abbott et al., 2020) We considered that the half-life of 6.0 may be detrimentally affected due to liver metabolism of the thiophene ring. Thiophene rings may form reactive metabolites (e.g., by S-oxidation), and substituted thiophenes are generally more stable to liver metabolizing enzymes. (Gramec et al., 2014) Moreover, 6.0 does not display any liver toxicity when treating mice at a maximum tolerated dose of 50 mg/kg by intraperitoneal (i.p.) administration daily over five days. (See also, Abbott et al., 2020) Thus, thiophene reactive metabolites leading to toxicity does not appear to be a limiting adverse effect. Prior to conducting in vivo studies with CHD1L inhibitors, we conducted in vitro mouse microsomal stability studies with select compound, including 6.0, 6.4, 6.5, 6.11, and 6.31. CHD1L inhibitor 6.11 proved to be the most metabolically stable of these compounds when exposed to microsomes with a 2-fold longer half-life of 130.3 minutes compared to 67.2 minutes for compound 6.0. Therefore, 6.11 was prioritized for in vivo evaluation.

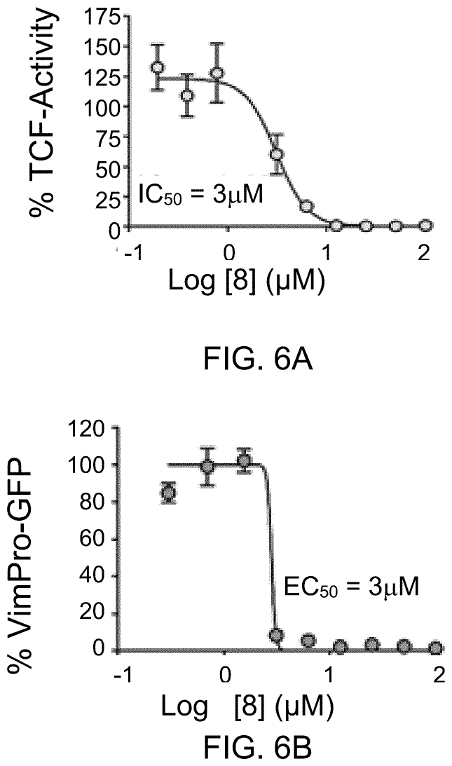

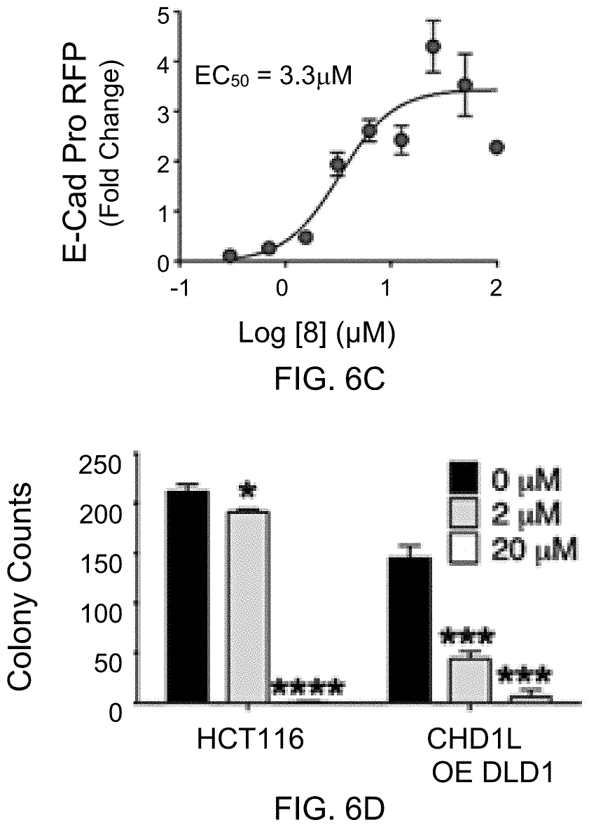

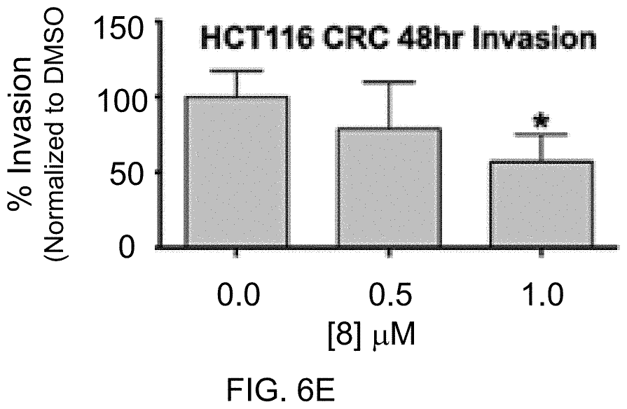

As described in more detail above, using CD-1 mice, we administered 6.11 by i.p. injection at a dose of 50 mg/kg and assessed the pharmacokinetics (PK) of 6.11, including elimination half-life (t_{1/2λ}) from plasma, and liver and fat tissues. The t_{1/2λ} of 6.11 in the plasma and tissues is 8 h, which is a 2.7-fold longer half-life than 6.0. Next, using the same dose, we assessed the oral bioavailability of 6.11 after oral gavage (p.o.) and found that 6.11 is oral bioavailable with 44% uptake in the plasma and a t_{1/2λ} of 8 h (FIG. 6B). The in vivo half-life 6.11 is consistent with its in vitro microsomal stability, indicating that the bromothiophene moiety of 6.11 significantly improves the in vivo PK by increasing its stability to liver enzymes compared to 6.0.

### Example 17: Experimental Methods

General Experimental Methods. All commercial chemicals were used as supplied unless otherwise stated. All solvents used were dried and distilled using standard procedures. Thin layer chromatography (TLC) was performed using Aluminum backed plates coated with 60A Silica gel F254 (Sorbent Technologies, Norcross, Ga., USA). Plates were visualized using a UV lamp (λmax=254 nm). Column chromatography was carried out using 230-400 mesh 60A silica gel or using a Teledyne Isco Combiflash next gen 300+ chromatography system with high performance gold columns. NMR spectra were recorded on a Bruker Avance III 400 (¹H 400 MHz, ¹³C 100 MHz). All chemical shifts are recorded in parts per million (ppm), referenced to residual solvent frequencies (¹H NMR: Me4Si=0, CDCl₃=7.26, D20=4.79, CD₃OD=4.87 or 3.31, DMSO-d₆=2.50, Acetone-d₆=2.05 and ¹³C NMR: CDCl₃=77.16; CD₃OD=49.0, DMSO-d₆=39.5, Acetone-d₆=29.9 Coupling constants (J) values are expressed in hertz (Hz). The following splitting abbreviations were used: s=singlet, d=doublet, t=triplet, q=quartet, p=pentet, m=multiplet, br=broad, dd=doublet of doublets, dt=doublet of triplets, td=triplet of doublets. Melting points (m.p.) were determined using a Stuart melting point apparatus (SMP20). Infrared (IR) spectra were recorded on a Bruker ALPHA platinum ATR (oils and solids were examined neat). Compounds purity (>95%) was measured using a Shimadzu prominence HPLC equipped with a photodiode array detector (PDA) and Luna^{R} Omega Polar C18 column (5 μm, 100 Å, 250 mm×4.6 mm). Using a flow rate of 0.525 mL/min, compounds were eluted with a gradient of water/methanol, with 0.1% TFA in water/0.1% TFA in methanol over 0 to 25 min. High resolution mass spectrometry (HRMS) were recorded using Q Exactive mass spectrometer (ThermoFisher, San Jose, Calif.) operated independently in positive or negative ion mode, scanning in full MS mode (2 μscans) from 150 to 1500 m/z at 140,000 resolution, with 4 kV spray voltage, 45 sheath gas, 15 auxiliary gas. Acquired data were then converted from raw to mzXML file format using Mass Matrix (Cleveland, Ohio).

CHD1L Enzyme ATPase Assay. CHD1L enzyme inhibition assay was performed as described previously (Abbott et al., 2020). All reactions were carried out using low volume nonbinding surface 384-well plates (Corning Inc., Corning, N.Y.). 800 nM cat-CHD1L, 200 nM mononucleosome (Active Motif, Carlsbad, Calif.), and various concentrations of inhibitors were preincubated at 37° C. for 10 min in 1× buffer containing 50 mmol/L Tris pH 7.5, 50 mmol/L NaCl, 5 mmol/L MgCl₂, 2 mmol/L DTT, and 5% glycerol. The reaction was initiated by the addition of 10 μmol/L ATP (New England Biolabs, Ipswich, Mass.) to a total volume of 10 μL and incubated at 37° C. for 1 hour. ATPase activity was measured by adding 500 nmol/L of Phosphate Sensor (ThermoFisher, Waltham, Mass.) measuring excitation (430 nm) and emission (450 nm) immediately on an Envision plate reader (PerkinElmer, Waltham, Mass.). Background signal was determined by using all assay components excluding the enzyme.

Cell lines. Cell lines were purchased directly from ATCC and used as indicated. Engineered cell lines previously reported were STR profiled for authenticity. All cell lines were tested for bacterial and mycoplasma contamination before use. Deidentified patient sample cells were obtained from the CU Cancer Center GI tissue bank.

Cell Culture. SW620 and HCT116 cell lines were obtained from American Type Culture Collection (ATCC) (Manassas, Va.) and grown in RPMI-1640 media supplemented with 5% fetal bovine serum (FBS) in a humidified incubator at 37° C. and 5% CO₂. Cells were expanded in 10 cm² tissue culture-treated dishes (ThermoFisher) following ATCC protocols. Epithelial-Mesenchymal transition (EMT) dual reporter cell lines (SW620 and HCT116 E, E/M, and M) were generated, characterized, and maintained as previously outlined (PMID: 33742123). Both wildtype and dual reporter cell lines were harvested and prepared for experiments by aspirating media, washing with 10 mL PBS, detaching with 1 mL of Trypsin-EDTA at 0.25% (ThermoFisher), and neutralizing with 4 mL of complete growth medium. Cells were counted using a Bio-Rad TC20 automated cell counter (Bio-Rad, Hercules, Calif.) by Trypan Blue (1:1) live/dead cell exclusion.

Cell Line Tumor Organoid Culture. Tumor organoids were prepared by plating at 2,000 cells/well into CellCarrier Spheroid Ultra-Low Attachment (ULA) 96-well plates (Cat. No. 6055330, PerkinElmer) or Clear Round Bottom ULA 96-well plates (Cat. No. 7007, Corning). Briefly, the plated cells were centrifuged at 1,000 RPM to promote cellular aggregation, afterwards a final concentration of 2% Matrigel (Corning) was added, and the plates were placed in a 37° C. and 5% CO₂ humidified incubator for 72 h to reach proper tumor organoid structure. Tumor organoids were then treated with CHD1Li compounds for an additional 72 h in dose response assays and analyzed for EMT reversal and cytotoxicity.

Patient Cell Tumor Organoid Culture. CRC048 and CRC102 patient cell samples were expanded and cultured as PDTOs using reported methodologies and reagents. (Drost et al., 2016; Sato et al., 2011) Briefly, patient cells were washed with PBS, digested with TrypLE, and filtered through 100 μm cell strainer before used in 3D cell culture. Patient cells were seeded at 5,000 cells per well in 96-well plates, coated with 2% Matrigel, and allowed to self-assemble as PDTOs over 72 h.

Tumor Organoid Cytotoxicity. CHD1Li cytotoxicity was assessed using CellTiter Glo 3D (Cat. No. G9681, Promega, Madison, Wis.). Tumor organoids were manually transferred from Clear Round Bottom ULA 96-well plates to Corning 96- or 384-well solid bottom white plates (Cat. No. 655083, Greiner Bio-One, Monroe, N.C.). CellTiter Glo 3D was added at a 1:1 ratio, incubated for 45 min at room temperature on an orbital shaker at 450 RPM. Finally, luminescence was quantified using an Envision Plate Reader (PerkinElmer). Cell cytotoxicity was normalized to 0.5% DMSO as vehicle control.

3D High-Content Imaging and Analysis of EMT Dual-Reporter Activity. Tumor organoids were imaged using the Opera Phenix high-content screening system (PerkinElmer) in confocal mode utilizing a 10× air objective (NA 0.3). The following excitation and emission (Ex/Em) wavelengths were employed: RFP (561/570-630) and GFP (488/500-550). Organoids were also imaged in Brightfield to segment and perform high-content analysis of dual reporter specific fluorescence intensity. RFP fluorescent signal correlates with E-Cadherin promoter activity, while GFP fluorescent signal is correlates with vimentin promoter activity. Both fluorescent signals were quantified and normalized as previously described. (Esquer, et al., 2021)

TCF-transcriptional Reporter Assay. Assay has been adapted and modified from (Esquer et al, 2021; Zhou et al., 2016; Yang et al., 2020) SW620 cells were plated into duplicate 96-well plates at 20,000 cells/well (HCT116 at 10,000 cells/well), one white solid bottom plate was used to assess TCF-transcriptional activity and one clear plate was used to measure total protein by BCA assay for normalization purpose. Cell lines were allowed to attach overnight and then were transiently transfected with TOPflash plasmid (Millipore, Billerica, Mass.) using TransIT-LT1 transfection reagent (Mirus Bio, Madison, Wis.) for 72 h. Afterwards, cells from the white solid bottom plate were carefully washed with PBS and a 1:1 ratio of PBS:One-Glo Luciferase Assay System (Promega) was added, incubated for 10 min, and luminescence was quantified on Envision plate reader (PerkinElmer) for TCF-activity. As a control, cells from the clear plate were lysed with Mammalian Cell Lysis Buffer (Promega) and the total protein from each well was quantified with BCA assay (ThermoFisher).

Clonogenic Assay. Cancer stem cell colony formation after CHD1Li treatment was assessed as previously described (Esquer et al., 2020). In brief, HCT116 cells were plated at an optimal cell concentration to avoid colonies merging over a growth period of 7-10 days. SW620 and HCT116 M-Phenotype cells were plated at 200 and 75 cells/well, respectively, into 96-well μClear CellStar black plates (Greiner Bio-One). Fresh media and drug treatments were replenished every three days. Images were acquired using the Opera Phenix HCS system and colony number, area (μm²), and confluence (μm²) were quantified and analyzed using the Harmony software. Experiments were replicated three times (n=3 for each condition).

In vivo PK studies. The PK studies were conducted using our previously published methods (Abraham et al., 2019) where CHD1Li 6.11 was administered by oral gavage (p.o.) at a dose of 50 mg/kg in a vehicle of 100% DMSO.

Statistical Analysis

All statistical analyses were performed using GraphPad Prism v9.0 (GraphPad Software Inc., La Jolla, Calif.). The data were collected using experimental replicates unless otherwise noted. All P-value significant is represented as *, p<0.05; **, p<0.01; ***, p<0.001; ****, p<0.0001.

Abbreviations

Chromodomain Helicase DNA Binding Protein 1 Like (CHD1L), also known as Amplified in Liver Cancer 1 (ALC1); T cell factor/lymphoid enhancer factor (TCF/LEF); epithelial-mesenchymal transition (EMT), mesenchymal-epithelial transition (MET); epithelial-mesenchymal plasticity (EMP); cancer stem cell (CSC); gastrointestinal (GI); colorectal cancer (CRC).

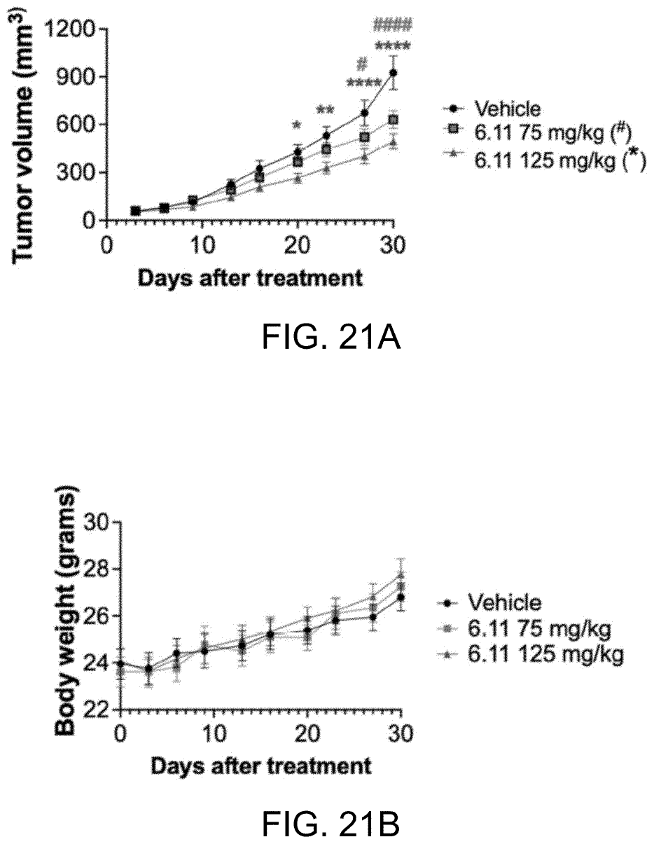

Example 18: In Vivo Anti-Tumor Activity of Compound 6.11 Administered by Oral Gavage 11-week-old athymic nude mice (35) where inoculated in the flanks (Flk) with isolated SW620 EMT dual-reporter quasi-mesenchymal cells (GFP+). (Esquer et al., 2021) Five days after cell injection, injected mice were randomized into three groups for treatment (Tx). Group 1 (12 mice) received control treatment of gavage vehicle (10% DMSO, 90% PEG 400 (polyethylene glycol 400) by volume). Group 2 (11 mice) received oral gavage of compound 6.11 at 75 mg/kg dissolved in vehicle. Group 3 (12 mice) received oral gavage of compound 6.11 at 125 mg/kg dissolved in vehicle. Mice were treated once a day, 5 days a week PO (via oral gavage). Mice were weighed and tumor volume assessed twice a week by caliper measurement as illustrated in FIGS. 21A and 21B. At sacrifice, tumors were weighed and measured. In addition, gross observations on condition of mice were made and samples of plasma, tumors, liver spleen and kidneys were collected for further assessment, e.g., drug quantification and toxicity assessment.

FIG. 21A is a graph of tumor volume (mm3) starting at 3 days after treatment was initiated. This graph shows a significant dose dependent decrease in tumor volume on oral treatment of mice with compound 6.11 over 27 days of treatment. FIG. 21B is a graph of average mouse body weight (grams) by treatment group as a function of days after treatment was initiated. This graph indicates no significant difference in body weight among the mice of the three treatment groups. Body weight loss is a general measure of treatment toxicity.

All animal studies were conducted in accordance with the animal protocol procedures approved by the Institutional Animal Care and Use Committee (IACUC) at the University of Colorado Denver Anschutz Medical Campus (Aurora, Colo.).

Example 19: Summary Table of Selected Biological Activity of CHD1L Inhibitors Table 6 provides a summary of Cat-CHD1L activity, 3D cytotocxicity data and microsomal stability data for certain CHD1L inhibitors described herein. Methods for measuring these biological activities are described in the Examples above. See also Abbott et al., 2020 and Prigaro et al., 2022 for additional detail and description of methods for assessing biological activity.

TABLE 6
Biological Activity Table

| | cat- | 3 D Cytotoxicity | | | |
|---|---|---|---|---|---|
| | CHD1L | 72 hr | | Microsomal stability | |
| | inhibition | IC₅₀ (μM) | | Human | Mouse |
| CHD1Li | IC₅₀ (μM) | SW620 | HCT116 | t_{1/2} (min) | t_{1/2} (min) |
| 6.33 | 0.3 | 4.0 | 11.6 | 38.24 | 75.98 |
| 6.41 | 0.5 | 13.5 | 21.3 | 171.32 | 62.03 |
| 6.46 | 0.7 | 12.5 | 8.3 | | |
| 6.47 | 0.8 | 13.8 | 9.3 | | |
| 6.18 | 1.0 | 1.5 | 3.0 | 88.09 | 141.06 |
| 6.21 | 1.0 | 1.7 | NA | 43.78 | 47.76 |
| 6.31 | 1.0 | 2.4 | NA | | |
| 6.59 | 1.0 | 14.8 | 11.90 | | |
| 6.44 | 1.2 | 22.7 | 14.0 | | |
| 6.5 | 1.2 | 1.2 | 2.2 | | 70.19 |
| 6.16 | 1.3 | 1.5 | 2.4 | 59.52 | 55.51 |
| 6.11 | 1.3 | 2.6 | 3.5 | 262.95 | 57.22 |
| 6.58 | 1.4 | 2.2 | 7.8 | | |
| 6.45 | 1.4 | 27.2 | 10.6 | | |
| 6.35 | 1.8 | 8.6 | NA | | |
| 6.38 | 2.1 | 6.1 | 7.6 | 199.01 | 96.62 |
| 6.49 | 2.3 | 17.0 | 14.7 | | |
| 6.57 | 2.4 | 14.6 | 10.8 | | |
| 6.56 | 2.5 | >40 | >40 | | |
| 6.34 | 3.0 | >20 | NA | | |
| 6.54 | 3.5 | >40 | >40 | | |
| 6.48 | 4.5 | 21.8 | 7.6 | | |
| 6.51 | 6.0 | 40.7 | 15.1 | | |
| 6.43 | 10.3 | 0.0 | 19.1 | | |
| 6.40 | 11.4 | 10.5 | 4.8 | | |
| 6.42 | 17.7 | 0.0 | 20.9 | | |
| 6.55 | 18.7 | >40 | >40 | | |
| 6.39 | >20 | 14.2 | 10.8 | 88.64 | 70.48 |
| 6.36 | >20 | >20 | NA | | |
| 6.50 | >20 | >40 | 26.2 | | |
| 6.52 | >20 | >20 | >20 | | |
| 6.53 | >20 | >40 | >40 | | |
| 6.20 | NA | 2.2 | NA | | |
| 6.24 | NA | 3.0 | NA | | |
| 6.27 | NA | 3.0 | NA | | |
| 6.3 | NA | 3.6 | 3.7 | | |
| 6.8 | NA | 4.3 | 6.8 | | |
| 6.26 | NA | 4.7 | NA | | |
| 6.29 | NA | 5.4 | NA | | |
| 6.17 | NA | 5.5 | 5.6 | | |
| 6.30 | NA | 6.1 | NA | | |
| 6.19 | NA | 7.7 | >40 | | |
| 6.9 | NA | 8.3 | 19.7 | | |
| 6.32 | NA | 5.0 | 5.0 | | |
| 6.14 | NA | 11.1 | 15.3 | | |
| 6.22 | NA | 11.3 | NA | | |
| 6.7 | NA | 13.0 | 17.6 | | |
| 6.15 | NA | 13.1 | 19.7 | | |
| 6.25 | NA | 17.6 | NA | | |
| 6.10 | NA | 24.1 | >40 | | |
| 6.12 | NA | >20 | >40 | | |
| 6.13 | NA | >20 | >40 | | |
| 6.1 | NA | >40 | >40 | | |
| 6.2 | NA | >40 | NA | | |
| 6.4 | NA | >40 | >40 | | |
| 6.6 | NA | >40 | >40 | | |

Compounds 57 (6.5), 52 (6.11), 54 (6.16), 28 (6.18), 31 (6.21), 75 (6.31), 118 (6.33), 120 (6.35), 123 (6.38) and 150 (6.58) exhibit good enzyme inhibition and below 10 uM IC50 of cytotoxicity in SW620 cells. Any one of compounds 57, 52, 54, 28, 31, 75, 118, 120, 123 and 150 is particularly useful in the methods of treatment, combination therapies, pharmaceutical compositions and pharmaceutical combinations of this invention.

### REFERENCES

1. Lorch Y, Maier-Davis B, Kornberg R D. Mechanism of chromatin remodeling. Proc Natl Acad Sci USA 2010; 107(8):3458-62.
2. Kumar R, Li D Q, Muller S, Knapp S. Epigenomic regulation of oncogenesis by chromatin remodeling. Oncogene 2016; 35(34):4423-36.
3. Swygert S G, Peterson C L. Chromatin dynamics: interplay between remodeling enzymes and histone modifications. Biochim Biophys Acta 2014; 1839(8):728-36 doi 10.1016/j.bbagrm.2014.02.013.
4. Erdel F, Krug J, Langst G, Rippe K. Targeting chromatin remodelers: signals and search mechanisms. Biochim Biophys Acta 2011; 1809(9):497-508.
5. Brownlee P M, Meisenberg C, Downs J A. The SWI/SNF chromatin remodelling complex: Its role in maintaining genome stability and preventing tumourigenesis.

DNA Repair (Amst) 2015; 32:127-33.
6. Zhang C, Lu J, Zhang P. The Roles of chromatin remodeling proteins in cancer. Curr Protein Pept Sci 2016; 17(5):446-54.
7. Valencia A M, Kadoch C. Chromatin regulatory mechanisms and therapeutic opportunities in cancer. Nat Cell Biol 2019; 21(2):152-61.
8. Ma N F, Hu L, Fung J M, Xie D, Zheng B J, Chen L, et al. Isolation and characterization of a novel oncogene, amplified in liver cancer 1, within a commonly amplified region at 1q21 in hepatocellular carcinoma. Hepatology 2008; 47(2):503-10.
9. Cheng W, Su Y, Xu F. CHD1L: a novel oncogene. Molecular Cancer 2013; 12(1):170.
10. Li Y, He L R, Gao Y, Zhou N N, Liu Y, Zhou X K, et al. CHD1L contributes to cisplatin resistance by upregulating the ABCB1-NF-kappaB axis in human non-small-cell lung cancer. Cell Death Dis 2019; 10(2):99.
11. Pines A, Vrouwe M G, Marteijn J A, Typas D, Luijsterburg M S, Cansoy M, et al. PARP1 promotes nucleotide excision repair through DDB2 stabilization and recruitment of ALC1. J Cell Biol 2012; 199(2):235-49.
12. Tsuda M, Cho K, Ooka M, Shimizu N, Watanabe R, Yasui 849 A, et al. ALC1/CHD1L, a chromatin-remodeling enzyme, is required for efficient base excision repair. PLoS One 2017; 12(11):e0188320.
13. Li Y, Chen L, Chan T H, Liu M, Kong K L, Qiu J L, et al. SPOCK1 is regulated by CHD1L and blocks apoptosis and promotes HCC cell invasiveness and metastasis in mice. Gastroenterology 2013; 144(1):179-91.
14. Chen L, Hu L, Chan T H, Tsao G S, Xie D, Huo K K, et al. Chromodomain helicase/adenosine triphosphatase DNA binding protein 1-like (CHD11) gene suppresses the nucleus-to-mitochondria translocation of nur77 to sustain hepatocellular carcinoma cell survival. Hepatology 2009; 50(1):122-9.
15. He L R, Ma N F, Chen J W, Li B K, Guan X Y, Liu M Z, et al. Overexpression of CHD1L is positively associated with metastasis of lung adenocarcinoma and predicts patients' poor survival. Oncotarget 2015; 6(31):31181-90.
16. Hyeon J, Ahn S, Park C K. CHD1L Is a marker for poor prognosis of hepatocellular carcinoma after surgical resection. Korean J Pathol 2013; 47(1):9-15.
17. Su Z, Zhao J, Xian G, Geng W, Rong Z, Wu Y, et al. CHD1L is a novel independent prognostic factor for gastric cancer. Clin Transl Oncol: 2014; 16(8):702-7 (Federation of Spanish Oncology Societies and of the National Cancer Institute of Mexico).
18. Wu J, Zong Y, Fei X, Chen X, Huang O, He J, et al. Presence of CHD1L over expression is associated with aggressive tumor biology and is a novel prognostic biomarker for patient survival in human breast cancer. PLoS One 2014; 9(8):e98673.
19. Kinzler K W, Vogelstein B. Lessons from hereditary colorectal cancer. Cell 1996; 87(2):159-70.
20. Cancer Genome Atlas Network. Comprehensive molecular characterization of human colon and rectal cancer. Nature 2012; 487(7407):330-7.
21 Shitashige M, Hirohashi S, Yamada T. Wnt signaling inside the nucleus. Cancer Sci 2008; 99(4):631-7.
22. Senchez-Till6 E, de Barrios O, Siles L, Cuatrecasas M, Castells A, Postigo A. β-catenin/TCF4 complex induces the epithelial-to-mesenchymal transition (EMT)-activator ZEB1 to regulate tumor invasiveness. Proc Natl Acad Sci USA 2011; 108(48):19204-9.
23. Zhou Q, Abraham A D, Li L, Babalmorad A, Bagby, Arcaroli J J, et al. Topoisomerase Ila mediates TCF-dependent epithelial-mesenchymal transition in colon cancer. Oncogene 2016:35(38):4990-9.
24. Abraham A D, Esquer H, Zhou Q, Tomlinson N, Hamill B D, Abbott J M, et al. Drug design targeting t-cell factor-driven epithelial-mesenchymal transition as a therapeutic strategy for colorectal cancer. J Med Chem 2019; 62(22):10182-203.
25. Chaffer C L, San Juan B P, Lim E, Weinberg R A. EMT, cell plasticity and metastasis. Cancer Metast Rev 2016; 35(4):645-54.
26. Lu B, Green B A, Farr J M, Lopes F C, Van Raay T J. Wnt drug discovery: weaving through the screens, patents and clinical trials. Cancers 2016; 8 (9).
27. Marisa L, de Reynies A, Duval A, Selves J, Gaub M P, Vescovo L, et al. Gene expression classification of colon cancer into molecular subtypes: characterization, validation, and prognostic value. PLoS Med 2013; 10 (5):e1001453.
28. Scott A J, Song E K, Bagby S, Purkey A, McCarter M, Gajdos C, et al. Evaluation of the efficacy of dasatinib, a Src/Abl inhibitor, in colorectal cancer cell lines and explant mouse model. PLoS One 2017; 12(11):e0187173.
29. Tentler J J, Tan A C, Weekes C D, Jimeno A, Leong S, Pitts™, et al. Patient derived tumour xenografts as models for oncology drug development. Nat Rev Clin Oncol 2012; 9(6):338-50.
30. Vlachogiannis G, Hedayat S, Vatsiou A, Jamin Y, Fernandez-Mateos J, Khan K, et al. Patient-derived organoids model treatment response of metastatic gastrointestinal cancers. Science 2018; 359(6378):920-6.
31. Drost J, Clevers H. Organoids in cancer research. Nat Rev Cancer 2018; 18; 407-18.
32. Ahel D, Horejsi Z, Wiechens N, Polo S E, Garcia-Wilson E, Ahel I, et al. Poly(ADP-ribose)-dependent regulation of DNA repair by the chromatin remodeling enzyme ALC1. Science 2009; 325(5945):1240-3.
33. Morin P J, Sparks A B, Korinek V, Barker N, Clevers H, Vogelstein B, et al. Activation of beta-catenin-Tcf signaling in colon cancer by mutations in beta catenin or APC. Science 1997; 275(5307):1787-90.
34. Idogawa M, Yamada T, Honda K, Sato S, Imai K, Hirohashi S. Poly(ADP-Ribose) polymerase-1 is a component of the oncogenic T-cell factor-4/β-catenin complex. Gastroenterology 924 2005; 128(7):1919-36.
35. Franken N A, Rodermond H M, Stap J, Haveman J, van Bree C. Clonogenic assay of cells in vitro. Nat Protoc 2006; 1(5):2315-9.
36. Ryan D P, Owen-Hughes T. Snf2-family proteins: chromatin remodellers for any occasion. Curr Opin Chem Biol 2011; 15(5):649-56.
37. Flaus A, Owen-Hughes T. Mechanisms for ATP-dependent chromatin remodelling: the means to the end. FEBS J 2011; 278(19):3579-95.
38. Lehmann L C, Hewitt G, Aibara S, Leitner A, Marklund E, Maslen S L, et al. Mechanistic Insights into Autoinhibition of the Oncogenic Chromatin Remodeler ALC1. Mol Cell 2017; 68(5):847-59.
39. Gottschalk A J, Timinszky G, Kong S E, Jin J, Cai Y, Swanson S K, et al. Poly(ADP-ribosyl)ation directs recruitment and activation of an ATP-dependent chromatin remodeler. Proc Natl Acad Sci USA 2009; 106(33):13770-4.
40. McDonald P C, Dedhar S. The role of epithelial-mesenchymal transition in cancer metastasis. In: Regad T, Sayers T J, Rees R C, editors. Principles of stem cell biology and cancer future applications and therapeutics. 1st ed. West Sussex, U K: John Wiley & Sons, Ltd; 2015. p.101-21.
41. Yun J A, Kim S H, Hong H K, Yun S H, Kim H C, Chun H K, et al. Loss of E-cadherin expression is associated with a poor prognosis in stage iii colorectal cancer. Oncology 2014; 86 (5-6):318-28.
42. Richardson F, Young G D, Sennello R, Wolf J, Argast G M, Mercado P, et al. The evaluation of E-Cadherin and vimentin as biomarkers of clinical outcomes among patients with non-small cell lung cancer treated with erlotinib as second- or third-line therapy. Anticancer Res 2012; 32(2):537-52.
43. Dhanasekaran S M, Barrette T R, Ghosh D, Shah R, Varambally S, Kurachi K, et al. Delineation of prognostic biomarkers in prostate cancer. Nature 2001; 412(6849):822-6.
44. Kashiwagi S, Yashiro M, Takashima T, Nomura S, Noda S, Kawajiri H, et al. Significance of E-cadherin expression in triple-negative breast cancer. Br J Cancer 2010; 103(2):249-55.
45. Toiyama Y, Yasuda H, Saigusa S, Tanaka K, Inoue Y, Goel A, et al. Increased expression of Slug and Vimentin as novel predictive biomarkers for lymph node metastasis and poor prognosis in colorectal 960 cancer. Carcinogenesis 2013; 34(11):2548-57.
46. Zeisberg M, Neilson E G. Biomarkers for epithelial-mesenchymal transitions. J Clin Invest 2009; 119(6):1429-37.
47. Sun J, Zhang L, Zhao H, Qiu X, Chen W, Wang D, et al. CHD1L regulates cell cycle, apoptosis, and migration in glioma. Cell Mol Neurobiol 2016; 36(4):565-767.
48. Lu M, Marsters S, Ye X, Luis E, Gonzalez L, Ashkenazi A. E-cadherin couples death receptors to the cytoskeleton to regulate apoptosis. Mol Cell 2014; 54(6):987-98.
49. Steinhusen U, Weiske J, Badock V, Tauber R, Bommert K, Huber O. Cleavage and shedding of E-cadherin after induction of apoptosis. J Biol Chem 2001; 276(7):4972-80.
50. Daina A, Michielin O, Zoete V. SwissADME: a free web tool to evaluate pharmacokinetics, drug-likeness and medicinal chemistry friendliness of small molecules. Sci Rep 2017; 7(1):1-13.
51. Mu Q. J. et al. Chromodomain Helicase/ATPase DNA-Binding Protein 1-Like Gene (CHD1L) Expression and Implications for Invasion and Metastasis of Breast Cancer. PLoS One 2015; 10(11), e0143030.
52. Su F R., Ding J H, Bo L, Liu X G. Chromodomain helicase/ATPase DNA binding protein 1-like protein expression predicts poor prognosis in nasopharyngeal carcinoma. Exp Ther Med 2014; 8, 1745-1750.
53. He, W P et al. CHD1L protein is overexpressed in human ovarian carcinomas and is a novel predictive biomarker for patients survival. BMC Cancer 2012; 12: 437 (8 pages).
54. Chen L. et al. CHD1L promotes hepatocellular carcinoma progression and metastasis in mice and is associated with these processes in human patients. J Clin Invest 2010; 120: 1178-1191.
55. Ji X. et al. CHD1L promotes tumor progression and predicts survival in colorectal carcinoma. J. Surg. Res. 2013; 185: 84-91.
56. Clevers H. Wnt/β-catenin signaling in development and disease. Cell 2006; 127: 469-480.
57. Korinek V. et al. Constitutive transcriptional activation by a β-catenin-TCF complex in APC-/-colon carcinoma. Science 1997; 275:1784-1787.
58. Hu Z. et al. Quantitative evidence for early metastatic seeding in colorectal cancer. Nat Genet 2019; 51:1113-1122.
59. Chaffer C L, Weinberg R A. A perspective on cancer cell metastasis. Science 2011; 33: 1559-1564.
60. Jemal A. et al. Global cancer statistics. C A Cancer J. Clin. 2011; 61: 69-90.
61. Fakih M G. Metastatic colorectal cancer: current state and future directions. J Clin Oncol 2015; 33:1809-1824.
62. Lee H J, Zhang X X, Zheng J J in Targeting the Wnt Pathway in Cancer (K H Goss, M Kahn, eds.) 2011; Ch. 9: 183-209 (Springer New York).
63. Polakis, P. Drugging Wnt signalling in cancer. EMBO J 2012; 31, 2737-2746.
64. Li L. et al. An improved high yield total synthesis and cytotoxicity study of the marine alkaloid neoamphimedine: an ATP-competitive inhibitor of topoisomerase II alpha and potent anticancer agent. Mar Drugs 2014a; 12: 4833-4850.
65. Li L. et al. Design of an amide N-glycoside derivative of beta-glucogallin: a stable, potent, and specific inhibitor of aldose reductase. J Med Chem 2014b; 57: 71-77.
66. LaBarbera D V, Bugni T S, Ireland C M. The total synthesis of neoamphimedine. J Org Chem 2007; 72: 8501-8505.
67. Kumar D et al. Synthesis, antimalarial activity, heme binding and docking studies of 4-aminoquinoline-pyrimidine based molecular hybrids. RSC Adv 2014; 4: 63655-63669.
68. Odingo J et al. Synthesis and evaluation of the 2,4-diaminoquinazoline series as anti-tubercular agents. Bioorg Med Chem 2014; 22, 6965-6979.
69. Scheel C, Weinberg R A. Cancer stem cells and epithelial-mesenchymal transition: Concepts and molecular links. Semin Cancer Biol. 2012; 2: 396-403.
70. Baell J B, Nissink J W. Seven year itch: pan-assay interference compounds (pains) in 2017- utility and limitations. ACS Chem Biol 2018; 13: 36-44.
71. Baell J B, Holloway G A. New Substructure filters for removal of pan assay interference compounds (pains) from screening libraries and for their exclusion in bioassays. J Med Chem 2010; 53 (7): 2719-2740.
72. Abbott J M et al., First-in-Class Inhibitors of Oncogenic CHD1L with Preclinical Activity against Colorectal Cancer, Mol Cancer Therapeutics. Jun. 2020, 19:1598-1612. Supplemental Data available at the web site https://aacrjournals.org/mct/article/19/8/1598/92871/First-in-Class-Inhibitors-of-Oncogenic-CHD1L-with
73. Di Veroli G Y et al. Combenefit: an interactive platform for the analysis and visualization of drug combinations Bioinformatics, 32(18), 2016, 2866.
74. Ivashkevich, A. et al. Use of the gamma-H2A X assay to monitor DNA damage and repair in translational cancer research. Cancer Lett, 2012, 327:123-133.
75. Ji, J. et al. Phosphorylated fraction of H2A X as a measurement for DNA damage in cancer cells and potential applications of a novel assay. PLoS One 2017, 12, e0171582.
76. Zhou et al. Polymerase Theta Inhibition Kills Homologous Recombination Deficient Tumors, May 2020 preprint DOI: 10.1101/2020.05.23.111658 available from the website bioRxiv.
77. Wahlberg E. et al. Family-wide chemical profiling and structural analysis of PARP and tankyrase inhibitors, Nature Biotech, 2012, 30(3):283-289.
78. D'Andrea A D, Mechanisms of PARP inhibitor sensitivity and resistance, DNA Repair, 2018, 71:172-176.
79. Rouleau M et al. PARP inhibition: PARP1 and beyond. Nature Review, 2010, 10:293-301.
80. Yi M et al. Advances and perspectives of PARP inhibitors. Ex Hematol Oncol, 2019, 8:29 (12 pages).
81. Hevener K E et al. Recent developments in topoisomerase-targeted cancer chemotherapy, Acta Pharma Sin B, 2018, 8(6):844-861.
82. Lipinski, C A et al. Experimental and computational approaches to estimate solubility and permeability in drug discovery and development settings, Adv. Drug Del. Rev., 2001, 46, 3-26.
83. Ghose, A K et al., A Knowledge-Based Approach in Designing Combinatorial or a Chemistry Libraries for Drug Discovery, J. Combin. Chem., 1999, 1, 55-68.
84. Wutts, P. G. and Greene, T. Green's Protecting Groups in Organic Synthesis (Fourth Edition), 2007, John Wiley & Sons, N.Y.
85. Jacques, et al., Enantiomers, Racemates and Resolutions. Wiley Interscience, New York, 1981.
86. Wilen, S. H., et al., Strategies in optical resolutions. Tetrahedron, 1977, 33:2725.
87. Eliel, E. L. Stereochemistry of Carbon Compounds. McGraw-Hill, N.Y., 1962.
88. Wilen, S. H. Tables of Resolving Agents and Optical Resolutions. In E. L. Eliel, Ed., Univ. of Notre Dame Press, Notre Dame, Ind., 1972, p. 268.
89. Wheate, N.J., et al. The status of platinum anticancer drugs in the clinic and in clinical trials. Dalton Transactions, 2010, 39(35):8097-8340.
90. Papamichael, D. The Use of Thymidylate Synthase Inhibitors in the Treatment of Advanced Colorectal Cancer: Current Status, 2009, Stem Cells, 18(3):166-175.
91. Lehman, N. Future potential of thymidylate synthase inhibitors in cancer therapy, 2002, Expert Opin Investig Drugs, 11(12):1775-1787.
92. Esquer, H. et al. Isolating and targeting the real-time plasticity and malignant properties of epithelial-mesenchymal transition in cancer, Oncogene, Mar. 19, 2021, doi.org/10.1038/s41388-021-01728-2 and Supplementary Information available from the publisher at doi.org/10.1038/s41388-021-01728-2.
93. Xiong, X.; Lai, X.; Li, A.; Liu, Z.; Ma, N. Diversity roles of CHD1L in normal cell function and tumorigenesis. Biomark Res 2021, 9, 16.
94. Verma, P.; Zhou, Y.; Cao, Z.; Deraska, P. V.; Deb, M.; Arai, E.; Li, W.; Shao, Y.; Puentes, L.; Li, Y.; Patankar, S.; Mach, R. H.; Faryabi, R. B.; Shi, J.; Greenberg, R. A. ALC1 links chromatin accessibility to PARP inhibitor response in homologous recombination-deficient cells. Nat Cell Biol 2021, 23, 160-171.
95. Juhasz, S.; Smith, R.; Schauer, T.; Spekhardt, D.; Mamar, H.; Zentout, S.; Chapuis, C.; Huet, S.; Timinszky, G. The chromatin remodeler ALC1 underlies resistance to PARP inhibitor treatment. Sci Adv 2020, 6.
96. van de Wetering, M.; Sancho, E.; Verweij, C.; de Lau, W.; Oving, I.; Hurlstone, A.; van der Horn, K.; Batlle, E.; Coudreuse, D.; Haramis, A. P.; Tjon-Pon-Fong, M.; Moerer, P.; van den Born, M.; Soete, G.; Pals, S.; Eilers, M.; Medema, R.; Clevers, H. The β-catenin/TCF-4 complex imposes a crypt progenitor phenotype on colorectal cancer cells. Cell 2002, 111, 241-250.
97. Ram Makena, M.; Gatla, H.; Verlekar, D.; Sukhavasi, S.; M, K. P.; K, C. P. Wnt/beta-Catenin Signaling: The Culprit in Pancreatic Carcinogenesis and Therapeutic Resistance. Int J Mol Sci 2019, 20.
98. Batlle, E.; Henderson, J. T.; Beghtel, H.; van den Born, M. M.; Sancho, E.; Huls, G.; Meeldijk, J.; Robertson, J.; van de Wetering, M.; Pawson, T.; Clevers, H. β-catenin and TCF mediate cell positioning in the intestinal epithelium by controlling the expression of EphB/EphrinB. Cell 2002, 111, 251-263.
99. He, S.; Tang, S. WNT/beta-catenin signaling in the development of liver cancers. Biomed. Pharmacother. 2020, 132, 110851.
100. Polakis, P. Wnt signaling in cancer. Cold Spring Harb Perspect Biol 2012, 4.
101. Yang, J.; Antin, P.; Berx, G.; Blanpain, C.; Brabletz, T.; Bronner, M.; Campbell, K.; Cano, A.; Casanova, J.; Christofori, G.; Dedhar, S.; Derynck, R.; Ford, H. L.; Fuxe, J.; Garcia de Herreros, A.; Goodall, G. J.; Hadjantonakis, A. K.; Huang, R. J. Y.; Kalcheim, C.; Kalluri, R.; Kang, Y.; Khew-Goodall, Y.; Levine, H.; Liu, J.; Longmore, G. D.; Mani, S. A.; Massague, J.; Mayor, R.; McClay, D.; Mostov, K. E.; Newgreen, D. F.; Nieto, M. A.; Puisieux, A.; Runyan, R.; Savagner, P.; Stanger, B.; Stemmler, M. P.; Takahashi, Y.; Takeichi, M.; Theveneau, E.; Thiery, J. P.; Thompson, E. W.; Weinberg, R. A.; Williams, E. D.; Xing, J.; Zhou, B. P.; Sheng, G.; Association, E. M. T. I. Guidelines and definitions for research on epithelial-mesenchymal transition. Nat Rev Mol Cell Biol 2020, 21, 341-352.
102. Kroger, C.; Afeyan, A.; Mraz, J.; Eaton, E. N.; Reinhardt, F.; Khodor, Y. L.; Thiru, P.; Bierie, B.; Ye, X.; Burge, C. B.; Weinberg, R. A. Acquisition of a hybrid E/M state is essential for tumorigenicity of basal breast cancer cells. Proc Natl Acad Sci USA 2019, 116, 7353-7362.
103. Esquer, H.; Zhou, Q.; Abraham, A. D.; LaBarbera, D. V. Advanced High-Content-Screening Applications of Clonogenicity in Cancer. SLAS Discov 2020, 25, 734-743.
104. Gramec, D.; Peterlin Masic, L.; Sollner Dolenc, M. Bioactivation potential of thiophene-containing drugs. Chem. Res. Toxicol. 2014, 27, 1344-58.
105. Drost, J.; Karthaus, W. R.; Gao, D.; Driehuis, E.; Sawyers, C. L.; Chen, Y.; Clevers, H. Organoid culture systems for prostate epithelial and cancer tissue. Nat Protoc 2016, 11, 347-58.
106. Sato, T.; Stange, D. E.; Ferrante, M.; Vries, R. G.; Van Es, J. H.; Van den Brink, S.; Van Houdt, W. J.; Pronk, A.; Van Gorp, J.; Siersema, P. D.; Clevers, H. Long-term expansion of epithelial organoids from human colon, adenoma, adenocarcinoma, and Barrett's epithelium. Gastroenterology 2011, 141, 1762-72.
107. Bailly, C. (2012) “Contemporary Challenges in the Design of Topoisomerase II Inhibitors for Cancer Chemotherapy,” Chem. Rev. 112:3611-3640.
108. Nitiss, J. (2009) “Targeting DNA topoisomerase II in cancer chemotherapy,” Nature Rev. Cancer, 9:338-350.
109. Prigaro, B. J.; Esquer, H.; Zhou, Q.; Pike, L. A.; Awolade, P.; Lai, X-H.; Abraham, A. D.; Abbott, J. M.; Matter, B.; Kompella, U. B.; Messersmith, W. A.; Gustafson, D. L.; LaBarbaera, D. V. (2022) “Design, Synthesis, and Biological Evaluation of the First Inhibitors of Oncogenic CHD1L,” J. Med. Chem. 65, 3943-3961. Supporting Information available at the web site pubs.acs.org/doi/10.1021/acs.jmedchem.1 c01778.

We claim:

1. A CHD1L inhibitor of formula I: 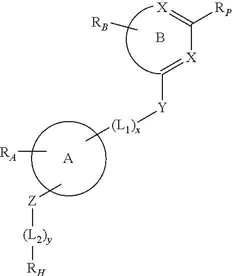
or salts, or solvates thereof,
where:
the B ring is an optionally-substituted at least divalent heteroaryl ring or ring system having one, two or three 5- or 6-member rings, any two or three of which can be fused rings, where the rings are carbocyclic, heterocyclic, aryl or heteroaryl rings and at least one of the rings is heteroaryl;
in the B ring, each X is independently selected from N or CH and at least one X is N;
R_{P} is an optionally-substituted primary or secondary amine group [—N(R₂)(R₃)] or is a -(M)_{X}-P group, where P is —N(R₂)(R₃) or an aryl or heteroaryl group, where x is 0 or 1 to indicate the absence or present of M and M is an optionally substituted linker —(CH₂)ₙ— or —N(R)(CH₂)ₙ—, where each n is independently an integer from 1-6 (inclusive);
Y is a divalent atom or group selected from the group consisting of —O—, —S—, —N(R₁)—, —CON(R₁)—, —N(R₁)CO—, —N(R₁)CON(R₁)—, —SO₂N(R₁)—, or —N(R₁)SO₂—;
L₁ is an optional 1-4 carbon linker that is optionally substituted and is saturated or contains a double bond (which can be cis or trans), where x is 0 or 1 to indicate the absence or presence of L₁;
the A ring is an optionally-substituted at least divalent carbocyclic or heterocyclic ring or ring system having one, two or three rings, two or three of which can be fused, each ring having 3-10 carbon atoms and optionally 1-6 heteroatoms and wherein each ring is optionally saturated, unsaturated or aromatic;
Z is a divalent group containing at least one nitrogen substituted with a R′ group, where in embodiments, Z is a divalent group selected from —N(R′)—, —CON(R′)—, —N(R′)CO—, —CSN(R′)—, —N(R′)CS—, —N(R′)CON(R′)—, —SO₂N(R′)—, —N(R′)SO₂—, —CH(CF₃)N(R′)—, —N(R′)CH(CF₃)—, —N(R′)CH₂CON(R′)CH₂—, —N(R′)COCH₂N(R′)CH₂—, 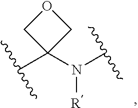
or the divalent Z group comprises a 5- or 6-member heterocyclic ring having at least one nitrogen ring member, for example, 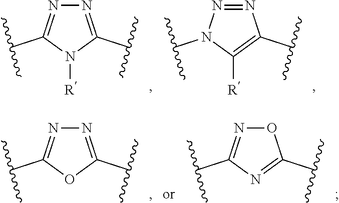
L2 is an optional 1-4 carbon linker that is optionally substituted and is saturated or contains a double bond (which can be cis or trans), where z is 0 or 1 to indicate the absence or presence of L2;
R is selected from the group consisting of hydrogen, an aliphatic group, a carbocyclyl group, an aryl group, a heterocyclyl group and a heteroaryl group, each of which groups is optionally substituted;
each R′ is independently selected from the group consisting of hydrogen, an aliphatic group, a carbocyclyl group, an aryl group, a heterocyclyl group and a heteroaryl group, each of which groups is optionally substituted;
R₁ is selected from the group consisting of hydrogen, an aliphatic group, a carbocyclyl group, an aryl group, a heterocyclyl group and a heteroaryl group, each of which groups is optionally substituted;
R₂ and R₃ are independently selected from the group consisting of hydrogen, an aliphatic group, a carbocyclyl group, an aryl group, a heterocyclyl group and a heteroaryl group, each of which groups is optionally substituted or
R₂ and R₃ together with the N to which they are attached form an optionally substituted 5- to 10-member heterocyclic ring which is a saturated, partially unsaturated or aromatic ring;
R_{A} and R_{B} represent hydrogens or 1-10 non-hydrogen substituents on the indicated A and B ring or ring systems, respectively, wherein R_{A} and R_{B} substituents are independently selected from hydrogen, halogen, hydroxyl, cyano, nitro, amino, mono- or disubstituted amino (—NR_{C}R_{D}), alkyl, alkenyl, cycloalkyl, cycloalkenyl, aryl, heterocyclyl, alkoxy, acyl, haloalkyl, —COOR_{C}, —OCOR_{C}, —CONR_{C}R_{D}, —OCONR_{C}R_{D}, —NR_{C}COR_{D}, —SR_{C}, —SOR_{C}, —SO₂R_{C}, and —SO₂NR_{C}R_{D}, where alkyl, alkenyl, cycloalkyl, cycloalkenyl, aryl, heterocyclyl, alkoxy, and acyl, are optionally substituted;
each R_{C} and R_{D} is selected from hydrogen, alkyl, alkenyl, cycloalkyl, cycloalkenyl, heterocyclyl, aryl, or heteroaryl, each of which groups is optionally substituted with one or more halogen, alkyl, alkenyl, haloalkyl, alkoxy, aryl, heteroaryl, heterocyclyl, aryl-substituted alkyl, or heterocyclyl-substituted alkyl; and
R_{H} is an optionally substituted aryl or heteroaryl group;
wherein optional substitution includes, substitution with one or more halogen, nitro, cyano, amino, mono- or di-C1-C3 alkyl substituted amino, C1-C3 alkyl, C2-C4 alkenyl, C3-C6 cycloalkyl, C3-C6-cycloalkenyl, C1-C3 haloalkyl, C1-C6 acyl. C1-C6 acyloxy, C1-C6 alkoxylcarbonyl. C6-C12 aryl, C5-C12 heteroaryl, C3-C12 heterocyclyl. C1-C3 alkoxy, C1-C6 acyl, —COOR_{E}, —OCOR_{E}, —CONR_{E}R_{F}, —OCONR_{E}RD, —NR_{E}COR_{F}, —SR_{E}, —SOR_{E}, —SO₂R_{E}, and —SO₂NR_{E}R_{F}, where alkyl, alkenyl, cycloalkyl, cycloalkenyl, aryl, heterocyclyl, alkoxy, and acyl, are optionally substituted and
each R_{E} and R_{F} is selected from hydrogen, C1-C3 alkyl, C2-C4 alkenyl, C3-C6 cycloalkyl, C3-C6-cycloalkenyl, C1-C3 haloalkyl, C6-C12 aryl, C5-C12 heteroaryl, C3-C12 heterocyclyl. C1-C3 alkoxy, C1-C6 acyl, each of which groups is optionally substituted with one or more halogen, nitro, cyano, amino, mono- or di-C1-C3 alkyl substituted amino, C1-C3 alkyl, C2-C4 alkenyl, C3-C6 cycloalkyl, C3-C6-cycloalkenyl, C1-C3 haloalkyl, C6-C12 aryl, C5-C12 heteroaryl, C3-C12 heterocyclyl. C1-C3 alkoxy, C1-C6 acyloxy, C1-C6 alkoxycarbonyl and C1-C6 acyl;
with the exception that the compound is not one of compounds 1-9.

2. The compound of claim 1, wherein the A ring is unsubstituted 1,4-phenylene or unsubstituted 2,5-pyridylene.

3. The compound of claim 1, wherein R_{P} is selected from one of the moieties R_{N}1-R_{N}-39.

4. The compound of claim 1, wherein R_{H} is selected from one of the moieties R12-1-R12-84.

5. The compound of claim 1, wherein Y is a group selected from the group consisting of —NH—, —CONH—, —NHCO—, or —NHCONH—; x is 0 or 1 and L₁, if present, is —CH₂—, —CH₂—CH₂— or CH₂—CH₂—CH₂—.

6. The compound of claim 1, wherein Z is a groups selected from the group consisting of —NH—, —CONH—, —NHCO—, or —NHCONH—; y is 0 or 1 and L2, if present, is —CH₂—, —CH₂—CH₂— or CH₂—CH₂—CH₂—.

7. The compound of claim 1 of formula XLVI: 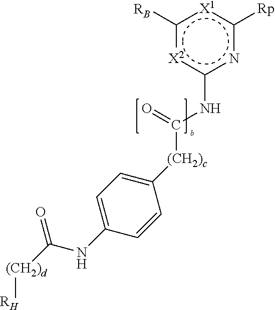
or salts or solvates thereof;
wherein:
X¹ and X² are independently CH or N;
R_{B} is hydrogen, C1-C3 alkyl or C1-C3 fluoroalkyl; and
b, c or d are zero or integers, where b is 0 or 1, c is 0 or 1, and d is 0 or 1;
R_{P} is selected from one of the moieties R_{N}1-R_{N}-39; and
R_{H} is selected from one of the moieties R12-1-R12-84.

8. The compound of claim 7, wherein Re is selected from R_{N}1, R_{N}3, R_{N}9, R_{N}11, R_{N}25, R_{N}26-R_{N}31, R_{N}33-R_{N}34; R_{N}37, R_{N}38, or R_{N}39.

9. The compound of claim 7, wherein R_{H} is selected from R12-5; R12-44; R12-45; R12-58; R12-62; R12-75, R12-79; or R12-80.

10. The compound of claim 7 wherein R_{P} is selected from R_{N}1, R_{N}3, R_{N}9, R_{N}11, R_{N}25, R_{N}26-R_{N}31, R_{N}33-R_{N}34; R_{N}37, R_{N}38, or R_{N}39 and R_{H} is selected from R12-5; R12-44; R12-45; R12-58; R12-62; R12-75, R12-79; or R12-80.

11. The compound of claim 7 of formula: 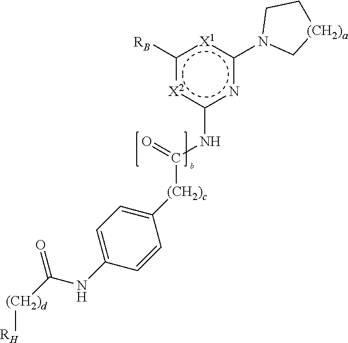
or salts or solvates thereof, wherein a is an integer which is 1 or 2.

12. The compound of claim 11, wherein a is 1.

13. The compound of claim 11, wherein R_{H} is selected from R12-5; R12-44; R12-45; R12-58; R12-62; R12-75, R12-79; or R12-80.

14. A compound of claim 1, selected from compounds: 1-177.

15. A compound of claim 1, which is a compound selected from compounds 28, 31, 52, 54, 57, 75, 118, 126, 131, 150 or 169

16. A compound of claim 1 which is compound 52.

17. A pharmaceutical composition comprising a compound, salt or solvate of claim 1 and a pharmaceutically acceptable excipient.

18. A pharmaceutical composition comprising a compound, salt or solvate of claim 11.

19. A pharmaceutical combination comprising a compound salt or solvate of claim 1 in combination with an alternative antineoplastic agent or cyctotoxicity agent.

20. A method for treatment of CHD1L-driven cancers which comprises administration to a patient in need thereof of a CHD1L inhibitor of claim 1 or a pharmaceutical composition comprising the CHD1L inhibitor administered is effective for CHD1L inhibition.

21. A method of preventing tumor growth, invasion and/or metastasis in CHD1L-driven, EMT-driven or TCF-transcription driven cancers by administering to a patient in need thereof an amount of a CHD1L inhibitor of claim 1 which is effective for CHD1L inhibition or inhibition of aberrant TCF transcription.

22. A combination method for treatment of cancer which comprises administration of a CHD1L inhibitor of claim 1 in combination with a PARP inhibitor, a topoisomerase inhibitor, a platinum-based antineoplastic agent or a thymidylate synthase inhibitor wherein the CHD1L and the PARP inhibitor, the topoisomerase inhibitor, the platinum-based antineoplastic agent or the thymidylate synthase inhibitor are present in the combination in a combined therapeutically effect amount.
